(19) United States
(12) Patent Application Publication
(10) Pub. No.: US 2021/0278408 A1
(43) Pub. Date: Sep. 9, 2021

(54) METHODS OF TREATMENT OF CANCER WITH SUBSTITUTED PYRROLE AND PYRAZOLE COMPOUNDS AND DIAGNOSIS OF CANCERS SUSCEPTIBLE TO TREATMENT WITH SUBSTITUTED PYRROLE AND PYRAZOLE COMPOUNDS

(71) Applicant: Bantam Pharmaceutical, LLC, New york, NY (US)

(72) Inventors: Matthew Kostura, Hillsborough, NC (US); Michael Luther, Andover, MA (US)

(21) Appl. No.: 16/972,489

(22) PCT Filed: Jun. 7, 2019
(86) PCT No.: PCT/US2019/035992

§ 371 Date: Dec. 4, 2020

Related U.S. Application Data
(60) Provisional application No. 62/682,132, filed on Jun. 7, 2018.

(51) Int. Cl. G01N 33/574; C07D 417/04; A61P 35/00

(52) U.S. Cl. CPC G01N 33/57426; A61P 35/00; C07D 417/04

(57) ABSTRACT

Provided are methods of treatment of cancer with substituted pyrrole and pyrazole compounds and diagnoses of cancers susceptible to treatment with substituted pyrrole and pyrazole compounds, based on certain biomarkers identified herein.

### CROSS-REFERENCE TO RELATED APPLICATIONS

This application claims the benefit of priority of U.S. Provisional Patent Application No. 62/682,132, filed Jun. 7, 2018, which is hereby incorporated herein by reference in its entirety.

### BACKGROUND OF THE DISCLOSURE

### Field

This disclosure relates to the diagnosis and treatment of cancer. This disclosure relates more particularly to diagnosis of cancers susceptible to treatment with certain compounds based on genetic profiling, and the treatment of cancers using particular compounds based on the genetic profile of the cancer.

### Technical Background

In the United States over 1,500,000 new cases of cancer are diagnosed annually with an estimated 500,000 deaths per year related to cancer. There are more than 200 different histopathological types of cancer largely classified into solid tumor and hematopoietic cancers. Current cancer therapies vary depending upon the localization and stage of the cancer but generally include a combination of surgery, systemic therapy, radiation therapy, and chemotherapy. Despite the effort that has been devoted to the development of anti-cancer strategies, many of them remain inefficacious for specific cancers.

Significant efforts have been put forth to generate effective treatment options for patients diagnosed with cancer. Treatment options for cancer have evolved from non-specific cyotoxic agents to molecular targeting of tumor specific processes. However, the genetic complexity of cancerous tumors makes targeting specific molecular entities an inefficient strategy. The low overall success rate and increasing need for stringent patient selection criteria for clinical trials reflects that inefficiency.

As such, approaches targeting common aspects of tumor biology are highly desirable to provide effective treatment options with improved patient outcomes. Successful discovery of effective cancer treatments depends on development of novel therapeutic agents targeting hallmarks of cancer. Pathway and marker driven approaches place pharmacological proof of concept and a detailed understanding of pharmacological mechanism of action at the center of therapeutic innovation.

### SUMMARY

Described herein, in various aspects, is the identification of novel target genetic biomarkers for hematopoietic cancers and for solid tumor cancers that correlate with the efficacy of treatment using the therapeutic compounds; the measurement of the quantitative change in such biomarkers in hematopoietic and solid tumor cancers; and the treatment of hematopoietic cancers and solid tumor cancers using compounds of the therapeutic compound.

### BRIEF DESCRIPTION OF THE FIGURES

FIG. 1 is a graph demonstrating that blood tumor cell lines (“blood”) are more likely to be responsive than solid tumor cell lines (“other”) to Compound A197 (Welch's t-test P-value-2.3e-16).

FIG. 2 is a graph of demonstrating that the average AUCs among different solid tumor types are not significantly different (One-way ANOVA P-value=0.54).

FIG. 3 is a schematic depiction of clusters of 57 blood tumor cell lines into two groups, with the average AUCs of 3.11 and 2.56, respectively. Results indicate that the AUC is not different between tumor types.

FIG. 4 is a graph of the AUCs for KIAA0125 amplified (“KIAA0125_amp”) and unamplified (“KIAA0125_notamp”) in cell lines. Amplified and unamplified cell lines have significantly different average AUCs (p=0.043).

FIG. 5 is a graph of the AUCs detected between HLA-B and HLA-C deleted (“del”) and undeleted (“notdel”) genes. Cell lines have significantly different AUCs (p<0.05).

FIGS. 6A and 6B are a schematic depictions showing the drug efficacy prediction result using 9 signature genes and known sensitivity in 112 training solid cell lines. A total of 67 cell lines had prediction results and 61 cell lines were correctly predicted.

FIG. 7 is a schematic depiction of the drug efficacy prediction result using 9 signature genes and known sensitivity in 54 test solid cell lines. A total of 28 cell lines had prediction results and 23 cell lines were correctly predicted.

FIG. 8 is a schematic depiction of how eIF2a phosphorylation regulates translation initiation and ATF4 mRNA translation.

FIG. 9 is a graph of mouse pharmacokinetics of a single rising dose PK of Compound A197 in female CD-1 mice. Compound was administered orally with the indicated dose. Blood was drawn by tail-bleed at the appropriate times and Compound A197 plasma levels determined by LC-MS. All values, mean±SD (n=3 mice per group).

FIG. 10 is a set of graphs of in vivo treatment results for Athymic Nude mice engrafted with the DLBCL line SU-DHL-10 (left panel) or the CRC line HCT-116 (right panel). After 14 days, groups of mice (n=10 per group) were randomized by tumor volume and treated with Compound B19 (left panel) or Compound A197 (right panel). All animals dosed p.o. q.d. (orally once per day). Tumor burden was measured over 21 days. Compound B19 efficacy was monitored for 21 days following last dose to determine relapse. All data plotted as the mean±SEM, n=10 mice per group.

FIG. 11 is a graph of expression of ATF4, ATF6, Xbp1, and cell cycle regulated genes in Compound A197 responsive cell lines. All cells were treated with 5 pJVI Compound A197 for 8 hrs, then RNA was isolated and specific gene expression determined by qPCR. All data are represented as Log₂ Fc. *=p<0.05, n=3.

FIG. 12 is a set of graphs of Compound A197-induced phosphorylation of eIF2a. HCT-116 cells were treated with 5 μM Compound A197. eIF2α and p-eIF2α levels were determined by Western blotting. All data are mean±SD (n=3). *=p<0.05; #p<0.005.

FIG. 13 is a set of graphs showing correlation between FAM210B expression and outcomes in renal cancer, lung cancer, and cervical cancer.

FIG. 14 is a graph showing Cancer Cell Line Encyclopedia (CCLE) FAM210B expression data versus AUCs for hematopoietic and solid tumor cell lines. Coefficient of regression/Spearman correlation R value=0.43 for solid tumors.

FIG. 15 is a graph showing FAM210B robust multi-array average (RMA) normalized expression for diffuse large B-cell lymphoma (DLBCL), Burkitts lymphoma, and myeloma.

FIG. 16 is a graph showing nominal logistic regression for genes identified in Example 2, demonstrating that two genes account for the bulk of the partitioning that predicts response to compounds of the disclosure.

FIG. 17 is a set of fluorescence micrographs of HCT-116 cells transfected with a vector expressing tGFP (left) or FAM210B-GFP (right).

FIG. 18 is a graph of ATF4 expression versus GFP expression for HCT-116 cells transfected with a vector expressing tGFP or FAM210B-GFP. ATF4 expression decreases in a dose-dependent manner with rising FAM210B expression.

### DETAILED DESCRIPTION

The present inventors have determined that while many cancers are responsive to certain pyrrole and pyrazole-based therapeutic compounds (themselves described in International Patent Application Publication No. 2015/196644, and International Patent Application No. PCT/US2017/063774, each of which is hereby incorporated herein by reference in its entirety), many other cancers are not as responsive. The present inventors have identified certain cancers that are responsive to the therapeutic compounds, and moreover have determined different correlations of gene expression patterns that are predictive of responsiveness for hematopoietic cancers and for solid tumors. Specifically, the present specification describes, in various aspects, the identification of novel target genetic biomarkers for hematopoietic cancers and for solid tumor cancers that correlate with the efficacy of treatment using the therapeutic compounds; the measurement of the quantitative change in such biomarkers in hematopoietic and solid tumor cancers; and the treatment of hematopoietic cancers and solid tumor cancers using compounds of the therapeutic compound.

As described below, without intending to be bound by theory, the present inventors believe that the therapeutic compounds described herein operate by activating the ATF4 pathway. Accordingly, another aspect of the disclosure is a method for activating the ATF pathway in a cancer cell, the method comprising contacting the cell with an effective amount of a therapeutic compound. In another aspect, the disclosure provides a method for treating a cancer in which ATF4 activation is repressed in a human individual, the method including administering to the human individual an effective amount of a therapeutic compound. In another aspect, the disclosure provides a method for treating a cancer in a human individual, the method including determining whether ATF4 activation is repressed in the cancer, and if ATF4 activation is repressed, administering to the human individual an effective amount of a therapeutic compound. Prostate cancer is an example of a cancer in which the ATF4 pathway is repressed; see, e.g., Y. Erzurumlu et al., Scientific Reports 7:40719 (2017); X. Sheng et al., EMBO Molecular Medicine, 7(6):788 (2015). In another aspect, the disclosure provides a method for determining whether the ATF4 pathway is activated in a cancer by a therapeutic compound, the method including comparing the expression of one or more genes selected from ASNS, DDIT3, DDIT4, PP1R15A, SARS and SLC7A11 without treatment with the therapeutic compound and with treatment with the therapeutic compound, and if one or more of the one or more genes exhibits a log 2 fold change in excess of 0.5 (e.g., in excess of 1), identifying the ATF4 pathway as being activated by the therapeutic compound. Such methods can be used therapeutically; after such identification, the method can further include administering to a human individual having the cancer an effective amount of the therapeutic compound.

In one aspect, the disclosure provides methods for treating a cancer in a human individual. The methods include determining the level of expression of a plurality of genes of the cancer; and determining a gene expression fold change as compared to the level of expression of the one or more genes in a reference cell. As used herein, a “gene expression fold change” is the quotient of the expression level of a gene in the cancer divided by the expression level of the gene in a reference cell. Accordingly, a gene expression fold change of 1.5 indicates that the gene expression level is 50% greater in the cancer as compared to the reference cell. Notably, if the gene expression fold change is significant with respect to a first number of the plurality of genes, the cancer is identified as being likely to be responsive to a therapeutic compound, and an effective amount of a therapeutic compound is administered to the human individual. In this aspect of the disclosure, the first number is five or more (i.e., five or more genes exhibit a significant gene expression fold change as compared to the reference cell).

The person of ordinary skill in the art will determine the level of significance necessary to provide a desired degree of accuracy to the determination. In certain embodiments as otherwise described herein, a gene expression fold change of at least 1.2 is a significant change in gene expression. For example, in certain embodiments, a gene expression fold change of at least 1.5 is a significant change in gene expression. In other embodiments, a gene expression fold change of at least 2, or even at least 3 is a significant change in gene expression. One skilled in the art will understand that gene expression fold change can be positive or negative indicating an upregulation or downregulation, respectively, versus reference cell; the “at least 1.2” gene expression fold change indicates upregulation.

The methods, compounds, and uses described herein can be employed with respect to a variety of different cancers or with respect to cells of a variety of different types of cancer.

Hematopoietic cancer is a cancer of the blood, bone marrow, lymph, lymph nodes, or lymphatic system. The circulating nature of many such cancers is especially unique and leads to over 50,000 deaths annually. Hematopoietic cancers can be broadly grouped into classes including myeloproliferative neoplasm, lymphoma, leukemia, and plasma cell neoplasm.

Solid tumor cancer is term of art that encompasses a large class of cancers. The majority of cancers are solid tumor cancers, with the breast, lung, and prostate being common sites of solid tumor cancers. Solid tumor cancers that metastasize or spread are associated with poor prognosis. Thus, early detection is key and effective drugs are key to reducing morbidity and mortality. In addition, novel treatment strategies are required.

Consistent with this, a novel approach to cancer treatment is to use changes in gene expression to identify cancers that are responsive to novel drugs. In particular hematopoietic cancer and solid tumors exhibit gene changes. This, application describes novel gene changes that can be used to identify hematopoietic and solid tumor cancers that are responsive to a therapeutic compound.

For example, in certain embodiments as otherwise described herein, the cancer is a hematopoietic cancer. In certain embodiments as otherwise described herein, the cancer is a chronic myeloproliferative neoplasm. In other embodiments as otherwise described herein, the cancer is a lymphoma (e.g., Burkitt's lymphoma, Hodgkin's lymphoma, non-Hodgkin's lymphoma, hairy cell lymphoma, mantle cell lymphoma, T-cell lymphoma, cutaneous T-cell lymphoma, B-cell lymphoma, diffuse large B-cell lymphoma, double-hit lymphoma, Waldenstrom macroglobulinemia, primary central nervous System (CNS) lymphoma, and intravascular large B-cell lymphoma (ILBCL)). In other such embodiments, the cancer is a leukemia (e.g., acute lymphoblastic leukemia (ALL), acute myeloid leukemia (AML), acute myeloblastic leukemia, chronic lymphocytic leukemia (CLL), chronic myelogenous leukemia (CML), chronic neutrophilic leukemia (CNL), chronic myelomonocytic leukaemia (CMML), aggressive NK-cell leukemia, acute biphenotypic leukaemia, and polycythemia vera), acute and chronic T-cell and B-cell leukemia). In other such embodiments, the cancer is a plasma cell neoplasm (e.g., multiple myeloma).

However, the person of ordinary skill in the art will appreciate from the disclosure provided herein that the methods, compounds and uses described herein can be employed with a variety of other types of cancer. For example, in certain embodiments of the methods, compounds and uses as otherwise described herein, the cancer is selected from appendix cancer, bone cancer (e.g., Ewing sarcoma, osteosarcoma and malignant fibrous histiocytoma), bronchial tumors, carcinoma of unknown primary, chronic myeloproliferative neoplasms, colon and rectal cancer, head and neck cancer (including head and neck squamous cell carcinoma (HNSCC)), leukemia (e.g., acute lymphoblastic leukemia (ALL), acute myeloid leukemia (AML), acute myeloblastic leukemia, chronic lymphocytic leukemia (CLL), chronic myelogenous leukemia (CML), chronic neutrophilic leukemia (CNL), chronic myelomonocytic leukaemia (CMML), aggressive NK-cell leukemia, acute biphenotypic leukaemia, and polycythemia vera), acute and chronic T-cell and B-cell leukemia), lymphoma (e.g., Burkitt lymphoma, Hodgkin's lymphoma, non-Hodgkin's lymphoma, hairy cell lymphoma, mantle cell lymphoma, T-cell lymphoma, cutaneous T-cell lymphoma, B-cell lymphoma, diffuse large B-cell lymphoma, double-hit lymphoma, Waldenstrom macroglobulinemia, primary central nervous System (CNS) lymphoma, and intravascular large B-cell lymphoma (ILBCL)), plasma cell neoplasms (e.g., multiple myeloma), myelodysplastic syndrome, myelodysplastic/myeloproliferative neoplasms and chronic myeloproliferative neoplasms, pancreatic cancer and pancreatic neuroendocrine tumors (e.g., islet cell tumors), small intestine cancer, soft tissue sarcoma, and squamous cell carcinoma.

And in other embodiments of the methods, compounds and uses as otherwise described herein, the cancer is selected from adrenocortical carcinoma, adrenal cortex cancer, AIDS-related cancers (e.g., as Kaposi sarcoma, AIDS-related lymphoma, Burkitt lymphoma, and primary CNS lymphoma), anal cancer, appendix cancer, astrocytomas (e.g., childhood cerebellar or cerebral), bile duct cancer (e.g., cholangiocarcinoma), bladder cancer, bone cancer (e.g., Ewing sarcoma, osteosarcoma and malignant fibrous histiocytoma), brain tumors (e.g., glioblastoma multiform, cerebellar astrocytoma, cerebral astrocytoma/malignant glioma, ependymoma, medulloblastoma, oligodendroglioma, supratentorial primitive neuroectodermal tumors, and visual pathway and hypothalamic glioma), brainstem glioma, breast cancer, bronchial tumors, gastrointestinal carcinoid tumor, carcinoid tumors, carcinoma of unknown primary, cardiac (heart) tumors, central nervous system caner (e.g., atypical teratoid/rhabdoid tumor, embryonal tumors, and germ cell tumors), cervical cancer, childhood cancers, chondrosarcoma, chronic myeloproliferative neoplasms, colon and rectal cancer, craniopharyngioma, desmoplastic small round cell tumor, ductal carcinoma in situ (DCIS), endometrial cancer, ependymoma, epitheliod hemangioendothelioma (EHE), esophageal cancer, esthesioneuroblastoma, extracranial germ cell tumor, extragonadal germ cell tumor, eye cancer (e.g., intraocular melanoma, and retinoblastoma), fallopian tube cancer, gallbladder cancer, gastric (stomach) cancer, gastrointestinal stromal tumors (GIST), gestational trophoblastic disease (GTD), gliomas, hairy cell leukemia, head and neck cancer (e.g., head and neck squamous cell carcinoma (HNSCC)), hepatocellular (liver) cancer, histiocytosis, langerhans cell, hypopharyngeal cancer, kidney cancer, langerhans cell histiocytosis, laryngeal cancer, laryngeal cancer and papillomatosis, leukemia (e.g., acute lymphoblastic leukemia (ALL), acute myeloid leukemia (AML), acute myeloblastic leukemia, chronic lymphocytic leukemia (CLL), chronic myelogenous leukemia (CML), chronic neutrophilic leukemia (CNL), chronic myelomonocytic leukaemia (CMML), aggressive NK-cell leukemia, acute biphenotypic leukaemia, and polycythemia vera), acute and chronic T-cell and B-cell leukemia), lip and oral cavity cancer, liver cancer, lung cancer (e.g., small cell lung cancer, non-small cell lung cancer (NSCLC), lung adenocarcinoma, carcinoma of the lung, and squamous carcinoma of the lung), lung carcinoid tumor, lymphoma (e.g., Burkitt lymphoma, Hodgkin's lymphoma, non-Hodgkin's lymphoma, hairy cell lymphoma, mantle cell lymphoma, T-cell lymphoma, cutaneous T-cell lymphoma, B-cell lymphoma, diffuse large B-cell lymphoma, double-hit lymphoma, Waldenstrom macroglobulinemia, primary central nervous System (CNS) lymphoma, and intravascular large B-cell lymphoma (ILBCL)), male breast cancer, meningiomas, mesothelioma, midline tract carcinoma involving NUT gene, mouth cancer, multiple endocrine neoplasia syndrome, plasma cell neoplasm (e.g., multiple myeloma), mycosis fungoides, myelodysplastic syndrome, myelodysplastic/myeloproliferative neoplasms and chronic myeloproliferative neoplasms, nasal cavity and paranasal sinus cancer, nasopharyngeal cancer (NPC), neuroblastoma, oral cancer, lip and oral cavity cancer and oropharyngeal cancer, ovarian cancer, pancreatic cancer and pancreatic neuroendocrine tumors (e.g., islet cell tumors), paraganglioma, parathyroid cancer, penile cancer, pharyngeal cancer, pheochromocytoma, pituitary tumor, pleuropulmonary blastoma, primary peritoneal cancer, prostate cancer, retinoblastoma, rhabdomyosarcoma, salivary gland cancer, Sezary syndrome, skin cancer (e.g., basal and squamous cell carcinoma, merkel cell carcinoma, and melanoma), small intestine cancer, soft tissue sarcoma, squamous cell carcinoma, stomach (gastric) cancer, testicular cancer, throat cancer, thymoma and thymic carcinoma, thyroid cancer, transitional cell cancer of the renal pelvis and ureter, urethral cancer, uterine cancer and uterine Sarcoma, vaginal cancer, vascular tumors, vulvar cancer, and Wilms tumor.

In other embodiments, the cancer is a solid tumor, e.g., of any type described herein. For example, in a few particular embodiments of methods, compounds and uses as otherwise described herein, the cancer is a solid tumor. The solid tumor can be in various embodiments, for example, a lung cancer, a colorectal cancer, or a pancreatic cancer.

In one particular embodiment of the methods, compounds and uses as otherwise described herein, the cancer is diffuse large B-cell lymphoma.

The person of ordinary skill in the art will appreciate that the classification of cancers above is broad, and that some cancers may be classified in one, both, or the opposite group in comparision to the classification herein. A hematopoietic or solid tumor cancer diagnosis is established by a helath care provider using commonly established criteria for cancer diagnosis.

The level of gene expression can be calculated using a variety of scientifically accepted techniques for reporting gene expression. Without wishing to be bound by any single method, in one embodiment quantitiative polymerase chain reaction is performed and the gene expression is calculated using real time PCR and the Δ ΔC_{T} method. In an alternate embodiment microarrays can be used to quantify RNA transcript and provide a quantitiative measure of gene expression. One skilled in the art will recognize that more than one method can be used to calculate gene expression and gene expression fold change. In addition, one or more housekeeping genes can be amplified as an internal experimental control. The internal experimental control allows for internal assessment of experimental parameters and normalization of target gene expression. Suitable housekeeping genes include 18s rRNA, 28s rRNA, α-tubulin, β-actin, ALB RPL32, TBP, CYCC, EF1A and GAPDH. A person of ordinary skill in the art will understand that the list of housekeeping genes herein is not exhaustive and other housekeeping genes can also be amplified as dictated by experimental conditions. Exemplary housekeeping gene accession numbers are in the table below:

| Gene | Accession Number |
|---|---|
| 18s rRNA | NR_146146.1 |
| 28s rRNA | NM_001033714.2 |
| α-tubulin | NM_006082.2 |
| β-actin | NM_001101.4 |
| Albumin (ALB) | NM_000477.6 |
| RPL32 | NM_000994.3 |
| TBP | NM_003194.4 |
| CYCC | NM_005190.3 |
| EF1A | NM_001402.5 |
| GAPDH | NM_001256799.2 |

Conventional sampling techniques can be used to isolate cancer cells from a human individual for analysis. For example, for a a solid tumor cancer, a biopsy can be obtained from a cancerous tissue. For a hematopoietic cancer, cancer cells can be isolated from a blood sample, a bone marrow sample, or another relevant tissue from the human individual.

The gene expression fold change is determined with respect to a reference cell. The person of ordinary skill in the art will select a suitable reference cell for a particular type of cancer. For example, in certain embodiments, the reference cell is a non-cancerous cell of the human individual (e.g., of the same type as the cancer, e.g., the hematopoietic cancer or the solid tumor cancer). For example, a non-cancerous control tissue biopsy can taken from the same organ or tissue in the human individual. For certain hematopoieitc cancers, a blood cell (e.g., a leukocyte) line can be cultured and the gene expression in the blood cell used as a control. In other embodiments, the reference cell is a non-cancerous cell of a different human (e.g., of the same type as the cancer, e.g., the hematopoietic cancer or the solid tumor cancer).

In other embodiments, a non-cancerous, tissue-specific cell line can be used as the reference cell. Desirably, the reference cell is of the same type as the cancer, but in some cases a different type of reference cell may serve as a useful control. The following cell lines are exemplary control cell lines: normal human lung fibroblasts, Human cervix epitheloid carcinoma (HeLa) cells, human umbilical vein epithelial cells, normal (non-cancerous) primary cell lines, COS7 cells, HEK cells, NIH 3T3 embryonic fibroblast cells, Human Embryonic Kidney (HEK) 293 cells, MRC-5 (PD-19) Human foetal lung cells, C2C12 Mouse C3H muscle myoblast, L929 Mouse C3H/connective tissue, NIH 3T3 Mouse Swiss NIH embryo, MRC-5 (PD 25) Human foetal lung, ACHO-K1 Hamster Chinese ovary, MDCK Canine Cocker Spaniel kidney, HUVEC Human Pre-screened Umbilical Vein Endothelial Cells (HUVEC), J774A.1 Mouse BALB/c monocyte macrophage, MC3T3-E1 Mouse C57BL/6 calvaria, J774.2 Mouse BALB/c monocyte macrophage, MA104 Monkey African Green kidney, BEAS-2B Human bronchial epithelium (normal), BHK21 (clone 13) Hamster Syrian kidney, MDCK-II Canine Cocker Spaniel Kidney, PNT2 Human prostate normal, immortalized, COS-7 Monkey African green kidney, SV40 transformed, MDCK Canine Cocker Spaniel kidney, HUVEC Human Umbilical Vein Endothelial Cells (HUVEC); RK 13 Rabbit kidney, BVDV negative, tsA201 Human embryonal kidney, SV40 transformed, CHO Hamster Chinese ovary, PANC-1 Human Caucasian pancreas, Nthy-ori 3-1 Human thyroid follicular epithelial, WI 38 Human Caucasian foetal lung.

The list of possible control cells herein is not exhaustive. One skilled in the art will recognize that the control cell line requires a stable expression of the gene of interest. Thus, a person having skill in the art will recognize that additional cell lines could be used as controls to calculate fold change.

In other embodiments, the reference cell is a cell from a cancer cell line having an IC₅₀ of at least 30 μM for the therapeutic compound. As described in more detail below, the present inventors have determined that expression of certain genes is correlated with sensitivity to the therapeutic compounds, with non-responsive cells having a different expression pattern than responsive cells. Accordingly, a relatively non-responsive cell line (i.e., with an IC₅₀ of at least 30 μM for the therapeutic compound) can be used as a control. The data provided herein identify a number of such cell lines; the person of ordinary skill in the art can choose a cell line from those identified in the experimental section as being non-responsive to the example compounds for use as a reference cell.

In certain embodiments as otherwise described herein, the cancer is a hematopoietic cancer and the plurality of genes is selected from CASP10, TMED1, PPP1CC, TMEM59, BRD7, CYB561, FAM210B, NDRG1, CTSB, MMAB, SETDB2, VPS37B, ELL3, and KIF13B. If the gene expression fold change is significant with respect to a first number (e.g., five or more) of the plurality of genes, the hematopoietic cancer is identified as being likely to be sensitive to the therapeutic compound. In certain such embodiments, an effective amount of the compound is administered to the human individual to treat the cancer. However, in other embodiments, the method is used simply to identify whether the cancer is responsive to a therapeutic compound.

For hematopoietic cancers, in certain embodiments, the first number is seven or more, i.e., a significant gene expression fold change in seven or more of the fourteen genes identified above is indicative of likely sensitivity of the cancer to a therapeutic compound. For example, the first number can be 8 or more, 9 or more, or 10 or more genes. In certain embodiments, the first number is 11 or more, 12 or more, or 13 or more. And in certain embodiments, the first number is 14, i.e., a significant gene expression fold change in each of the fourteen genes identified above is indicative of likely sensitivity of the cancer to a therapeutic compound.

The person of ordinary skill in the art will appreciate that various combinations and permutations of the nine genes described above can be used in the practice of the methods described herein. For example, in certain embodiments as otherwise described herein, at least one of the plurality of genes is CASP10 (e.g., CASP10 is one of the first number of genes). In certain embodiments as otherwise described herein, at least one of the plurality of genes is TMED1 (e.g., TMED1 is one of the first number of genes). In certain embodiments as otherwise described herein, at least one of the plurality of genes is PPP1CC (e.g., PPP1CC is one of the first number of genes). In certain embodiments as otherwise described herein, at least one of the plurality of genes is TMEM59 (e.g., TMEM59 is one of the first number of genes). In certain embodiments as otherwise described herein, at least one of the plurality of genes is BRD7 (e.g., BRD7 is one of the first number of genes). In certain embodiments as otherwise described herein, at least one of the plurality of genes is CYB561 (e.g., CYB561 is one of the first number of genes). In certain embodiments as otherwise described herein, at least one of the plurality of genes is FAM210B (e.g., FAM210B is one of the first number of genes). In certain embodiments as otherwise described herein, at least one of the plurality of genes is NDRG1 (e.g., NDRG1 is one of the first number of genes). In certain embodiments as otherwise described herein, at least one of the plurality of genes is CTSB (e.g., CTSB is one of the first number of genes). In certain embodiments as otherwise described herein, at least one of the plurality of genes is MMAB (e.g., MMAB is one of the first number of genes). In certain embodiments as otherwise described herein, at least one of the plurality of genes is SETDB2 (e.g., SETDB2 is one of the first number of genes). In certain embodiments as otherwise described herein, at least one of the plurality of genes is VPS37B (e.g., VPS37B is one of the first number of genes). In certain embodiments as otherwise described herein, at least one of the plurality of genes is ELL3 (e.g., ELL3 is one of the first number of genes). In certain embodiments as otherwise described herein, at least one of the plurality of genes is KIF13B (e.g., KIF13B is one of the first number of genes).

In certain embodiments as otherwise described herein, the cancer is a hematopoietic cancer and the plurality of genes is selected from LAMC3, FAM210B, SENP8, ITGB3BP, NUDT2, HNRNPCL1, C20orf43, FRMD8, and STX16. If the gene expression fold change is significant with respect to a first number (e.g., five or more) of the plurality of genes, the solid tumor cancer is identified as being likely to be sensitive to the therapeutic compound. In certain such embodiments, an effective amount of the compound is administered to the human individual to treat the cancer. However, in other embodiments, the method is used simply to identify whether the cancer is responsive to a therapeutic compound.

For solid tumor cancers, in certain embodiments, the first number is five or more, i.e., a significant gene expression fold change in five or more of the fourteen genes identified above is indicative of likely sensitivity of the cancer to a therapeutic compound. For example, the first number can be 6 or more. In certain embodiments, the first number is 7 or more or 8 or more. And in certain embodiments, the first number is 9, i.e., a significant gene expression fold change in each of the nine genes identified above is indicative of likely sensitivity of the cancer to a therapeutic compound.

The person of ordinary skill in the art will appreciate that various combinations and permutations of the nine genes described above can be used in the practice of the methods described herein. For example, in certain embodiments as otherwise described herein, at least one of the plurality of genes is LAMC3 (e.g., LAMC3 is one of the first number of genes). In certain embodiments as otherwise described herein, at least one of the plurality of genes is FAM210B (e.g., FAM210B is one of the first number of genes). In certain embodiments as otherwise described herein, at least one of the plurality of genes is SENP8 (e.g., SENP8 is one of the first number of genes). In certain embodiments as otherwise described herein, at least one of the plurality of genes is ITGB3BP (e.g., ITGB3BP is one of the first number of genes). In certain embodiments as otherwise described herein, at least one of the plurality of genes is NUDT2 (e.g., NUDT2 is one of the first number of genes). In certain embodiments as otherwise described herein, at least one of the plurality of genes is HNRNPCL1 (e.g., HNRNPCL1 is one of the first number of genes). In certain embodiments as otherwise described herein, at least one of the plurality of genes is C20orf43 (e.g., C20orf43 is one of the first number of genes). In certain embodiments as otherwise described herein, at least one of the plurality of genes is FRMD8 (e.g., FRMD8 is one of the first number of genes). In certain embodiments as otherwise described herein, at least one of the plurality of genes is STX16 (e.g., STX16 is one of the first number of genes).

Another aspect of the disclosure is a method for treating a hematopoietic cancer in a human individual. The method includes determining a gene copy number for KIAA0125 of the hematopoietic cancer; and if the gene copy number is at least a second number (e.g., at least 2, or at least 4), identifying the hematopoieic cancer as likely to be responsive to a therapeutic compound. The method can further include administering an effective amount of a therapeutic compound to the human individual. But in other embodiments, the method can be used to identify whether the cancer is responsive to the therapeutic compound. Such methods can otherwise be performed as described elsewhere herein. The accession number for KIAA0125 is NM_014792.2.

Another aspect of the disclosure is a method for treating a hematopoietic cancer in a human individual. The method includes determining a gene copy number for HLA-B and/or HLA-C of the hematopoietic cancer; and if the gene copy number is no more than a third number (e.g., no more than 0.4, no more than 0.1, or no more than 0.07), identifying the hematopoietic cancer as likely to be responsive to a therapeutic compound. The method can further include administering an effective amount of a therapeutic compound to the human individual. But in other embodiments, the method can be used to identify whether the cancer is responsive to the therapeutic compound. Such methods can otherwise be performed as described elsewhere herein. The accession numbers for HLA-B and HLA-C are, respectively, NM_005514 and NM_001243042.1.

Another aspect of the disclosure is a method for treating a cancer in a human individual, the method comprising administering to the human individual an effective amount of the therapeutic compound.

In certain embodiments, the cancer is a hematopoietic cancer that exhibits a significant gene expression fold change as compared to a reference cell with respect to a first number of a plurality of genes selected from CASP10, TMED1, PPP1CC, TMEM59, BRD7, CYB561, FAM210B, NDRG1, CTSB, MMAB, SETDB2, VPS37B, ELL3, and KIF13B, wherein the first number is at least five. The applicable details of the gene expression fold changes, including the identity of the genes, the first number, the reference cell, the plurality of genes, and the level of significance can be as described above with respect to the hematopoietic cancer embodiments.

In certain embodiments, the cancer is a solid tumor cancer that exhibits a significant gene expression fold change as compared to a reference cell with respect to a first number of a plurality of genes selected from LAMC3, FAM210B, SENP8, ITGB3BP, NUDT2, HNRNPCL1, C20orf43, FRMD8, and STX16, wherein the first number is at least five. The applicable details of the gene expression fold changes, including the identity of the genes, the first number, the reference cell, the plurality of genes, and the level of significance can be as described above with respect to the solid tumor cancer embodiments.

In certain embodiments, the cancer is a hematopoietic cancer than exhibits a gene copy number for HLA-B and/or HLA-C that is no more than 0.10 (e.g., no more than 0.07).

In certain embodiments, the cancer is a hematopoietic cancer that exhibits a gene copy number for KIAA0125 that is at least 2 (e.g., at least 4).

The data described in Table 1 below demonstrates particular cancers for which the methods described herein can be especially useful. Accordingly, in certain embodiments as otherwise described herein, the cancer is acute lymphoblastic leukemia, acute promyelocytic leukemia, adrenal cortex carcinoma, acute monocytic leukemia, acute myeloid leukemia, B acute lymphoblastic leukemia, amelanotic melanoma, anaplastic large cell lymphoma, astrocytoma, B-cell prolymphocytic leukemia, biphasic synovial sarcoma, bladder carcinoma, chronic myeloid leukemia, breast adenocarcinoma, breast carcinoma, Burkitt's lymphoma, cecum adenocarcinoma, cervical carcinoma, cervical squamous cell carcinoma, T acute lymphoblastic leukemia, chronic eosinophilic leukemia, chronic myelogenous leukemia, colon adenocarcinoma, colon carcinoma, cutaneous melanoma, diffuse gastric adenocarcinoma, diffuse large B-cell lymphoma, diffuse large B-cell lymphoma activated B-cell type, diffuse large B-cell lymphoma germinal center B-Cell type, ductal breast carcinoma, duodenal adenocarcinoma, embryonal rhabdomyosarcoma, endometrial adenocarcinoma, endometrial adenosquamous carcinoma, Epstein-Barr virus-related Burkitt lymphoma, erythroleukemia, esophageal squamous cell carcinoma, Ewing sarcoma, fibrosarcoma, follicular lymphoma, gallbladder carcinoma, gastric adenocarcinoma, gastric adenosquamous carcinoma, gastric carcinoma, gastric tubular adenocarcinoma, gestational choriocarcinoma, glioblastoma, head and neck squamous cell carcinoma, hepatoblastoma, hepatocellular carcinoma, thyroid gland medullary carcinoma, ovarian serous adenocarcinoma, human papillomavirus-related cervical squamous cell carcinoma, human papillomavirus-related endocervical adenocarcinoma, hypopharyngeal squamous cell carcinoma, thyroid gland undifferentiated (anaplastic) carcinoma, inflammatory breast carcinoma, intrahepatic cholangiocarcinoma, invasive ductal carcinoma, large B-cell lymphoma, large cell lung carcinoma, lung adenocarcinoma, mantle cell lymphoma, melanoma, minimally invasive lung adenocarcinoma, nasopharyngeal carcinoma, natural killer cell lymphoblastic leukemia/lymphoma, neuroblastoma, non-small cell lung carcinoma, osteosarcoma, ovarian clear cell adenocarcinoma, ovarian endometrioid adenocarcinoma, ovarian serous cystadenocarcinoma, pancreatic adenocarcinoma, pancreatic carcinoma, pancreatic ductal adenocarcinoma, papillary lung adenocarcinoma, papillary renal cell carcinoma, plasma cell myeloma, plasmacytoma, pleomorphic breast carcinoma, pleural biphasic mesothelioma, pleural epithelioid mesothelioma, prostate carcinoma, rectal adenocarcinoma, rectosigmoid adenocarcinoma, renal cell carcinoma, Sezary Syndrome, signet ring cell gastric adenocarcinoma, small cell lung carcinoma, squamous cell lung carcinoma, thyroid gland follicular carcinoma, thyroid gland papillary carcinoma, thyroid gland squamous cell carcinoma, thyroid gland undifferentiated (anaplastic) carcinoma, tongue squamous cell carcinoma, uterine corpus sarcoma, or vulvar squamous cell carcinoma. In certain embodiments as otherwise described herein, the cancer is acute promyelocytic leukemia, acute monocytic leukemia, acute myeloid leukemia, B acute lymphoblastic leukemia, Anaplastic large cell lymphoma, B-cell prolymphocytic leukemia, chronic myeloid leukemia, Burkitt lymphoma, chronic eosinophilic leukemia, chronic myelogenous leukemia, diffuse large B-cell lymphoma, diffuse large B-cell lymphoma activated B-cell type, diffuse large B-cell lymphoma germinal center B-Cell type, Epstein-Barr virus-related Burkitt lymphoma, erythroleukemia, follicular lymphoma, large B-cell lymphoma acute lymphoblastic leukemia, mantle cell lymphoma, natural killer cell lymphoblastic leukemia/lymphoma plasma cell myeloma, plasmacytoma, or Sezary syndrome.

In another aspect, the disclosure provides methods for diagnosing and treating treating solid tumor cancers in a human individual. The present inventors have determined that solid tumor cancers exhibiting decreased FAM210B expression are especially susceptible to treatment by the therapeutic compounds described herein.

For example, in one aspect, a method for treating a solid tumor cancer includes determining the level of expression of FAM210B of the cancer; and determining a FAM210B expression fold change as compared to the level of FAM210B expression in a reference cell. Notably, if the FAM210B gene expression fold change is significant, and if FAM210B expression in the cancer cell is lower than FAM210B expression in the reference cell, the cancer is identified as being likely to be responsive to a therapeutic compound of the disclosure, and an effective amount of the therapeutic compound is administered to the human individual. Significance of FAM210B expression fold change can be determined as described with regard to other aspects of the disclosure.

In another aspect, a method for treating a solid tumor cancer in a human individual is provided. The solid tumor cancer exhibits a significant FAM210B expression fold change (e.g., as otherwise described herein) as compared to the level of expression of FAM210B in a reference cell. The method includes administering to the human individual an effective amount of a therapeutic compound as described herein.

The person of ordinary skill in the art will determine effective amounts and dosages of the compounds described herein based on this disclosure, as well as the disclosures of International Application Publication No. WO 2016/196644 and U.S. Application Publication No. 2018/0100457, the disclosures of which are incorporated by reference herein.

Unless defined otherwise, all technical and scientific terms used herein have the same meaning as is commonly understood by one of ordinary skill in the art. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). These references are intended to be exemplary and illustrative and not limiting as to the source of information known to the worker of ordinary skill in this art. As used herein, the following terms have the meanings ascribed to them below, unless specified otherwise.

It is noted here that as used in this specification and the appended claims, the singular forms “a,” “an,” and “the” also include plural reference, unless the context clarity dictates otherwise.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

Diagnostic or informative alteration or change in a biomarker is meant as an increase or decrease in the expression levels or activity of a gene or gene product as detected by conventional methods known in the art such as those described herein.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “housekeeping gene” is used to refer to a gene used as an internal control in a PCR experiment. A housekeeping gene demonstrates minimal variability in gene expression between a blood sample from a human individual with a hematopoietic cancer and gene expression in a blood sample from a healthy individual human or a cell line. A housekeeping gene also demonstrates minimal variability in gene expression in tissue from a human individual with a solid tumor cancer and a non-cancerous tissue sample from a healthy individual or cell line. Thus, housekeeping gene expression is minimally impacted by cancer.

A variety of therapeutic compounds can be used in the practice of the methods of the disclosure, generally selected from any embodiment or genus of International Patent Application Publication No. 2015/196644, or of International Patent Application Publication No. 2018/102453, each of which is hereby incorporated herein by reference in its entirety.

For example, in certain embodiments, the therapeutic compound is a compound as generally described in any genus, subgenus or embodiment of International Patent Application Publication no. 2015/196644.

In certain embodiments, the therapeutic compound is of formula (I),

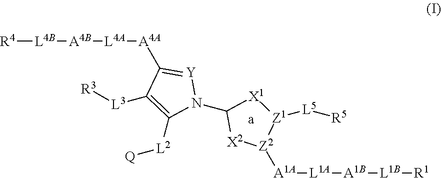

in which formula (I) the ring system denoted by “a” is defined as being heteroaromatic, optionally in the form of a pharmaceutically acceptable salt or N-oxide, or a solvate or hydrate, wherein
A^{1A} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
L^{1A} is selected from the group consisting of a bond, unsubstituted C₁-C₄ alkylene, unsubstituted C₁-C₄ alkenylene and unsubstituted C₁-C₄ alkynylene;
A^{1B} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
L^{1B} is selected from the group consisting of a bond, unsubstituted C₁-C₄ alkylene, unsubstituted C₁-C₄ alkenylene and unsubstituted C₁-C₄ alkynylene;
R¹ is selected from the group consisting of hydrogen,
   optionally substituted C₁-C₈ alkyl, optionally-substituted C₁-C₈ alkenyl and optionally substituted C₁-C₈ alkynyl,
   cycloalkyl and heterocycloalkyl, each optionally substituted with 1-5 R^{1E}, and aryl and heteroaryl, each optionally substituted with 1-5 R^{1E},
   in which
      each R^{1E} is independently selected from oxo, optionally-substituted C₁-C₄ alkyl, C₁-C₄ fluoroalkyl, halogen, —CN, SF₅, —N₃, —C(O)R^{1F}, —SR^{1F}, —S(O)₁₋₂R^{1F}, —OR^{1F}, —NR^{1G}R^{1F}, —C(O)R^{1F}, —C(O)NR^{1G}R^{1F}, —NR^{1G}C(O)R^{1F}, —C(S)NR^{1G}R^{1F}, —NR^{1G}C(S)R^{1F}, —C(O)OR^{1F}, —OC(O)R^{1F}, —C(O)SR^{1F}, —SC(O)R^{1F}, —C(S)OR^{1F}, —OC(S)R^{1F}, —C(S)SR^{1F}, —SC(S)R^{1F}, —S(O)₁₋₂OR^{1F}, —OS(O)₁₋₂R^{1F}, —S(O)₁₋₂NR^{1G}R^{1F}, —NR^{1G}S(O)₁₋₂R^{1F};
      each R^{1F} is independently selected from H, C₁-C₃ alkyl, C₁-C₃ fluoroalkyl, C₁-C₃ hydroxyalkyl and (C₁-C₃ alkoxy)C₁-C₃ alkyl, (C₁-C₃ alkoxy(C₁-C₃ alkoxy))C₁-C₃ alkyl, (C₁-C₃ alkoxy(C₁-C₃ alkoxy(C₁-C₃ alkoxy)))C₁-C₃ alkyl, and
      each R^{1G} is independently selected from H, C₁-C₃ alkyl, C₁-C₃ fluoroalkyl, C₁-C₃ hydroxyalkyl, (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl);
L² is selected from the group consisting of a bond, —CH₂—, —CH(CH₃)— or —CH₂CH₂—; Q is selected from the group consisting of H, —CH₂OH, —C(O)OH, —C(O)OR^{2A}, —C(O)NR^{2B}R^{2A}, —C(O)NR^{2B}S(O)₂R^{2A}, —C(O)NR^{2B}S(O)₂NR^{2B}R^{2A}, —C(O)R^{2A}, —S(O)₂OH, —P(O)(OH)₂, —C(OH)(CF₃)₂, S(O)₂R^{2A}, —N(R^{2B})S(O)₂R^{2A}, —S(O)₂NR^{2B}R^{2A}, —C(O)NHOH, —C(O)NH—O(C₁-C₃ alkyl), —CO(NH)CN,

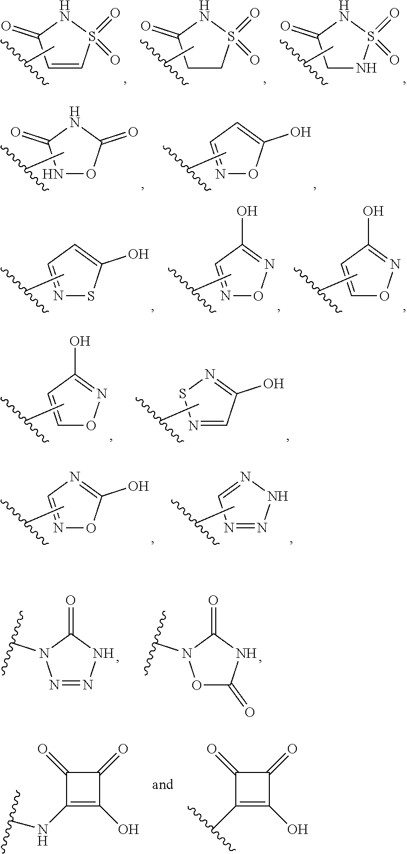

in which
   each R^{2A} is independently selected from H, C₁-C₃ alkyl, C₁-C₃ fluoroalkyl, C₁-C₃ hydroxyalkyl C₁-C₃ aminoalkyl, C₁-C₃ thioalkyl, (C₁-C₃ alkoxy)C₁-C₃ alkyl, —(CH₂CH₂O)₂₋₅-(optionally substituted C₁-C₃ alkyl)- and heteroaryl optionally substituted with 1-2 groups selected from substituents selected from C₁-C₃ alkyl, C₁-C₃ fluoroalkyl, hydroxyl, amino, thio, C₁-C₃ hydroxyalkyl, C₁-C₃ aminoalkyl, C₁-C₃ thioalkyl and —C(O)C₁-C₃ alkyl, and
   each R^{2B} is independently selected from H, C₁-C₃ alkyl, C₁-C₃ fluoroalkyl, C₁-C₃ hydroxyalkyl, C₁-C₃ aminoalkyl, C₁-C₃ thioalkyl, (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl), or R^{2A} and R^{2B} come together with a nitrogen to which they are both directly bound to form a heterocycloalkyl optionally substituted with 1-3 substituents selected from C₁-C₃ alkyl, C₁-C₃ fluoroalkyl, hydroxyl, amino, thio, C₁-C₃ hydroxyalkyl, C₁-C₃ aminoalkyl, C₁-C₃ thioalkyl and —C(O)C₁-C₃ alkyl;
L³ is a bond, —C(O)—, —S—, —S(O)₁₋₂—, —O—, —NR⁶—, —CH₂—, —CH(CH₃)(OH)—, —CH(OH)—, —CH₂CH₂—, —C(O)NR⁶—, —NR⁶C(O)—, —C(S)NR⁶—, —NR⁶C(S)—, —C(O)O—, —OC(O)—, —S(O)₁₋₂O—, —OS(O)₁₋₂—, —S(O)₁₋₂NR⁶— or —NR⁶S(O)₁₋₂—;
R³ is selected from the group consisting of
   cycloalkyl and heterocycloalkyl, each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E}, and
   aryl and heteroaryl each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E},
   in which
      each L^{3C} is a bond, methylene,
         ethylene, —C(O)—, —S—, —S(O)₁₋₂—, —O— or —NR^{3G}—;
      each R^{3D} is independently selected from optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F}, —OC(O)OR^{3F}, —OC(O)NR^{3G}R^{3F}, —NR^{3G}C(O)OR^{3F}, —NR^{3G}C(O)NR^{3G}R^{3F}, —SC(O)OR^{3F}, —OC(O)SR^{3F}, —SC(O)SR^{3F}, —SC(O)NR^{3G}R^{3F}, —NR^{3G}C(O)SR^{3F}, —OC(S)OR^{3F}, —OC(S)NR^{3G}R^{3F}, —NR^{3G}C(S)OR^{3F}, —NR^{3G}C(S)NR^{3G}R^{3F}, —SC(S)OR^{3F}, —OC(S)SR^{3F}, —SC(S)SR^{3F}, —SC(S)NR^{3G}R^{3F}, —NR^{3G}C(S)SR^{3F}, —NR^{3G}C(NR^{3G})NR^{3G}R^{3F} and —NR^{3G}S(O)₁₋₂NR^{3G}R^{3F};
      each R^{3E} is independently selected from oxo, optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F}, —OC(O)OR^{3F}, —OC(O)NR^{3G}R^{3F}, —NR^{3G}C(O)OR^{3F}, —NR^{3G}C(O)NR^{3G}R^{3F}, —SC(O)OR^{3F}, —OC(O)SR^{3F}, —SC(O)SR^{3F}, —SC(O)NR^{3G}R^{3F}, —NR^{3G}C(O)SR^{3F}, —OC(S)OR^{3F}, —OC(S)NR^{3G}R^{3F}, —NR^{3G}C(S)OR^{3F}, —NR^{3G}C(S)NR^{3G}R^{3F}, —SC(S)OR^{3F}, —OC(S)SR^{3F}, —SC(S)SR^{3F}, —SC(S)NR^{3G}R^{3F}, —NR^{3G}C(S)SR^{3F}, —NR^{3G}C(NR^{3G})NR^{3G}R^{3F} and —NR^{3G}S(O)₁₋₂NR^{3G}R^{3F};
      each R^{3F} is independently selected from H, C₁-C₃ alkyl, C₁-C₃ fluoroalkyl and C₁-C₃ hydroxyalkyl and each R^{3G} is independently selected from H, C₁-C₃ alkyl, C₁-C₃ fluoroalkyl, C₁-C₃ hydroxyalkyl, —S(O)₁₋₂(C₁-C₃ alkyl), —C(O)(C₁-C₃ alkyl) and —C(O)O(C₁-C₃ alkyl);
A^{4A} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
L^{4A} is selected from the group consisting of a bond, unsubstituted C₁-C₄ alkylene, unsubstituted C₁-C₄ alkenylene and unsubstituted C₁-C₄ alkynylene;
A^{4B} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
L^{4B} is selected from the group consisting of a bond, unsubstituted C₁-C₄ alkylene, unsubstituted C₁-C₄ alkenylene and unsubstituted C₁-C₄ alkynylene;
R⁴ is selected from the group consisting of hydrogen,
   optionally substituted C₁-C₈ alkyl, optionally-substituted C₁-C₈ alkenyl and
   optionally substituted C₁-C₈ alkynyl,
   cycloalkyl and heterocycloalkyl, each optionally substituted with 1-5 R^{4E}, and
   in which
      each R^{4E} is independently selected from oxo, optionally-substituted C₁-C₄ alkyl, C₁-C₄ fluoroalkyl, halogen, —CN, —SF₅, —N₃, —C(O)R^{4F}, —SR^{4F}, —S(O)₁₋₂R^{4F}, —OR^{4F}, —NR^{4G}R^{4F}, —C(O)R^{4F}, —C(O)NR^{4G}R^{4F}, —NR^{4G}C(O)R^{4F}, —C(S)NR^{4G}R^{4F}, —NR^{1G}C(S)R^{4F}, —C(O)OR^{4F}, —OC(O)R^{4F}, B—C(O)SR^{4F}, —SC(O)R^{4F}, —C(S)OR^{4F}, —OC(S)R^{4F}, —C(S)SR^{4F}, —SC(S)R^{4F}, —S(O)₁₋₂OR^{4F}, —OS(O)₁₋₂R^{4F}, —S(O)₁₋₂NR^{4G}R^{4F}, —NR^{4G}S(O)₁₋₂R^{4F}, —OC(O)OR^{4F}, —OC(O)NR^{4G}R^{4F}, —NR^{4G}C(O)OR^{4F}, —NR^{4G}C(O)NR^{4G}R^{4F}, —SC(O)OR^{4F}, —OC(O)SR^{4F}, —SC(O)SR^{4F}, —SC(O)NR^{4G}R^{4F}, —NR^{4G}C(O)SR^{4F}, —OC(S)OR^{4F}, —OC(S)NR^{4G}R^{4F}, —NR^{4G} C(S)OR^{4F}, —NR^{4G}C(S)NR^{4G}R^{4F}, —SC(S)OR^{4F}, —OC(S)SR^{4F}, —SC(S)SR^{4F}, —SC(S)NR^{4G}R^{4F}, —NR^{4G}C(S)SR^{4F}, —NR^{4G}C(NR^{4G})NR^{4G}R^{4F} and —NR^{4G}S(O)₁₋₂NR^{4G}R^{4F};
      each R^{4F} is independently selected from H, C₁-C₃ alkyl, C₁-C₃ fluoroalkyl, C₁-C₃ hydroxyalkyl and (C₁-C₃ alkoxy)C₁-C₃ alkyl and
      each R^{4G} is independently selected from H, C₁-C₃ alkyl, C₁-C₃ fluoroalkyl, C₁-C₃ hydroxyalkyl, and (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl);
L⁵ is —C(O)—, —S—, —S(O)₁₋₂—, —O—, —NR⁶—, —CH₂—, —CH(CH₃)(OH)—, —CH(OH)—, —CH₂CH₂—, —C(O)NR⁶—, —NR⁶C(O)—, —C(S)NR⁶—, —NR⁶C(S)—, —C(O)O—, —OC(O)—, —S(O)₁₋₂O—, —OS(O)₁₋₂—, —S(O)₁₋₂NR⁶— or —NR⁶S(O)₁₋₂—;
R⁵ is selected from the group consisting of
   cycloalkyl and heterocycloalkyl, each optionally substituted with 1-5 R^{5E}, and aryl and heteroaryl each optionally substituted with 1-5 R^{5E},
   in which
      each R^{5E} is independently selected from oxo, optionally-substituted C₁-C₄ alkyl, C₁-C₄ fluoroalkyl, halogen, —CN, —SF₅, —N₃, —C(O)R^{5F}, —SR^{5F}, —S(O)₁₋₂R^{5F}, —OR^{5F}, —NR^{5G}R^{5F}, —C(O)R^{5F}, —C(O)NR^{5G}R^{5F}, —NR^{5G}C(O)R^{5F}, —C(S)NR^{5G}R^{5F}, —NR^{1G}C(S)R^{5F}, —C(O)OR^{5F}, —OC(O)R^{5F}, —C(O)SR^{5F}, —SC(O)R^{5F}, —C(S)OR^{5F}, —OC(S)R^{5F}, —C(S)SR^{5F}, —SC(S)R^{5F}, —S(O)₁₋₂OR^{5F}, —OS(O)₁₋₂R^{5F}, —S(O)₁₋₂NR^{5G}R^{5F}, —NR^{5G}S(O)₁₋₂R^{5F}, —OC(O)OR^{5F}, —OC(O)NR^{5G}R^{5F}, —NR^{5G}C(O)OR^{5F}, —NR^{5G}C(O)NR^{5G}R^{5F}, —SC(O)OR^{5F}, —OC(O)SR^{5F}, —SC(O)SR^{5F}, —SC(O)NR^{5G}R^{5F}, —NR^{5G}C(O)SR^{5F}, —OC(S)OR^{5F}, —OC(S)NR^{5G}R^{5F}, —NR^{5G} C(S)OR^{5F}, —NR^{5G}C(S)NR^{5G}R^{5F}, —SC(S)OR^{5F}, —OC(S)SR^{5F}, —SC(S)SR^{5F}, —SC(S)NR^{5G}R^{5F}, —NR^{5G}C(S)SR^{5F}, —NR^{5G}C(NR^{5G})NR^{5G}R^{5F} and —NR^{5G}S(O)₁₋₂NR^{5G}R^{5F};
      each R^{5F} is independently selected from H, C₁-C₃ alkyl, C₁-C₃ fluoroalkyl, C₁-C₃ hydroxyalkyl and (C₁-C₃ alkoxy)C₁-C₃ alkyl and each R^{5G} is independently selected from H, C₁-C₃ alkyl, C₁-C₃ fluoroalkyl, C₁-C₃ hydroxyalkyl, and (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl);
X¹ is selected from the group consisting of CR^{XA}, S, O, NRX^{B} and N and
X² is selected from the group consisting of CR^{XA}, S, O, NRX^{B} and N in which
   each is independently selected from the group consisting of hydrogen, C₁-C₄ alkyl, C₁-C₄ fluoroalkyl, C₁-C₃ hydroxyalkyl, (C₁-C₃ alkoxy)C₁-C₃ alkyl, halo, —CN, oxo, —SF₅, —N₃, —C(O)R^{XC}, —SR^{XC}, —S(O)₁₋₂R^{XC}, —OR^{XC}, —NR^{XD}R^{XC}, in which each R^{XC} is independently selected from H, C₁-C₃ alkyl, C₁-C₃ fluoroalkyl, C₁-C₃ hydroxyalkyl and (C₁-C₃ alkoxy)C₁-C₃ alkyl and each R^{XD} is independently selected from H, C₁-C₃ alkyl, C₁-C₃ fluoroalkyl, C₁-C₃ hydroxyalkyl, and (C₁-C₃ alkoxy)C₁-C₃ alkyl, —C(O)(C₁-C₃ alkyl) and —C(O)O(C₁-C₃ alkyl);
   each R^{XB} is independently selected from the group consisting of H, C₁-C₃ alkyl, C₁-C₃ fluoroalkyl, C₁-C₃ hydroxyalkyl, (C₁-C₃ alkoxy)C₁-C₃ alkyl, —C(O)(C₁-C₃ alkyl) and —C(O)O(C₁-C₃ alkyl and C₁-C₄ alkyl-S(O)₁₋₂—;
   Z¹ and Z² are independently selected from C and N; and
Y is CR^{Y} or N, in which R^{Y} is selected from the group consisting of hydrogen, C₁-C₃ alkyl, C₁-C₃ fluoroalkyl, C₁-C₃ hydroxyalkyl, C₁-C₃ aminoalkyl, C₁-C₃ thioalkyl, (C₁-C₃ alkoxy)C₁-C₃ alkyl, —S(O)₁₋₂(C₁-C₃ alkyl), halogen, —CN, —SF₅, —N₃, —C(O)R^{YC}, —SR^{YC}, —S(O)₁₋₂R^{YC}, —OR^{YC} and —NR^{YD}R^{YC}, in which each R^{YC} is independently selected from H, C₁-C₃ alkyl and C₁-C₃ fluoroalkyl, and each R^{YD} is independently selected from H, C₁-C₃ alkyl and C₁-C₃ fluoroalkyl;
   wherein
each R⁶ is selected from the group consisting of hydrogen, C₁-C₃ alkyl, C₁-C₃ fluoroalkyl, C₁-C₃ hydroxyalkyl, C₁-C₃ aminoalkyl, C₁-C₃ thioalkyl, (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl);
each alkyl, alkylene, alkenyl, alkenylene, alkynyl and alkynylene is straight-chain or branched;
each optionally substituted alkyl, alkenyl, alkynyl, alkylene, alkenylene and alkynylene is unsubstituted or substituted with 1-5 substituents independently selected from oxo, halogen, —CN, —SF₅, —N₃, —C(O)R⁸, —SR⁸, —S(O)₁₋₂R⁸, —OR⁸, —NR⁹R⁸, —C(O)NR⁹R⁸, —NR⁹C(O)R⁸, —C(S)NR⁹R⁸, —NR⁹C(S)R⁸, —C(O)OR⁸, —OC(O)R⁸, —C(O)SR⁸, —SC(O)R⁸, —C(S)OR⁸, —OC(S)R⁸, —C(S)SR⁸, —SC(S)R⁸, —S(O)₁₋₂OR⁸, —OS(O)₁₋₂R⁸, —S(O)₁₋₂NR⁹R⁸, —NR⁹S(O)₁₋₂R⁸, —OC(O)OR⁸, —OC(O)NR⁹R⁸, —NR⁹C(O)OR⁸, —NR⁹C(O)NR⁹R⁸, —SC(O)OR⁸, —OC(O)SR⁸, SC(O)SR⁸, —SC(O)NR⁹R⁸, —NR⁹C(O)SR⁸, —OC(S)OR⁸, —OC(S)NR⁹R⁸, —NR⁹C(S)OR⁸, —NR⁹C(S)NR⁹R⁸, —SC(S)OR⁸, —OC(S)SR⁸, —SC(S)SR⁸, —SC(S)NR⁹R⁸, —NR⁹C(S)SR⁸, —NR⁹C(NR⁹)NR⁹R⁸ and —NR⁹S(O)₁₋₂NR⁹R⁸, in which
   each R⁸ is independently selected from H, C₁-C₃ alkyl, C₁-C₃ fluoroalkyl, C₁-C₃ hydroxyalkyl and (C₁-C₃ alkoxy)C₁-C₃ alkyl and
   each R⁹ is independently selected from H, C₁-C₃ alkyl, C₁-C₃ fluoroalkyl, C₁-C₃ hydroxyalkyl, and (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl);
each cycloalkyl has 3-10 ring carbons and is unsaturated or partially unsaturated, and optionally includes one or two fused cycloalkyl rings, each fused ring having 3-8 ring members;
each heterocylcloalkyl has 3-10 ring members and 1-3 heteroatoms independently selected from nitrogen, oxygen and sulfur and is unsaturated or partially unsaturated, and optionally includes one or two fused cycloalkyl rings, each having 3-8 ring members;
each aryl is a phenyl or a naphthyl, and optionally includes one or two fused cycloalkyl or heterocycloalkyl rings, each fused cycloalkyl or heterocycloalkyl ring having 4-8 ring members;
each heteroaryl is a 5-6 membered monocyclic heteroaryl ring having 1-4 heteroatoms independently selected from nitrogen, oxygen and sulfur or a 8-10 membered bicyclic heteroaryl having 1-5 heteroatoms independently selected from nitrogen, oxygen or sulfur, and optionally includes one or two fused cycloalkyl or heterocycloalkyl rings, each fused cycloalkyl or heterocycloalkyl ring having 4-8 ring members.
   In certain such embodiments, each and every optionally substituted alkyl, alkylene, alkenyl, alkenylene, alkynyl and alkynylene is unsubstituted or fluorinated. For example, in certain such embodiments, each and every optionally substituted alkyl, alkylene, alkenyl, alkenylene, alkynyl and alkynylene is unsubstituted.

In certain embodiments, structural formula (I) is one of formulae (Ia)-(Ic):

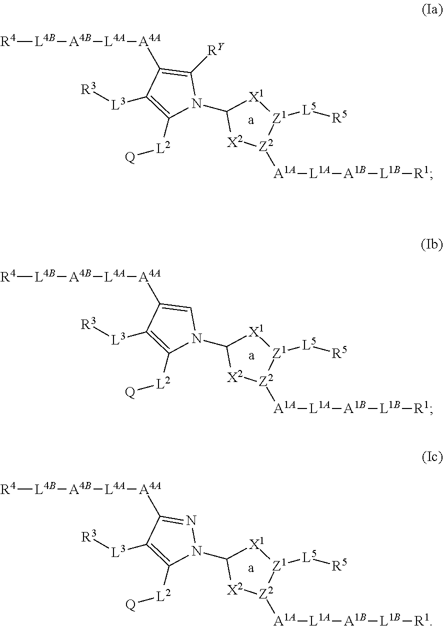

In certain embodiments as otherwise described herein. X¹ is selected from one of the following groups (1a)-(1i)
(1a) X¹ is selected from the group consisting of CR^{XA}, S, O, N and NR^{XB};
(1b) X¹ is selected from the group consisting of S, O, N and NR^{XB};
(1c) X¹ is O;
(1 d) X¹ is S;
(1e) X¹ is N or NR^{XB};
(1f) X¹ is N or NR^{XB}, wherein NR^{XB} is hydrogen or optionally substituted C₁-C₄ alkyl;
(1g) X¹ is N;
(1h) X¹ is CR^{XA};
(1i) X¹ is CR^{XA} wherein R^{XA} is hydrogen or optionally substituted C₁-C₄ alkyl;
   In certain such embodiments, each optionally substituted alkyl, alkenyl and alkynyl of X¹ (including those of R^{XA} and R^{XB}) is unsubstituted or fluorinated. For example, in certain such embodiments each optionally substituted alkyl, alkenyl and alkynyl of X¹ (including those of R^{XA} and R^{XB}) is unsubstituted. In certain embodiments each RX^{A} and RX^{B} is hydrogen.

In certain embodiments as otherwise described herein. X² is selected from one of the following groups (2a)-(2i)
(2a) X² is selected from the group consisting of CR^{XA}, S, O, N and NR^{XB};
(2b) X² is selected from the group consisting of S, O, N and NR^{XB};
(2c) X² is O;
(2d) X² is S;
(2e) X² is selected from N and NR^{XB};
(2f) X² is selected from N and NR^{XB}, wherein NR^{XB} is hydrogen or optionally substituted C₁-C₄ alkyl;
(2g) X² is N;
(2h) X² is CR^{XA};
(2i) X² is CR^{XA} wherein R^{XA} is hydrogen or optionally substituted C₁-C₄ alkyl.
   In certain such embodiments, each optionally substituted alkyl, alkenyl and alkynyl of X² (including those of R^{XA} and R^{XB}) is unsubstituted or fluorinated. For example, in certain such embodiments each optionally substituted alkyl, alkenyl and alkynyl of X² (including those of R^{XA} and R^{XB}) is unsubstituted. In certain embodiments each RX^{A} and RX^{B} is hydrogen.

In certain embodiments as otherwise described herein. Z¹ is selected from one of the following groups (3a)-(3c)
(3a) Z¹ is selected from C and N;
(3b) Z¹ is C;
(3c) Z¹ is N.

In certain embodiments as otherwise described herein. Z² is selected from one of the following groups (4a)-(4c)
(4a) Z² is selected from C and N;
(4b) Z² is C;
(4c) Z² is N.

In certain embodiment as otherwise described herein as the ring system denoted by “a” is one of the following groups (5a)-(5h):
(5a) the ring system denoted by “a” is heteroaromatic (i.e., at least one of X¹, X², Z¹ and Z² is not C or CR^{XA});
(5b) the ring system denoted by “a” is thiazole;
(5c) the ring system denoted by “a” is thiazole and the compound is of formula (Id):

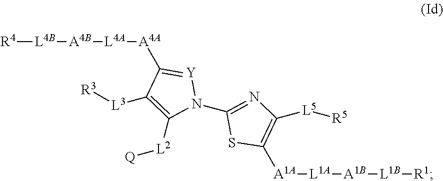

(5d) the ring system denoted by “a” is thiazole, and the compound is of formula (Ie):

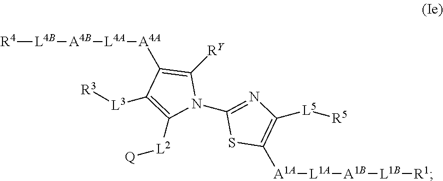

(5e) the ring system denoted by “a” is thiazole, and the compound is of formula (If):

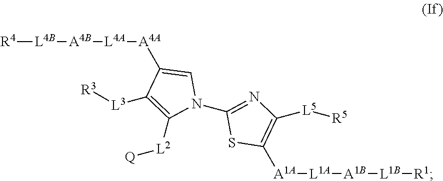

(5f) the ring system denoted by “a” is thiazole, and the compound is of formula (Ig):

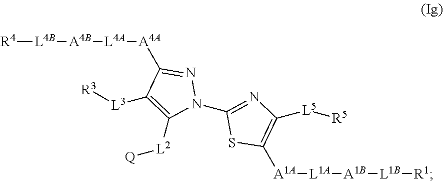

(5g) the ring system denoted by “a” is oxazole, imidazole, pyrazole, or triazole, e.g., having one of the following the structural formula:

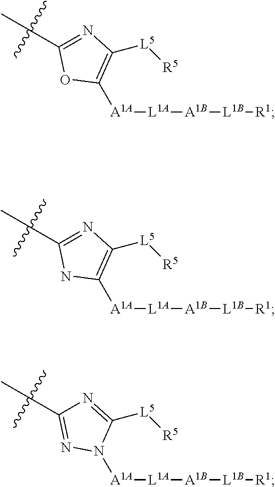

(5h) the compound is of any of formulae (Ia)-(Ic), in which the ring system denoted by “a” is thiazole and the thiazole moiety has the structural formula:

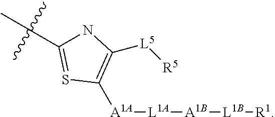

In certain embodiments according to embodiments (5a) and (5b), each and R^{XB} is hydrogen.

In certain embodiments as otherwise described herein, the compound is of one of the following structural formulae:
(id) in which the variables are as defined in any combination of groups (6h) et seq., (7e) et seq., (8d) et seq., (9g) et seq., (10k) et seq., (11e) et seq., (12k) et seq., (13j) et seq., (14l) et seq., (15l) et seq., (16e) et seq., (17d) et seq., (18h) et seq., (19k) et seq., (20g) et seq., and (21h ) et seq. defined hereinbelow;
(Ie) in which the variables are as defined in any combination of groups (6h) et seq., (7e) et seq., (8d) et seq., (9g) et seq., (10k) et seq., (11e) et seq., (12k) et seq., (13j) et seq., (14l) et seq., (15l) et seq., (16e) et seq., (17d) et seq., (18h) et seq., (19k) et seq., (20g) et seq., and (21 h ) et seq. defined hereinbelow;
(If) in which the variables are as defined in any combination of groups (6h) et seq., (7e) et seq., (8d) et seq., (9g) et seq., (10k) et seq., (11e) et seq., (12k) et seq., (13j) et seq., (14l) et seq., (15l) et seq., (16e) et seq., (17d) et seq., (18h) et seq., (19k) et seq., (20g) et seq., and (21h ) et seq. defined hereinbelow;
(Ig) in which the variables are as defined in any combination of groups (6h) et seq., (7e) et seq., (8d) et seq., (9g) et seq., (10k) et seq., (11e) et seq., (12k) et seq., (13j) et seq., (14l) et seq., (15l) et seq., (16e) et seq., (17d) et seq., (18h) et seq., (19k) et seq., (20g) et seq., and (21h ) et seq. defined hereinbelow;
(Ih) in which the variables are as defined in any combination of groups (6h) et seq., (7e) et seq., (8d) et seq., (9g) et seq., (10k) et seq., (11e) et seq., (12k) et seq., (13j) et seq., (14l) et seq., (15l) et seq., (16e) et seq., (17d) et seq., (18h) et seq., (19k) et seq., (20g) et seq., and (21 h ) et seq. defined hereinbelow;
(Ii), in which (Ii) is formula (I) with the ring system denoted by “a” being oxazole, imidazole, pyrazole or triazole (e.g., in one of the following configurations:

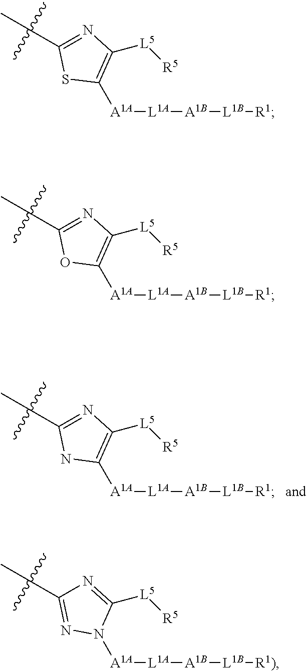

and in which the variables are otherwise as defined in any combination of groups (6h) et seq., (7e) et seq., (8d) et seq., (9g) et seq., (10k) et seq., (11e) et seq., (12k) et seq., (13j) et seq., (14l) et seq., (15l) et seq., (16e) et seq., (17d) et seq., (18h) et seq., (19k) et seq., (20g) et seq., and (21h ) et seq. defined hereinbelow;
(Ij), in which (Ij) is formula (Ic) with the ring system denoted by “a” being oxazole, imidazole, pyrazole. or triazole (e.g., in one of the following configurations:

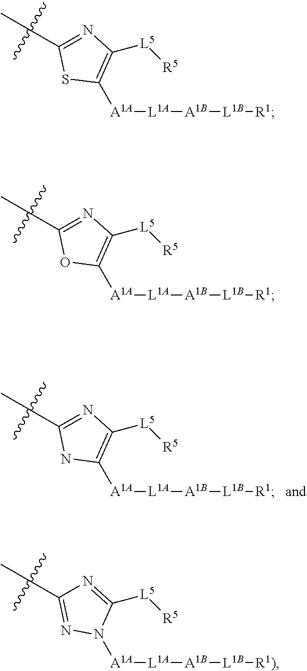

in which the variables are otherwise as defined in any combination of groups (6h) et seq., (7e) et seq., (8d) et seq., (9g) et seq., (10k) et seq., (11e) et seq., (12k) et seq., (13j) et seq., (14l) et seq., (15l) et seq., (16e) et seq., (17d) et seq., (18h) et seq., (19k) et seq., (20g) et seq., and (21h ) et seq. defined hereinbelow.
In certain embodiments, when the compound is of one of formulae (Id), (Ie), (Ih) and (Ii) as described above, R^{Y} is H, —C(O)—C₁-C₃ alkyl, —C(O)—C₁-C₃ fluoroalkyl, —C₁-C₃ alkyl, —C₁-C₃ fluoroalkyl, —CN or halogen. In certain embodiments according to formulae (Id)-(Ij), each RX^{A} and RX^{B} is hydrogen.

The disclosure also provides a variety of subgenera of compounds of any of formulae (I) or (Ia)-(Ih) in which R¹, A^{1A}, L^{1b}, A^{1b}, L^{1a}, L², Q, L³, R³, A^{4A}, L^{4B}, A^{4B}, L^{4A}, R⁴, L⁵, and R⁵ are optionally independently selected from the groups (6h) et seq., (7e) et seq., (8d) et seq., (9g) et seq., (10k) et seq., (11e) et seq., (12k) et seq., (13j) et seq., (14l) et seq., (15l) et seq., (16e) et seq., (17d) et seq., (18h) et seq., (19k) et seq., (20g) et seq., and (21h) et seq. defined hereinbelow. Definitions of the variables can be made from any combination of groups (6h) et seq., (7e) et seq., (8d) et seq., (9g) et seq., (10k) et seq., (11e) et seq., (12k) et seq., (13j) et seq., (14l) et seq., (15l) et seq., (16e) et seq., (17d) et seq., (18h) et seq., (19k) et seq., (20g) et seq., and (21h) et seq. defined hereinbelow that is not logically or chemically inconsistent.

In certain embodiments as otherwise described herein. R¹ is selected from one of the following groups (6h)-(6p)
(6h) R¹ is selected from the group consisting of hydrogen, optionally substituted C₁-C₈ alkyl, optionally substituted C₁-C₈ alkenyl, optionally substituted C₁-C₈ alkynyl, cycloalkyl and heterocycloalkyl, wherein cycloalkyl and heterocycloalkyl are optionally substituted with 1-5 R^{1E};
(6i) R¹ is selected from the group consisting of hydrogen, optionally substituted C₁-C₈ alkyl, optionally substituted C₁-C₈ alkenyl and optionally substituted C₁-C₈ alkynyl;
(6j) R¹ is selected from optionally substituted C₁-C₈ alkyl, optionally-substituted C₁-C₈ alkenyl, and optionally substituted C₁-C₈ alkynyl;
(6k) R¹ is selected from the group consisting of hydrogen, unsubstituted C₁-C₈ alkyl, unsubstituted C₁-C₈ alkenyl and unsubstituted C₁-C₈ alkynyl (for example, methyl, ethyl, propyl or butyl);
(6l) R¹ is selected from the group consisting of unsubstituted C₁-C₈ alkyl, unsubstituted C₁-C₈ alkenyl and unsubstituted C₁-C₈ alkynyl (for example, methyl, ethyl, propyl, butenyl or butyl);
(6m) R¹ is cycloalkyl or heterocycloalkyl (e.g., cyclobutyl, cyclopentyl, cyclohexyl or cycloheptyl), each optionally substituted with 1-5 R^{1E}, for example, 1-5 alkyl groups;
(6n) R¹ is cycloalkyl optionally substituted with 1-5 R^{1E};
(6o) R¹ is hydrogen, optionally substituted C₁-C₈ alkyl, or cycloalkyl optionally substituted with 1-5 R^{1E};
(6p) R¹ is hydrogen or optionally substituted C₁-C₆ alkyl (e.g., ethyl, propyl, or butyl).
In certain such embodiments, each optionally substituted alkyl, alkenyl and alkynyl of R¹ (including those of R^{1E}) is unsubstituted or fluorinated. For example, in certain such embodiments each optionally substituted alkyl, alkenyl and alkynyl of R¹ (including those of R^{1E}) is unsubstituted.

In certain embodiments as otherwise described herein. A^{1A} is selected from one of the following groups (7e)-(7h)
(7e) A^{1A} is selected from the group consisting of a bond, —C(O)—, —S—, —S(O)₁₋₂—, —O—, —NR⁶—, —C(O)NR⁶—, —NR⁶C(O)—, and —C(O)O—;
(7f) A^{1A} is a bond;
(7g) A^{1A} is selected from the group consisting of —C(O)—, —S—, —S(O)₁₋₂—, —O—, and —NR⁶—;
(7h) A^{1A} is O.

In certain embodiments as otherwise described herein. L^{1B} is selected from one of the following groups (8d)-(8f)
(8d) L^{1B} is selected from a bond and optionally substituted C₁-C₄ alkylene;
(8e) L^{1B} is a bond;
(8f) L^{1B} is unsubstituted C₁-C₄ alkylene.
   In certain such embodiments, each optionally substituted alkylene of L^{1B} is unsubstituted or fluorinated. For example, in certain such embodiments each optionally substituted alkylene of L^{1B} is unsubstituted.

In certain embodiments as otherwise described herein. A^{1B} is selected from one of the following groups (9g)-(9l)
(9g) A^{1B} is selected from the group consisting of —C(O)—, —S—, —S(O)₁₋₂—, —O—, —NR⁶—, —C(O)NR⁶—, —NR⁶C(O)—, —OC(O)— and —C(O)O—;
(9h) A^{1B} is selected from the group consisting of —C(O)—, —S—, —S(O)₁₋₂—, —O—, and —C(O)O—;
(9i) A^{1B} is —S—;
(9j) A^{1B} is selected from —C(O)—, —S(O)—, —S(O)₂—, —OC(O)— and —C(O)O—;
(9k) A^{1B} is —O—;
(9l) A^{1B} is a bond.

In certain embodiments as otherwise described herein. L^{1A} is selected from one of the following groups (10k)-(10m)
(10k) L^{1A} is selected from a bond and optionally substituted C₁-C₄ alkylene;
(10l) L^{1A} is a bond;
(10m) L^{1A} is optionally substituted C₁-C₄ alkylene.
   In certain such embodiments, each optionally substituted alkylene of L^{1A} is unsubstituted or fluorinated. For example, in certain such embodiments each optionally substituted alkylene of L^{1A} is unsubstituted.

In certain embodiments as otherwise described herein. A^{1A}-L^{1A}-A^{1B}-L^{1B} (i.e., -L¹-) is selected from one of the following groups (10n)-(10v)
(10n) A^{1A}-L^{1A}-A^{1B}-L^{1b}, wherein A^{1A} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—; L^{1A} is selected from the group consisting of a bond, unsubstituted C₁-C₄ alkylene, unsubstituted C₁-C₄ alkenylene and unsubstituted C₁-C₄ alkynylene; A^{1B} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—; and L^{1B} is a bond;
(10o) A^{1A}-L^{1A}-A^{1B}-L^{1b}, wherein A^{1A}, L^{1A} and L^{1B} are a bond, and A^{1B} as defined in formula (I) or in (10n);
(10p) A^{1A}-L^{1A}-A^{1B}-L^{1B} is selected from a bond, optionally substituted C₁-C₄ alkylene, —C(O)—, —S—, —S(O)₁₋₂—, —O—, and —NR⁶—;
(10q) A^{1A}-L^{1A}-A^{1B}-L^{1B} is selected from a bond, —CH₂—, —CH(CH₃)—, —CH₂CH₂—, —C(O)—, —S—, —S(O)₁₋₂—, —O—, and —NR⁶—;
(10r) A^{1A}-L^{1A}-A^{1B}-L^{1B} is —O— or —S—.
(10s) A^{1A}-L^{1A}-A^{1B}-L^{1B} is unsubstituted C₁-C₄ alkylene;
(10t) A^{1A}-L^{1A}-A^{1B}-L^{1B} is selected from —C(O)—, —S(O)— and —S(O)₂—;
(10u) A^{1A}-L^{1A}-A^{1B}-L^{1B} is selected from —CH₂—, —CH(CH₃)— and —CH₂CH₂—;
(10v) A^{1A}-L^{1A}-A^{1B}-L^{1B} is a bond.
   In certain such embodiments, each optionally substituted alkyl, alkylene, alkenyl, alkenylene, alkynyl and alkynylene of A^{1A}-L^{1A}-A^{1B}-L^{1B} (including those of R⁶) is unsubstituted or fluorinated. For example, in certain such embodiments each optionally substituted alkyl, alkylene, alkenyl, alkenylene, alkynyl and alkynylene of A^{1A}-L^{1A}-A^{1B}-L^{1B} (including those of R⁶) is unsubstituted.

In certain embodiments as otherwise described herein. L² is selected from one of the following groups (11e)-(11h)
(11e) L² is selected from a bond and optionally substituted C₁-C₄ alkylene;
(11f) L² is selected from a unsubstituted C₁-C₄ alkylene;
(11g) L² is a bond, —CH₂—, —CH(CH₃)— or —CH₂CH₂—;
(11h) L² is a bond.
   In certain such embodiments, each optionally substituted alkylene of L² is unsubstituted or fluorinated. For example, in certain such embodiments each optionally substituted alkylene of L² is unsubstituted.

In certain embodiments as otherwise described herein. Q is selected from one of the following groups (12k)-(12t)
(12k) Q is selected from the group consisting of —CH₂OH, —C(O)OH, —C(O)OR^{2A}, —C(O)NR^{2B}R^{2A}, —C(O)NR^{2B}S(O)₂R^{2A}, —C(O)NR^{2B}S(O)₂NR^{2B}R^{2A}, —C(O)R^{2A}, —S(O)₂OH, —P(O)(OH)₂, —C(OH)(CF₃)₂, —S(O)₂R^{2A}, —N(R^{2B})S(O)₂R^{2A}, —S(O)₂NR^{2B}R^{2A}, —C(O)NH—O(C₁-C₃ alkyl), —C(O)NHOH, —CO(NH)CN,

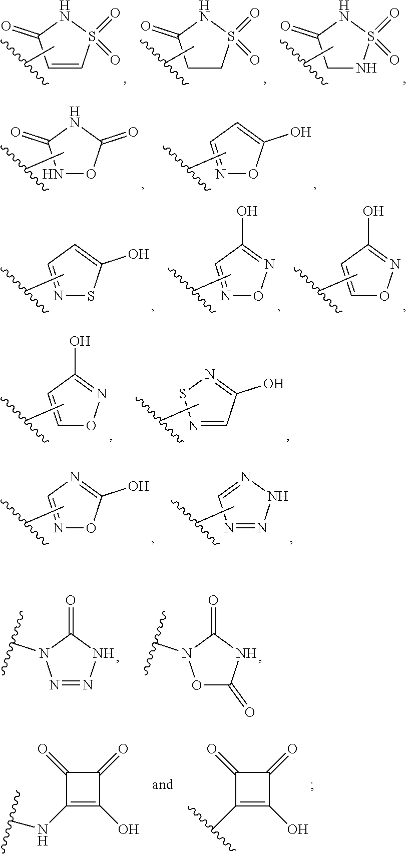

(12l) Q is selected from the group consisting of —C(O)OH, —C(O)OR^{2A}, —C(O)NR^{2B}R^{2A}, —C(O)NR^{2B}S(O)₂R^{2A}, —C(O)NR^{2B}S(O)₂NR^{2B}R^{2A}, —C(O)R^{2A}, —S(O)₂OH, —P(O)(OH)₂, —C(OH)(CF₃)₂, —S(O)₂R^{2A}, —S(O)₂NR^{2B}R^{2A}, —C(O)NHOH, —CO(NH)CN,

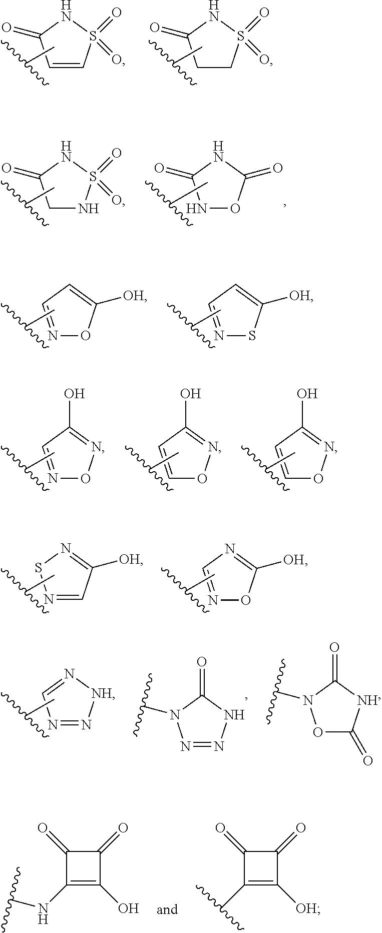

(12m) Q is selected from the group consisting of —CH₂OH, —C(O)OH, —C(O)OR^{2A}, —C(O)NR^{2B}R^{2A}, —C(O)R^{2A}, —S(O)₂OH, —P(O)(OH)₂, —S(O)₂R^{2A}, —S(O)₂NR^{2B}R^{2A}, and —C(O)NHOH;
(12n) Q is selected from —C(O)OH, —C(O)OR^{2A}, and —C(O)NR^{2B}R^{2A};
(12o) Q is selected from —C(O)OH and —C(O)O(C₁-C₃ alkyl);
(12p) Q is —C(O)OH;
(12q) Q is —C(O)O(C₁-C₃ alkyl);
(12r) Q is —C(O)NR^{2B}R^{2A}, in which R^{2A} is C₁-C₃ alkyl, C₁-C₃ hydroxyalkyl, C₁-C₃ aminoalkyl or C₁-C₃ thioalkyl and R^{2B} is H or C₁-C₃ alkyl;
(12s) Q is —C(O)NR^{2B}R^{2A}, in which R^{2A} and R^{2B} come together with a nitrogen to which they are both directly bound to form a heterocycloalkyl optionally substituted with 1-3 substituents selected from C₁-C₃ alkyl, C₁-C₃ fluoroalkyl, hydroxyl, amino, thio, C₁-C₃ hydroxyalkyl, C₁-C₃ aminoalkyl, C₁-C₃ thioalkyl and —C(O)C₁-C₃ alkyl;
(12t) Q is —C(O)NR^{2B}R^{2A}, in which R^{2A} is —S(O)₁₋₂(C₁-C₃ alkyl), —S(O)₁₋₂(C₁-C₃ fluoroalkyl), or heteroaryl optionally substituted with 1-2 groups selected from substituents selected from C₁-C₃ alkyl, C₁-C₃ fluoroalkyl, hydroxyl, amino, thio, C₁-C₃ hydroxyalkyl, C₁-C₃ aminoalkyl, C₁-C₃ thioalkyl and —C(O)C₁-C₃ alkyl and R^{2B} is H or C₁-C₃ alkyl.

In certain embodiments as otherwise described herein. L³ is selected from one of the following groups (13i)-(13r)
(13j) L³ is selected from a bond (i.e., L³ is -L^{3A}-A^{3A}- wherein both A^{3A} and L^{3A} are a bond, or L³ is -A^{3B}-L^{3B}- wherein both A^{3B} and L^{3B} are a bond) and optionally substituted C₁-C₄ alkylene (e.g., L³ is -L^{3A}-A^{3A}- wherein A^{3A} is a bond and L^{3A} is and optionally substituted C₁-C₄ alkylene);
(13k) L³ is a bond;
(13l) L³ is optionally substituted C₁-C₄ alkylene (e.g., A^{3A} is a bond and L^{3A} is and optionally substituted C₁-C₄ alkylene); (13m) L³ is -L^{3A}-A^{3A}-, wherein A^{3A} is a bond and L^{3A} is optionally substituted C₁-C₄ alkylene, optionally substituted C₁-C₄ alkenylene or optionally substituted C₁-C₄ alkynylene;
(13n) L³ is unsubstituted C₁-C₄ alkylene;
(13o) L³ is C₁-C₃ alkylene, optionally substituted with a hydroxyl;
(13p) L³ is —C(O)—, —S—, —S(O)₁₋₂—, —O—, —NR⁶—;
(13q) L³ is —CH₂—, —CH(CH₃)—, —CH₂CH₂—, —CH(CH₃)(OH)— or —CH(OH)—;
(13r) L³ is selected from —CH₂—, —CH(CH₃)—, and —CH₂CH₂—.
   In certain such embodiments, each optionally substituted alkyl, alkylene, alkenyl, alkenylene, alkynyl and alkynylene of L³ (including those of R⁶) is unsubstituted orfluorinated. For example, in certain such embodiments each optionally substituted alkyl, alkylene, alkenyl, alkenylene, alkynyl and alkynylene of L³ (including those of R⁶) is unsubstituted.

In certain embodiments as otherwise described herein. R³ is selected from one of the following groups (14l)-(14v)
(14l) R³ is aryl or heteroaryl each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E};
(14m) R³ is aryl (e.g., a phenyl, a benzodioxole, or a dihydro-1H-isoquinoline) optionally substituted with 1-5 R^{3E};
(14n) R³ is aryl (e.g., a phenyl, a benzodioxole, or a dihydro-1H-isoquinoline) (i) substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E};
(14o) R³ is aryl (e.g., a phenyl, a benzodioxole, ora dihydro-1H-isoquinoline) (i) substituted with a single substituent selected from -L^{3C}-(phenyl optionally substituted with 1-5 R^{3D}), -L^{3C}-(monocyclic heteroaryl optionally substituted with 1-5 R^{3D}), -L^{3C}-(monocyclic cycloalkyl optionally substituted with 1-5 R^{3E}), -L^{3C}-(monocyclic heterocycloalkyl optionally substituted with 1-5 R^{3E}) and (ii) optionally substituted with 1-5 R^{3E};
(14p) R³ is as defined in (14k)-(14n), wherein the aryl is not substituted with any R^{3E};
(14q) R³ is heteroaryl (e.g., an isothiazole, a pyridone, a thiadiazole, a pyrazine, a pyrazolopyrimidine, a pyrazolopyridine, an imidazole, a benzofuran, an indole, an imidazopyridine, a pyridine, a pyrazole, an isoxazole, a triazolopyridine, a benzimidazole, a thiophene, a benzothiophene, a furan or a pyrimidine) optionally substituted with 1-5 R^{3E};
(14r) R³ is heteroaryl (e.g., an isothiazole, a pyridone, a thiadiazole, a pyrazine, a pyrazolopyrimidine, a pyrazolopyridine, an imidazole, a benzofuran, an indole, an imidazopyridine, a pyridine, a pyrazole, an isoxazole, a triazolopyridine, a benzimidazole, a thiophene, a benzothiophene, a furan or a pyrimidine) (i) substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E};
(14s) R³ is heteroaryl (e.g., an isothiazole, a pyridone, a thiadiazole, a pyrazine, a pyrazolopyrimidine, a pyrazolopyridine, an imidazole, a benzofuran, an indole, an imidazopyridine, a pyridine, a pyrazole, an isoxazole, a triazolopyridine, a benzimidazole, a thiophene, a benzothiophene, a furan or a pyrimidine) (i) substituted with a single substituent selected from -L^{3C}-(phenyl optionally substituted with 1-5 R^{3D}), -L^{3C}-(monocyclic heteroaryl optionally substituted with 1-5 R^{3D}), -L^{3C}-(monocyclic cycloalkyl optionally substituted with 1-5 R^{3E}), -L^{3C}-(monocyclic heterocycloalkyl optionally substituted with 1-5 R^{3E}) and (ii) optionally substituted with 1-5 R^{3E};
(14t) R³ is as defined in (14p)-(14r), wherein the heteroaryl is not substituted with any R^{3E};
(14u) R³ is selected from the group consisting of: phenyl, benzodioxolyl, dihydro-1H-isoquinolinyl, imidazolyl, oxazolyl, isoxazolyl, isothiazolyl, thiazolyl, pyridinyl, and pyrazinyl, pyridonyl, thiadiazolyl, pyrazolopyrimidinyl, pyrazolopyridinyl, benzofuranyl, indolyl, imidazopyridinyl, pyrazolyl, triazolopyridinyl, benzimidazolyl, a benzimidazolyl, a thienyl, a benzothienyl, a furanyl and pyrimidinyl, each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E};
(14v) R³ is selected from the group consisting of phenyl and monocyclic heteroaryl (e.g., pyridyl, pyrazolyl), optionally substituted with 1-5 R^{3E}.

In certain such embodiments, each optionally substituted alkyl, alkenyl and alkynyl of R³ (including those of R^{3D} and R^{3E}) is unsubstituted or fluorinated. For example, in certain such embodiments each optionally substituted alkyl, alkenyl and alkynyl of R³ (including those of R^{3D} and R^{3E}) is unsubstituted.

In certain embodiments as otherwise described herein. R⁴ is selected from one of the following groups (15l)-(15y)
(15l) R⁴ is selected from hydrogen, optionally substituted C₁-C₈ alkyl, optionally-substituted C₁-C₈ alkenyl and optionally substituted C₁-C₈ alkynyl;
(15m) R⁴ is selected from hydrogen, unsubstituted C₁-C₈ alkyl, unsubstituted C₁-C₈ alkenyl and unsubstituted C₁-C₈ alkynyl;
(15n) R⁴ is selected from hydrogen, optionally substituted C₁-C₆ alkyl, optionally-substituted C₁-C₆ alkenyl and optionally substituted C₁-C₆ alkynyl;
(15o) R⁴ is optionally substituted C₁-C₈ alkyl, optionally-substituted C₁-C₈ alkenyl or optionally substituted C₁-C₈ alkynyl;
(15p) R⁴ is selected from hydrogen, unsubstituted C₁-C₆ alkyl, optionally-substituted C₁-C₈ alkenyl, and optionally-substituted C₁-C₈ alkynyl.
(15q) R⁴ is selected from hydrogen, unsubstituted C₁-C₆ alkyl, unsubstituted C₁-C₆ alkenyl and unsubstituted C₁-C₆ alkynyl (for example, methyl, ethyl, propyl, butyl or pentyl);
(15r) R⁴ is hydrogen or optionally substituted C₁-C₆ alkyl;
(15s) R⁴ is hydrogen or unsubstituted C₁-C₆ alkyl;
(15t) R⁴ is hydrogen or optionally substituted C₁-C₃ alkyl;
(15u) R⁴ is hydrogen or unsubstituted C₁-C₃ alkyl;
(15v) R⁴ is optionally substituted C₁-C₃ alkyl;
(15w) R⁴ is unsubstituted C₁-C₃ alkyl;
(15x) R⁴ is methyl;
(15y) R⁴ is hydrogen.

In certain embodiments as otherwise described herein. A^{4A} is selected from one of the following groups (16e)-(16h)
(16e) A^{4A} is selected from the group consisting of a bond, —C(O)—, —S—, —S(O)₁₋₂—, —O—, —NR⁶—, —C(O)NR⁶—, —NR⁶C(O)—, and —C(O)O—;
(16f) A^{4A} is a bond;
(16g) A^{4A} is selected from the group consisting of —C(O)—, —S—, —S(O)₁₋₂—, —O— and —NR⁶—;
(16h) A^{4A} is —O—.

In certain embodiments as otherwise described herein. L^{4B} is selected from one of the following groups (17d)-(17f)
(17d) L^{4B} is selected from a bond and optionally substituted C₁-C₄ alkylene;
(17e) L^{4B} is a bond;
(17f) L^{4B} is optionally substituted C₁-C₄ alkylene.
   In certain such embodiments, each optionally substituted alkylene of L^{4B} is unsubstituted or fluorinated. For example, in certain such embodiments each optionally substituted alkylene of L^{4B} is unsubstituted.

In certain embodiments as otherwise described herein. A^{4B} is selected from one of the following groups (18h)-(18n)
(18h) A^{4A} is selected from the group consisting of a bond, —C(O)—, —S—, —S(O)₁₋₂—, —O—, —NR⁶—, —C(O)NR⁶—, —NR⁶C(O)—, —OC(O)— and —C(O)O—;
(18i) A^{4A} is selected from the group consisting of —C(O)—, —S—, —S(O)₁₋₂—, —O—, —NR⁶—, —C(O)NR⁶—, —NR⁶C(O)—, —OC(O)— and —C(O)O—;
(18j) A^{4A} is selected from the group consisting of —C(O)—, —S—, —S(O)₁₋₂—, —O—, and —C(O)O—;
(18k) A^{4A} is selected from —NR⁶—, —C(O)NR⁶— and —NR⁶C(O)—;
(181) A^{4A} is selected from —C(O)—, —OC(O)—, and —C(O)O—;
(18m) A^{4A} is a bond;
(18n) A^{4A} is —O—.

In certain embodiments as otherwise described herein. L^{4A} is selected from one of the following groups (19k)-(19m)
(19k) L^{4A} is selected from a bond and optionally substituted C₁-C₄ alkylene;
(191) L^{4A} is a bond;
(19m) L^{4A} is optionally substituted C₁-C₄ alkylene.
   In certain such embodiments, each optionally substituted alkylene of L^{4A} is unsubstituted or fluorinated. For example, in certain such embodiments each optionally substituted alkylene of L^{4A} is unsubstituted.

In certain embodiments as otherwise described herein. L^{4B}-A^{4B}-L^{4A}-A^{4A} is selected from one of the following groups (19n)-(19v)
(19n) L^{4B}-A^{4B}-L^{4A}-A^{4A}, wherein A^{4A} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—; L^{4A} is selected from the group consisting of a bond, unsubstituted C₁-C₄ alkylene, unsubstituted C₁-C₄ alkenylene and unsubstituted C₁-C₄ alkynylene; A^{4B} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—; and L^{4B} is a bond;
(19o) L^{4B}-A^{4B}-L^{4A}-A^{4A}, wherein, wherein A^{4A}, L^{4A} and L^{4B} are a bond, and wherein A^{4B} are as defined in formula (I) or in (19n);
(19p) L^{4B}-A^{4B}-L^{4A}-A^{4A} is selected from a bond, optionally substituted C₁-C₄ alkylene, —C(O)—, —S—, —S(O)₁₋₂—, —O—, and —NR⁶—;
(19q) L^{4B}-A^{4B}-L^{4A}-A^{4A} is selected from a bond, —CH₂—, —CH(CH₃)—, —CH₂CH₂—, —C(O)—, —S—, —S(O)₁₋₂—, —O—, and —NR⁶— (e.g., a bond);
(19r) L^{4B}-A^{4B}-L^{4A}-A^{4A} is —O— or —S—;
(19s) L^{4B}-A^{4B}-L^{4A}-A^{4A} is unsubstituted C₁-C₄ alkylene;
(19t) L^{4B}-A^{4B}-L^{4A}-A^{4A} is selected from —C(O)—, —S(O)— and —S(O)₂—;
(19u) L^{4B}-A^{4B}-L^{4A}-A^{4A} is selected from —CH₂—, —CH(CH₃)—, and —CH₂CH₂—;
(19v) L^{4B}-A^{4B}-L^{4A}-A^{4A} is a bond.
   In certain such embodiments, each optionally substituted alkyl, alkylene, alkenyl, alkenylene, alkynyl and alkynylene of L^{4B}-A^{4B}-L^{4A}-A^{4A} (including those of R⁶) is unsubstituted or fluorinated. For example, in certain such embodiments each optionally substituted alkyl, alkylene, alkenyl, alkenylene, alkynyl and alkynylene of L^{4B}-A^{4B}-L^{4A}-A^{4A} (including those of R⁶) is unsubstituted.

In certain embodiments as otherwise described herein. L⁵ is selected from one of the following groups (20g)-(20l)
(20g) L⁵ is selected from a bond (i.e., L⁵ is -L^{5A}-A^{5A}- wherein both A^{5A} and L^{5A} are a bond, or L⁵ is -A^{5B}-L^{5B}- wherein both A^{5B} and L^{5B} are a bond) and optionally substituted C₁-C₄ alkylene (e.g., L⁵ is -L^{5A}-A^{5A}- wherein A^{5A} is a bond and L^{5A} is and optionally substituted C₁-C₄ alkylene);
(20h) L⁵ is a bond (e.g., both A^{5A} and L^{5A} are a bond);
(20i) L⁵ is selected from the group consisting of —C(O)—, —S—, —S(O)₁₋₂—, —O— and —NR⁶—;
(20j) L⁵ is selected from the group consisting of a bond, —CH₂—, —CH(CH₃)—, —CH₂CH₂—, —CH═CH—, —C_{S}C—, —C(O)—, —S—, —S(O)₁₋₂—, —O—, and —C(O)O— (e.g., a bond);
(20k) L⁵ is selected from —S— and —O—;
(20l) L⁵ is selected from —C(O)—, —S(O)₁₋₂—, and —C(O)O—.
   In certain such embodiments, each optionally substituted alkyl, alkylene, alkenyl, alkenylene, alkynyl and alkynylene of L⁵ (including those of R⁶) is unsubstituted or fluorinated. For example, in certain such embodiments each optionally substituted alkyl, alkylene, alkenyl, alkenylene, alkynyl and alkynylene of L⁵ (including those of R⁶) is unsubstituted.

In certain embodiments as otherwise described herein. R⁵ is selected from one of the following groups (21 h)-(21 n)
(21 h) R⁵ is aryl (e.g., phenyl) (i) optionally substituted with a single substituent selected from -L^{5C}-(aryl optionally substituted with 1-5 R^{5D}), -L^{5C}-(heteroaryl optionally substituted with 1-5 R^{5D}), -L^{5C}-(cycloalkyl optionally substituted with 1-5 R^{5e}), -L^{5C}-(heterocycloalkyl optionally substituted with 1-5 R^{5E}) and (ii) optionally substituted with 1-5 R^{5E};
(21i) R⁵ is aryl (e.g., phenyl) optionally substituted with 1-5 R^{5E};
(21j) R⁵ is phenyl, optionally substituted with 1-5 R^{5E}, wherein each R^{5E} is independently selected from optionally-substituted C₁-C₄ alkyl, C₁-C₄ fluoroalkyl, halogen, —OR^{5F}, and —NR^{5G}R^{5F};
(21k) R⁵ is heteroaryl (e.g., an isoxazolyl, a pyridyl, an imidazopyridyl, a pyrazolyl, a benzoxazole, an indolyl, a pyrimidinyl) (i) optionally substituted with a single substituent selected from -L^{5C}-(aryl optionally substituted with 1-5 R^{5D}), -L^{5C}-(heteroaryl optionally substituted with 1-5 R^{5D}), -L^{5C}-(cycloalkyl optionally substituted with 1-5 R^{5E}), -L^{5C}-(heterocycloalkyl optionally substituted with 1-5 R^{5E}) and (ii) optionally substituted with 1-5 R^{5E};
(21l) R⁵ is heteroaryl (e.g., an isoxazolyl, a pyridyl, an imidazopyridyl, a pyrazolyl) optionally substituted with 1-5 R^{5E};
(21m) R⁵ is selected from the group consisting of phenyl, isoxazolyl, pyridyl, imidazopyridyl, and pyrazolyl, each (i) optionally substituted with a single substituent selected from -L^{5C}-(aryl optionally substituted with 1-5 R^{5D}), -L^{5C}-(heteroaryl optionally substituted with 1-5 R^{5D}), -L^{5C}-(cycloalkyl optionally substituted with 1-5 R^{5E}), -L^{5C}-(heterocycloalkyl optionally substituted with 1-5 R^{5E}) and (ii) optionally substituted with 1-5 R^{5E};
(21 n) R⁵ is selected from the group consisting of phenyl, isoxazolyl, pyridyl, imidazopyridyl, and pyrazolyl, each optionally substituted with 1-5 R^{5E}.
   In certain such embodiments, each optionally substituted alkyl, alkenyl and alkynyl of R⁵ (including those of R^{5D} and R^{5E}) is unsubstituted or fluorinated. For example, in certain such embodiments each optionally substituted alkyl, alkenyl and alkynyl of R⁵ (including those of R^{5D} and R^{5E}) is unsubstituted.

In certain embodiments, the therapeutic compound is of any of formula (Ik), (Im), (In) or (Io) below:

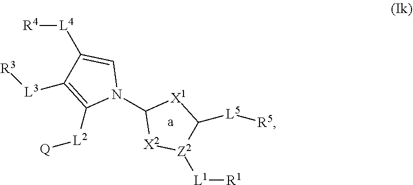

in which formula (Ik) the ring system denoted by “a” is heteroaromatic,

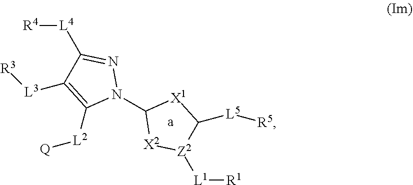

in which formula (Im) the ring system denoted by “a” is heteroaromatic,

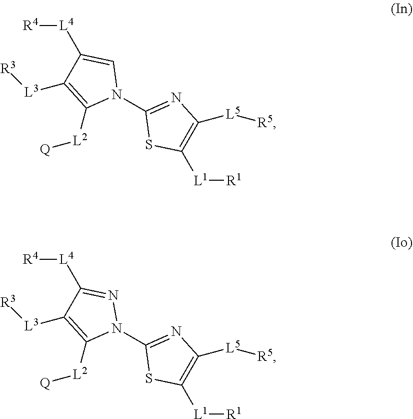

optionally in the form of a pharmaceutically acceptable salt or N-oxide, or a solvate or hydrate, wherein
L¹ is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
R¹ is selected from the group consisting of
   hydrogen,
   C₁-C₈ alkyl, C₁-C₈ alkenyl and C₁-C₈ alkynyl, each unsubstituted orfluorinated, cycloalkyl and heterocycloalkyl, each optionally substituted with 1-2 R^{1E}, and aryl and heteroaryl, each optionally substituted with 1-5 R^{1E}, in which
      each R^{1E} is independently selected from oxo, optionally-substituted C₁-C₄ alkyl, C₁-C₄ fluoroalkyl, halogen, —CN, SF₅, —N₃, —C(O)R^{1F}, —SR^{1F}, —S(O)₁₋₂R^{1F}, —OR^{1F}, —NR^{1G}R^{1F} and —C(O)R^{1F};
      each R^{1F} is independently selected from H, C₁-C₃ alkyl and C₁-C₃ fluoroalkyl and
      each R^{1G} is independently selected from H and C₁-C₃ alkyl, or
or A^{1A}, L^{1a}, A^{1b}, A^{1b}, and R¹ are absent;
L² is selected from the group consisting of a bond, —CH₂—, —CH(CH₃)— or —CH₂CH₂—; Q is selected from the group consisting of H, —CH₂OH, —C(O)OH, —C(O)OR^{2A}, —C(O)NR^{2B}R^{2A}, —C(O)NR^{2B}S(O)₂R^{2A}, —C(O)NR^{2B}S(O)₂NR^{2B}R^{2A}, —C(O)R^{2A}, —S(O)₂OH, —P(O)(OH)₂, —C(OH)(CF₃)₂, S(O)₂R^{2A}, —N(R^{2B})S(O)₂R^{2A}, —S(O)₂NR^{2B}R^{2A}, —C(O)NHOH, —C(O)NH—O(C₁-C₃ alkyl), and —CO(NH)CN, in which
   each R^{2A} is independently selected from H and C₁-C₃ alkyl, and
   each R^{2B} is independently selected from H and C₁-C₃ alkyl;
L³ is a bond, —C(O)—, —S—, —S(O)₁₋₂—, —O—, —NR⁶—, —CH₂—, —CH(CH₃)(OH)— or —CH(OH)—;
R³ is aryl or heteroaryl each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E},
   in which
      each L^{3C} is a bond, methylene, ethylene, —C(O)—, —S—, —S(O)₁₋₂—, —O— or —NR^{3G}—;
      each R^{3D} is independently selected from optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F} and —NR^{3G}S(O)₁₋₂R^{3F};
      each R^{3E} is independently selected from oxo, optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F};
      each R^{3F} is independently selected from H, C₁-C₃ alkyl and C₁-C₃ fluoroalkyl and
      each R^{3G} is independently selected from H and C₁-C₃ alkyl, C₁-C₃ fluoroalkyl;
L⁴ is is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
R⁴ is selected from the group consisting of hydrogen, optionally substituted C₁-C₈ alkyl, optionally-substituted C₁-C₈ alkenyl and optionally substituted C₁-C₈ alkynyl, L⁵ is a bond, —C(O)—, —S—, —S(O)₁₋₂—, —O—, —NR⁶—, —CH₂CH₂—, —CH═CH—, —C═C—, —CH₂—, —CH(CH₃)(OH)— or —CH(OH)—;
R⁵ is aryl, heteroaryl, cycloalkyl or heterocycloalkyl, each optionally substituted with 1-5 R^{5E},
   in which
      each R^{5E} is independently selected from oxo, optionally-substituted C₁-C₄ alkyl, C₁-C₄ fluoroalkyl, halogen, —CN, —SF₅, —N₃, —C(O)R^{5F}, —SR^{5F}, —S(O)₁₋₂R^{5F}, —OR^{5F}, —NR^{5G}R^{5F}, —C(O)R^{5F}, —C(O)NR^{5G}R^{5F}, —NR^{5G}C(O)R^{5F}, —C(S)NR^{5G}R^{5F}, —NR^{1G}C(S)R^{5F}, —C(O)OR^{5F}, —OC(O)R^{5F}, —C(O)SR^{5F}, —SC(O)R^{5F}, —C(S)OR^{5F}, —OC(S)R^{5F}, —C(S)SR^{5F}, —SC(S)R^{5F}, —S(O)₁₋₂OR^{5F}, —OS(O)₁₋₂R^{5F}, —S(O)₁₋₂NR^{5G}R^{5F} and —NR^{5G}S(O)₁₋₂R^{5F};
      each R^{5F} is independently selected from H, C₁-C₃ alkyl and C₁-C₃ fluoroalkyl and
      each R^{5G} is independently selected from H and C₁-C₃ alkyl;
Y is CR^{Y} or N, in which R^{Y} is selected from the group consisting of hydrogen, C₁-C₃ alkyl and C₁-C₃ fluoroalkyl;
X¹ is selected from the group consisting of CR^{XA}, S, O, NRX^{B} and N and
X² is selected from the group consisting of CR^{XA}, S, O, NRX^{B} and N in which
   each R^{XA} is independently selected from the group consisting of hydrogen, C₁-C₄ alkyl and C₁-C₄ fluoroalkyl; and
      each R^{XB} is independently selected from the group consisting of hydrogen, C₁-C₄ alkyl and C₁-C₄ fluoroalkyl, C₁-C₄ alkyl-C(O)—, C₁-C₄ alkyl-S(O)₁₋₂—; Z¹ and Z² are independently selected from C and N;
         wherein
when Z¹ is N and is bound in the ring system denoted by “a” by a double bond, A^{1A}, L^{1a}, A^{1b}, A^{1b}, and R¹ are absent;
each R⁶ is selected from the group consisting of hydrogen, C₁-C₃ alkyl and —C(O)(C₁-C₃ alkyl);
each optionally substituted alkyl, alkenyl and alkynyl is unsubstituted, fluorinated or substituted with one or two hydroxyl groups;
each cycloalkyl has 3-10 ring carbons and is unsaturated or partially unsaturated, and optionally includes one or two fused cycloalkyl rings, each fused ring having 3-8 ring members;
each heterocylcloalkyl has 3-10 ring members and 1-3 heteroatoms independently selected from nitrogen, oxygen and sulfur and is unsaturated or partially unsaturated, and optionally includes one or two fused cycloalkyl rings, each having 3-8 ring members;
each aryl is a phenyl or a naphthyl, and optionally includes one or two fused cycloalkyl or heterocycloalkyl rings, each fused cycloalkyl or heterocycloalkyl ring having 4-8 ring members;
each heteroaryl is a 5-6 membered monocyclic heteroaryl ring having 1-4 heteroatoms independently selected from nitrogen, oxygen and sulfur or a 8-10 membered bicyclic heteroaryl having 1-5 heteroatoms independently selected from nitrogen, oxygen or sulfur, and optionally includes one or two fused cycloalkyl or heterocycloalkyl rings, each fused cycloalkyl or heterocycloalkyl ring having 4-8 ring members.
   In certain such embodiments, each and every optionally substituted alkyl, alkylene, alkenyl, alkenylene, alkynyl and alkynylene is unsubstituted or fluorinated. For example, in certain such embodiments, each and every optionally substituted alkyl, alkylene, alkenyl, alkenylene, alkynyl and alkynylene is unsubstituted.

The disclosure also provides a variety of subgenera of compounds of any of formulae (1k)-(1o) in which R¹, L¹, L², Q, L³, R³, L⁴, R⁴, L⁵, and R⁵ are optionally independently selected from the groups (6q) et seq., (10w) et seq., (11i) et seq., (12u) et seq., (13s) et seq., (14w) et seq., (15z) et seq., (19w) et seq., (20m) et seq., and (21o) et seq. defined hereinbelow (e.g., wherein the compound is as defined in any combination of the embodiments below). Definitions of the variables can be made from any combination of groups (6q) et seq., (10w) et seq., (11i) et seq., (12u) et seq., (13s) et seq., (14w) et seq., (15z) et seq., (19w) et seq., (20m) et seq., and (21o) et seq. defined hereinbelow that is not logically or chemically inconsistent.

In certain embodiments, the compound is one of the following structural formulae:
(Ik) in which the variables are as defined in any combination of groups (6q) et seq., (10w) et seq., (11i) et seq., (12u) et seq., (13s) et seq., (14w) et seq., (15z) et seq., (19w) et seq., (20m) et seq., and (21o) et seq. defined hereinbelow;
(Im); in which the variables are as defined in any combination of groups (6q) et seq., (10w) et seq., (11i) et seq., (12u) et seq., (13s) et seq., (14w) et seq., (15z) et seq., (19w) et seq., (20m) et seq., and (21o) et seq. defined hereinbelow;
(In) in which the variables are as defined in any combination of groups (6q) et seq., (10w) et seq., (11i) et seq., (12u) et seq., (13s) et seq., (14w) et seq., (15z) et seq., (19w) et seq., (20m) et seq., and (21o) et seq. defined hereinbelow;
(Io) in which the variables are as defined in any combination of groups (6q) et seq., (10w) et seq., (11i) et seq., (12u) et seq., (13s) et seq., (14w) et seq., (15z) et seq., (19w) et seq., (20m) et seq., and (21o) et seq. defined hereinbelow.

In certain embodiments as otherwise described herein. R¹ is selected from one of the following groups (6q)-(6u)
(6q) R¹ is selected from the group consisting of hydrogen, optionally substituted C₁-C₈ alkyl and cycloalkyl optionally substituted with 1-5 R^{1E};
(6r) R¹ is hydrogen;
(6s) R¹ is optionally substituted C₁-C₈ alkyl;
(6t) R¹ is unsubstituted C₁-C₈ alkyl or fluorinated C₁-C₈ alkyl;
(6u) R¹ is unsubstituted cycloalkyl;
In certain such embodiments, each optionally substituted alkyl of R¹ (including those of R^{1E}) is unsubstituted or fluorinated. For example, in certain such embodiments each optionally substituted alkyl, alkenyl and alkynyl of R¹ (including those of R^{1E}) is unsubstituted.

In certain embodiments as otherwise described herein. L¹ is selected from one of the following groups (10w)-(10v)
(10w) L¹ is a bond, —S—, —S(O)— or —S(O)₂—;
(10x) L¹ is selected from a bond, —CH₂—, —CH(CH₃)—, —CH₂CH₂—, —C(O)—, —S—, —S(O)₁₋₂—, —O—, and —NR⁶—;
(10y) L¹ is —O— or —S—;

In certain embodiments as otherwise described herein. L² is selected from one of the following groups (11i)-(11k)
(11i) L² is —CH₂—, —CH(CH₃)— or —CH₂CH₂—;
(11j) L² is a bond;
(11k) L² is a bond or —CH₂—.

In certain embodiments as otherwise described herein. Q is selected from one of the following groups (12u)-(12x)
(12u) Q is selected from the group consisting of —CH₂OH, —C(O)OH, —C(O)OR^{2A}, —C(O)NR^{2B}R^{2A}, —C(O)NR^{2B}S(O)₂R^{2A}, —C(O)NR^{2B}S(O)₂NR^{2B}R^{2A}, —C(O)R^{2A}, —S(O)₂OH, —P(O)(OH)₂, —C(OH)(CF₃)₂, S(O)₂R^{2A}, —N(R^{2B})S(O)₂R^{2A}, —S(O)₂NR^{2B}R^{2A}, —C(O)NH—O(C₁-C₃ alkyl), —C(O)NHOH and —CO(NH)CN;
(12v) Q is selected from the group consisting of —CH₂OH, —C(O)OH, —C(O)OR^{2A}, —C(O)NR^{2B}R^{2A}, —C(O)NR^{2B}S(O)₂R^{2A}, —C(O)NR^{2B}S(O)₂NR^{2B}R^{2A}, —C(O)R^{2A}, —S(O)₂OH, —P(O)(OH)₂.
(12w) Q is —CH₂OH, —C(O)OH or —C(O)OR^{2A};
(12x) Qis-COOH.

In certain embodiments as otherwise described herein. L³ is selected from one of the following groups (13s)-(13u)
(13s) L³ is a bond, —C(O)—, —S—, —S(O)₁₋₂—, —O—, —NR⁶—, —CH₂—, —CH(CH₃)(OH)— or —CH(OH)—;
(13t) L³ is a bond;
(13u) L³ is a bond, —CH₂—, —CH(CH₃)(OH)— or —CH(OH)—.

In certain embodiments as otherwise described herein. R³ is selected from one of the following groups (14w)-(14gg)
(14w) R³ is aryl or heteroaryl each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E};
(14x) R³ is aryl (e.g., a phenyl, a benzodioxole, or a dihydro-1H-isoquinoline) optionally substituted with 1-5 R^{3E};
(14y) R³ is aryl (e.g., a phenyl, a benzodioxole, ora dihydro-1H-isoquinoline) (i) substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E};
(14z) R³ is aryl (e.g., a phenyl, a benzodioxole, ora dihydro-1H-isoquinoline) (i) substituted with a single substituent selected from -L^{3C}-(phenyl optionally substituted with 1-5 R^{3D}), -L^{3C}-(monocyclic heteroaryl optionally substituted with 1-5 R^{3D}), -L^{3C}-(monocyclic cycloalkyl optionally substituted with 1-5 R^{3E}), -L^{3C}-(monocyclic heterocycloalkyl optionally substituted with 1-5 R^{3E}) and (ii) optionally substituted with 1-5 R^{3E};
(14aa) R³ is as defined in (14u)-(14x), wherein the aryl is not substituted with any R^{3E};
(14bb) R³ is heteroaryl (e.g., an isothiazole, a pyridone, a thiadiazole, a pyrazine, a pyrazolopyrimidine, a pyrazolopyridine, an imidazole, a benzofuran, an indole, an imidazopyridine, a pyridine, a pyrazole, an isoxazole, a triazolopyridine, a benzimidazole, a thiophene, a benzothiophene, a furan or a pyrimidine) optionally substituted with 1-5 R^{3E};
(14cc) R³ is heteroaryl (e.g., an isothiazole, a pyridone, a thiadiazole, a pyrazine, a pyrazolopyrimidine, a pyrazolopyridine, an imidazole, a benzofuran, an indole, an imidazopyridine, a pyridine, a pyrazole, an isoxazole, a triazolopyridine, a benzimidazole, a thiophene, a benzothiophene, a furan or a pyrimidine) (i) substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E};
(14dd) R³ is heteroaryl (e.g., an isothiazole, a pyridone, a thiadiazole, a pyrazine, a pyrazolopyrimidine, a pyrazolopyridine, an imidazole, a benzofuran, an indole, an imidazopyridine, a pyridine, a pyrazole, an isoxazole, a triazolopyridine, a benzimidazole, a thiophene, a benzothiophene, a furan or a pyrimidine) (i) substituted with a single substituent selected from -L^{3C}-(phenyl optionally substituted with 1-5 R^{3D}), -L^{3C}-(monocyclic heteroaryl optionally substituted with 1-5 R^{3D}), -L^{3C}-(monocyclic cycloalkyl optionally substituted with 1-5 R^{3E}), -L^{3C}-(monocyclic heterocycloalkyl optionally substituted with 1-5 R^{3E}) and (ii) optionally substituted with 1-5 R^{3E};
(14ee) R³ is as defined in (14z)-(14bb), wherein the heteroaryl is not substituted with any R^{3E};
(14ff) R³ is selected from the group consisting of: phenyl, benzodioxolyl, dihydro-1H-isoquinolinyl, imidazolyl, oxazolyl, isoxazolyl, isothiazolyl, thiazolyl, pyridinyl, and pyrazinyl, pyridonyl, thiadiazolyl, pyrazolopyrimidinyl, pyrazolopyridinyl, benzofuranyl, indolyl, imidazopyridinyl, pyrazolyl, triazolopyridinyl, benzimidazolyl, a benzimidazolyl, a thienyl, a benzothienyl, a furanyl and pyrimidinyl, each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E}.
(14gg) R³ is selected from the group consisting of phenyl and monocyclic heteroaryl (e.g., pyridyl, pyrazolyl), optionally substituted with 1-5 R^{3E}.
   In certain such embodiments, each optionally substituted alkyl, alkenyl and alkynyl of R³ (including those of R^{3D} and R^{3E}) is unsubstituted or fluorinated. For example, in certain such embodiments each optionally substituted alkyl, alkenyl and alkynyl of R³ (including those of R^{3D} and R^{3E}) is unsubstituted.

In certain embodiments as otherwise described herein. R⁴ is selected from one of the following groups (15z)-(15cc)
(15z) R⁴ is hydrogen;
(15aa) R⁴ is optionally substituted C₁-C₈ alkyl, optionally-substituted C₁-C₈ alkenyl or optionally substituted C₁-C₈ alkynyl;
(15bb) R⁴ is hydrogen or unsubstituted C₁-C₆ alkyl;
(15cc) R⁴ is unsubstituted C₁-C₃ alkyl.
   In certain such embodiments, each optionally substituted alkyl, alkenyl and alkynyl of R⁴ is unsubstituted or fluorinated. For example, in certain such embodiments each optionally substituted alkyl, alkenyl and alkynyl of R⁴ is unsubstituted.

In certain embodiments. L⁴ is selected from one of the following groups (19w)-(19x)
(19w) L⁴ is selected from a bond, —C(O)—, —S—, —S(O)₁₋₂—, —O—, and —NR⁶— (e.g., a bond);
(19x) L⁴ is a bond.

In certain embodiments. L⁵ is selected from one of the following groups
(20m)-(20n)
(20m) L⁵ is a bond, —C(O)—, —S—, —S(O)₁₋₂—, —O—, —NR⁶—, —CH₂CH₂—, —CH₂—, —CH(CH₃)(OH)— or —CH(OH)—;
(20n) L⁵ is a bond.

In certain embodiments as otherwise described herein. R⁵ is selected from one of the following groups (21o)-(21 q)
(21o) R⁵ is aryl (e.g., phenyl) or heteroaryl (e.g., an isoxazolyl, a pyridyl, an imidazopyridyl, a pyrazolyl), each optionally substituted with 1-5 R^{5E};
(21p) R⁵ is phenyl optionally substituted with 1-5 R^{5E};
(21 q) R⁵ is selected from the group consisting of phenyl, isoxazolyl, pyridyl, imidazopyridyl, and pyrazolyl, each optionally substituted with 1-5 R^{5E};
   In certain such embodiments, each optionally substituted alkyl, alkenyl and alkynyl of R⁵ (including those of R^{5D} and R^{5E}) is unsubstituted or fluorinated. For example, in certain such embodiments each optionally substituted alkyl, alkenyl and alkynyl of R⁵ (including those of R^{5D} and R^{5E}) is unsubstituted.

In certain additional embodiments, including any of the embodiments described with reference to formulae (I)-(Io) above, each optionally substituted alkylene, alkenylene, and alkynylene recited in any one of the preceding embodiments is unsubstituted. In alternative additional embodiments, including any of the embodiments described with reference to formulae (I)-(Io) above, each optionally substituted alkylene, alkenylene, and alkynylene recited in any one of the preceding embodiments is unsubstituted or fluorinated.

In certain additional embodiments, including any of the embodiments described with reference to formulae (I)-(Io) above and any embodiment described in the paragraph immediately above, each optionally substituted alkyl, alkenyl, and alkynyl recited in any one of preceding embodiments is unsubstituted. In alternative additional embodiments, including any of the embodiments described with reference to formulae (I)-(Io) above and any embodiment described in the paragraph immediately above, each optionally substituted alkyl, alkenyl, and alkynyl recited in any one of preceding embodiments is unsubstituted.

In certain additional embodiments, including any of the embodiments described with reference to formulae (I)-(Io) above and any embodiment described in the two paragraphs immediately above, each cycloalkyl recited in any one of the preceding embodiments is a 3-7 membered monocyclic cycloalkyl. For example, in certain particular embodiments, including any of the embodiments described with reference to formulae (I)-(Io) above and any embodiment described in the two paragraphs immediately above, each cycloalkyl recited in any one of the preceding embodiments is a cyclopropyl, a cyclobutyl, a cyclopentyl, a cyclopentenyl, a cyclohexyl or a cyclohexenyl.

In certain additional embodiments, including any of the embodiments described with reference to formulae (I)-(Io) above and any embodiment described in the three paragraphs immediately above, each heterocycloalkyl recited in any one of the preceding embodiments is a 4-7 membered monocyclic heterocycloalkyl having 1-2 heteroatoms selected from O, S and N. For example, in certain particular embodiments, including any of the embodiments described with reference to formulae (I)-(Io) above and any embodiment described in the three paragraphs immediately above, each heterocycloalkyl recited in any one of the preceding embodiments is a pyrrolidinyl, a tetrahydrofuranyl, a tetrahydrothienyl, a piperidinyl, a piperazinyl, a morpholinyl, a thiomorpholinyl, a tetrahydro-2H-pyranyl, or a tetrahydro-2H-thiopyranyl.

In certain additional embodiments, including any of the embodiments described with reference to formulae (I)-(Io) above and any embodiment described in the four paragraphs immediately above, each heteroaryl is a 5-6 membered monocyclic heteroaryl having 1-3 heteroatoms selected from O, S and N. For example, in certain particular embodiments, including any of the embodiments described with reference to formulae (I)-(Io) above and any embodiment described in the four paragraphs immediately above, each heteroaryl is a furanyl, a thienyl, a pyrrolyl, a pyrazolyl, an imidazolyl, an oxazolyl or a thiazolyl.

In certain additional embodiments, including any of the embodiments described with reference to formulae (I)-(Io) above and any embodiment described in the four paragraphs immediately above, each aryl is phenyl.

In certain additional embodiments as described above, including any of the embodiments described with reference to formulae (I)-(Io) above and any embodiment described in the five paragraphs immediately above, R⁵ is substituted with 1, 2 or 3 substituents selected from halogen (e.g., chloro- or fluoro-) and fluorinated C₁-C₃ alkyl (e.g., trifluoromethyl, difluoromethyl, fluoromethyl, pentafluoroethyl, trifluoroethyl). For example, in certain embodiment as described above, R⁵ is phenyl substituted (e.g., 3-substituted, 4-substituted, 3,4-disubstituted, 2,4-disubstituted, or 2,5-disubstituted) with one or two substitutents selected from trifluoromethyl, fluorine and chlorine. For example, in particular embodiments, R⁵ can be dichlorophenyl, e.g., 3,4-dichlorophenyl, or trifluoromethylphenyl, e.g., 4-trifluoromethylphenyl.

In certain embodiments, the therapeutic compound is one of the compounds of the compound table below. BJAB cell proliferation data is presented in the table; “A” indicates a measured EC₅₀ less than or equal to 1 μM; “B” indicates a measured EC₅₀ greater than 1 μM and less than or equal to 5 μM; “C” indicates a measured EC₅₀ greater than 5 μM and less than or equal to 10 μM; “D” indicates a measured EC₅₀ greater than 10 μM and less than or equal to 25 μM; “E” indicates a measured EC₅₀ greater than 25 μM and less than or equal to 50 μM; “F” indicates a measured EC₅₀ greater than 50 μM and less than or equal to 100 μM; “G” indicates that in the experiments performed there was no measured EC₅₀ less than or equal to 80 μM; “H” indicates that in the experiments performed there was no measured EC₅₀ less than or equal to 50 μM; “I” indicates that in the experiments performed there was no measured EC₅₀ less than or equal to 40 μM; “J” indicates that in the experiments performed there was no measured EC₅₀ less than or equal to 25 μM; and “K” indicates that in the experiments performed there was no measured EC₅₀ less than or equal to 20 μM. In certain embodiments, the therapeutic compound is a compound having an activity as “A,” “B” or “C” in the table below. In certain embodiments, the therapeutic compound is a compound having an activity as “A” or “B” in the table below. In certain embodiments, the therapeutic compound is a compound having an activity as “A” in the table below.

| Cpd | Name | EC₅₀ |
|---|---|---|
| A1 | methyl 1-(4-(3,4-dichlorophenyl)thiazol-2-yl)-3-methyl-4-(2-nitrobenzyl)- | H |
| | 1H-pyrazole-5-carboxylate | |
| A2 | 1-(4-(3,4-dichlorophenyl)thiazol-2-yl)-3-methyl-4-(2-nitrobenzyl)-1H- | H |
| | pyrazole-5-carboxylic acid | |
| A3 | methyl 1-(4-(3,4-dichlorophenyl)-5-(ethylthio)thiazol-2-yl)-3-methyl-4-(2- | G |
| | nitrobenzyl)-1H-pyrazole-5-carboxylate | |
| A4 | 1-(4-(3,4-dichlorophenyl)-5-(ethylthio)thiazol-2-yl)-3-methyl-4-(2- | F |
| | nitrobenzyl)-1H-pyrazole-5-carboxylic acid | |
| A5 | methyl 1-(4-(3,4-dichlorophenyl)-5-(isopropylthio)thiazol-2-yl)-3-methyl-4- | G |
| | (2-nitrobenzyl)-1H-pyrazole-5-carboxylate | |
| A6 | 1-(4-(3,4-dichlorophenyl)-5-(isopropylthio)thiazol-2-yl)-3-methyl-4-(2- | E |
| | nitrobenzyl)-1H-pyrazole-5-carboxylic acid | |
| A7 | methyl 1-(4-(3,4-dichlorophenyl)thiazol-2-yl)-3-methyl-4-(2- | G |
| | (methylsulfonamido)benzyl)-1H-pyrazole-5-carboxylate | |
| A8 | 1-(4-(3,4-dichlorophenyl)thiazol-2-yl)-3-methyl-4-(2- | G |
| | (methylsulfonamido)benzyl)-1H-pyrazole-5-carboxylic acid | |
| A9 | methyl 1-(4-(3,4-dichlorophenyl)-5-(propylthio)thiazol-2-yl)-3-methyl-4-(2- | G |
| | nitrobenzyl)-1H-pyrazole-5-carboxylate | |
| A10 | 1-(4-(3,4-dichlorophenyl)-5-(propylthio)thiazol-2-yl)-3-methyl-4-(2- | E |
| | nitrobenzyl)-1H-pyrazole-5-carboxylic acid | |
| A11 | methyl 1-(5-(butylthio)-4-(3,4-dichlorophenyl)thiazol-2-yl)-3-methyl-4-(2- | G |
| | nitrobenzyl)-1H-pyrazole-5-carboxylate | |
| A12 | 1-(5-(butylthio)-4-(3,4-dichlorophenyl)thiazol-2-yl)-3-methyl-4-(2- | G |
| | nitrobenzyl)-1H-pyrazole-5-carboxylic acid | |
| A13 | 1-(4-(3,4-dichlorophenyl)-5-(propylthio)thiazol-2-yl)-3-methyl-4-(2- | E |
| | (methylsulfonamido)benzyl)-1H-pyrazole-5-carboxylic acid | |
| A14 | N-(2-((5-(4-(aminomethyl)piperidine-1-carbonyl)-1-(4-(3,4- | D |
| | dichlorophenyl)-5-(propylthio)thiazol-2-yl)-3-methyl-1H-pyrazol-4- | |
| | yl)methyl)phenyl)methanesulfonamide | |
| A15 | 4-bromo-1-(4-(3,4-dichlorophenyl)-5-(isopropylthio)thiazol-2-yl)-3-methyl- | D |
| | 1H-pyrazole-5-carboxylic acid | |
| A16 | 1-(4-(3,4-dichlorophenyl)-5-(isopropylthio)thiazol-2-yl)-3-methyl-1H- | F |
| | pyrazole-5-carboxylic acid | |
| A17 | 1-(4-(3,4-dichlorophenyl)-5-(isopropylthio)thiazol-2-yl)-4-(imidazo[1,2- | D |
| | a]pyridin-6-yl)-3-methyl-1H-pyrazole-5-carboxylic acid | |
| A18 | 1-(4-(3,4-dichlorophenyl)-5-(isopropylthio)thiazol-2-yl)-3-methyl-4-phenyl- | B |
| | 1H-pyrazole-5-carboxylic acid | |
| A19 | 1-(5-(cyclohexylthio)-4-(3,4-dichlorophenyl)thiazol-2-yl)-3-methyl-4-(2- | D |
| | nitrobenzyl)-1H-pyrazole-5-carboxylic acid | |
| A20 | 4-(benzofuran-2-yl)-1-(4-(3,4-dichlorophenyl)-5-(isopropylthio)thiazol-2- | C |
| | yl)-3-methyl-1H-pyrazole-5-carboxylic acid | |
| A21 | 1-(4-(3,4-dichlorophenyl)-5-(isopropylthio)thiazol-2-yl)-4-(2-fluoropyridin- | C |
| | 4-yl)-3-methyl-1H-pyrazole-5-carboxylic acid | |
| A22 | 2-(4-(3,4-dichlorophenyl)-5-(isopropylthio)thiazol-2-yl)-2′,5,5′-trimethyl- | D |
| | 4,4′-bi(2H-pyrazole)-3-carboxylic acid | |
| A23 | 1-(4-(3,4-dichlorophenyl)-5-(isopropylthio)thiazol-2-yl)-4-(2,6- | A |
| | dimethylpyridin-4-yl)-3-methyl-1H-pyrazole-5-carboxylic acid | |
| A24 | 1-(4-(3,4-dichlorophenyl)-5-(isopropylthio)thiazol-2-yl)-4-(3,5- | B |
| | difluorophenyl)-3-methyl-1H-pyrazole-5-carboxylic acid | |
| A25 | 1-(4-(5-chloro-2-fluorophenyl)-5-(isopropylthio)thiazol-2-yl)-4-(3,5- | B |
| | dichlorophenyl)-3-methyl-1H-pyrazole-5-carboxylic acid | |
| A26 | 1-(4-(5-chloro-2-fluorophenyl)-5-(isopropylthio)thiazol-2-yl)-4-(2,6- | C |
| | dimethylpyridin-4-yl)-3-methyl-1H-pyrazole-5-carboxylic acid | |
| A27 | 4-(3,5-dichlorophenyl)-1-(4-(3,5-dimethylisoxazol-4-yl)-5- | J |
| | (isopropylthio)thiazol-2-yl)-3-methyl-1H-pyrazole-5-carboxylic acid | |
| A28 | 4-(3,5-dichlorophenyl)-1-(5-(isopropylthio)-4-(1-methyl-1H-pyrazol-4- | J |
| | yl)thiazol-2-yl)-3-methyl-1H-pyrazole-5-carboxylic acid | |
| A29 | 4-(3,5-dichlorophenyl)-1-(5-(isopropylthio)-4-(pyridin-4-yl)thiazol-2-yl)-3- | J |
| | methyl-1H-pyrazole-5-carboxylic acid | |
| A30 | 4-(3,5-dichlorophenyl)-1-(5-(isopropylthio)-4-(pyridin-3-yl)thiazol-2-yl)-3- | J |
| | methyl-1H-pyrazole-5-carboxylic acid | |
| A31 | 4-(3,5-dichlorophenyl)-1-(4-(imidazo[1,2-a]pyridin-6-yl)-5- | J |
| | (isopropylthio)thiazol-2-yl)-3-methyl-1H-pyrazole-5-carboxylic acid | |
| A32a | methyl 1-(5-(sec-butylthio)-4-(3,4-dichlorophenyl)thiazol-2-yl)-4-(3,5- | J |
| | dichlorophenyl)-3-methyl-1H-pyrazole-5-carboxylate | |
| A32b | 1-(5-(sec-butylthio)-4-(3,4-dichlorophenyl)thiazol-2-yl)-4-(3,5- | B |
| | dichlorophenyl)-3-methyl-1H-pyrazole-5-carboxylic acid | |
| A33a | methyl 1-(5-(sec-butylthio)-4-(3,4-dichlorophenyl)thiazol-2-yl)-4-(2,6- | J |
| | dimethylpyridin-4-yl)-3-methyl-1H-pyrazole-5-carboxylate | |
| A33b | 1-(5-(sec-butylthio)-4-(3,4-dichlorophenyl)thiazol-2-yl)-4-(2,6- | A |
| | dimethylpyridin-4-yl)-3-methyl-1H-pyrazole-5-carboxylic acid | |
| A34 | 4-(2,6-dimethylpyridin-4-yl)-1-(5-(isopropylthio)-4-(2- | C |
| | methoxyphenyl)thiazol-2-yl)-3-methyl-1H-pyrazole-5-carboxylic acid | |
| A35 | 4-(2,6-dimethylpyridin-4-yl)-1-(5-(isopropylthio)-4-(3- | B |
| | methoxyphenyl)thiazol-2-yl)-3-methyl-1H-pyrazole-5-carboxylic acid | |
| A36 | 4-(2,6-dimethylpyridin-4-yl)-1-(5-(isopropylthio)-4-(4- | D |
| | methoxyphenyl)thiazol-2-yl)-3-methyl-1H-pyrazole-5-carboxylic acid | |
| A37 | 4-(2,6-dimethylpyridin-4-yl)-1-(5-(isopropylthio)-4-phenylthiazol-2-yl)-3- | C |
| | methyl-1H-pyrazole-5-carboxylic acid | |
| A38 | 1-(4-(3,4-dichlorophenyl)-5-(isopropylthio)thiazol-2-yl)-4-(3- | H |
| | (hydroxymethyl)-5-methylisoxazol-4-yl)-3-methyl-1H-pyrazole-5- | |
| | carboxylic acid | |
| A39 | 1-(4-(3,4-dichlorophenyl)-5-(isopropylthio)thiazol-2-yl)-4-(3- | D |
| | (methoxymethyl)-5-methylisoxazol-4-yl)-3-methyl-1H-pyrazole-5- | |
| | carboxylic acid | |
| A40 | 4-(2,6-dimethylpyridin-4-yl)-1-(5-(isopropylthio)-4-p-tolylthiazol-2-yl)-3- | B |
| | methyl-1H-pyrazole-5-carboxylic acid | |
| A41 | 4-(2,6-dimethylpyridin-4-yl)-1-(5-(isopropylthio)-4-m-tolylthiazol-2-yl)-3- | C |
| | methyl-1H-pyrazole-5-carboxylic acid | |
| A42 | 4-(2,6-dimethylpyridin-4-yl)-1-(5-(isopropylthio)-4-o-tolylthiazol-2-yl)-3- | B |
| | methyl-1H-pyrazole-5-carboxylic acid | |
| A43 | 4-(2,6-dimethylpyridin-4-yl)-1-(5-(isopropylthio)-4-(phenylethynyl)thiazol- | C |
| | 2-yl)-3-methyl-1H-pyrazole-5-carboxylic acid | |
| A44 | 1-(4-(3,4-dichlorophenyl)-5-(isopropylthio)thiazol-2-yl)-3-methyl-4- | D |
| | (pyridin-3-yl)-1H-pyrazole-5-carboxylic acid | |
| A45 | 1-(4-(3,4-dichlorophenyl)-5-(methylthio)thiazol-2-yl)-3-methyl-4-(2- | E |
| | nitrobenzyl)-1H-pyrazole-5-carboxylic acid | |
| A46 | 1-(4-(3,4-dichlorophenyl)-5-(isopropylthio)thiazol-2-yl)-4-(3- | D |
| | (hydroxymethyl)phenyl)-3-methyl-1H-pyrazole-5-carboxylic acid | |
| A47 | 1-(4-(3,4-dichlorophenyl)-5-(isopropylthio)thiazol-2-yl)-3-methyl-4- | C |
| | (pyridin-4-yl)-1H-pyrazole-5-carboxylic acid | |
| A48 | 4-(3-(aminomethyl)phenyl)-1-(4-(3,4-dichlorophenyl)-5- | E |
| | (isopropylthio)thiazol-2-yl)-3-methyl-1H-pyrazole-5-carboxylic acid | |
| A49 | 1-(4-(3,4-dichlorophenyl)-5-(isopropylthio)thiazol-2-yl)-4-(4- | H |
| | (hydroxymethyl)phenyl)-3-methyl-1H-pyrazole-5-carboxylic acid | |
| A50 | 1-(4-(3,4-dichlorophenyl)-5-(isopropylthio)thiazol-2-yl)-3-methyl-4-(2- | C |
| | (trifluoromethyl)phenyl)-1H-pyrazole-5-carboxylic acid | |
| A51 | 1-(4-(3,4-dichlorophenyl)-5-(isopropylthio)thiazol-2-yl)-3-methyl-4-(3- | B |
| | (trifluoromethyl)phenyl)-1H-pyrazole-5-carboxylic acid | |
| A52 | 1-(4-(3,4-dichlorophenyl)-5-(isopropylthio)thiazol-2-yl)-3-methyl-4-(4- | D |
| | (trifluoromethyl)phenyl)-1H-pyrazole-5-carboxylic acid | |
| A53 | 1-(4-(3,4-dichlorophenyl)-5-(isopropylthio)thiazol-2-yl)-4- | H |
| | (hydroxy(phenyl)methyl)-3-methyl-1H-pyrazole-5-carboxylic acid | |
| A54 | 1-(4-(3,4-dichlorophenyl)-5-(isopropylsulfinyl)thiazol-2-yl)-3-methyl-4-(2- | E |
| | nitrobenzyl)-1H-pyrazole-5-carboxylic acid | |
| A55 | 1-(4-(3,4-dichlorophenyl)-5-(isopropylsulfonyl)thiazol-2-yl)-3-methyl-4-(2- | D |
| | nitrobenzyl)-1H-pyrazole-5-carboxylic acid | |
| A56 | 4-([1,2,4]triazolo[4,3-a]pyridin-3-yl)-1-(4-(3,4-dichlorophenyl)-5- | H |
| | (isopropylthio)thiazol-2-yl)-3-methyl-1H-pyrazole-5-carboxylic acid | |
| A57 | 1-(4-(3,4-dichlorophenyl)-5-(isopropylthio)thiazol-2-yl)-4-(3,5- | D |
| | dimethylisoxazol-4-yl)-3-methyl-1H-pyrazole-5-carboxylic acid | |
| A58 | 4-(1H-benzo[d]imidazol-2-yl)-1-(4-(3,4-dichlorophenyl)-5- | E |
| | (isopropylthio)thiazol-2-yl)-3-methyl-1H-pyrazole-5-carboxylic acid | |
| A59 | 4-(3-chloro-2-methylphenyl)-1-(4-(3,4-dichlorophenyl)-5- | C |
| | (isopropylthio)thiazol-2-yl)-3-methyl-1H-pyrazole-5-carboxylic acid | |
| A60 | 4-(3,5-bis(trifluoromethyl)phenyl)-1-(4-(3,4-dichlorophenyl)-5- | B |
| | (isopropylthio)thiazol-2-yl)-3-methyl-1H-pyrazole-5-carboxylic acid | |
| A61 | 1-(4-(3,4-dichlorophenyl)-5-(isopropylthio)thiazol-2-yl)-4-(3- | B |
| | isopropylphenyl)-3-methyl-1H-pyrazole-5-carboxylic acid | |
| A62 | 1-(4-(3,4-dichlorophenyl)-5-(isopropylthio)thiazol-2-yl)-4-(2-methoxy-5- | C |
| | methylphenyl)-3-methyl-1H-pyrazole-5-carboxylic acid | |
| A63 | 1-(4-(3,4-dichlorophenyl)-5-(isopropylthio)thiazol-2-yl)-4-(1H-imidazol-2- | D |
| | yl)-3-methyl-1H-pyrazole-5-carboxylic acid | |
| A64 | 1-(4-(3,4-dichlorophenyl)-5-(isopropylthio)thiazol-2-yl)-3-methyl-4- | D |
| | (pyrazolo[1,5-a]pyridin-3-yl)-1H-pyrazole-5-carboxylic acid | |
| A65 | 1-(4-(3,4-dichlorophenyl)-5-(isopropylthio)thiazol-2-yl)-3-methyl-4- | E |
| | (pyrazolo[1,5-a]pyrimidin-3-yl)-1H-pyrazole-5-carboxylic acid | |
| A66 | 2-(4-(3,4-dichlorophenyl)-5-(isopropylthio)thiazol-2-yl)-2′,5-dimethyl-4,4′- | C |
| | bi(2H-pyrazole)-3-carboxylic acid | |
| A67 | 2-(4-(3,4-dichlorophenyl)-5-(isopropylsulfonyl)thiazol-2-yl)-2′,5-dimethyl- | E |
| | 4,4′-bi(2H-pyrazole)-3-carboxylic acid | |
| A68 | 1-(4-(3,4-dichlorophenyl)-5-(isopropylthio)thiazol-2-yl)-3-methyl-4-(1- | H |
| | methyl-1H-benzo[d]imidazol-2-yl)-1H-pyrazole-5-carboxylic acid | |
| A69 | 4-(5-cyanopyridin-3-yl)-1-(4-(3,4-dichlorophenyl)-5-(isopropylthio)thiazol- | D |
| | 2-yl)-3-methyl-1H-pyrazole-5-carboxylic acid | |
| A70 | 1-(4-(3,4-dichlorophenyl)-5-(isopropylthio)thiazol-2-yl)-4-(2-ethoxypyridin- | D |
| | 3-yl)-3-methyl-1H-pyrazole-5-carboxylic acid | |
| A71 | 2′-(4-(3,4-dichlorophenyl)-5-(isopropylthio)thiazol-2-yl)-2,5,5′-trimethyl- | D |
| | 3,4′-bi(2H-pyrazole)-3′-carboxylic acid | |
| A72 | 1-(4-(3,4-dichlorophenyl)-5-(isopropylthio)thiazol-2-yl)-3-methyl-4- | D |
| | (pyrimidin-5-yl)-1H-pyrazole-5-carboxylic acid | |
| A73 | 4-(3,5-bis(trifluoromethyl)phenyl)-1-(4-(3,4-dichlorophenyl)-5- | I |
| | (isopropylthio)thiazol-2-yl)-3-methyl-1H-pyrazole-5-carboxamide | |
| A74 | 1-(4-(3,4-dichlorophenyl)-5-(isopropylthio)thiazol-2-yl)-4-(4,6- | E |
| | dimethylpyrimidin-2-yl)-3-methyl-1H-pyrazole-5-carboxylic acid | |
| A75 | 4-(3,5-bis(trifluoromethyl)phenyl)-1-(4-(3,4-dichlorophenyl)-5- | I |
| | (isopropylthio)thiazol-2-yl)-3-methyl-N-(pyridin-2-ylmethyl)-1H-pyrazole-5- | |
| | carboxamide | |
| A76 | 4-(3,5-bis(trifluoromethyl)phenyl)-1-(4-(3,4-dichlorophenyl)-5- | I |
| | (isopropylthio)thiazol-2-yl)-N-(2-hydroxyethyl)-N,3-dimethyl-1H-pyrazole- | |
| | 5-carboxamide | |
| A77 | 4-(3,5-bis(trifluoromethyl)phenyl)-1-(4-(3,4-dichlorophenyl)-5- | I |
| | (isopropylthio)thiazol-2-yl)-N,3-dimethyl-N-(5-(trifluoromethyl)-1,3,4- | |
| | thiadiazol-2-yl)-1H-pyrazole-5-carboxamide | |
| A78 | (4-(3,5-bis(trifluoromethyl)phenyl)-1-(4-(3,4-dichlorophenyl)-5- | I |
| | (isopropylthio)thiazol-2-yl)-3-methyl-1H-pyrazol-5-yl)(3- | |
| | (diethylamino)pyrrolidin-1-yl)methanone | |
| A79 | 1-(4-(3,4-dichlorophenyl)-5-(isopropylthio)thiazol-2-yl)-3-methyl-4- | D |
| | (pyrazin-2-yl)-1H-pyrazole-5-carboxylic acid | |
| A80 | 4-(3-cyano-5-methylphenyl)-1-(4-(3,4-dichlorophenyl)-5- | B |
| | (isopropylthio)thiazol-2-yl)-3-methyl-1H-pyrazole-5-carboxylic acid | |
| A81 | 4-(3,5-bis(trifluoromethyl)phenyl)-1-(4-(3,4-dichlorophenyl)-5- | I |
| | (isopropylthio)thiazol-2-yl)-3-methyl-N-(1H-tetrazol-5-yl)-1H-pyrazole-5- | |
| | carboxamide | |
| A82 | 1-(4-(3,4-dichlorophenyl)-5-(isopropylthio)thiazol-2-yl)-3-methyl-4-(5- | I |
| | methyl-1,3,4-thiadiazol-2-yl)-1H-pyrazole-5-carboxylic acid | |
| A83 | 4-(3-cyano-5-methoxyphenyl)-1-(4-(3,4-dichlorophenyl)-5- | C |
| | (isopropylthio)thiazol-2-yl)-3-methyl-1H-pyrazole-5-carboxylic acid | |
| A84 | 4-(3-cyano-5-(trifluoromethyl)phenyl)-1-(4-(3,4-dichlorophenyl)-5- | B |
| | (isopropylthio)thiazol-2-yl)-3-methyl-1H-pyrazole-5-carboxylic acid | |
| A85 | 1-(4-(3,4-dichlorophenyl)-5-(isopropylthio)thiazol-2-yl)-4-(3- | C |
| | (methoxymethyl)phenyl)-3-methyl-1H-pyrazole-5-carboxylic acid | |
| A86 | 4-(3-benzyl-5-methylisoxazol-4-yl)-1-(4-(3,4-dichlorophenyl)-5- | B |
| | (isopropylthio)thiazol-2-yl)-3-methyl-1H-pyrazole-5-carboxylic acid | |
| A87 | 1-(4-(3,4-dichlorophenyl)-5-(isopropylthio)thiazol-2-yl)-4-(3- | J |
| | ((dimethylamino)methyl)phenyl)-3-methyl-1H-pyrazole-5-carboxylic acid | |
| A88 | (1-(4-(3,4-dichlorophenyl)-5-(isopropylthio)thiazol-2-yl)-3-methyl-4- | J |
| | phenyl-1H-pyrazol-5-yl)MeOH | |
| A89 | 4-(benzo[d][1,3]dioxol-5-yl)-1-(4-(3,4-dichlorophenyl)-5- | C |
| | (isopropylthio)thiazol-2-yl)-3-methyl-1H-pyrazole-5-carboxylic acid | |
| A90 | 1-(4-(3,4-dichlorophenyl)-5-(isopropylthio)thiazol-2-yl)-4-(4- | J |
| | methoxypyrimidin-2-yl)-3-methyl-1H-pyrazole-5-carboxylic acid | |
| A91 | 1-(4-(3,4-dichlorophenyl)-5-(isopropylthio)thiazol-2-yl)-4-(isothiazol-4-yl)- | C |
| | 3-methyl-1H-pyrazole-5-carboxylic acid | |
| A92 | 1-(4-(3,4-dichlorophenyl)-5-(isopropylthio)thiazol-2-yl)-3-methyl-4-(3- | B |
| | methylisothiazol-5-yl)-1H-pyrazole-5-carboxylic acid | |
| A93 | 1-(4-(3,4-dichlorophenyl)-5-(isopropylthio)thiazol-2-yl)-3-methyl-4-(1- | J |
| | methyl-2-oxo-1,2-dihydropyridin-4-yl)-1H-pyrazole-5-carboxylic acid | |
| A94 | 1-(4-(3,4-dichlorophenyl)-5-(isopropylthio)thiazol-2-yl)-4-(1,5-dimethyl-6- | J |
| | oxo-1,6-dihydropyridin-3-yl)-3-methyl-1H-pyrazole-5-carboxylic acid | |
| A95 | 1-(4-(3,4-dichlorophenyl)-5-(isopropylthio)thiazol-2-yl)-4-(3-fluoro-5- | B |
| | methylphenyl)-3-methyl-1H-pyrazole-5-carboxylic acid | |
| A96 | 1-(4-(3,4-dichlorophenyl)-5-(isopropylthio)thiazol-2-yl)-4-(3-hydroxy-5- | J |
| | methylphenyl)-3-methyl-1H-pyrazole-5-carboxylic acid | |
| A97 | 1-(4-(3,4-dichlorophenyl)-5-(isopropylthio)thiazol-2-yl)-4-(3-isopropoxy-5- | B |
| | methylphenyl)-3-methyl-1H-pyrazole-5-carboxylic acid | |
| A98 | 1-(4-(3,4-dichlorophenyl)-5-(isopropylthio)thiazol-2-yl)-3-methyl-4-(3- | B |
| | methyl-5-(oxetan-3-yloxy)phenyl)-1H-pyrazole-5-carboxylic acid | |
| A99 | 1-(4-(3,4-dichlorophenyl)-5-(isopropylthio)thiazol-2-yl)-4-(3- | C |
| | (dimethylamino)-5-methylphenyl)-3-methyl-1H-pyrazole-5-carboxylic acid | |
| A100 | 4-(3-(1H-imidazol-1-yl)-5-methylphenyl)-1-(4-(3,4-dichlorophenyl)-5- | B |
| | (isopropylthio)thiazol-2-yl)-3-methyl-1H-pyrazole-5-carboxylic acid | |
| A101 | 4-(2-(azetidin-1-yl)-6-methylpyridin-4-yl)-1-(4-(3,4-dichlorophenyl)-5- | B |
| | (isopropylthio)thiazol-2-yl)-3-methyl-1H-pyrazole-5-carboxylic acid | |
| A102 | 1-(4-(3,4-dichlorophenyl)-5-(isopropylthio)thiazol-2-yl)-3-methyl-4-(2- | C |
| | methyl-6-morpholinopyridin-4-yl)-1H-pyrazole-5-carboxylic acid | |
| A103 | 1-(5-(isopropylthio)-4-(3-methoxyphenyl)thiazol-2-yl)-3-methyl-4-(2- | G |
| | nitrobenzyl)-1H-pyrazole-5-carboxylic acid | |
| A104 | 1-(4-(3,4-dichlorophenyl)-5-(isopentylthio)thiazol-2-yl)-3-methyl-4-(2- | E |
| | nitrobenzyl)-1H-pyrazole-5-carboxylic acid | |
| A105 | 1-(5-(isopropylthio)-4-(3-methoxyphenyl)thiazol-2-yl)-3-methyl-4-(2- | G |
| | (methylsulfonamido)benzyl)-1H-pyrazole-5-carboxylic acid | |
| A106 | 1-(5-(sec-butylthio)-4-(3,4-dichlorophenyl)thiazol-2-yl)-3-methyl-4-(2- | D |
| | nitrobenzyl)-1H-pyrazole-5-carboxylic acid | |
| A107 | 1-(4-(3,4-dichlorophenyl)-5-(isopropylthio)thiazol-2-yl)-4-(1H-indol-6-yl)-3- | E |
| | methyl-1H-pyrazole-5-carboxylic acid | |
| A108 | 1-(4-(3,4-dichlorophenyl)-5-(isopropylthio)thiazol-2-yl)-3-methyl-4-(3- | C |
| | nitrophenyl)-1H-pyrazole-5-carboxylic acid | |
| A109 | 2-(4-(3,4-dichlorophenyl)-5-(isopropylthio)thiazol-2-yl)-5-methyl-4,4′- | E |
| | bi(2H-pyrazole)-3-carboxylic acid | |
| A110 | 1-(4-(3,4-dichlorophenyl)-5-(isopropylthio)thiazol-2-yl)-4-(1H-indol-3-yl)-3- | D |
| | methyl-1H-pyrazole-5-carboxylic acid | |
| A111 | 4-(2-chlorophenyl)-1-(4-(3,4-dichlorophenyl)-5-(isopropylthio)thiazol-2-yl)- | D |
| | 3-methyl-1H-pyrazole-5-carboxylic acid | |
| A112 | 4-(3-chlorophenyl)-1-(4-(3,4-dichlorophenyl)-5-(isopropylthio)thiazol-2-yl)- | B |
| | 3-methyl-1H-pyrazole-5-carboxylic acid | |
| A113 | 4-(4-chlorophenyl)-1-(4-(3,4-dichlorophenyl)-5-(isopropylthio)thiazol-2-yl)- | D |
| | 3-methyl-1H-pyrazole-5-carboxylic acid | |
| A114 | 1-(4-(3,4-dichlorophenyl)-5-(isopropylthio)thiazol-2-yl)-4-(2- | D |
| | methoxyphenyl)-3-methyl-1H-pyrazole-5-carboxylic acid | |
| A115 | 1-(4-(3,4-dichlorophenyl)-5-(isopropylthio)thiazol-2-yl)-4-(3- | B |
| | methoxyphenyl)-3-methyl-1H-pyrazole-5-carboxylic acid | |
| A116 | 1-(4-(3,4-dichlorophenyl)-5-(isopropylthio)thiazol-2-yl)-4-(4- | D |
| | methoxyphenyl)-3-methyl-1H-pyrazole-5-carboxylic acid | |
| A117 | 1-(4-(3,4-dichlorophenyl)-5-(isopropylthio)thiazol-2-yl)-3-methyl-4-o-tolyl- | C |
| | 1H-pyrazole-5-carboxylic acid | |
| A118 | 1-(4-(3,4-dichlorophenyl)-5-(isopropylthio)thiazol-2-yl)-3-methyl-4-m-tolyl- | B |
| | 1H-pyrazole-5-carboxylic acid | |
| A119 | 1-(4-(3,4-dichlorophenyl)-5-(isopropylthio)thiazol-2-yl)-3-methyl-4-p-tolyl- | D |
| | 1H-pyrazole-5-carboxylic acid | |
| A120 | 4-(4-acetamidophenyl)-1-(4-(3,4-dichlorophenyl)-5-(isopropylthio)thiazol- | H |
| | 2-yl)-3-methyl-1H-pyrazole-5-carboxylic acid | |
| A121 | 1-(4-(3,4-dichlorophenyl)-5-(isopropylthio)thiazol-2-yl)-3-methyl-4-(2- | H |
| | (methylsulfonamido)benzyl)-1H-pyrazole-5-carboxylic acid | |
| A122 | 1-(4-(3,4-dichlorophenyl)-5-(isopropylthio)thiazol-2-yl)-4-(2-(N,N- | D |
| | dimethylsulfamoylamino)benzyl)-3-methyl-1H-pyrazole-5-carboxylic acid | |
| A123 | 4-(4-aminophenyl)-1-(4-(3,4-dichlorophenyl)-5-(isopropylthio)thiazol-2-yl)- | E |
| | 3-methyl-1H-pyrazole-5-carboxylic acid | |
| A124 | 1-(4-(3,4-dichlorophenyl)-5-(isopropylthio)thiazol-2-yl)-3-methyl-4-(4- | H |
| | (methylsulfonamido)phenyl)-1H-pyrazole-5-carboxylic acid | |
| A125 | 1-(4-(3,4-dichlorophenyl)-5-(isopropylthio)thiazol-2-yl)-3-methyl-4-(3- | H |
| | (methylsulfonamido)phenyl)-1H-pyrazole-5-carboxylic acid | |
| A126 | 2-(4-(3,4-dichlorophenyl)-5-(isopropylthio)thiazol-2-yl)-2′-(2- | D |
| | methoxyethyl)-5-methyl-4,4′-bi(2H-pyrazole)-3-carboxylic acid | |
| A127 | 1-(4-(3,4-dichlorophenyl)-5-(isopropylthio)thiazol-2-yl)-4-(1H-indol-7-yl)-3- | C |
| | methyl-1H-pyrazole-5-carboxylic acid | |
| A128 | 2-(4-(1H-indol-7-yl)-3-methyl-1H-pyrazol-1-yl)-4-(3,4-dichlorophenyl)-5- | D |
| | (isopropylthio)thiazole | |
| A129 | 2-(4-(3,4-dichlorophenyl)-5-(isopropylthio)thiazol-2-yl)-2′-ethyl-5-methyl- | C |
| | 4,4′-bi(2H-pyrazole)-3-carboxylic acid | |
| A130 | 1-(4-(3,4-dichlorophenyl)-5-(isopropylthio)thiazol-2-yl)-N-(2- | H |
| | methoxyethyl)-3-methyl-4-m-tolyl-1H-pyrazole-5-carboxamide | |
| A131 | (4-(aminomethyl)piperidin-1-yl)(1-(4-(3,4-dichlorophenyl)-5- | B |
| | (isopropylthio)thiazol-2-yl)-3-methyl-4-o-tolyl-1H-pyrazol-5-yl)methanone | |
| A132 | 1-(4-(3,4-dichlorophenyl)-5-(isopropylthio)thiazol-2-yl)-4-(5- | D |
| | methoxypyridin-3-yl)-3-methyl-1H-pyrazole-5-carboxylic acid | |
| A133 | 2-(4-(3,4-dichlorophenyl)-5-(isopropylthio)thiazol-2-yl)-2′-isobutyl-5- | D |
| | methyl-4,4′-bi(2H-pyrazole)-3-carboxylic acid | |
| A134 | 1-(4-(3,4-dichlorophenyl)-5-(isopropylthio)thiazol-2-yl)-3-methyl-4-(2- | D |
| | (methylamino)pyridin-4-yl)-1H-pyrazole-5-carboxylic acid | |
| A135 | 1-(4-(3,4-dichlorophenyl)-5-(isopropylthio)thiazol-2-yl)-1′- | E |
| | (dimethylcarbamoyl)-3-methyl-1H,1′H-[4,4′-bipyrazole]-5-carboxylic acid | |
| A136 | (4-(aminomethyl)piperidin-1-yl)(4-(3,5-bis(trifluoromethyl)phenyl)-1-(4- | B |
| | (3,4-dichlorophenyl)-5-(isopropylthio)thiazol-2-yl)-3-methyl-1H-pyrazol-5- | |
| | yl)methanone | |
| A137 | N-(2-aminoethyl)-4-(3,5-bis(trifluoromethyl)phenyl)-1-(4-(3,4- | B |
| | dichlorophenyl)-5-(isopropylthio)thiazol-2-yl)-N,3-dimethyl-1H-pyrazole-5- | |
| | carboxamide | |
| A138 | (3-aminoazetidin-1-yl)(4-(3,5-bis(trifluoromethyl)phenyl)-1-(4-(3,4- | D |
| | dichlorophenyl)-5-(isopropylthio)thiazol-2-yl)-3-methyl-1H-pyrazol-5- | |
| | yl)methanone | |
| A139 | (4-(3,5-bis(trifluoromethyl)phenyl)-1-(4-(3,4-dichlorophenyl)-5- | D |
| | (isopropylthio)thiazol-2-yl)-3-methyl-1H-pyrazol-5- | |
| | yl)(morpholino)methanone | |
| A140 | 4-(3,5-bis(trifluoromethyl)phenyl)-1-(4-(3,4-dichlorophenyl)-5- | I |
| | (isopropylthio)thiazol-2-yl)-N,N,3-trimethyl-1H-pyrazole-5-carboxamide | |
| A141 | (4-(3,5-bis(trifluoromethyl)phenyl)-1-(4-(3,4-dichlorophenyl)-5- | I |
| | (isopropylthio)thiazol-2-yl)-3-methyl-1H-pyrazol-5-yl)(4- | |
| | (hydroxymethyl)piperidin-1-yl)methanone | |
| A142 | 1-(4-(3,4-dichlorophenyl)-5-isobutylthiazol-2-yl)-1′,3-dimethyl-1H,1′H- | B |
| | [4,4′-bipyrazole]-5-carboxylic acid | |
| A143 | 1-(4-(3,4-dichlorophenyl)-5-(isopropylthio)thiazol-2-yl)-4-(3,5- | B |
| | dimethylphenyl)-3-methyl-1H-pyrazole-5-carboxylic acid | |
| A144 | 4-(3,5-dichlorophenyl)-1-(4-(3,4-dichlorophenyl)-5-(isopropylthio)thiazol- | A |
| | 2-yl)-3-methyl-1H-pyrazole-5-carboxylic acid | |
| A145 | 4-(3-chloro-5-methoxyphenyl)-1-(4-(3,4-dichlorophenyl)-5- | B |
| | (isopropylthio)thiazol-2-yl)-3-methyl-1H-pyrazole-5-carboxylic acid | |
| A146 | 1-(4-(3,4-dichlorophenyl)-5-(isopropylthio)thiazol-2-yl)-4-(3-methoxy-5- | B |
| | (trifluoromethyl)phenyl)-3-methyl-1H-pyrazole-5-carboxylic acid | |
| 147 | 4-(3-chloro-5-methylphenyl)-1-(4-(3,4-dichlorophenyl)-5- | A |
| | (isopropylthio)thiazol-2-yl)-3-methyl-1H-pyrazole-5-carboxylic acid | |
| A148 | 4-(3,5-bis(trifluoromethyl)phenyl)-1-(4-(3,4-dichlorophenyl)-5- | I |
| | (isopropylthio)thiazol-2-yl)-3-methyl-N-(methylsulfonyl)-1H-pyrazole-5- | |
| | carboxamide | |
| A149 | 1-(4-(3,4-dichlorophenyl)-5-(isopropylthio)thiazol-2-yl)-4-(imidazo[1,2- | C |
| | a]pyridin-3-yl)-3-methyl-1H-pyrazole-5-carboxylic acid | |
| A150 | 4-(3,5-dichlorophenyl)-1-(4-(3,5-dichlorophenyl)-5-(isopropylthio)thiazol- | B |
| | 2-yl)-3-methyl-1H-pyrazole-5-carboxylic acid | |
| A151 | 1-(4-(3,4-dichlorophenyl)-5-(isopropylthio)thiazol-2-yl)-4-(3-fluorophenyl)- | A, B |
| | 3-methyl-1H-pyrazole-5-carboxylic acid | |
| A152 | 1-(4-(3,4-dichlorophenyl)-5-(isopropylthio)thiazol-2-yl)-4-(3-fluoro-5- | C |
| | hydroxyphenyl)-3-methyl-1H-pyrazole-5-carboxylic acid | |
| A153 | 4-(3-amino-5-methylphenyl)-1-(4-(3,4-dichlorophenyl)-5- | J |
| | (isopropylthio)thiazol-2-yl)-3-methyl-1H-pyrazole-5-carboxylic acid | |
| A154 | 4-(3-chloro-5-hydroxyphenyl)-1-(4-(3,4-dichlorophenyl)-5- | D |
| | (isopropylthio)thiazol-2-yl)-3-methyl-1H-pyrazole-5-carboxylic acid | |
| A155 | 1-(4-(3,4-dichlorophenyl)-5-(isopropylthio)thiazol-2-yl)-3-methyl-4-(1- | C |
| | methyl-1H-indol-7-yl)-1H-pyrazole-5-carboxylic acid | |
| A156 | 4-(3,4-dichlorophenyl)-5-(isopropylthio)-2-(1H-pyrazol-1-yl)thiazole | J |
| A157 | 1-(4-(2-chlorophenyl)-5-(isopropylthio)thiazol-2-yl)-4-(2,6-dimethylpyridin- | B |
| | 4-yl)-3-methyl-1H-pyrazole-5-carboxylic acid | |
| A158 | 1-(4-(3-chlorophenyl)-5-(isopropylthio)thiazol-2-yl)-4-(2,6-dimethylpyridin- | C |
| | 4-yl)-3-methyl-1H-pyrazole-5-carboxylic acid | |
| A159 | 1-(4-(4-chlorophenyl)-5-(isopropylthio)thiazol-2-yl)-4-(2,6-dimethylpyridin- | A |
| | 4-yl)-3-methyl-1H-pyrazole-5-carboxylic acid | |
| A160 | 1-(4-(2,4-dichlorophenyl)-5-(isopropylthio)thiazol-2-yl)-4-(2,6- | A |
| | dimethylpyridin-4-yl)-3-methyl-1H-pyrazole-5-carboxylic acid | |
| A161 | 1-(4-(2,5-dichlorophenyl)-5-(isopropylthio)thiazol-2-yl)-4-(2,6- | C |
| | dimethylpyridin-4-yl)-3-methyl-1H-pyrazole-5-carboxylic acid | |
| A162 | 4-(2,6-dimethylpyridin-4-yl)-1-(5-(isopropylthio)-4-(phenylethynyl)thiazol- | C |
| | 2-yl)-3-methyl-1H-pyrazole-5-carboxylic acid | |
| A163 | 1-(4-benzyl-5-(isopropylthio)thiazol-2-yl)-4-(2,6-dimethylpyridin-4-yl)-3- | D |
| | methyl-1H-pyrazole-5-carboxylic acid | |
| A164 | 1-(4-(3,4-dichlorophenyl)-5-(isopropylthio)thiazol-2-yl)-4-(3-fluoro-5- | B |
| | methoxyphenyl)-3-methyl-1H-pyrazole-5-carboxylic acid | |
| A165 | 1-(4-(3,4-dichlorophenyl)-5-(isopropylthio)thiazol-2-yl)-4-(3-methoxy-5- | B |
| | methylphenyl)-3-methyl-1H-pyrazole-5-carboxylic acid | |
| A166 | 1-(4-(3,4-dichlorophenyl)-5-(isopropylthio)thiazol-2-yl)-4-(2- | C |
| | methoxypyridin-4-yl)-3-methyl-1H-pyrazole-5-carboxylic acid | |
| A167 | 1-(4-(3,4-dichlorophenyl)-5-(isopropylthio)thiazol-2-yl)-3-methyl-4-(2- | B |
| | methylpyridin-4-yl)-1H-pyrazole-5-carboxylic acid | |
| A168 | 1-(4-(1H-indol-4-yl)-5-(isopropylthio)thiazol-2-yl)-4-(3-fluorophenyl)-3- | J |
| | methyl-1H-pyrazole-5-carboxylic acid | |
| A169 | 1-(4-(3-fluorophenyl)-5-(isopropylthio)thiazol-2-yl)-4-(1H-indol-4-yl)-3- | J |
| | methyl-1H-pyrazole-5-carboxylic acid | |
| A170 | 1-(4-(1H-indol-5-yl)-5-(isopropylthio)thiazol-2-yl)-4-(3-fluorophenyl)-3- | J |
| | methyl-1H-pyrazole-5-carboxylic acid | |
| A171 | 1-(4-(cyclopropylethynyl)-5-(isopropylthio)thiazol-2-yl)-4-(3-fluorophenyl)- | J |
| | 3-methyl-1H-pyrazole-5-carboxylic acid | |
| A172 | 4-cyclopropyl-1-(4-cyclopropyl-5-(isopropylthio)thiazol-2-yl)-3-methyl-1H- | J |
| | pyrazole-5-carboxylic acid | |
| A173 | 4-cyclopropyl-1-(4-(3,4-dichlorophenyl)-5-(isopropylthio)thiazol-2-yl)-3- | D |
| | methyl-1H-pyrazole-5-carboxylic acid | |
| A174 | 4-(3-chloro-5-isopropoxyphenyl)-1-(4-(3,4-dichlorophenyl)-5- | B |
| | (isopropylthio)thiazol-2-yl)-3-methyl-1H-pyrazole-5-carboxylic acid | |
| A175 | 4-(3-chloro-5-(2-methoxyethoxy)phenyl)-1-(4-(3,4-dichlorophenyl)-5- | B |
| | (isopropylthio)thiazol-2-yl)-3-methyl-1H-pyrazole-5-carboxylic acid | |
| A176 | 1-(4-(3-chlorophenyl)-5-(isopropylthio)thiazol-2-yl)-4-(3-fluorophenyl)-3- | C |
| | methyl-1H-pyrazole-5-carboxylic acid | |
| A177 | 1-(4-(4-chlorophenyl)-5-(isopropylthio)thiazol-2-yl)-4-(3-fluorophenyl)-3- | A |
| | methyl-1H-pyrazole-5-carboxylic acid | |
| A178 | 1-(4-(2,4-dichlorophenyl)-5-(isopropylthio)thiazol-2-yl)-4-(3-fluorophenyl)- | B |
| | 3-methyl-1H-pyrazole-5-carboxylic acid | |
| A179 | 4-bromo-1-(4-(6-(3-fluorophenyl)pyridin-3-yl)-5-(isopropylthio)thiazol-2- | E |
| | yl)-3-methyl-1H-pyrazole-5-carboxylic acid | |
| A180 | (R)-1-(4-(3,4-dichlorophenyl)-5-(isopropylthio)thiazol-2-yl)-3-methyl-4-(2- | D |
| | methyl-6-((tetrahydrofuran-3-yl)oxy)pyridin-4-yl)-1H-pyrazole-5-carboxylic | |
| | acid | |
| A181 | 1-(4-(3,4-dichlorophenyl)-5-(isopropylthio)thiazol-2-yl)-4-(2-(2- | D |
| | methoxyethoxy)-6-methylpyridin-4-yl)-3-methyl-1H-pyrazole-5-carboxylic | |
| | acid | |
| A182 | (S)-1-(4-(3,4-dichlorophenyl)-5-(isopropylthio)thiazol-2-yl)-3-methyl-4-(2- | D |
| | methyl-6-((tetrahydrofuran-3-yl)oxy)pyridin-4-yl)-1H-pyrazole-5-carboxylic | |
| | acid | |
| A183 | 1-(4-(cyclohex-1-en-1-yl)-5-(isopropylthio)thiazol-2-yl)-4-(3-fluorophenyl)- | C |
| | 3-methyl-1H-pyrazole-5-carboxylic acid | |
| A184 | 1-(4-(cyclopent-1-en-1-yl)-5-(isopropylthio)thiazol-2-yl)-4-(3-fluorophenyl)- | D |
| | 3-methyl-1H-pyrazole-5-carboxylic acid | |
| A185 | 4-(3-chloro-5-methoxyphenyl)-1-(5-(isopropylthio)-4-phenylthiazol-2-yl)-3- | D |
| | methyl-1H-pyrazole-5-carboxylic acid | |
| A186 | 4-(3-fluorophenyl)-1-(4-(4-fluorophenyl)-5-(isopropylthio)thiazol-2-yl)-3- | B |
| | methyl-1H-pyrazole-5-carboxylic acid | |
| A187 | 4-(3-fluorophenyl)-1-(5-(isopropylthio)-4-(4-methoxyphenyl)thiazol-2-yl)-3- | D |
| | methyl-1H-pyrazole-5-carboxylic acid | |
| A188 | 4-(3-fluorophenyl)-1-(5-(isopropylthio)-4-(4- | I |
| | (methylsulfonyl)phenyl)thiazol-2-yl)-3-methyl-1H-pyrazole-5-carboxylic | |
| | acid | |
| A189 | 1-(4-(4-fluoro-3-methoxyphenyl)-5-(isopropylthio)thiazol-2-yl)-4-(3- | D |
| | fluorophenyl)-3-methyl-1H-pyrazole-5-carboxylic acid | |
| A190 | 1-(4-(4-chloro-3-methylphenyl)-5-(isopropylthio)thiazol-2-yl)-4-(2,6- | B |
| | dimethylpyridin-4-yl)-3-methyl-1H-pyrazole-5-carboxylic acid | |
| A191 | 1-(4-(4-chloro-3-methylphenyl)-5-(isopropylthio)thiazol-2-yl)-4-(3- | B |
| | fluorophenyl)-3-methyl-1H-pyrazole-5-carboxylic acid | |
| A192 | 1-(4-(4-chloro-3,5-difluorophenyl)-5-(isopropylthio)thiazol-2-yl)-4-(2,6- | B |
| | dimethylpyridin-4-yl)-3-methyl-1H-pyrazole-5-carboxylic acid | |
| A193 | 1-(4-(4-chloro-3,5-difluorophenyl)-5-(isopropylthio)thiazol-2-yl)-4-(3- | B |
| | fluorophenyl)-3-methyl-1H-pyrazole-5-carboxylic acid | |
| A194 | 1-(4-(4-chloro-3-methoxyphenyl)-5-(isopropylthio)thiazol-2-yl)-4-(2,6- | B |
| | dimethylpyridin-4-yl)-3-methyl-1H-pyrazole-5-carboxylic acid | |
| A195 | 1-(4-(4-chloro-3-methoxyphenyl)-5-(isopropylthio)thiazol-2-yl)-4-(3- | C |
| | fluorophenyl)-3-methyl-1H-pyrazole-5-carboxylic acid | |
| A196 | 4-(2,6-dimethylpyridin-4-yl)-1-(5-(isopropylthio)-4-(4- | B |
| | (trifluoromethyl)phenyl)thiazol-2-yl)-3-methyl-1H-pyrazole-5-carboxylic | |
| | acid | |
| A197 | 4-(3-fluorophenyl)-1-(5-(isopropylthio)-4-(4-(trifluoromethyl)phenyl)thiazol- | A |
| | 2-yl)-3-methyl-1H-pyrazole-5-carboxylic acid | |
| A198 | 1-(4-(3,4-dichlorophenyl)-5-(isopropylthio)thiazol-2-yl)-3-methyl-4-(3- | B |
| | methyl-5-(oxetan-3-yloxy)phenyl)-1H-pyrazole-5-carboxylic acid | |
| A199 | 1-(4-(3,4-dichlorophenyl)-5-(isopropylthio)thiazol-2-yl)-4-(2-methoxy-6- | B |
| | methylpyridin-4-yl)-3-methyl-1H-pyrazole-5-carboxylic acid | |
| A200 | 1-(4-(3,4-dichlorophenyl)-5-(isopropylthio)thiazol-2-yl)-4-(3-fluoro-5- | B |
| | (oxetan-3-yloxy)phenyl)-3-methyl-1H-pyrazole-5-carboxylic acid | |
| A201a | 4-(3-fluorophenyl)-1-(4-(3-fluorophenyl)-5-(isopropylthio)thiazol-2-yl)-3- | E |
| | methyl-1H-pyrazole-5-carboxylic acid | |
| A201b | 1-(4-(benzofuran-2-yl)-5-(isopropylthio)thiazol-2-yl)-4-(3-fluorophenyl)-3- | D |
| | methyl-1H-pyrazole-5-carboxylic acid | |
| A202 | 4-(3-fluorophenyl)-1-(5-(isopropylthio)-4-phenylthiazol-2-yl)-3-methyl-1H- | D |
| | pyrazole-5-carboxylic acid | |
| A203 | 1-(5-(isopropylthio)-4-phenylthiazol-2-yl)-1′,3-dimethyl-1H,1′H-[4,4′- | I |
| | bipyrazole]-5-carboxylic acid | |
| A204 | 4-(2,6-dimethylpyridin-4-yl)-1-(4-(4-fluorophenyl)-5-(isopropylthio)thiazol- | B |
| | 2-yl)-3-methyl-1H-pyrazole-5-carboxylic acid | |
| A205 | 1-(4-(4-chloro-2-methoxyphenyl)-5-(isopropylthio)thiazol-2-yl)-4-(3- | B |
| | fluorophenyl)-3-methyl-1H-pyrazole-5-carboxylic acid | |
| A206 | 1-(4-(4-chloro-2-methoxyphenyl)-5-(isopropylthio)thiazol-2-yl)-4-(2,6- | B |
| | dimethylpyridin-4-yl)-3-methyl-1H-pyrazole-5-carboxylic acid | |
| A207 | 1-(4-(3-chloro-4-methylphenyl)-5-(isopropylthio)thiazol-2-yl)-4-(3- | C |
| | fluorophenyl)-3-methyl-1H-pyrazole-5-carboxylic acid | |
| A208 | 1-(4-(1,5-dimethyl-6-oxo-1,6-dihydropyridin-3-yl)-5-(isopropylthio)thiazol- | I |
| | 2-yl)-4-(3-fluorophenyl)-3-methyl-1H-pyrazole-5-carboxylic acid | |
| A209 | 1-(4-(2-chloro-5-(trifluoromethoxy)phenyl)-5-(isopropylthio)thiazol-2-yl)-4- | D |
| | (3-fluorophenyl)-3-methyl-1H-pyrazole-5-carboxylic acid | |
| A210 | 1-(4-(5-cyanopyridin-3-yl)-5-(isopropylthio)thiazol-2-yl)-4-(3-fluorophenyl)- | I |
| | 3-methyl-1H-pyrazole-5-carboxylic acid | |
| A211 | 1-(4-(1,3-dimethyl-1H-pyrazol-5-yl)-5-(isopropylthio)thiazol-2-yl)-4-(3- | I |
| | fluorophenyl)-3-methyl-1H-pyrazole-5-carboxylic acid | |
| A212 | 4-(3-fluorophenyl)-1-(5-(isopropylthio)-4-(pyrimidin-5-yl)thiazol-2-yl)-3- | I |
| | methyl-1H-pyrazole-5-carboxylic acid | |
| A213 | 1-(4-(4-cyanophenyl)-5-(isopropylthio)thiazol-2-yl)-4-(3-fluorophenyl)-3- | C- |
| | methyl-1H-pyrazole-5-carboxylic acid | |
| A214 | 1-(4-(3-fluoro-4-methylphenyl)-5-(isopropylthio)thiazol-2-yl)-4-(3- | B |
| | fluorophenyl)-3-methyl-1H-pyrazole-5-carboxylic acid | |
| A215 | 1-(4-(2-cyanophenyl)-5-(isopropylthio)thiazol-2-yl)-4-(3-fluorophenyl)-3- | E |
| | methyl-1H-pyrazole-5-carboxylic acid | |
| A216 | 1-(4-(4-chloro-2-methylphenyl)-5-(isopropylthio)thiazol-2-yl)-4-(3- | A |
| | fluorophenyl)-3-methyl-1H-pyrazole-5-carboxylic acid | |
| A217 | 4-(3-fluorophenyl)-1-(5-(isopropylthio)-4-(2-methyl-4- | A |
| | (trifluoromethyl)phenyl)thiazol-2-yl)-3-methyl-1H-pyrazole-5-carboxylic | |
| | acid | |
| A218 | 1-(4-(4-chloro-3-cyanophenyl)-5-(isopropylthio)thiazol-2-yl)-4-(3- | C |
| | fluorophenyl)-3-methyl-1H-pyrazole-5-carboxylic acid | |
| A219 | 4-(3,5-dichlorophenyl)-1-(5-(isopropylthio)-4-(4- | A |
| | (trifluoromethyl)phenyl)thiazol-2-yl)-3-methyl-1H-pyrazole-5-carboxylic | |
| | acid | |
| A220 | 4-(3-fluorophenyl)-1-(5-(isopropylthio)-4-(4-((2- | I |
| | methoxyethyl)carbamoyl)phenyl)thiazol-2-yl)-3-methyl-1H-pyrazole-5- | |
| | carboxylic acid | |
| A221 | 1-(4-(4-(dimethylcarbamoyl)phenyl)-5-(isopropylthio)thiazol-2-yl)-4-(3- | I |
| | fluorophenyl)-3-methyl-1H-pyrazole-5-carboxylic acid | |
| A222 | 4-(3-chloro-5-methoxyphenyl)-1-(5-(isopropylthio)-4-(4- | A |
| | (trifluoromethyl)phenyl)thiazol-2-yl)-3-methyl-1H-pyrazole-5-carboxylic | |
| | acid | |
| A223 | 4-(3-fluorophenyl)-1-(5-(isopropylthio)-4-(4- | B |
| | (trifluoromethoxy)phenyl)thiazol-2-yl)-3-methyl-1H-pyrazole-5-carboxylic | |
| | acid | |
| A224 | 4-(3-fluorophenyl)-1-(5-(isopropylthio)-4-(2-methyl-4- | A |
| | (trifluoromethoxy)phenyl)thiazol-2-yl)-3-methyl-1H-pyrazole-5-carboxylic | |
| | acid | |
| A225 | 4-(3-chloro-5-fluorophenyl)-1-(4-(3,4-dichlorophenyl)-5- | A |
| | (isopropylthio)thiazol-2-yl)-3-methyl-1H-pyrazole-5-carboxylic acid | |
| A226 | 1-(4-(4-cyano-3-methylphenyl)-5-(isopropylthio)thiazol-2-yl)-4-(3- | C |
| | fluorophenyl)-3-methyl-1H-pyrazole-5-carboxylic acid | |
| A227 | 1-(4-(3,4-dichlorophenyl)-5-fluorothiazol-2-yl)-4-(3-fluorophenyl)-3- | D |
| | methyl-1H-pyrazole-5-carboxylic acid | |
| A228 | 1-(4-(3,4-dichlorophenyl)-5-(isopropylthio)thiazol-2-yl)-4-(3-fluorophenyl)- | B |
| | 3-isopropyl-1H-pyrazole-5-carboxylic acid | |
| A229 | 1-(4-(4-(difluoromethyl)phenyl)-5-(isopropylthio)thiazol-2-yl)-4-(3- | B |
| | fluorophenyl)-3-methyl-1H-pyrazole-5-carboxylic acid | |
| A230 | 1-(4-(3,4-dichlorophenyl)-5-isopropoxythiazol-2-yl)-4-(3-fluorophenyl)-3- | C |
| | methyl-1H-pyrazole-5- | |
| | carboxylic acid | |
| A231 | 1-(4-(4-ethylphenyl)-5-(isopropylthio)thiazol-2-yl)-4-(3-fluorophenyl)-3- | B |
| | methyl-1H-pyrazole-5-carboxylic acid | |
| A232 | 2-(4-(2,6-dimethylpyridin-4-yl)-3,5-dimethyl-1H-pyrazol-1-yl)-5- | E |
| | (isopropylthio)-4-(4-(trifluoromethyl)phenyl)thiazole | |
| A233 | 4-(3-fluorophenyl)-1-(4-(5-fluoropyridin-3-yl)-5-(isopropylthio)thiazol-2-yl)- | I |
| | 3-methyl-1H-pyrazole-5-carboxylic acid | |
| A234 | 1-(4-(benzofuran-3-yl)-5-(isopropylthio)thiazol-2-yl)-4-(3-fluorophenyl)-3- | I |
| | methyl-1H-pyrazole-5-carboxylic acid | |
| A235 | 4-(3,4-difluorophenyl)-1-(5-(isopropylthio)-4-(4- | A |
| | (trifluoromethyl)phenyl)thiazol-2-yl)-3-methyl-1H-pyrazole-5-carboxylic | |
| | acid | |
| A236 | 4-(4-fluoro-3-methylphenyl)-1-(5-(isopropylthio)-4-(4- | B |
| | (trifluoromethyl)phenyl)thiazol-2-yl)-3-methyl-1H-pyrazole-5-carboxylic | |
| | acid | |
| A237 | 4-(4-fluorophenyl)-1-(5-(isopropylthio)-4-(4-(trifluoromethyl)phenyl)thiazol- | B |
| | 2-yl)-3-methyl-1H-pyrazole-5-carboxylic acid | |
| A238 | 4-(4-fluoro-3-methoxyphenyl)-1-(5-(isopropylthio)-4-(4- | B |
| | (trifluoromethyl)phenyl)thiazol-2-yl)-3-methyl-1H-pyrazole-5-carboxylic | |
| | acid | |
| A239 | 1-(4-(4-chloro-2,6-dimethylphenyl)-5-(isopropylthio)thiazol-2-yl)-4-(3- | C |
| | fluorophenyl)-3-methyl-1H-pyrazole-5-carboxylic acid | |
| A240 | 1-(4-(4-(1,1-difluoroethyl)phenyl)-5-(isopropylthio)thiazol-2-yl)-4-(3- | B |
| | fluorophenyl)-3-methyl-1H-pyrazole-5-carboxylic acid | |
| A241 | 4-(4-fluoro-3,5-dimethylphenyl)-1-(5-(isopropylthio)-4-(4- | A |
| | (trifluoromethyl)phenyl)thiazol-2-yl)-3-methyl-1H-pyrazole-5-carboxylic | |
| | acid | |
| A242 | 4-(3-fluorophenyl)-1-(5-(isopropylthio)-4-(2-(trifluoromethyl)pyrimidin-5- | C |
| | yl)thiazol-2-yl)-3-methyl-1H-pyrazole-5-carboxylic acid | |
| A243 | 1-(4-(4-chloro-3-(ethylcarbamoyl)phenyl)-5-(isopropylthio)thiazol-2-yl)-4- | I |
| | (3-fluorophenyl)-3-methyl-1H-pyrazole-5-carboxylic acid | |
| A244 | 1-(4-(2-amino-4-(trifluoromethyl)phenyl)-5-(isopropylthio)thiazol-2-yl)-4- | B |
| | (3-fluorophenyl)-3-methyl-1H-pyrazole-5-carboxylic acid | |
| A245 | 4-(3-fluorophenyl)-1-(5-(isopropylthio)-4-((4- | D |
| | (trifluoromethyl)phenyl)ethynyl)thiazol-2-yl)-3-methyl-1H-pyrazole-5- | |
| | carboxylic acid | |
| A246 | 4-(3-fluorophenyl)-1-(5-(isopropylthio)-4-(5-(trifluoromethyl)pyrimidin-2- | D |
| | yl)thiazol-2-yl)-3-methyl-1H-pyrazole-5-carboxylic acid | |
| A247 | 4-(3-fluorophenyl)-3-methyl-1-(4-(4-(trifluoromethyl)phenyl)thiazol-2-yl)- | D |
| | 1H-pyrazole-5-carboxylic acid | |
| A248 | 4-(3-fluorophenyl)-1-(5-(isopropylthio)-4-(4-(trifluoromethyl)phenyl)thiazol- | K |
| | 2-yl)-N-(2-methoxyethyl)-N,3-dimethyl-1H-pyrazole-5-carboxamide | |
| A249 | N-(2-(2-(2-(2-azidoethoxy)ethoxy)ethoxy)ethyl)-4-(3-fluorophenyl)-1-(5- | K |
| | (isopropylthio)-4-(4-(trifluoromethyl)phenyl)thiazol-2-yl)-3-methyl-1H- | |
| | pyrazole-5-carboxamide | |
| A250 | 4-(3-fluorophenyl)-1-(5-(isopropylthio)-4-(4-(trifluoromethyl)phenyl)thiazol- | D |
| | 2-yl)-N-(2-methoxyethyl)-3-methyl-1H-pyrazole-5-carboxamide | |
| A251 | 4-(3-fluorophenyl)-1-(5-(isopropylthio)-4-(4-(trifluoromethyl)phenyl)thiazol- | K |
| | 2-yl)-3-methyl-N-(propylsulfonyl)-1H-pyrazole-5-carboxamide | |
| A252 | 4-(3-fluorophenyl)-1-(5-(isopropylthio)-4-(2-(trifluoromethyl)phenyl)thiazol- | D |
| | 2-yl)-3-methyl-1H-pyrazole-5-carboxylic acid | |
| A253 | 4-(3-fluorophenyl)-1-(5-(isopropylthio)-4-(3-(trifluoromethyl)phenyl)thiazol- | C |
| | 2-yl)-3-methyl-1H-pyrazole-5-carboxylic acid | |
| A254 | 4-(3-fluorophenyl)-1-(5-(isopropylamino)-4-(4- | C |
| | (trifluoromethyl)phenyl)thiazol-2-yl)-3-methyl-1H-pyrazole-5-carboxylic | |
| | acid | |
| A255 | 4-(3-fluorophenyl)-1-(5-(isopropylthio)-4-(4-(pentafluoro-λ⁶- | A |
| | sulfaneyl)phenyl)thiazol-2-yl)-3-methyl-1H-pyrazole-5-carboxylic acid | |
| A256 | 4-(3-fluorophenyl)-1-(5-(isopropylthio)-4-(4-(trifluoromethyl)phenyl)thiazol- | D |
| | 2-yl)-N-methoxy-3-methyl-1H-pyrazole-5-carboxamide | |
| A257 | 4-(3-fluorophenyl)-1-(5-(isopropylthio)-4-(4-(2,2,2- | B |
| | trifluoroethyl)phenyl)thiazol-2-yl)-3-methyl-1H-pyrazole-5-carboxylic acid | |
| A258 | 4-(3-fluorophenyl)-1-(5-(isopropylthio)-4-(6-(trifluoromethyl)pyridin-3- | B |
| | yl)thiazol-2-yl)-3-methyl-1H-pyrazole-5-carboxylic acid | |
| A259 | 4-(3-fluorophenyl)-1-(5-(isopropylthio)-4-(1-(2,2,2-trifluoroethyl)-1H- | D |
| | pyrazol-4-yl)thiazol-2-yl)-3-methyl-1H-pyrazole-5-carboxylic acid | |

And in certain embodiments, the therapeutic compound is a compound as generally described in any genus, subgenus or embodiment of International Patent Application Publication no. 2018/012453. For example, other suitable therapeutic compounds can include the compounds having any of structural formulae (IIa)-(IIe):

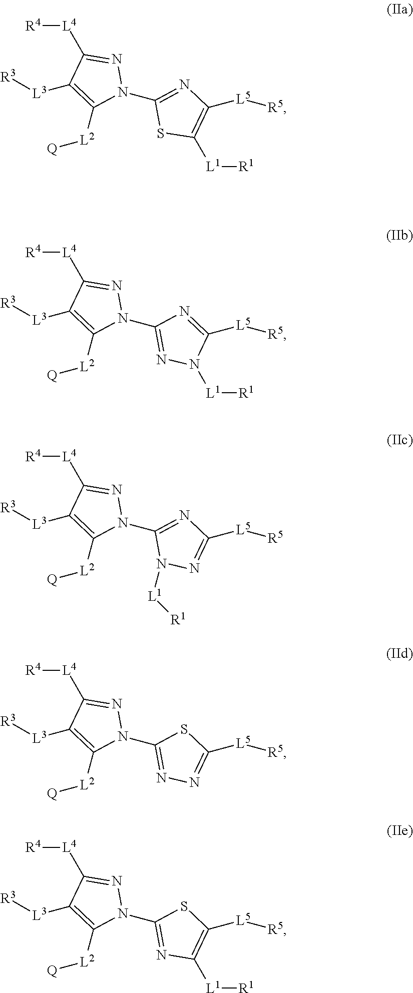

optionally in the form of a pharmaceutically acceptable salt or N-oxide, and/or a solvate or hydrate, wherein
L¹ is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
R¹ is selected from the group consisting of
   hydrogen,
   C₁-C₈ alkyl, C₁-C₈ alkenyl and C₁-C₈ alkynyl, each unsubstituted orfluorinated, cycloalkyl and heterocycloalkyl, each optionally substituted with 1-2 R^{1E}, and phenyl and monocyclic heteroaryl, each optionally substituted with 1-5 R^{1E}, in which
      each R^{1E} is independently selected from oxo, optionally-substituted C₁-C₄ alkyl, C₁-C₄ fluoroalkyl, halogen, —CN, SF₅, —N₃, —C(O)R^{1F}, —SR^{1F}, —S(O)₁₋₂R^{1F}, —OR^{1F}, —(OCH₂CH₂O)ₙ—R^{1G} in which n is 1-4, —N(R^{1G})C(O)CH₂—O—(CH₂CH₂O)ₙR^{1G} in which n is 0-3, —C(O)NR^{1G}(CH₂CH₂O)ₙR^{1G}, —NR^{1G}R^{1F} and —C(O)R^{1F};
      each R^{1F} is independently selected from H, C₁-C₃ alkyl and C₁-C₃ fluoroalkyl and each R^{1G} is independently selected from H and C₁-C₃ alkyl;
L² is selected from the group consisting of a bond, —CH₂—, —CH(CH₃)— or —CH₂CH₂—;
Q is selected from the group consisting of H, —CH₂OH, —C(O)OH, —C(O)OR^{2A}, —C(O)NR^{2B}R^{2A}, —C(O)NR^{2B}S(O)₂R^{2A}, —C(O)NR^{2B}S(O)₂NR^{2B}R^{2A}, —C(O)R^{2A}, —S(O)₂OH, —P(O)(OH)₂, —C(OH)(CF₃)₂, S(O)₂R^{2A}, —N(R^{2B})S(O)₂R^{2A}, —S(O)₂NR^{2B}R^{2A}, —C(O)NHOH, —C(O)NH—O(C₁-C₃ alkyl), and —CO(NH)CN, in which
   each R^{2A} is independently selected from H and C₁-C₃ alkyl, and
   each R^{2B} is independently selected from H and C₁-C₃ alkyl;
L³ is a bond, —C(O)—, —S—, —S(O)₁₋₂—, —O—, —NR⁶—, —CH₂—, —CH(CH₃)(OH)— or —CH(OH)—;
R³ is aryl or heteroaryl each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3D}), -L^{3C}-(heterocycloalkyl optionally substituted with 1-5 R^{3D}) and (ii) optionally substituted with 1-5 R^{3E},
   in which
      each L^{3C} is a bond, methylene, ethylene, —C(O)—, —S—, —S(O)₁₋₂—, —O— or —NR^{3G}—;
      each R^{3D} is independently selected from oxo optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F} and —NR^{3G}S(O)₁₋₂R^{3F};
      each R^{3E} is independently selected from oxo, optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F};
      each R^{3F} is independently selected from H, C₁-C₃ alkyl and C₁-C₃ fluoroalkyl and
      each R^{3G} is independently selected from H and C₁-C₃ alkyl, C₁-C₃ fluoroalkyl;
L⁴ is is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
R⁴ is selected from the group consisting of hydrogen, optionally substituted C₁-C₈ alkyl, optionally-substituted C₁-C₈ alkenyl and optionally substituted C₁-C₈ alkynyl;
L⁵ is a bond, —C(O)—, —S—, —S(O)₁₋₂—, —O—, —NR⁶—, —CH₂CH₂—, —CH═CH—, —C═C—, —CH₂—, —CH(CH₃)(OH)— or —CH(OH)—; and
R⁵ is aryl, heteroaryl, cycloalkyl or heterocycloalkyl, each (i) optionally substituted with a single substituent selected from -L^{5C}-(phenyl optionally substituted with 1-5 R^{5D}), -L^{5C}-(monocyclic heteroaryl optionally substituted with 1-5 R^{5D}), and -L^{5C}-(monocyclic cycloalkyl optionally substituted with 1-5 R^{5D}), -L^{5C}-(monocyclic heterocycloalkyl optionally substituted with 1-5 R^{5D}) and (ii) optionally substituted with 1-5 R^{5E},
   in which
      each L^{5C} is a bond, methylene, ethylene, —C(O)—, —S—, —S(O)₁₋₂—, —O— or —NR^{3G}—;
      each R^{5D} is independently selected from oxo, optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F} and —NR^{3G}S(O)₁₋₂R^{3F};
      each R^{5E} is independently selected from oxo, optionally-substituted C₁-C₄ alkyl, C₁-C₄ fluoroalkyl, halogen, —CN, —SF₅, —N₃, —C(O)R^{5F}, —SR^{5F}, —S(O)₁₋₂R^{5F}, —OR^{5F}, —NR^{5G}R^{5F}, —C(O)R^{5F}, —C(O)NR^{5G}R^{5F}, —NR^{5G}C(O)R^{5F}, —C(S)NR^{5G}R^{5F}, —NR^{1G}C(S)R^{5F}, —C(O)OR^{5F}, —OC(O)R^{5F}, —C(O)SR^{5F}, —SC(O)R^{5F}, —C(S)OR^{5F}, —OC(S)R^{5F}, —C(S)SR^{5F}, —SC(S)R^{5F}, —S(O)₁₋₂OR^{5F}, —OS(O)₁₋₂R^{5F}, —S(O)₁₋₂NR^{5G}R^{5F} and —NR^{5G}S(O)₁₋₂R^{5F};
      each R^{5F} is independently selected from H, C₁-C₃ alkyl and C₁-C₃ fluoroalkyl and
      each R^{5G} is independently selected from H and C₁-C₃ alkyl;
         wherein
each R⁶ is selected from the group consisting of hydrogen, C₁-C₃ alkyl and —C(O)(C₁-C₃ alkyl);
each optionally substituted alkyl, alkenyl and alkynyl is unsubstituted, fluorinated or substituted with one or two hydroxyl groups;
each cycloalkyl has 3-10 ring carbons and is unsaturated or partially unsaturated, and optionally includes one or two fused cycloalkyl rings, each fused ring having 3-8 ring members;
each heterocylcloalkyl has 3-10 ring members and 1-3 heteroatoms independently selected from nitrogen, oxygen and sulfur and is unsaturated or partially unsaturated, and optionally includes one or two fused cycloalkyl rings, each having 3-8 ring members;
each aryl is a phenyl or a naphthyl, and optionally includes one or two fused cycloalkyl or heterocycloalkyl rings, each fused cycloalkyl or heterocycloalkyl ring having 4-8 ring members;
each heteroaryl is a 5-6 membered monocyclic heteroaryl ring having 1-4 heteroatoms independently selected from nitrogen, oxygen and sulfur or a 8-10 membered bicyclic heteroaryl having 1-5 heteroatoms independently selected from nitrogen, oxygen or sulfur, and optionally includes one or two fused cycloalkyl or heterocycloalkyl rings, each fused cycloalkyl or heterocycloalkyl ring having 4-8 ring members.
In certain such embodiments, each and every optionally substituted alkyl, alkylene, alkenyl, alkenylene, alkynyl and alkynylene is unsubstituted orfluorinated. For example, in certain such embodiments, each and every optionally substituted alkyl, alkylene, alkenyl, alkenylene, alkynyl and alkynylene is unsubstituted.

Such a therapeutic compound can be defined generically as with respect to any of formulae (IIa), (IIb), (IIe) and (IId) above, or in various subgenera compounds in which the structural formula, R¹, L¹, L², Q, L³, R³, L⁴, R⁴, L⁵, and R⁵ are optionally independently selected from the groups (ii-1a) et seq., (ii-2a) et seq., (ii-3a) et seq., (ii-4a) et seq., (ii-5a) et seq., (ii-6a) et seq., (ii-7a) et seq., (ii-8a) et seq., (ii-9a) et seq., and (ii-10a) et seq., defined hereinbelow (e.g., wherein the compound is of a structural formula as defined in any combination of the embodiments below). Definitions of the variables can be made from any combination of groups (ii-1a) et seq., (ii-2a) et seq., (ii-3a) et seq., (ii-4a) et seq., (ii-5a) et seq., (ii-6a) et seq., (ii-7a) et seq., (ii-8a) et seq., (ii-9a) et seq., and (ii-10a) et seq., defined hereinbelow that is not logically or chemically inconsistent.

In certain embodiments of the compounds as otherwise described herein, the compound has one of the following structural formulae:
(IIa) in which the variables are as defined in any combination of groups (ii-1a) et seq., (ii-2a) et seq., (ii-3a) et seq., (ii-4a) et seq., (ii-5a) et seq., (ii-6a) et seq., (ii-7a) et seq., (ii-8a) et seq., (ii-9a) et seq., and (ii-10a) et seq. defined hereinbelow;
(IIb) in which the variables are as defined in any combination of groups (ii-1a) et seq., (ii-2a) et seq., (ii-3a) et seq., (ii-4a) et seq., (ii-5a) et seq., (ii-6a) et seq., (ii-7a) et seq., (ii-8a) et seq., (ii-9a) et seq., and (ii-10a) et seq. defined hereinbelow;
(IIe) in which the variables are as defined in any combination of groups (ii-1a) et seq., (ii-2a) et seq., (ii-3a) et seq., (ii-4a) et seq., (ii-5a) et seq., (ii-6a) et seq., (ii-7a) et seq., (ii-8a) et seq., (ii-9a) et seq., and (ii-10a) et seq. defined hereinbelow;
(IId) in which the variables are as defined in any combination of groups (ii-1a) et seq., (ii-2a) et seq., (ii-3a) et seq., (ii-4a) et seq., (ii-5a) et seq., (ii-6a) et seq., (ii-7a) et seq., (ii-8a) et seq., (ii-9a) et seq., and (ii-10a) et seq. defined hereinbelow;
(IIe) in which the variables are as defined in any combination of groups (ii-1a) et seq., (ii-2a) et seq., (ii-3a) et seq., (ii-4a) et seq., (ii-5a) et seq., (ii-6a) et seq., (ii-7a) et seq., (ii-8a) et seq., (ii-9a) et seq., and (ii-10a) et seq. defined hereinbelow.

In certain embodiments of the compounds as otherwise described herein. R¹ is selected from one of the following groups (ii-1a)-(ii-1k):
(ii-1a) R¹ is selected from the group consisting of hydrogen, optionally substituted C₁-C₈ alkyl and cycloalkyl optionally substituted with 1-5 R^{1E};
(ii-1 b) R¹ is hydrogen;
(ii-1c) R¹ is optionally substituted C₁-C₈ alkyl;
(ii-1 d) R¹ is unsubstituted C₁-C₈ alkyl or fluorinated C₁-C₈ alkyl, e.g., propyl or butyl;
(ii-1e) R¹ is unsubstituted cycoalkyl;
(ii-1f) R¹ is optionally substituted C₁-C₈ alkenyl;
(ii-1g) R¹ is phenyl optionally substituted with 1-5 R^{E};
(ii-1 h) R¹ is propyl, butyl, or butenyl;
(ii-1i) R¹ is trifluoromethyl-substituted phenyl, methoxy-substituted phenyl or fluoro-substituted phenyl.
(ii-1j) R¹ is phenyl substituted with —(OCH₂CH₂O)ₙ—R^{1G} in which n is 1-4, —N(R^{1G})C(O)CH₂—O—(CH₂CH₂O)ₙR^{1G} in which n is 0-3, or —C(O)NR^{1G}(CH₂CH₂O)ₙR^{1G};
(ii-1k) R¹ is hydroxymethyl, methoxymethyl, hydroxyethyl or methoxyethyl.
   In certain such embodiments, each optionally substituted alkyl of R¹ (including those of R^{1E}) is unsubstituted or fluorinated. For example, in certain such embodiments each optionally substituted alkyl, alkenyl and alkynyl of R¹ (including those of R^{1E}) is unsubstituted.

In certain embodiments of the compounds as otherwise described herein.

L¹ is selected from one of the following groups (ii-2a)-(ii-2e)
(ii-2a) L¹ is a bond, —S—, —S(O)— or —S(O)₂—;
(ii-2b) L¹ is selected from a bond, —CH₂—, —CH(CH₃)—, —CH₂CH₂—, —C(O)—, —S—, —S(O)₁₋₂—, —O—, and —NR⁶—;
(ii-2c) L¹ is —O— or —S—.
(ii-2d) L¹ is a bond (e.g., when R¹ is (ii-1 d), (ii-1f), (ii-1 g), (ii-1i), (ii-1j) or (ii-1k) above);
(ii-2e) L¹ is —NR⁶—.

In certain embodiments of the compounds as otherwise described herein. L² is selected from one of the following groups (ii-3a)-(ii-3c)
(ii-3a) L² is —CH₂—, —CH(CH₃)— or —CH₂CH₂—;
(ii-3b) L² is a bond;
(ii-3c) L² is a bond or —CH₂—.

In certain embodiments of the compounds as otherwise described herein. Q is selected from one of the following groups (ii-4a)-(ii-4d)
(ii-4a) Q is selected from the group consisting of —CH₂OH, —C(O)OH, —C(O)OR^{2A}, —C(O)NR^{2B}R^{2A}, —C(O)NR^{2B}S(O)₂R^{2A}, —C(O)NR^{2B}S(O)₂NR^{2B}R^{2A}, —C(O)R^{2A}, —S(O)₂OH, —P(O)(OH)₂, —C(OH)(CF₃)₂, S(O)₂R^{2A}, —N(R^{2B})S(O)₂R^{2A}, —S(O)₂NR^{2B}R^{2A}, —C(O)NH—O(C₁-C₃ alkyl), —C(O)NHOH and —CO(NH)CN;
(ii-4b) Q is selected from the group consisting of —CH₂OH, —C(O)OH, —C(O)OR^{2A}, —C(O)NR^{2B}R^{2A}, —C(O)NR^{2B}S(O)₂R^{2A}, —C(O)NR^{2B}S(O)₂NR^{2B}R^{2A}, —C(O)R^{2A}, —S(O)₂OH, —P(O)(OH)₂.
(ii-4c) Q is —CH₂OH, —C(O)OH or —C(O)OR^{2A};
(ii-4d) Qis-COOH.

In certain embodiments of the compounds as otherwise described herein. L³ is selected from one of the following groups (ii-5a)-(ii-5c)
(ii-5a) L³ is a bond, —C(O)—, —S—, —S(O)₁₋₂—, —O—, —NR⁶—, —CH₂—, —CH(CH₃)(OH)— or —CH(OH)—;
(ii-5b) L³ is a bond;
(ii-5c) L³ is a bond, —CH₂—, —CH(CH₃)(OH)— or —CH(OH)—.

In certain embodiments of the compounds as otherwise described herein. R³ is selected from one of the following groups (ii-6a)-(ii-6k)
(ii-6a) R³ is aryl (e.g., phenyl) or heteroaryl (e.g., monocyclic heteroaryl) each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3D}), -L^{3C}-(heterocycloalkyl optionally substituted with 1-5 R^{3D}) and (ii) optionally substituted with 1-5 R^{3E};
(ii-6b) R³ is aryl (e.g., a phenyl, a benzodioxole, or a dihydro-1H-isoquinoline) optionally substituted with 1-5 R^{3E};
(ii-6c) R³ is aryl (e.g., a phenyl, a benzodioxole, or a dihydro-1H-isoquinoline) (i) substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3D}), -L^{3C}-(heterocycloalkyl optionally substituted with 1-5 R^{3D}) and (ii) optionally substituted with 1-5 R^{3E};
(ii-6d) R³ is aryl (e.g., a phenyl, a benzodioxole, or a dihydro-1H-isoquinoline) (i) substituted with a single substituent selected from -L^{3C}-(phenyl optionally substituted with 1-5 R^{3D}), -L^{3C}-(monocyclic heteroaryl optionally substituted with 1-5 R^{3D}), -L^{3C}-(monocyclic cycloalkyl optionally substituted with 1-5 R^{3D}), -L^{3C}-(monocyclic heterocycloalkyl optionally substituted with 1-5 R^{3D}) and (ii) optionally substituted with 1-5 R^{3E};
(ii-6e) R³ is as defined in (6a)-(6d), wherein the aryl is not substituted with any R^{3E};
(ii-6f)R³ is heteroaryl (e.g., an isothiazole, a pyridone, a thiadiazole, a pyrazine, a pyrazolopyrimidine, a pyrazolopyridine, an imidazole, a benzofuran, an indole, an imidazopyridine, a pyridine, a pyrazole, an isoxazole, a triazolopyridine, a benzimidazole, a thiophene, a benzothiophene, a furan or a pyrimidine) optionally substituted with 1-5 R^{3E};
(ii-6g) R³ is heteroaryl (e.g., an isothiazole, a pyridone, a thiadiazole, a pyrazine, a pyrazolopyrimidine, a pyrazolopyridine, an imidazole, a benzofuran, an indole, an imidazopyridine, a pyridine, a pyrazole, an isoxazole, a triazolopyridine, a benzimidazole, a thiophene, a benzothiophene, a furan or a pyrimidine) (i) substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3D}), -L^{3C}-(heterocycloalkyl optionally substituted with 1-5 R^{3D}) and (ii) optionally substituted with 1-5 R^{3E};
(ii-6h) R³ is heteroaryl (e.g., an isothiazole, a pyridone, a thiadiazole, a pyrazine, a pyrazolopyrimidine, a pyrazolopyridine, an imidazole, a benzofuran, an indole, an imidazopyridine, a pyridine, a pyrazole, an isoxazole, a triazolopyridine, a benzimidazole, a thiophene, a benzothiophene, a furan or a pyrimidine) (i) substituted with a single substituent selected from -L^{3C}-(phenyl optionally substituted with 1-5 R^{3D}), -L^{3C}-(monocyclic heteroaryl optionally substituted with 1-5 R^{3D}), -L^{3C}-(monocyclic cycloalkyl optionally substituted with 1-5 R^{3D}), -L^{3C}-(monocyclic heterocycloalkyl optionally substituted with 1-5 R^{3D}) and (ii) optionally substituted with 1-5 R^{3E};
(ii-6i)R³ is as defined in (6f)-(6h), wherein the heteroaryl is not substituted with any R^{3E};
(ii-6j) R³ is selected from the group consisting of: phenyl, benzodioxolyl, dihydro-1H-isoquinolinyl, imidazolyl, oxazolyl, isoxazolyl, isothiazolyl, thiazolyl, pyridinyl, and pyrazinyl, pyridonyl, thiadiazolyl, pyrazolopyrimidinyl, pyrazolopyridinyl, benzofuranyl, indolyl, imidazopyridinyl, pyrazolyl, triazolopyridinyl, benzimidazolyl, a benzimidazolyl, a thienyl, a benzothienyl, a furanyl and pyrimidinyl, each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3D}), -L^{3C}-(heterocycloalkyl optionally substituted with 1-5 R^{3D}) and (ii) optionally substituted with 1-5 R^{3E}.
(ii-6k) R³ is selected from the group consisting of phenyl and monocyclic heteroaryl (e.g., pyridyl, pyrazolyl), optionally substituted with 1-5 R^{3E}.
   In certain such embodiments, each optionally substituted alkyl, alkenyl and alkynyl of R³ (including those of R^{3D} and R^{3E}) is unsubstituted or fluorinated. For example, in certain such embodiments each optionally substituted alkyl, alkenyl and alkynyl of R³ (including those of R^{3D} and R^{3E}) is unsubstituted. In certain such embodiments, L^{3C} is methylene or —O—. In certain such embodiments, the optional number of R3E substituents is 1-3, or 1-2.

In certain embodiments of the compounds as otherwise described herein. R⁴ is selected from one of the following groups (ii-7a)-(ii-7d)
(ii-7a) R⁴ is hydrogen;
(ii-7b) R⁴ is optionally substituted C₁-C₈ alkyl, optionally-substituted C₁-C₈ alkenyl or optionally substituted C₁-C₈ alkynyl;
(ii-7c) R⁴ is hydrogen or unsubstituted C₁-C₆ alkyl;
(ii-7d) R⁴ is unsubstituted C₁-C₃ alkyl.
   In certain such embodiments, each optionally substituted alkyl, alkenyl and alkynyl of R⁴ is unsubstituted or fluorinated. For example, in certain such embodiments each optionally substituted alkyl, alkenyl and alkynyl of R⁴ is unsubstituted.

In certain embodiments of the compounds as otherwise described herein.

L⁴ is selected from one of the following groups (ii-8a)-(ii-8c)
ii-(8a) L⁴ is selected from a bond, —C(O)—, —S—, —S(O)₁₋₂—, —O—, and —NR⁶—;
(ii-8b) L⁴ is a bond;
(ii-8c) L⁴ is —O— (e.g., when R⁴ is any of (ii-7a), (ii-7b), (ii-7c) or (ii-7d) above).

In certain embodiments of the compounds as otherwise described herein. L⁵ is selected from one of the following groups (ii-9a)-(ii-9c)
(ii-9a) L⁵ is a bond, —C(O)—, —S—, —S(O)₁₋₂—, —O—, —NR⁶—, —CH₂CH₂—, —CH₂—, —CH(CH₃)(OH)— or —CH(OH)—;
(ii-9b) L⁵ is a bond;
(ii-9c) L⁵ is a bond, —O—, —S—, —C(O)— or —S(O)₁₋₂—.

In certain embodiments of the compounds as otherwise described herein. R⁵ is selected from one of the following groups (ii-10a)-(ii-10s)
(ii-10a) R⁵ is aryl (e.g., phenyl) or heteroaryl (e.g., an isoxazolyl, a pyridyl, an imidazopyridyl, a pyrazolyl), each optionally substituted with 1-5 R^{5E};
(ii-10b) R⁵ is phenyl optionally substituted with 1-5 R^{5E};
(ii-10c)R⁵ is selected from the group consisting of phenyl, isoxazolyl, pyridyl, imidazopyridyl, and pyrazolyl, each optionally substituted with 1-5 R^{5E}.
(ii-1d) R⁵ is phenyl substituted with a single substituent selected from -L^{5C}-(phenyl optionally substituted with 1-5 R^{5D}), -L^{5C}-(monocyclic heteroaryl optionally substituted with 1-5 R^{5D}), and -L^{5C}-(monocyclic cycloalkyl optionally substituted with 1-5 R^{5D}) -L^{5C}-(monocyclic heterocycloalkyl optionally substituted with 1-5 R^{5D}) and (ii) optionally substituted with 1-5 R^{5E};
(ii-10e)R⁵ is phenyl substituted with a single -L^{5C}-(monocyclic heteroaryl optionally substituted with 1-5 R^{5D}) substituent and (ii) optionally substituted with 1-5 R^{5E};
(ii-10f) R⁵ is phenyl substituted with a single -L^{5C}-(monocyclic heterocycloalkyl optionally substituted with 1-5 R^{5D}) substituent and (ii) optionally substituted with 1-5 R^{5E};
(ii-10g) (ii-1 Od), (ii-10e) or (ii-10f) above, in which L^{5C} is a bond;
(ii-10h) (ii-1 Od), (ii-10e) or (ii-10f) above, in which L^{5C} is —O— or —C(O)—;
(ii-10h) R⁵ is heterocycloalkyl optionally substituted with 1-5 R^{5E};
(ii-10i) R⁵ is heterocycloalkyl substituted with a single -L^{5C}-(monocyclic cycloalkyl optionally substituted with 1-5 R^{5D}) substituent and (ii) optionally substituted with 1-5 R^{5E};
(ii-10j) (ii-10h) or (ii-10i) above, in which the heterocycloalkyl is a nitrogen-containing heterocycloalkyl, attached to the -L⁵- through a nitrogen atom;
(ii-10k)(ii-10h), (ii-10i) or (ii-10j) above, in which the heterocycloalkyl is monocyclic;
(ii-10l) (ii-10h), (ii-10i) or (ii-10j) above, in which the heterocycloalkyl is bicyclic;
(ii-10m) any of (ii-10h)-(ii-10l) above, in which the heterocycloalkyl is saturated;
(ii-10n) R⁵ is cycloalkyl optionally substituted with 1-5 R^{5E};
(ii-10o) (ii-10n) above, in which the cycloalkyl is substituted with 1-5 R^{5E};
(ii-10p) (ii-10n) or (ii-10o) above, in which the cycloalkyl is monocyclic;
(ii-10q) any of (ii-10n), (ii-10o) or (ii-10p) above, in which the cycloalkyl is saturated;
(ii-10r) any of (ii-10n), (ii-10o) or (ii-10p) above, in which the cycloalkyl is unsaturated, e.g., singly unsaturated;
(ii-10s) any of (ii-10n), (ii-10o) or (ii-10p) above, in which the cycloalkyl is cyclohexen-1-yl;
   In certain such embodiments, each optionally substituted alkyl, alkenyl and alkynyl of R⁵ (including those of R^{5D} and R^{5E}) is unsubstituted or fluorinated. For example, in certain such embodiments each optionally substituted alkyl, alkenyl and alkynyl of R⁵ (including those of R^{5D} and R^{5E}) is unsubstituted.

Other embodiments of the compounds as otherwise described herein have any of the structural formulae (IIa)-(IIe) above, for example, structural formula (IIa), in which the variables are as otherwise described in any embodiment herein (e.g., with respect to any of the alternative definitions of the variables L¹, R¹, L², Q, L³, R³, L⁵ and R⁵ as described herein), and in which -L⁴-R⁴ is —OH or —O— (unsubstituted or fluorinated C₁-C₈ alkyl), e.g., methoxy.

Other embodiments of the compounds as otherwise described herein have any of the structural formulae (IIa)-(IIe) above, for example, structural formula (IIa), in which the variables are as otherwise described in any embodiment herein (e.g., with respect to any of the alternative definitions of the variables L¹, R¹, L², Q, L³, R³, L⁴ and R⁴ as described herein), and in which -L⁵-R⁵ is phenyl substituted with a single substituent selected from -L^{5C}-(phenyl optionally substituted with 1-5 R^{5D}), -L^{5C}-(monocyclic heteroaryl optionally substituted with 1-5 R^{5D}), and -L^{5C}-(monocyclic cycloalkyl optionally substituted with 1-5 R^{5E}) -L^{5C}-(monocylclic heterocycloalkyl optionally substituted with 1-5 R^{5E}) and (ii) optionally substituted with 1-5 R^{5E}.

Other embodiments of the compounds as otherwise described herein have any of the structural formulae (IIa)-(IIe) above, for example, structural formula (IIa), in which the variables are as otherwise described in any embodiment herein (e.g., with respect to any of the alternative definitions of the variables L¹, R¹, L², Q, L³, R³, L⁴ and R⁴ as described herein), and in which -L⁵-R⁵ is R⁵ is phenyl substituted with a single -L^{5C}-(monocyclic heteroaryl optionally substituted with 1-5 R^{5D}) substituent and (ii) optionally substituted with 1-5 R^{5E}. The monocyclic heteroaryl can be, for example, an oxadiazole.

Other embodiments of the compounds as otherwise described herein have any of the structural formulae (IIa)-(IIe) above, for example, structural formula (IIa), in which the variables are as otherwise described in any embodiment herein (e.g., with respect to any of the alternative definitions of the variables L¹, R¹, L², Q, L³, R³, L⁴ and R⁴ as described herein), and in which -L⁵-R⁵ is R⁵ is phenyl substituted with a single -L^{5C}-(monocyclic heterocycloalkyl optionally substituted with 1-5 R^{5E}) substituent and (ii) optionally substituted with 1-5 R^{5E}. The monocyclic heterocycloalkyl can be, for example, an morpholinyl, e.g., a morpholin-1-yl, or a oxetanyl, e.g., an oxetan-3-yl.

Other embodiments of the compounds as otherwise described herein have any of the structural formulae (IIa)-(IIe) above, for example, structural formula (IIa), in which the variables are as otherwise described in any embodiment herein (e.g., with respect to any of the alternative definitions of the variables L¹, R¹, L², Q, L³, R³, L⁴ and R⁴ as described herein), and in which -L⁵-R⁵ is heterocycloalkyl optionally substituted with 1-5 R^{5E}. The heterocycloalkyl can be, for example, a nitrogen-containing heterocycloalkyl, attached to the -L⁵- through a nitrogen atom. In certain such embodiments, the heterocycloalkyl is moncyclic. In other such embodiments, the heterocycloalkyl is bicyclic. In certain such embodiments, the heterocycloalkyl is saturated. In various embodiments as otherwise described herein, the heterocycloalkyl is a morpholinyl (e.g., a morpholin-1-yl), a 1,4-dioxaspiro[4,5]dec-enyl (e.g., 1,4-dioxaspiro[4,5]dec-en-8-yl), a piperidinyl (e.g., a piperidin-1-yl), an azabicyclo[3.2.1]octanyl (e.g., an azabicyclo[3.2.1]octan-8-yl), a piperazinyl (e.g., a piperazin-1-yl), a pyrrolidinyl (e.g., a pyrrolidin-1-yl), or an azaspiro[2.5]octanyl (e.g., an azaspiro[2.5]octan-6-yl).

Other embodiments of the compounds as otherwise described herein have any of the structural formulae (IIa)-(IIe) above, for example, structural formula (IIa), in which the variables are as otherwise described in any embodiment herein (e.g., with respect to any of the alternative definitions of the variables L¹, R¹, L², Q, L³, R³, L⁴ and R⁴ as described herein), and in which -L⁵-R⁵ is is heterocycloalkyl substituted with a single -L^{5C}-(monocyclic cycloalkyl optionally substituted with 1-5 R^{5E}) substituent and (ii) optionally substituted with 1-5 R^{5E}. The heterocycloalkyl can be, for example, a nitrogen-containing heterocycloalkyl, attached to the -L⁵- through a nitrogen atom. In certain such embodiments, the heterocycloalkyl is moncyclic. In other such embodiments, the heterocycloalkyl is bicyclic. In certain such embodiments, the heterocycloalkyl is saturated. In various embodiments as otherwise described herein, the heterocycloalkyl is a morpholinyl (e.g., a morpholin-1-yl), a 1,4-dioxaspiro[4,5]dec-enyl (e.g., 1,4-dioxaspiro[4,5]dec-en-8-yl), a piperidinyl (e.g., a piperidin-1-yl), an azabicyclo[3.2.1]octanyl (e.g., an azabicyclo[3.2.1]octan-8-yl), a piperazinyl (e.g., a piperazin-1-yl), a pyrrolidinyl (e.g., a pyrrolidin-1-yl), or an azaspiro[2.5]octanyl (e.g., an azaspiro[2.5]octan-6-yl). The cycloalkyl can be, for example, a saturated cycloalkyl, such as a saturated C₃-C₅ cycloalkyl, e.g., cyclopropyl.

Other embodiments of the compounds as otherwise described herein have any of the structural formulae (IIa)-(IIe) above, for example, structural formula (IIa), in which the variables are as otherwise described in any embodiment herein (e.g., with respect to any of the alternative definitions of the variables L¹, R¹, L², Q, L³, R³, L⁴ and R⁴ as described herein), and in which -L⁵-R⁵ is cycloalkyl optionally substituted with 1-5 R^{5E}. In certain such embodiments, the cycloalkyl is moncyclic. In other such embodiments, the cycloalkyl is bicyclic. In certain such embodiments, the cycloalkyl is saturated. In various embodiments as otherwise described herein, the cycloalkyl is a cyclohexenyl (e.g., a cyclohexen-1-yl, for example, 4-trifluoromethylcyclohexen-1-yl), or a cyclohexyl.

Other embodiments of the compounds as otherwise described herein have any of the structural formulae (IIa)-(IIe) above, for example, structural formula (IIa), in which the variables are as otherwise described in any embodiment herein (e.g., with respect to any of the alternative definitions of the variables L¹, R¹, L², Q, L³, R³, L⁴ and R⁴ as described herein), and in which -L⁵-R⁵ is phenyl substituted with one, two or three substituents each independently selected from fluoro, chloro, nitro, methyl, methoxy, ethyl, ethoxy, trifluoromethyl, difluoromethyl, fluoromethyl, trifluoromethoxy, pentafluoroethyl and 2,2,2-trifluoroethoxy. In certain such embodiments, L⁵ is a bond.

In certain additional embodiments, including any of the embodiments described with reference to formulae (IIa)-(IIe), each optionally substituted alkylene, alkenylene, and alkynylene recited in any one of the preceding embodiments is unsubstituted. In alternative additional embodiments, including any of the embodiments described with reference to formulae (IIa)-(IIe), each optionally substituted alkylene, alkenylene, and alkynylene recited in any one of the preceding embodiments is unsubstituted orfluorinated.

In certain additional embodiments, including any of the embodiments described with reference to formulae (IIa)-(IIe), and any embodiment described in the paragraph immediately above, each optionally substituted alkyl, alkenyl, and alkynyl recited in any one of preceding embodiments is unsubstituted. In alternative additional embodiments, including any of the embodiments described with reference to formulae (IIa)-(IIe), and any embodiment described in the paragraph immediately above, each optionally substituted alkyl, alkenyl, and alkynyl recited in any one of preceding embodiments is unsubstituted.

In certain additional embodiments, including any of the embodiments described with reference to formulae (IIa)-(IIe), and any embodiment described in the two paragraphs immediately above, each cycloalkyl recited in any one of the preceding embodiments is a 3-7 membered monocyclic cycloalkyl. For example, in certain particular embodiments, including any of the embodiments described with reference to formulae (IIa)-(IIe), and any embodiment described in the two paragraphs immediately above, each cycloalkyl recited in any one of the preceding embodiments is a cyclopropyl, a cyclobutyl, a cyclopentyl, a cyclopentenyl, a cyclohexyl or a cyclohexenyl.

In certain additional embodiments, including any of the embodiments described with reference to formulae (IIa)-(IIe), and any embodiment described in the three paragraphs immediately above, each heterocycloalkyl recited in any one of the preceding embodiments is a 4-7 membered monocyclic heterocycloalkyl having 1-2 heteroatoms selected from O, S and N. For example, in certain particular embodiments, including any of the embodiments described with reference to formulae (IIa)-(IIe), and any embodiment described in the three paragraphs immediately above, each heterocycloalkyl recited in any one of the preceding embodiments is a pyrrolidinyl, a tetrahydrofuranyl, a tetrahydrothienyl, a piperidinyl, a piperazinyl, a morpholinyl, a thiomorpholinyl, a tetrahydro-2H-pyranyl, or a tetrahydro-2H-thiopyranyl.

In certain additional embodiments, including any of the embodiments described with reference to formulae (IIa)-(IIe), and any embodiment described in the four paragraphs immediately above, each heteroaryl is a 5-6 membered monocyclic heteroaryl having 1-3 heteroatoms selected from O, S and N. For example, in certain particular embodiments, including any of the embodiments described with reference to formulae (IIa)-(IIe), and any embodiment described in the four paragraphs immediately above, each heteroaryl is a furanyl, a thienyl, a pyrrolyl, a pyrazolyl, an imidazolyl, an oxazolyl or a thiazolyl.

In certain additional embodiments, including any of the embodiments described with reference to formulae (IIa)-(IIe), and any embodiment described in the four paragraphs immediately above, each aryl is phenyl.

In certain additional embodiments as described above, including any of the embodiments described with reference to formulae (IIa)-(IIe), and any embodiment described in the five paragraphs immediately above, R⁵ is substituted with 1, 2 or 3 substituents selected from halogen (e.g., chloro- or fluoro-) and fluorinated C₁-C₃ alkyl (e.g., trifluoromethyl, difluoromethyl, fluoromethyl, pentafluoroethyl, trifluoroethyl). For example, in certain embodiment as described above, R⁵ is phenyl substituted (e.g., 3-substituted, 4-substituted, 3,4-disubstituted, 2,4-disubstituted, or 2,5-disubstituted) with one or two substitutents selected from trifluoromethyl, fluorine and chlorine. For example, in particular embodiments, R⁵ can be dichlorophenyl, e.g., 3,4-dichlorophenyl, or trifluoromethylphenyl, e.g., 4-trifluoromethylphenyl.

In certain embodiments, the therapeutic compound is one of the compounds of the compound table below, optionally provided as a pharmaceutically-acceptable salt or N-oxide, and/or a solvate or hydrate. BJAB (malignant human B-cell-line) cell proliferation data is presented in the table; “A” indicates a measured EC₅₀ less than or equal to 1 μM; “B” indicates a measured EC₅₀ greater than 1 μM and less than or equal to 5 μM; “C” indicates a measured EC₅₀ greater than 5 μM and less than or equal to 10 μM; “D” indicates a measured EC₅₀ greater than 10 μM and less than or equal to 25 μM; “E” indicates a measured EC₅₀ greater than 25 μM and less than or equal to 50 μM; “F” indicates a measured EC₅₀ greater than 50 μM and less than or equal to 100 μM; “G” indicates that in the experiments performed there was no measured EC₅₀ less than or equal to 80 μM; “H” indicates that in the experiments performed there was no measured EC₅₀ less than or equal to 50 μM; “I” indicates that in the experiments performed there was no measured EC₅₀ less than or equal to 40 μM; “J” indicates that in the experiments performed there was no measured EC₅₀ less than or equal to 25 μM; “K” indicates that in the experiments performed there was no measured EC₅₀ less than or equal to 20 μM; and “L” indicates that in the experiments performed there was no measured EC₅₀ less than or equal to 5 μM. In certain embodiments, the therapeutic compound is a compound having an activity as “A,” “B” or “C” in the table below. In certain embodiments, the therapeutic compound is a compound having an activity as “A” or “B” in the table below. In certain embodiments, the therapeutic compound is a compound having an activity as “A” in the table below.

| Cpd | Name | BJAB |
|---|---|---|
| B1 | 1-(4-(4-chloro-2-(oxetan-3-yloxy)phenyl)-5-(isopropylthio)thiazol-2- | I |
| | yl)-3-methyl-1H-pyrazole-5-carboxylic acid | |
| B2 | 1-(4-(4-chloro-3-(oxetan-3-yloxy)phenyl)-5-(isopropylthio)thiazol-2- | I |
| | yl)-3-methyl-1H-pyrazole-5-carboxylic acid | |
| B3 | 4-(3-fluorophenyl)-1-(5-(isopropylthio)-4-(4-(5-methyl-1,3,4- | E |
| | oxadiazol-2-yl)phenyl)thiazol-2-yl)-3-methyl-1H-pyrazole-5- | |
| | carboxylic acid | |
| B4 | 4-(3-fluorophenyl)-1-(5-(isopropylthio)-4-(4-methylcyclohex-1-en-1- | B |
| | yl)thiazol-2-yl)-3-methyl-1H-pyrazole-5-carboxylic acid | |
| B5 | 4-(3-fluorophenyl)-1-(5-(isopropylthio)-4-(4- | A |
| | (trifluoromethyl)cyclohex-1-en-1-yl)thiazol-2-yl)-3-methyl-1H- | |
| | pyrazole-5-carboxylic acid | |
| B6 | 1-(4-(4,4-dimethylcyclohex-1-en-1-yl)-5-(isopropylthio)thiazol-2-yl)- | A |
| | 4-(3-fluorophenyl)-3-methyl-1H-pyrazole-5-carboxylic acid | |
| B7 | 4-(3-fluorophenyl)-1-(5-(isopropylthio)-4-(1,4-dioxaspiro[4.5]dec-7- | E |
| | en-8-yl)thiazol-2-yl)-3-methyl-1H-pyrazole-5-carboxylic acid | |
| B8 | 1-(4-(4-chloro-3-(morpholine-4-carbonyl)phenyl)-5- | I |
| | (isopropylthio)thiazol-2-yl)-4-(3-fluorophenyl)-3-methyl-1H-pyrazole- | |
| | 5-carboxylic acid | |
| B9 | 4-(3,4-dichlorophenyl)-2-(4-(2,6-dimethylpyridin-4-yl)-3-methyl-1H- | A, C, L |
| | pyrazol-1-yl)-5-(isopropylthio)thiazole | |
| B10 | 2-(4-(3-fluorophenyl)-3,5-dimethyl-1H-pyrazol-1-yl)-5- | I |
| | (isopropylthio)-4-(4-(trifluoromethyl)phenyl)thiazole | |
| B11 | 4-(3-fluorophenyl)-1-(5-(isopropylthio)-4-(piperidin-1-yl)thiazol-2-yl)- | D |
| | 3-methyl-1H-pyrazole-5-carboxylic acid | |
| B12 | 4-(3-fluorophenyl)-1-(5-(isopropylthio)-4-(4- | A |
| | (trifluoromethyl)piperidin-1-yl)thiazol-2-yl)-3-methyl-1H-pyrazole-5- | |
| | carboxylic acid | |
| B13 | 4-(3-fluorophenyl)-1-(5-(isopropylthio)-4-(4- | B |
| | (trifluoromethyl)phenyl)thiazol-2-yl)-3-methoxy-1H-pyrazole-5- | |
| | carboxylic acid | |
| B14 | 4-(3-fluorophenyl)-1-(5-(isopropylthio)-4-morpholinothiazol-2-yl)-3- | I |
| | methyl-1H-pyrazole-5-carboxylic acid | |
| B15 | 4-(3-fluorophenyl)-3-hydroxy-1-(5-(isopropylthio)-4-(4- | K |
| | (trifluoromethyl)phenyl)thiazol-2-yl)-1H-pyrazole-5-carboxylic acid | |
| B16 | 1-(5-(3,4-dichlorophenyl)-1-isobutyl-1H-1,2,4-triazol-3-yl)-4-(3- | K |
| | fluorophenyl)-3-methyl-1H-pyrazole-5-carboxylic acid | |
| B17 | 1-(3-(3,4-dichlorophenyl)-1-isobutyl-1H-1,2,4-triazol-5-yl)-4-(3- | K |
| | fluorophenyl)-3-methyl-1H-pyrazole-5-carboxylic acid | |
| B18 | 1-(4-(4,4-difluoropiperidin-1-yl)-5-(isopropylthio)thiazol-2-yl)-4-(3- | B |
| | fluorophenyl)-3-methyl-1H-pyrazole-5-carboxylic acid | |
| B19 | methyl 4-(3-fluorophenyl)-1-(5-(isopropylthio)-4-(4- | A |
| | (trifluoromethyl)cyclohex-1-en-1-yl)thiazol-2-yl)-3-methyl-1H- | |
| | pyrazole-5-carboxylate (enantiopure - unknown stereochemistry) | |
| B20 | methyl 4-(3-fluorophenyl)-1-(5-(isopropylthio)-4-(4- | A |
| | (trifluoromethyl)cyclohex-1-en-1-yl)thiazol-2-yl)-3-methyl-1H- | |
| | pyrazole-5-carboxylate (enantiopure - unknown stereochemistry) | |
| B21 | 4-(3-fluorophenyl)-3-methyl-1-(4-(4- | K |
| | (trifluoromethyl)cyclohexyl)thiazol-2-yl)-1H-pyrazole-5-carboxylic | |
| | acid | |
| B22 | 4-(3-fluorophenyl)-1-(5-isobutyl-4-(4-(trifluoromethyl)phenyl)thiazol- | A |
| | 2-yl)-3-methyl-1H-pyrazole-5-carboxylic acid | |
| B23 | 4-(3-fluorophenyl)-1-(5-(isopropylthio)-4-(4-(2,2,2- | C |
| | trifluoroethyl)piperazin-1-yl)thiazol-2-yl)-3-methyl-1H-pyrazole-5- | |
| | carboxylic acid | |
| B24 | 1-(4-(4-cyanopiperidin-1-yl)-5-(isopropylthio)thiazol-2-yl)-4-(3- | D |
| | fluorophenyl)-3-methyl-1H-pyrazole-5-carboxylic acid | |
| B25 | 1-(4-(4-cyclopropylpiperazin-1-yl)-5-(isopropylthio)thiazol-2-yl)-4-(3- | C |
| | fluorophenyl)-3-methyl-1H-pyrazole-5-carboxylic acid | |
| B26 | 1-(4-(4-ethylpiperazin-1-yl)-5-(isopropylthio)thiazol-2-yl)-4-(3- | K |
| | fluorophenyl)-3-methyl-1H-pyrazole-5-carboxylic acid | |
| B27 | 1-(4-(4-acetylpiperazin-1-yl)-5-(isopropylthio)thiazol-2-yl)-4-(3- | K |
| | fluorophenyl)-3-methyl-1H-pyrazole-5-carboxylic acid | |
| B28 | 4-(3-fluorophenyl)-1-(5-(isopropylthio)-4-(4-methylpiperidin-1- | B |
| | yl)thiazol-2-yl)-3-methyl-1H-pyrazole-5-carboxylic acid | |
| B29 | 4-(3-fluorophenyl)-1-(5-(isopropylthio)-4-(4-methylpiperazin-1- | |
| | yl)thiazol-2-yl)-3-methyl-1H-pyrazole-5-carboxylic acid | |
| B30 | 4-(3-fluorophenyl)-3-methyl-1-(5-(4-(trifluoromethyl)phenyl)-1,3,4- | K |
| | thiadiazol-2-yl)-1H-pyrazole-5-carboxylic acid | |
| B31 | 4-(3-fluorophenyl)-1-(5-((2-methoxyethyl)(methyl)amino)-4-(4- | B |
| | (trifluoromethyl)phenyl)thiazol-2-yl)-3-methyl-1H-pyrazole-5- | |
| | carboxylic acid | |
| B32 | 1-(4-(4,4-dimethylpiperidin-1-yl)-5-(isopropylthio)thiazol-2-yl)-4-(3- | A |
| | fluorophenyl)-3-methyl-1H-pyrazole-5-carboxylic acid | |
| B33 | 1-(4-(4-(tert-butoxycarbonyl)piperazin-1-yl)-5-(isopropylthio)thiazol- | K |
| | 2-yl)-4-(3-fluorophenyl)-3-methyl-1H-pyrazole-5-carboxylic acid | |
| B34 | 4-(3-fluorophenyl)-1-(5-(isopropylthio)-4-(3- | B |
| | (trifluoromethyl)pyrrolidin-1-yl)thiazol-2-yl)-3-methyl-1H-pyrazole-5- | |
| | carboxylic acid | |
| B35 | 4-(3-fluorophenyl)-1-(5-(isopropylthio)-4-(piperazin-1-yl)thiazol-2-yl)- | K |
| | 3-methyl-1H-pyrazole-5-carboxylic acid hydrochloric acid salt | |
| B36 | 4-(3-fluorophenyl)-3-methyl-1-(5-(2-methylprop-1-en-1-yl)-4-(4- | A |
| | (trifluoromethyl)phenyl)thiazol-2-yl)-1H-pyrazole-5-carboxylic acid | |
| B37 | 4-(3-fluorophenyl)-3-methyl-1-(4-(2-methylprop-1-en-1-yl)-5-(4- | D |
| | (trifluoromethyl)phenyl)thiazol-2-yl)-1H-pyrazole-5-carboxylic acid | |
| B38 | 1-(4,5-bis(4-(trifluoromethyl)phenyl)thiazol-2-yl)-4-(3-fluorophenyl)- | A |
| | 3-methyl-1H-pyrazole-5-carboxylic acid | |
| B39 | 2-(4-(3-fluorophenyl)-3-methyl-1H-pyrazol-1-yl)-4,5-bis(4- | K |
| | (trifluoromethyl)phenyl)thiazole | |
| B40 | 1-(4-(4-(tert-butyl)piperidin-1-yl)-5-(isopropylthio)thiazol-2-yl)-4-(3- | A |
| | fluorophenyl)-3-methyl-1H-pyrazole-5-carboxylic acid | |
| B41 | 4-(3-fluorophenyl)-1-(5-(isopropylthio)-4-(6-azaspiro[2.5]octan-6- | B |
| | yl)thiazol-2-yl)-3-methyl-1H-pyrazole-5-carboxylic acid | |
| B42 | 4-(3-fluorophenyl)-1-(5-(isopropylthio)-4-(4-methoxy-4- | A |
| | (trifluoromethyl)piperidin-1-yl)thiazol-2-yl)-3-methyl-1H-pyrazole-5- | |
| | carboxylic acid | |
| B43 | 4-(3-fluorophenyl)-1-(4-(4-methoxyphenyl)-5-(4- | B |
| | (trifluoromethyl)phenyl)thiazol-2-yl)-3-methyl-1H-pyrazole-5- | |
| | carboxylic acid | |
| B44 | 1-(4,5-bis(4-methoxyphenyl)thiazol-2-yl)-4-(3-fluorophenyl)-3- | D |
| | methyl-1H-pyrazole-5-carboxylic acid | |
| B45 | 4-(3-fluorophenyl)-1-(5-(4-methoxyphenyl)-4-(4- | A |
| | (trifluoromethyl)phenyl)thiazol-2-yl)-3-methyl-1H-pyrazole-5- | |
| | carboxylic acid | |
| B46 | 1-(4-(4-(tert-butyl)-3-oxopiperazin-1-yl)-5-(isopropylthio)thiazol-2-yl)- | K |
| | 4-(3-fluorophenyl)-3-methyl-1H-pyrazole-5-carboxylic acid | |
| B47 | 4-(3-fluorophenyl)-3-methyl-1-(5-(3-(methylamino)-3-oxopropyl)-4- | K |
| | (4-(trifluoromethyl)phenyl)thiazol-2-yl)-1H-pyrazole-5-carboxylic | |
| | acid | |
| B48 | 4-(3-fluorophenyl)-1-(5-(isopropylthio)-4-(4-(2-methoxyethoxy)-4- | B |
| | (trifluoromethyl)piperidin-1-yl)thiazol-2-yl)-3-methyl-1H-pyrazole-5- | |
| | carboxylic acid | |
| B49 | 4-(3-fluorophenyl)-1-(5-(4-((2-methoxyethyl)carbamoyl)phenyl)-4-(4- | K |
| | (trifluoromethyl)phenyl)thiazol-2-yl)-3-methyl-1H-pyrazole-5- | |
| | carboxylic acid | |
| B50 | 4-(3-fluorophenyl)-1-(5-(4-((2- | K |
| | methoxyethyl)(methyl)carbamoyl)phenyl)-4-(4- | |
| | (trifluoromethyl)phenyl)thiazol-2-yl)-3-methyl-1H-pyrazole-5- | |
| | carboxylic acid | |
| B51 | 4-(3-fluorophenyl)-1-(5-(4-(2-methoxyacetamido)phenyl)-4-(4- | D |
| | (trifluoromethyl)phenyl)thiazol-2-yl)-3-methyl-1H-pyrazole-5- | |
| | carboxylic acid | |
| B52 | 4-(3-fluorophenyl)-1-(5-(4-(2-(2-methoxyethoxy)acetamido)phenyl)- | D |
| | 4-(4-(trifluoromethyl)phenyl)thiazol-2-yl)-3-methyl-1H-pyrazole-5- | |
| | carboxylic acid | |
| B53 | 4-(3-fluorophenyl)-1-(5-(3-((2-methoxyethyl)amino)-3-oxopropyl)-4- | K |
| | (4-(trifluoromethyl)phenyl)thiazol-2-yl)-3-methyl-1H-pyrazole-5- | |
| | carboxylic acid | |
| B54 | 4-(3-fluorophenyl)-1-(5-(3-((2-methoxyethyl)(methyl)amino)-3- | K |
| | oxopropyl)-4-(4-(trifluoromethyl)phenyl)thiazol-2-yl)-3-methyl-1H- | |
| | pyrazole-5-carboxylic acid | |
| B55 | 4-(3-fluorophenyl)-1-(5-(4-(2-methoxy-N-methylacetamido)phenyl)- | K |
| | 4-(4-(trifluoromethyl)phenyl)thiazol-2-yl)-3-methyl-1H-pyrazole-5- | |
| | carboxylic acid | |
| B56 | 4-(3-fluorophenyl)-1-(5-(4-(2-(2-methoxyethoxy)-N- | K |
| | methylacetamido)phenyl)-4-(4-(trifluoromethyl)phenyl)thiazol-2-yl)- | |
| | 3-methyl-1H-pyrazole-5-carboxylic acid | |
| B57 | 4-(3-fluorophenyl)-1-(5-(methoxymethyl)-4-(4- | C |
| | (trifluoromethyl)phenyl)thiazol-2-yl)-3-methyl-1H-pyrazole-5- | |
| | carboxylic acid | |
| B58 | 1-(5-(4-(2-(2-ethoxyethoxy)ethoxy)phenyl)-4-(4- | B |
| | (trifluoromethyl)phenyl)thiazol-2-yl)-4-(3-fluorophenyl)-3-methyl-1H- | |
| | pyrazole-5-carboxylic acid | |
| B59 | 4-(3-fluorophenyl)-1-(5-(3-fluorophenyl)-4-(4- | A |
| | (trifluoromethyl)phenyl)thiazol-2-yl)-3-methyl-1H-pyrazole-5- | |
| | carboxylic acid | |
| B60 | 4-(3-fluorophenyl)-1-(5-(hydroxymethyl)-4-(4- | K |
| | (trifluoromethyl)phenyl)thiazol-2-yl)-3-methyl-1H-pyrazole-5- | |
| | carboxylic acid | |
| B61 | 4-(3-fluorophenyl)-3-methyl-1-(5-(4-(trifluoromethyl)phenyl)-4-(4- | A |
| | (trifluoromethyl)piperidin-1-yl)thiazol-2-yl)-1H-pyrazole-5-carboxylic | |
| | acid | |
| B62 | 4-(3-fluorophenyl)-1-(4-(3-fluorophenyl)-5-(4- | C |
| | (trifluoromethyl)phenyl)thiazol-2-yl)-3-methyl-1H-pyrazole-5- | |
| | carboxylic acid | |
| B63 | 4-(3-fluorophenyl)-1-(5-(1-hydroxyethyl)-4-(4- | K |
| | (trifluoromethyl)phenyl)thiazol-2-yl)-3-methyl-1H-pyrazole-5- | |
| | carboxylic acid | |
| B64 | 4-(3-fluorophenyl)-1-(5-(2-hydroxyethyl)-4-(4- | K |
| | (trifluoromethyl)phenyl)thiazol-2-yl)-3-methyl-1H-pyrazole-5- | |
| | carboxylic acid | |
| B65 | 4-(3-fluorophenyl)-1-(5-(4-(2-methoxyethoxy)phenyl)-4-(4- | B |
| | (trifluoromethyl)phenyl)thiazol-2-yl)-3-methyl-1H-pyrazole-5- | |
| | carboxylic acid | |
| B66 | 4-(3-fluorophenyl)-1-(5-(1-methoxyethyl)-4-(4- | C |
| | (trifluoromethyl)phenyl)thiazol-2-yl)-3-methyl-1H-pyrazole-5- | |
| | carboxylic acid | |
| B67 | 4-(3-fluorophenyl)-1-(4-(4-isopropylpiperidin-1-yl)-5- | B |
| | (isopropylthio)thiazol-2-yl)-3-methyl-1H-pyrazole-5-carboxylic acid | |
| B68 | 4-(3-fluorophenyl)-1-(5-(isopropylthio)-4-(3-methoxy-3- | — |
| | (trifluoromethyl)-8-azabicyclo[3.2.1]octan-8-yl)thiazol-2-yl)-3-methyl- | |
| | 1H-pyrazole-5-carboxylic acid | |

Terms used herein may be preceded and/or followed by a single dash, or a double dash, “═”, to indicate the bond order of the bond between the named substituent and its parent moiety; a single dash indicates a single bond and a double dash indicates a double bond or a pair of single bonds in the case of a spiro-substituent. In the absence of a single or double dash it is understood that a single bond is formed between the substituent and its parent moiety; further, substituents are intended to be read “left to right” with reference to the chemical structure referred to unless a dash indicates otherwise. For example, arylalkyl, arylalkyl-, and -alkylaryl indicate the same functionality.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety can refer to a monovalent radical (e.g. CH₃—CH₂—), in some circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for O, and 2, 4, or 6 for S, depending on the oxidation state of the S). Nitrogens in the presently disclosed compounds can be hypervalent, e.g., an N-oxide or tetrasubstituted ammonium salt. On occasion a moiety may be defined, for example, as -B-(A)ₐ, wherein a is 0 or 1. In such instances, when a is 0 the moiety is —B and when a is 1 the moiety is -B-A.

As used herein, the term “alkyl” includes a saturated hydrocarbon having a designed number of carbon atoms, such as 1 to 10 carbons (i.e., inclusive of 1 and 10), 1 to 8 carbons, 1 to 6 carbons, 1 to 3 carbons, or 1, 2, 3, 4, 5 or 6. Alkyl group may be straight or branched and depending on context, may be a monovalent radical or a divalent radical (i.e., an alkylene group). For example, the moiety “—(C₁-C₆alkyl)-O—” signifies connection of an oxygen through an alkylene bridge having from 1 to 6 carbons and C₁-C₃alkyl represents methyl, ethyl, and propyl moieties. Examples of “alkyl” include, for example, methyl, ethyl, propyl, isopropyl, butyl, iso-, sec- and tert-butyl, pentyl, and hexyl.

The term “alkoxy” represents an alkyl group of indicated number of carbon atoms attached to the parent molecular moiety through an oxygen bridge. Examples of “alkoxy” include, for example, methoxy, ethoxy, propoxy, and isopropoxy.

The term “alkenyl” as used herein, unsaturated hydrocarbon containing from 2 to 10 carbons (i.e., inclusive of 2 and 10), 2 to 8 carbons, 2 to 6 carbons, or 2, 3, 4, 5 or 6, unless otherwise specified, and containing at least one carbon-carbon double bond. Alkenyl group may be straight or branched and depending on context, may be a monovalent radical or a divalent radical (i.e., an alkenylene group). For example, the moiety “—(C₂-C₆alkenyl)-O—” signifies connection of an oxygen through an alkenylene bridge having from 2 to 6 carbons. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkynyl” as used herein, unsaturated hydrocarbon containing from 2 to 10 carbons (i.e., inclusive of 2 and 10), 2 to 8 carbons, 2 to 6 carbons, or 2, 3, 4, 5 or 6 unless otherwise specified, and containing at least one carbon-carbon triple bond. Alkynyl group may be straight or branched and depending on context, may be a monovalent radical or a divalent radical (i.e., an alkynylene group). For example, the moiety “—(C₂-C₆alkynyl)-O—” signifies connection of an oxygen through an alkynylene bridge having from 2 to 6 carbons. Representative examples of alkynyl include, but are not limited to, acetylenyl, 1-propynyl, 2-propynyl, 3-butynyl, 2-pentynyl, and 1-butynyl.

The term “aryl” represents an aromatic ring system having a single ring (e.g., phenyl) which is optionally fused to other aromatic hydrocarbon rings or non-aromatic hydrocarbon or heterocycle rings. “Aryl” includes ring systems having multiple condensed rings and in which at least one is carbocyclic and aromatic, (e.g., 1,2,3,4-tetrahydronaphthyl, naphthyl). Examples of aryl groups include phenyl, 1-naphthyl, 2-naphthyl, indanyl, indenyl, dihydronaphthyl, fluorenyl, tetralinyl, and 6,7,8,9-tetrahydro-5H-benzo[a]cycloheptenyl. “Aryl” also includes ring systems having a first carbocyclic, aromatic ring fused to a nonaromatic heterocycle, for example, 1H-2,3-dihydrobenzofuranyl and tetrahydroisoquinolinyl. The aryl groups herein are unsubstituted or, when specified as “optionally substituted”, can unless stated otherwise be substituted in one or more substitutable positions with various groups as indicated.

The terms “halogen” or “halo” indicate fluorine, chlorine, bromine, and iodine. In certain embodiments of each and every embodiment described herein, the term “halogen” or “halo” refers to fluorine or chlorine. In certain embodiments of each and every embodiment described herein, the term “halogen” or “halo” refers to fluorine.

The term “heteroaryl” refers to an aromatic ring system containing at least one aromatic heteroatom selected from nitrogen, oxygen and sulfur in an aromatic ring. Most commonly, the heteroaryl groups will have 1, 2, 3, or 4 heteroatoms. The heteroaryl may be fused to one or more non-aromatic rings, for example, cycloalkyl or heterocycloalkyl rings, wherein the cycloalkyl and heterocycloalkyl rings are described herein. In one embodiment of the present compounds the heteroaryl group is bonded to the remainder of the structure through an atom in a heteroaryl group aromatic ring. In another embodiment, the heteroaryl group is bonded to the remainder of the structure through a non-aromatic ring atom. Examples of heteroaryl groups include, for example, pyridyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, benzo[1,4]oxazinyl, triazolyl, tetrazolyl, isothiazolyl, naphthyridinyl, isochromanyl, chromanyl, isoindolinyl, isobenzothienyl, benzoxazolyl, pyridopyridinyl, purinyl, benzodioxolyl, triazinyl, pteridinyl, benzothiazolyl, imidazopyridinyl, imidazothiazolyl, benzisoxazinyl, benzoxazinyl, benzopyranyl, benzothiopyranyl, chromonyl, chromanonyl, pyridinyl-N-oxide, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Preferred heteroaryl groups include pyridyl, pyrimidyl, quinolinyl, indolyl, pyrrolyl, furanyl, thienyl and imidazolyl, pyrazolyl, indazolyl, thiazolyl and benzothiazolyl. In certain embodiments, each heteroaryl is selected from pyridyl, pyrimidinyl, pyridazinyl, pyrazinyl, imidazolyl, isoxazolyl, pyrazolyl, oxazolyl, thiazolyl, furanyl, thienyl, pyrrolyl, oxadiazolyl, thiadiazolyl, triazolyl, tetrazolyl, isothiazolyl, pyridinyl-N-oxide, pyrrolyl N-oxide, pyrimidinyl N-oxide, pyridazinyl N-oxide, pyrazinyl N-oxide, imidazolyl N-oxide, isoxazolyl N-oxide, oxazolyl N-oxide, thiazolyl N-oxide, pyrrolyl N-oxide, oxadiazolyl N-oxide, thiadiazolyl N-oxide, triazolyl N-oxide, and tetrazolyl N-oxide. Preferred heteroaryl groups include pyridyl, pyrimidyl, quinolinyl, indolyl, pyrrolyl, furanyl, thienyl, imidazolyl, pyrazolyl, indazolyl, thiazolyl and benzothiazolyl. The heteroaryl groups herein are unsubstituted or, when specified as “optionally substituted”, can unless stated otherwise be substituted in one or more substitutable positions with various groups, as indicated.

The term “heterocycloalkyl” refers to a non-aromatic ring or ring system containing at least one heteroatom that is preferably selected from nitrogen, oxygen and sulfur, wherein said heteroatom is in a non-aromatic ring. The heterocycloalkyl may have 1, 2, 3 or 4 heteroatoms. The heterocycloalkyl may be saturated (i.e., a heterocycloalkyl) or partially unsaturated (i.e., a heterocycloalkenyl). Heterocycloalkyl includes monocyclic groups of three to eight annular atoms as well as bicyclic and polycyclic ring systems, including bridged and fused systems, wherein each ring includes three to eight annular atoms. The heterocycloalkyl ring is optionally fused to other heterocycloalkyl rings and/or non-aromatic hydrocarbon rings. In certain embodiments, the heterocycloalkyl groups have from 3 to 7 members in a single ring. In other embodiments, heterocycloalkyl groups have 5 or 6 members in a single ring. In some embodiments, the heterocycloalkyl groups have 3, 4, 5, 6 or 7 members in a single ring. Examples of heterocycloalkyl groups include, for example, azabicyclo[2.2.2]octyl (in each case also “quinuclidinyl” or a quinuclidine derivative), azabicyclo[3.2.1]octyl, 2,5-diazabicyclo[2.2.1]heptyl, morpholinyl, thiomorpholinyl, thiomorpholinyl S-oxide, thiomorpholinyl S,S-dioxide, 2-oxazolidonyl, piperazinyl, homopiperazinyl, piperazinonyl, pyrrolidinyl, azepanyl, azetidinyl, pyrrolinyl, tetrahydropyranyl, piperidinyl, tetrahydrofuranyl, tetrahydrothienyl, 3,4-dihydroisoquinolin-2(1H)-yl, isoindolindionyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, imidazolidonyl, tetrahydrothienyl S-oxide, tetrahydrothienyl S,S-dioxide and homothiomorpholinyl S-oxide. Especially desirable heterocycloalkyl groups include morpholinyl, 3,4-dihydroisoquinolin-2(1H)-yl, tetrahydropyranyl, piperidinyl, aza-bicyclo[2.2.2]octyl, γ-butyrolactonyl (i.e., an oxo-substituted tetrahydrofuranyl), γ-butryolactamyl (i.e., an oxo-substituted pyrrolidine), pyrrolidinyl, piperazinyl, azepanyl, azetidinyl, thiomorpholinyl, thiomorpholinyl S,S-dioxide, 2-oxazolidonyl, imidazolidonyl, isoindolindionyl, piperazinonyl. The heterocycloalkyl groups herein are unsubstituted or, when specified as “optionally substituted”, can unless stated otherwise be substituted in one or more substitutable positions with various groups, as indicated.

The term “cycloalkyl” refers to a non-aromatic carbocyclic ring or ring system, which may be saturated (i.e., a cycloalkyl) or partially unsaturated (i.e., a cycloalkenyl). The cycloalkyl ring optionally fused to or otherwise attached (e.g., bridged systems) to other cycloalkyl rings. Certain examples of cycloalkyl groups present in the disclosed compounds have from 3 to 7 members in a single ring, such as having 5 or 6 members in a single ring. In some embodiments, the cycloalkyl groups have 3, 4, 5, 6 or 7 members in a single ring. Examples of cycloalkyl groups include, for example, cyclohexyl, cyclopentyl, cyclobutyl, cyclopropyl, tetrahydronaphthyl and bicyclo[2.2.1]heptane. The cycloalkyl groups herein are unsubstituted or, when specified as “optionally substituted”, may be substituted in one or more substitutable positions with various groups, as indicated.

The term “ring system” encompasses monocycles, as well as fused and/or bridged polycycles.

The term “oxo” means a doubly bonded oxygen, sometimes designated as ═O or for example in describing a carbonyl “C(O)” may be used to show an oxo substituted carbon.

The term “substituted,” when used to modify a specified group or radical, means that one or more hydrogen atoms of the specified group or radical are each, independently of one another, replaced with the same or different substituent groups as defined below, unless specified otherwise.

As used herein, the phrase “pharmaceutically acceptable salt” refers to both pharmaceutically acceptable acid and base addition salts and solvates. Such pharmaceutically acceptable salts include salts of acids such as hydrochloric, phosphoric, hydrobromic, sulfuric, sulfinic, formic, toluenesulfonic, methanesulfonic, nitric, benzoic, citric, tartaric, maleic, hydroiodic, alkanoic such as acetic, HOOC—(CH₂)ₙ—COOH where n is 0-4, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

One of ordinary skill in the art of medicinal chemistry also will appreciate that the disclosed structures are intended to include isotopically enriched forms of the present compounds. As used herein “isotopes” includes those atoms having the same atomic number but different mass numbers. As is known to those of skill in the art, certain atoms, such as hydrogen occur in different isotopic forms. For example, hydrogen includes three isotopic forms, protium, deuterium and tritium. As will be apparent to those of skill in the art upon consideration of the present compounds, certain compounds can be enriched at a given position with a particular isotope of the atom at that position. For example, compounds having a fluorine atom, may be synthesized in a form enriched in the radioactive fluorine isotope ¹⁸F. Similarly, compounds may be enriched in the heavy isotopes of hydrogen: deuterium and tritium; and similarly can be enriched in a radioactive isotope of carbon, such as ¹³C. Such isotopic variant compounds undergo different metabolic pathways and can be useful, for example, in studying the ubiquitination pathway and its role in disease. Of course, in certain embodiments, the compound has substantially the same isotopic character as naturally-occurring materials.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the terms “individual,” “patient,” or “subject” are used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” or “effective amount” refers to the amount of active compound or pharmaceutical agent that elicits the biological or medicinal response that is being sought in a tissue, system, animal, individual or human by a researcher, veterinarian, medical doctor or other clinician.

In certain embodiments, a therapeutically effective amount can be an amount suitable for

(1) preventing the disease; for example, preventing a disease, condition or disorder in an individual who may be predisposed or otherwise susceptible to the disease, condition or disorder but does not yet experience or display the pathology or symptomatology of the disease;

(2) inhibiting the disease; for example, inhibiting a disease, condition or disorder in an individual who is experiencing or displaying the pathology or symptomatology of the disease, condition or disorder;

(3) ameliorating the disease (including a symptom thereof); for example, ameliorating a disease, condition or disorder in an individual who is experiencing or displaying the pathology or symptomatology of the disease, condition or disorder (i.e., reversing the pathology and/or symptomatology) such as decreasing the severity of disease; or

(4) eliciting a referenced biological effect, e.g., inhibiting the initiation of translation. Such biological effect need not be complete, i.e., an inhibition of the initiation of translation need not be complete inhibition in order for the amount of compound administered to be therapeutically effective.

As used here, the terms “treatment” and “treating” means (i) ameliorating the referenced disease state, condition, or disorder (or a symptom thereof), such as, for example, ameliorating a disease, condition or disorder in an individual who is experiencing or displaying the pathology or symptomatology of the disease, condition or disorder (i.e., reversing or improving the pathology and/or symptomatology) such as decreasing the severity of disease or symptom thereof; or (ii) eliciting the referenced biological effect (e.g., inhibiting the initiation of translation).

### Pharmaceutical Formulations and Dosage Forms

The compounds of the disclosure can be administered, for example, orally, topically, parenterally, by inhalation or spray or rectally in dosage unit formulations containing one or more pharmaceutically acceptable carriers, diluents or excipients. The term parenteral as used herein includes percutaneous, subcutaneous, intravascular (e.g., intravenous), intramuscular, or intrathecal injection or infusion techniques and the like.

Pharmaceutical compositions can be made using the presently disclosed compounds. For example, in one embodiment, a pharmaceutical composition includes a pharmaceutically acceptable carrier, diluent or excipient, and compound as described above with reference to any one of structural formulae.

In the pharmaceutical compositions disclosed herein, one or more compounds of the disclosure may be present in association with one or more pharmaceutically acceptable carriers, diluents or excipients, and, if desired, other active ingredients. The pharmaceutical compositions containing compounds of the disclosure may be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any suitable method for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by suitable techniques to delay disintegration and absorption in the gastrointestinal tract and thereby provide a sustained action over a longer period. For example, a time delay material such as glyceryl monostearate or glyceryl distearate can be employed.

Formulations for oral use can also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Formulations for oral use can also be presented as lozenges.

Aqueous suspensions contain the active materials in admixture with excipients suitable for the manufacture of aqueous suspensions. Such excipients can be suspending agents, for example sodium carboxymethylcellulose, methylcellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

In some embodiments, the pharmaceutically acceptable carrier, diluent, or excipient is not water. In other embodiments, the water comprises less than 50% of the composition. In some embodiments, compositions comprising less than 50% water have at least 1%, 2%, 3%, 4% or 5% water. In other embodiments, the water content is present in the composition in a trace amount.

In some embodiments, the pharmaceutically acceptable carrier, diluent, or excipient is not alcohol. In other embodiments, the alcohol comprises less than 50% of the composition. In some embodiments, compositions comprising less than 50% alcohol have at least 1%, 2%, 3%, 4% or 5% alcohol. In other embodiments, the alcohol content is present in the composition in a trace amount.

Syrups and elixirs can be formulated with sweetening agents, for example glycerol, propylene glycol, sorbitol, glucose or sucrose. Such formulations can also contain a demulcent, a preservative, flavoring, and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils can be employed as a solvent or suspending medium. For this purpose, any bland fixed oil can be employed including synthetic mono- or diglycerides. In addition, fatty acids such as oleic acid find use in the preparation of injectables.

Compounds of the disclosure can also be administered in the form of suppositories, e.g., for rectal administration of the drug. These compositions can be prepared by mixing the compound with a suitable non-irritating excipient that is solid at ordinary temperatures but liquid at the rectal temperature and will therefore melt in the rectum to release the drug. Such materials include cocoa butter and polyethylene glycols.

Compounds of the disclosure can also be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

The compositions can be formulated in a unit dosage form of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound described herein. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of a compound described herein.

The tablets or pills can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds can vary according to, for example, the particular use for which the treatment is made, the manner of administration of the compound, the health and condition of the patient, and the judgment of the prescribing physician. The proportion or concentration of a compound described herein in a pharmaceutical composition can vary depending upon a number of factors including dosage, chemical characteristics (e.g., hydrophobicity), and the route of administration. For example, the compounds described herein can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds described herein can also be formulated in combination with one or more additional active ingredients which can include any pharmaceutical agent such as anti-viral agents, vaccines, antibodies, immune enhancers, immune suppressants, anti-inflammatory agents and the like.

The person of ordinary skill in the art will formulate a compound as described into pharmaceutical formulations herein, for example, based on the physicochemical properties of the compound, the amount of the compound needed for a pharmaceutically effective amount, and the desired route of administration.

### EXAMPLES

### Example 1. Gene Quantification and Treatment

Cancer can be identified via the use of nucleic acid isolation and real time PCR analysis. In one embodiment a blood, cell, tissue or saliva sample is obtained from a human individual with a hematopoietic cancer, a human individual with the solid tumor cancer, and/or from a healthy individual human or a cell line. Nucleic acids are isolated using standard procedures widely known in the art.

The RNA or mRNA is then reverse transcribed to cDNA and gene specific primers are used to amplify a segment of cDNA corresponding to the gene of interest. In one embodiment (i.e., with respect to solid tumor cancers), primers for a plurality of the target genes LAMC3, FAM210B, SENP8, ITGB3BP, NUDT2, HNRNPCL1, C20orf43, FRMD8, and STX16 are used to amplify the genes of interest using standard PCR techniques. In an alternative embodiment, (i.e., with respect to hematopoietic cancers) primers for the target genes CASP10, TMED1, PPP1CC, TMEM59, BRD7, CYB561, FAM210B, NDRG1, CTSB, MMAB, SETDB2, VPS37B, ELL3, and KIF13B are used to amplify the gene of interest using standard PCR techniques.

In addition, primers for one or more housekeeping genes (e.g., one or more of 18s rRNA, 28s rRNA, α-tubulin, β-actin, ALB RPL32, TBP, CYCC, EF1A and GAPDH) can be included in the run as internal controls.

In certain embodiments, quantification of the gene expression is tracked in real time via the use of fluorescent probes and changes in gene expression quantified.

Gene expression values are used to calculate fold change compared to expression of the same gene in a reference cell (e.g., from a blood sample or non-cancerous tissue from a healthy individual human or a cell line). Fold change is calculated as:

2^{-ΔΔCt}

where

ΔΔCt=ΔCt (treated sample)−ΔCt (untreated sample) and

ΔCt=Ct (gene of interest)−Ct (housekeeping gene).

Microarray analysis or alternative quantitative gene analysis studies can also be performed.

### Example 2: Predictive Biomarker Discovery in OmniScreen™

Predictive biomarkers for genes indicating responsiveness to a therapeutic compound can be determined using a cell-based screening technique, such as that offered under the name OmniScreen™ (Crown Biosciences).

In one experimental study, predictive biomarkers were determined for Compound A197 efficacy on 406 cancer cell lines, 73 blood tumor cell lines and 333 solid tumor cell lines. Genomic data for the cell lines was downloaded from the Cancer Cell Line Encyclopedia Project (CCLE) website. Driver mutations were predicted on the Cancer Genome Interpreter Database; only driver mutations were used in the mutation related analysis. Spearman correlation was used to detect genes whose expression was significantly correlated with AUC.

Signature genes were selected using Boruta package in R. A linear predictor score (LPS) for each cell line of the form was calculated as:

$$LPS ( X ) = {\sum }_{j} {a}_{j} {X}_{j}$$

where Xᵢ represents the gene expression of gene j, and a, is the t-statistics generated by t-test between sensitive and insensitive cell lines. The mean and variance of the LPS distribution in sensitive and insensitive groups were estimated LPS distribution in sensitive and insensitive groups were estimated, and the likelihood that a cell line in which group (sensitive or insensitive) was estimated by applying Bayes' rule so that

$$P ( X in group 1 ) = \frac{∅ ( LPS ( X ) ; {μ}_{1} , {σ}_{1}^{2} )}{∅ ( LPS ( X ) ; {μ}_{1} , {σ}_{1}^{2} ) + ∅ ( LPS ( X ) ; {μ}_{2} , {σ}_{2}^{2} )} ,$$

where Ø(x; μ,σ²) represents the normal density function with mean p and variance a2, and μ1, σ₁², μ₂, σ₂² are the observed mean and variance of the LPSs within group 1 and group 2, respectively.

Welch's t-test was used to evaluate the association between gene amplification, deletion, mutation status and AUCs.

Dose-response curves were fitted by the 4-parameter model:

$$Inhibition \% = Bottom + \frac{Top - Bottom}{1 + {10}^{( {logEC}_{50} - x ) * Hillscope}}$$

in which top and bottom are the two asymptotes of the sigmoidal curve, EC50 is the relative IC50, and concentration x is in log−10 scale. To accommodate experimental errors, Bottom was allowed to go down to −20%, and Top was allowed to go up to 120%. The fitting error of a model is measured by

$$\frac{{σ}_{EC 50}}{EC 50}$$

σECS0 is the standard error of EC50. In general, such fitting error should be less than 40% for a model to be considered acceptable. The fitted area under curve (AUC) is calculated by

$$AUC = {\int }_{a}^{b} ( Bottom + \frac{Top - Bottom}{1 + {10}^{( {logEC}_{50} - x ) * Hillscope}} ) dx$$

where a=log(3, 10) and b=log(30000,10). AUC data for a variety of cell lines are provided in the table below.

Summary of Drug efficacy results of cell lines

| Cell line | Tissue | Specific Cancer | AUCs | IC50(nm) |
|---|---|---|---|---|
| SW-13 | Adrenal gland | Adrenal cortex | 2.85 | 361.57 |
| | | carcinoma | | |
| 5637 | Bladder | Bladder carcinoma | 2.81 | 475.61 |
| RT4 | Bladder | Bladder carcinoma | 3.03 | 815.37 |
| UM-UC-3 | Bladder | Bladder carcinoma | 2.77 | 930.97 |
| J82 | Bladder | Bladder carcinoma | 2.98 | 1111.39 |
| RT112/84 | Bladder | Bladder carcinoma | 3.23 | 13876.08 |
| SW780 | Bladder | Bladder carcinoma | 3.58 | 46515.42 |
| T24 | Bladder | Blladder carcinoma | 4.26 | 50809.37 |
| NB4 | Blood | Acute promyelocytic | 1.63 | 110.16 |
| | | leukemia | | |
| Pfeiffer | Blood | Diffuse large B-cell | 1.78 | 179.67 |
| | | lymphoma germinal | | |
| | | center B-Cell type | | |
| THP-1 | Blood | Childhood acute | 2.35 | 204.1 |
| | | monocytic leukemia | | |
| ST486 | Blood | Burkitt lymphoma | 2.09 | 216.95 |
| JVM-3 | Blood | B-cell | 2.2 | 265.72 |
| | | prolymphocytic | | |
| | | leukemia | | |
| Reh | Blood | Childhood B acute | 2.11 | 305.93 |
| | | lymphoblastic | | |
| | | leukemia | | |
| MV-4-11 | Blood | Childhood acute | 2.44 | 311.92 |
| | | monocytic leukemia | | |
| DOHH-2 | Blood | Diffuse large B-cell | 2.12 | 312.63 |
| | | lymphoma | | |
| SU-DHL-1 | Blood | Anaplastic large cell | 2.48 | 329.95 |
| | | lymphoma | | |
| MOLM-16 | Blood | Adult acute myeloid | 2.72 | 352.04 |
| | | leukemia | | |
| JVM-13 | Blood | Mantle cell | 2.62 | 382.58 |
| | | lymphoma | | |
| U-937 | Blood | Adult acute | 2.65 | 394.06 |
| | | monocytic leukemia | | |
| OPM2 | Blood | Plasma cell | 2.48 | 416.87 |
| | | myeloma | | |
| SU-DHL-5 | Blood | Diffuse large B-cell | 2.19 | 418.87 |
| | | lymphoma | | |
| Raji | Blood | Epstein-Barr virus- | 2.33 | 455.75 |
| | | related Burkitt | | |
| | | lymphoma | | |
| U-2932 | Blood | Diffuse large B-cell | 2.59 | 455.78 |
| | | lymphoma activated | | |
| | | B-cell type | | |
| ML-2 | Blood | Adult acute myeloid | 2.35 | 467.69 |
| | | leukemia | | |
| NAMALWA/ | Blood | Epstein-Barr virus- | 2.49 | 487.21 |
| CSN70 | | related Burkitt | | |
| | | lymphoma | | |
| WSU-NHL | Blood | Follicular lymphoma | 2.31 | 489.09 |
| LP-1 | Blood | Plasma cell | 2.71 | 500.68 |
| | | myeloma | | |
| NAMALWA | Blood | Epstein-Barr virus- | 2.74 | 508.78 |
| | | related Burkitt | | |
| | | lymphoma | | |
| K-562 | Blood | bone marrow - | 2.94 | 574.21 |
| | | chronic myeloid | | |
| | | leukemia | | |
| CCRF-CEM | Blood | Childhood T acute | 2.56 | 586.1 |
| | | lymphoblastic | | |
| | | leukemia | | |
| CA46 | Blood | Burkitt's lymphoma | 2.54 | 605.34 |
| Molt-4 | Blood | acute lymphoblastic | 2.69 | 607.19 |
| | | leukemia | | |
| HuT 78 | Blood | Sezary Syndrome | 2.62 | 630.87 |
| KMS-11 | Blood | Plasma cell | 2.68 | 631.79 |
| | | myeloma | | |
| Jurkat clone | Blood | Childhood T acute | 2.52 | 651.82 |
| E6-1 | | lymphoblastic | | |
| | | leukemia | | |
| WSU-DLCL2 | Blood | Diffuse large B-cell | 2.43 | 674.52 |
| | | lymphoma | | |
| RS4; 11 | Blood | Adult B acute | 2.7 | 684.63 |
| | | lymphoblastic | | |
| | | leukemia | | |
| JVM-2 | Blood | Mantle cell | 2.73 | 697.26 |
| | | lymphoma | | |
| NALM-6 | Blood | Adult B acute | 2.64 | 700.31 |
| | | lymphoblastic | | |
| | | leukemia | | |
| HBL-1 | Blood | large B-cell | 2.75 | 706.09 |
| | | lymphoma | | |
| EOL-1 | Blood | Chronic eosinophilic | 2.65 | 735.77 |
| | | leukemia | | |
| RPMI 8226 | Blood | Plasma cell | 2.72 | 745.92 |
| | | myeloma | | |
| Ramos | Blood | Burkitt lymphoma | 2.43 | 779.62 |
| JeKo-1 | Blood | Mantle cell | 2.58 | 792.43 |
| | | lymphoma | | |
| Daudi | Blood | Epstein-Barr virus- | 2.42 | 793.35 |
| | | related Burkitt | | |
| | | lymphoma | | |
| MOLM-13 | Blood | Adult acute myeloid | 2.85 | 873.41 |
| | | leukemia | | |
| KARPAS-422 | Blood | Diffuse large B-cell | 2.57 | 890.76 |
| | | lymphoma | | |
| L-363 | Blood | Plasma cell | 2.93 | 926.45 |
| | | myeloma | | |
| OCI-LY-19 | Blood | Diffuse large B-cell | 2.86 | 948.84 |
| | | lymphoma | | |
| HL-60 | Blood | Adult acute myeloid | 2.69 | 951.98 |
| | | leukemia | | |
| MOLP8 | Blood | Plasma cell | 3.26 | 960.61 |
| | | myeloma | | |
| KG-1 | Blood | Adult acute myeloid | 3.33 | 970.33 |
| | | leukemia | | |
| GRANTA-519 | Blood | Mantle cell | 3.16 | 1001.56 |
| | | lymphoma | | |
| SUP-B15 | Blood | Childhood B acute | 2.74 | 1027.35 |
| | | lymphoblastic | | |
| | | leukemia | | |
| SU-DHL-6 | Blood | Diffuse large B-cell | 2.67 | 1048.46 |
| | | lymphoma germinal | | |
| | | center B-Cell type | | |
| SU-DHL-4 | Blood | Diffuse large B-cell | 2.81 | 1084.62 |
| | | lymphoma germinal | | |
| | | center B-Cell type | | |
| MM.1R | Blood | Plasma cell | 3.17 | 1187.85 |
| | | myeloma | | |
| JJN-3 | Blood | Plasma cell | 2.92 | 1331.4 |
| | | myeloma | | |
| Toledo | Blood | Diffuse large B-cell | 3.02 | 1469.52 |
| | | lymphoma | | |
| MEG-01 | Blood | Chronic | 3.22 | 1613.62 |
| | | myelogenous | | |
| | | leukemia | | |
| PEER | Blood | Childhood T acute | 2.93 | 1889.93 |
| | | lymphoblastic | | |
| | | leukemia | | |
| AMO-1 | Blood | Plasma cell | 3.33 | 1951.87 |
| | | myeloma | | |
| EJM | Blood | Plasma cell | 3.17 | 2718.7 |
| | | myeloma | | |
| Kasumi-1 | Blood | Childhood acute | 2.93 | 3004.8 |
| | | myeloid leukemia | | |
| KARPAS-299 | Blood | Anaplastic large cell | 3.13 | 10320.38 |
| | | lymphoma | | |
| HEL 92.1.7 | Blood/Leukemia | Erythroleukemia | 2.34 | 373.58 |
| OCI-LY7 | Blood/Lymphoma | Diffuse large B-cell | 2.33 | 645.92 |
| | | lymphoma | | |
| SU-DHL-8 | Blood/Lymphoma | Diffuse large B-cell | 2.38 | 899.42 |
| | | lymphoma germinal | | |
| | | center B-Cell type | | |
| Z-138 | Blood/Lymphoma | Mantle cell | 2.71 | 1538.73 |
| | | lymphoma | | |
| NK-92MI | Blood/Lymphoma | Natural killer cell | 3.39 | 6599.9 |
| | | lymphoblastic | | |
| | | leukemia/lymphoma | | |
| MC/CAR | Blood/Myeloma | Plasmacytoma | 3.4 | 4238.86 |
| A3/KAW | Blood/Stomach | Diffuse large B-cell | 2.14 | 293.48 |
| | | lymphoma | | |
| CADO-ES1 | Bone | Ewing sarcoma | 2.47 | 202.64 |
| Saos-2 | Bone | Osteosarcoma | 2.8 | 788.75 |
| G-292 clone | Bone | Osteosarcoma | 3.05 | 1883.57 |
| A141B1 | | | | |
| 143B | Bone | Osteosarcoma | 3.99 | 32678.02 |
| HOS | Bone | Osteosarcoma | 3.45 | 103962.06 |
| SJSA-1 | Bone | Osteosarcoma | 4.3 | 276250.57 |
| RD-ES | Bone | Ewing sarcoma | 3.84 | 468115.52 |
| U251 | Brain&Nerves | Astrocytoma | 2.47 | 280.33 |
| H4 | Brain&Nerves | Astrocytoma | 2.6 | 723.93 |
| SF-763 | Brain&Nerves | Astrocytoma | 2.89 | 770.29 |
| IMR-32 | Brain&Nerves | Neuroblastoma | 2.92 | 790.32 |
| SK-N-FI | Brain&Nerves | Neuroblastoma | 3.17 | 1150.88 |
| M059K | Brain&Nerves | Glioblastoma | 3.16 | 1646.48 |
| LN-18 | Brain&Nerves | Glioblastoma | 2.77 | 2478.54 |
| U-87 MG | Brain&Nerves | Glioblastoma | 3.56 | 27030.11 |
| SK-N-SH | Brain&Nerves | Neuroblastoma | 3.9 | 33728.49 |
| SH-SY5Y | Brain&Nerves | Neuroblastoma | 4.33 | 53786.43 |
| KPL-4 | Breast | Inflammatory breast | 2.06 | 253.05 |
| | | carcinoma | | |
| DU4475 | Breast | Breast carcinoma | 2.8 | 311.23 |
| BT-549 | Breast | Invasive ductal | 2.95 | 339.78 |
| | | carcinoma | | |
| MDA-MB-468 | Breast | Breast | 2.53 | 507.02 |
| | | adenocarcinoma | | |
| EFM-192A | Breast | Breast carcinoma | 2.82 | 550.54 |
| CAMA-1 | Breast | luminal-type human | 2.9 | 597.27 |
| | | breast cancer cell lin | | |
| CAL-51 | Breast | Breast carcinoma | 2.94 | 1698.86 |
| AU565 | Breast | Breast | 3.51 | 10861.13 |
| | | adenocarcinoma | | |
| ZR-75-1 | Breast | Invasive ductal | 3.2 | 11305.48 |
| | | carcinoma, not | | |
| | | otherwise specified | | |
| MX-1 | Breast | Breast carcinoma | 2.92 | 15463.19 |
| SUM159PT | Breast | Pleomorphic breast | 3.53 | 16437.04 |
| | | carcinoma | | |
| SK-BR-3 | Breast | Amelanotic melanom | 3.87 | 28425.07 |
| HCC1428 | Breast | Breast | 3.88 | 30591.58 |
| | | adenocarcinoma | | |
| CAL-120 | Breast | Breast carcinoma | 3.41 | 31779 |
| HCC38 | Breast | Ductal breast | 3.93 | 34604.08 |
| | | carcinoma | | |
| HCC1569 | Breast | Ductal breast | 3.9 | 73065.12 |
| | | carcinoma | | |
| MDA-MB-231 | Breast | Breast | 4.09 | 192167.19 |
| | | adenocarcinoma | | |
| BT474 | Breast | Invasive ductal | 3.9 | 410484.52 |
| | | carcinoma | | |
| HeLa | Cervix | Human | 3.14 | 2398.48 |
| | | papillomavirus- | | |
| | | related endocervical | | |
| | | adenocarcinoma | | |
| SiHa | Cervix | Human | 3.93 | 429675.12 |
| | | papillomavirus- | | |
| | | related cervical | | |
| | | squamous cell | | |
| | | carcinoma | | |
| HCT-15 | Colorectum | Colon | 2.32 | 295.89 |
| | | adenocarcinoma | | |
| COLO | Colorectum | Colon | 2.73 | 400.66 |
| 320DM | | adenocarcinoma | | |
| DLD-1 | Colorectum | Colon | 2.41 | 413.31 |
| | | adenocarcinoma | | |
| LS411N | Colorectum | Cecum | 2.92 | 699.84 |
| | | adenocarcinoma | | |
| LS513 | Colorectum | Cecum | 2.97 | 707.37 |
| | | adenocarcinoma | | |
| HCT-8 | Colorectum | Colon | 2.74 | 775.85 |
| | | adenocarcinoma | | |
| HCT-116 | Colorectum | Colon carcinoma | 2.74 | 927.8 |
| SK-CO-1 | Colorectum | Colon | 3.16 | 1015.11 |
| | | adenocarcinoma | | |
| COLO 205 | Colorectum | Colon | 3.07 | 1134.9 |
| | | adenocarcinoma | | |
| HT-29 | Colorectum | Rectosigmoid | 3.14 | 1152.92 |
| | | adenocarcinoma | | |
| NCI-H716 | Colorectum | Cecum | 2.93 | 1444.96 |
| | | adenocarcinoma | | |
| NCI-H508 | Colorectum | Cecum | 3.08 | 1511.53 |
| | | adenocarcinoma | | |
| RKO | Colorectum | Colon carcinoma | 3.52 | 24391.51 |
| LS 174T | Colorectum | Colon | 4.2 | 33120.85 |
| | | adenocarcinoma | | |
| SW1417 | Colorectum | Colon | 3.92 | 42631 |
| | | adenocarcinoma | | |
| LoVo | Colorectum | Colon | 3.16 | 62541.3 |
| | | adenocarcinoma | | |
| HM-7 | Colorectum | Colon | 3.31 | 70477.8 |
| | | adenocarcinoma | | |
| Caco-2 | Colorectum | Colon | 3.77 | 92082.15 |
| | | adenocarcinoma | | |
| SW837 | Colorectum | Rectal | 3.56 | 221464.38 |
| | | adenocarcinoma | | |
| KYSE-70 | Esophagus | Esophageal | 2.72 | 460.3 |
| | | squamous cell | | |
| | | carcinoma | | |
| KYSE-150 | Esophagus | esophageal | 2.38 | 594.73 |
| | | squamous cell | | |
| | | carcinoma | | |
| KYSE-270 | Esophagus | Esophageal | 3.06 | 3782.71 |
| | | squamous cell | | |
| | | carcinoma | | |
| T.Tn | H&N/Esophagus | Esophageal | 2.86 | 838.88 |
| | | squamous cell | | |
| | | carcinoma | | |
| FTC-133 | H&N/Thyroid | Thyroid gland | 2.9 | 811.57 |
| | | follicular carcinoma | | |
| IHH-4 | H&N/Thyroid | Thyroid gland | 2.78 | 937.25 |
| | | papillary carcinoma | | |
| KMH-2 | H&N/Thyroid | Thyroid gland | 3.28 | 9075.99 |
| | | undifferentiated | | |
| | | (anaplastic) | | |
| | | carcinoma | | |
| 8305C | H&N/Thyroid | hyroid gland | 3.91 | 41946.69 |
| | | undifferentiated | | |
| | | (anaplastic) | | |
| | | carcinoma | | |
| ACHN | Kidney | Papillary renal cell | 2.83 | 789.11 |
| | | carcinoma | | |
| A498 | Kidney | Renal cell | 2.77 | 862.5 |
| | | carcinoma | | |
| SK-NEP-1 | Kidney | Ewing sarcoma | 3.06 | 986.24 |
| 769-P | Kidney | Renal cell carcinoma | 2.8 | 1017.74 |
| 786-0 | Kidney | Renal cell carcinoma | 3.05 | 1129.41 |
| SW 156 | Kidney | Renal cell carcinoma | 3.11 | 2348.85 |
| OS-RC-2 | Kidney | Renal cell carcinoma | 3.78 | 112607.7 |
| CCK-81 | Large | Colon | 2.4 | 317.52 |
| | intestine/Colorectum | adenocarcinoma | | |
| SNU-C2A | Large | Cecum | 2.72 | 581.48 |
| | intestine/Colorectum | adenocarcinoma | | |
| LS 180 | Large | Colon | 3.59 | 793174.65 |
| | intestine/Colorectum | adenocarcinoma | | |
| SNU-398 | Liver | Hepatocellular | 2.18 | 224.88 |
| | | carcinoma | | |
| JHH-7 | Liver | Hepatocellular | 2.68 | 666.42 |
| | | carcinoma | | |
| NOZ | Liver | Gallbladder | 2.79 | 677.78 |
| | | carcinoma | | |
| Li-7 | Liver | Hepatocellular | 2.62 | 838.19 |
| | | carcinoma | | |
| SNU-761 | Liver | Hepatocellular | 3.32 | 967.1 |
| | | carcinoma | | |
| HUH-7 | Liver | Hepatocellular | 3.1 | 1023.53 |
| | | carcinoma | | |
| PLC/PRF/5 | Liver | Hepatocellular | 3.34 | 1202.09 |
| | | carcinoma | | |
| OCUG-1 | Liver | Gallbladder | 2.81 | 1227.53 |
| | | carcinoma | | |
| JHH-4 | Liver | Hepatocellular | 2.77 | 1506.18 |
| | | carcinoma | | |
| SNU-475 | Liver | Hepatocellular | 3.01 | 2459.41 |
| | | carcinoma | | |
| MHCC97-H | Liver | Hepatocellular | 3.3 | 3216.8 |
| | | carcinoma | | |
| JHH-6 | Liver | Hepatocellular | 3.16 | 20595.21 |
| | | carcinoma | | |
| SNU-739 | Liver | Hepatocellular | 3.71 | 26417.79 |
| | | carcinoma | | |
| SNU-449 | Liver | Hepatocellular | 3.55 | 29396.76 |
| | | carcinoma | | |
| JHH-1 | Liver | Hepatocellular | 4.03 | 50934.55 |
| | | carcinoma | | |
| SNU-387 | Liver | Hepatocellular | 3.93 | 61405.09 |
| | | carcinoma | | |
| Hep G2/C3A | Liver | Hepatoblastoma | 4.02 | 150355.28 |
| OZ | Liver | Intrahepatic | 3.79 | 382288.96 |
| | | Cholangiocarcinoma | | |
| SNU-368 | Liver | Hepatocellular | 4.06 | 401997.6 |
| | | carcinoma | | |
| HUH-6 | Liver | Hepatoblastoma | 3.79 | 555328.6 |
| CLONE5 | | | | |
| Hep G2 | Liver | Hepatoblastoma | 4.23 | 561844.54 |
| RBE | Liver/Bile duct | Intrahepatic | 2.77 | 851.19 |
| | | Cholangiocarcinoma | | |
| NCI-H2122 | Lung | Lung | 2.45 | 266.15 |
| | | adenocarcinoma | | |
| NCI-H1568 | Lung | Lung | 2.38 | 273.26 |
| | | adenocarcinoma | | |
| NCI-H322 | Lung | Minimally invasive | 2.46 | 289.89 |
| | | lung | | |
| | | adenocarcinoma | | |
| NCI-H520 | Lung | Squamous cell lung | 2.37 | 299.27 |
| | | carcinoma | | |
| NCI-H1437 | Lung | Lung | 2.42 | 305.01 |
| | | adenocarcinoma | | |
| HCC-78 | Lung | Lung | 2.86 | 313.76 |
| | | adenocarcinoma | | |
| NCI-H3122 | Lung | Lung | 2.95 | 325.15 |
| | | adenocarcinoma | | |
| NCI-H820 | Lung | Papillary lung | 2.41 | 348.11 |
| | | adenocarcinoma | | |
| NCI-H1703 | Lung | Squamous cell lung | 2.33 | 359.14 |
| | | carcinoma | | |
| NCI-H1395 | Lung | Lung | 2.74 | 361.95 |
| | | adenocarcinoma | | |
| Calu-1 | Lung | Squamous cell lung | 2.86 | 366.37 |
| | | carcinoma | | |
| NCI-H358 | Lung | Minimally invasive | 2.64 | 370.36 |
| | | lung | | |
| | | adenocarcinoma | | |
| NCI-H1435 | Lung | Non-small cell lung | 2.72 | 382.78 |
| | | carcinoma | | |
| NCI-H1155 | Lung | Large cell lung | 2.55 | 453.04 |
| | | carcinoma | | |
| NCI-H2170 | Lung | Squamous cell lung | 2.65 | 464.26 |
| | | carcinoma | | |
| HCC4006 | Lung | Lung | 2.71 | 483.77 |
| | | adenocarcinoma | | |
| NCI-H1573 | Lung | Lung | 2.76 | 497.39 |
| | | adenocarcinoma | | |
| MsTo 211H | Lung | Pleural biphasic | 2.7 | 528.4 |
| | | mesothelioma | | |
| NCI-H2228 | Lung | Lung | 2.96 | 613.04 |
| | | adenocarcinoma | | |
| NCI-H82 | Lung | Small cell lung | 2.62 | 754.16 |
| | | carcinoma | | |
| NCI-H23 | Lung | Lung | 2.84 | 758.36 |
| | | adenocarcinoma | | |
| SNU-2535 | Lung | Non-small cell lung | 2.99 | 773.4 |
| | | carcinoma | | |
| NCI-H1299 | Lung | Large cell lung | 2.68 | 783.64 |
| | | carcinoma | | |
| NCI-H1373 | Lung | Lung | 2.66 | 793.27 |
| | | adenocarcinoma | | |
| NCI-H1651 | Lung | Lung | 2.83 | 845.93 |
| | | adenocarcinoma | | |
| HCC827 | Lung | Lung | 3.08 | 1010.61 |
| | | adenocarcinoma | | |
| SK-MES-1 | Lung | Squamous cell lung | 3.17 | 1036.22 |
| | | carcinoma | | |
| SK-LU-1 | Lung | Lung | 3.08 | 1039.7 |
| | | adenocarcinoma | | |
| A549 | Lung | Lung | 2.87 | 1042 |
| | | adenocarcinoma | | |
| DMS 53 | Lung | Small cell lung | 3.01 | 1068.34 |
| | | carcinoma | | |
| PC-9 | Lung | Lung | 3.17 | 1116.14 |
| | | adenocarcinoma | | |
| Calu-3 | Lung | Lung | 3.22 | 1118.77 |
| | | adenocarcinoma | | |
| NCI-H1993 | Lung | Lung | 2.99 | 1193.51 |
| | | adenocarcinoma | | |
| NCI-H1792 | Lung | Lung | 3.11 | 1270.18 |
| | | adenocarcinoma | | |
| NCI-H226 | Lung | Pleural epithelioid | 2.98 | 2234.63 |
| | | mesothelioma | | |
| NCI-H187 | Lung | Small cell lung | 2.89 | 8360.07 |
| | | carcinoma | | |
| NCI-H446 | Lung | Small cell lung | 3.83 | 30535.83 |
| | | carcinoma | | |
| NCI-H146 | Lung | Small cell lung | 3.86 | 42590.26 |
| | | carcinoma | | |
| NCI-H2452 | Lung | Pleural biphasic | 3.72 | 70792.47 |
| | | mesothelioma | | |
| NCI-H1648 | Lung | Lung | 3.56 | 72932.43 |
| | | adenocarcinoma | | |
| NCI-H526 | Lung | Small cell lung | 3.6 | 212022.23 |
| | | carcinoma | | |
| DMS 79 | Lung | Small cell lung | 4.09 | 420290.27 |
| | | carcinoma | | |
| SW1271 | Lung | Small cell lung | 3.9 | 498336.38 |
| | | carcinoma | | |
| A-673 | Muscle | Ewing sarcoma | 2.78 | 700.79 |
| A-204 | Muscle | Embryonal | 3.66 | 183955.12 |
| | | rhabdomyosarcoma | | |
| RPMI-2650 | Nose | Head and neck | 3.04 | 843.22 |
| | | squamous cell | | |
| | | carcinoma | | |
| C666-1 | Nose | Nasopharyngeal | 3.15 | 24602.9 |
| | | carcinoma | | |
| A2780cis | Ovary | Ovarian | 2.68 | 540.91 |
| | | endometrioid | | |
| | | adenocarcinoma | | |
| A2780 | Ovary | Ovarian | 2.43 | 862.93 |
| | | endometrioid | | |
| | | adenocarcinoma | | |
| OVCAR-8 | Ovary | High grade ovarian | 3.05 | 1016.32 |
| | | serous | | |
| | | adenocarcinoma | | |
| SW756 | Ovary | Human | 3.75 | 37931.18 |
| | | papillomavirus- | | |
| | | related cervical | | |
| | | squamous cell | | |
| | | carcinoma | | |
| ES-2 | Ovary | Ovarian clear cell | 3.75 | 70774.45 |
| | | adenocarcinoma | | |
| SK-OV-3 | Ovary | Ovarian serous | 3.53 | 84897.86 |
| | | cystadenocarcinoma | | |
| SW626 | Ovary | Colon | 3.89 | 414946.39 |
| | | adenocarcinoma | | |
| BxPC-3 | Pancreas | Pancreatic ductal | 2.97 | 350.92 |
| | | adenocarcinoma | | |
| HPAC | Pancreas | Pancreatic | 2.98 | 467.2 |
| | | adenocarcinoma | | |
| KP4 | Pancreas | Pancreatic | 2.84 | 519.77 |
| | | carcinoma | | |
| PL45 | Pancreas | Pancreatic ductal | 3.37 | 992.52 |
| | | adenocarcinoma | | |
| PANC-1 | Pancreas | Pancreatic ductal | 3.13 | 2605.87 |
| | | adenocarcinoma | | |
| MIA PaCa-2 | Pancreas | Pancreatic ductal | 3.15 | 2911.46 |
| | | adenocarcinoma | | |
| FaDu | Pharynx | Hypopharyngeal | 2.9 | 341.02 |
| | | squamous cell | | |
| | | carcinoma | | |
| JAR | Placenta | Gestational | 2.57 | 672.99 |
| | | choriocarcinoma | | |
| JEG-3 | Placenta | Gestational | 3.2 | 992.81 |
| | | choriocarcinoma | | |
| LNCaP clone | Prostate | Prostate carcinoma | 2.48 | 172.26 |
| FGC | | | | |
| DU 145 | Prostate | Prostate carcinoma | 2.99 | 834.85 |
| 22Rv1 | Prostate | Prostate carcinoma | 2.52 | 1091.02 |
| A-431 | Skin | Vulvar squamous | 2.96 | 1107.28 |
| | | cell carcinoma | | |
| A-375 | Skin | Amelanotic | 3.42 | 28059.1 |
| | | melanoma | | |
| WM-266-4 | Skin | Melanoma | 3.87 | 32449.76 |
| SK-MEL-5 | Skin | Cutaneous | 4.12 | 75059.71 |
| | | melanoma | | |
| SK-MEL-28 | Skin | Cutaneous | 4.18 | 105375.51 |
| | | melanoma | | |
| Hutu 80 | Small | Duodenal | 2.69 | 879.99 |
| | intestine/Duodenum | adenocarcinoma | | |
| HT-1080 | Soft tissue | Fibrosarcoma | 3.07 | 990.46 |
| SW982 | Soft tissue | Biphasic synovial | 3.99 | 66016.56 |
| | | sarcoma | | |
| SNU-601 | Stomach/Gastric | Signet ring cell | 2.89 | 330.58 |
| | | gastric | | |
| | | adenocarcinoma | | |
| HGC-27 | Stomach/Gastric | Gastric carcinoma | 2.39 | 512.88 |
| IM95m | Stomach/Gastric | Gastric | 2.9 | 600.63 |
| | | adenocarcinoma | | |
| OCUM-2M | Stomach/Gastric | Diffuse gastric | 2.88 | 847.86 |
| | | adenocarcinoma | | |
| OCUM-2D | Stomach/Gastric | Diffuse gastric | 3.12 | 1142.99 |
| | | adenocarcinoma | | |
| NUGC-4 | Stomach/Gastric | Signet ring cell | 3.11 | 1347.85 |
| | | gastric | | |
| | | adenocarcinoma | | |
| SNU-5 | Stomach/Gastric | Gastric carcinoma | 3.11 | 1946.83 |
| MKN1 | Stomach/Gastric | Gastric | 3.21 | 5363.89 |
| | | adenosquamous | | |
| | | carcinoma | | |
| YCC-6 | Stomach/Gastric | Gastric | 2.95 | 6317.64 |
| | | adenocarcinoma | | |
| SNU-1 | Stomach/Gastric | Gastric carcinoma | 3.25 | 12609.72 |
| NUGC-3 | Stomach/Gastric | Gastric | 3.64 | 26498.71 |
| | | adenocarcinoma | | |
| OCUM-1 | Stomach/Gastric | Signet ring cell | 3.57 | 68296.84 |
| | | gastric | | |
| | | adenocarcinoma | | |
| SNU-484 | Stomach/Gastric | Gastric | 3.64 | 74844.98 |
| | | adenocarcinoma | | |
| SNU-216 | Stomach/Gastric | Gastric tubular | 3.7 | 123871.99 |
| | | adenocarcinoma | | |
| AGS | Stomach/Gastric | Gastric | 3.63 | 204800.21 |
| | | adenocarcinoma | | |
| YCC-10 | Stomach/Gastric | Gastric | 3.64 | 411544.94 |
| | | adenocarcinoma | | |
| SNU-719 | Stomach/Gastric | Gastric tubular | 3.58 | 1316065.92 |
| | | adenocarcinoma | | |
| TT | Thyroid | Hereditary thyroid | 3.19 | 2170.59 |
| | | gland medullary | | |
| | | carcinoma | | |
| SW579 | Thyroid | Thyroid gland | 3.73 | 50468.43 |
| | | squamous cell | | |
| | | carcinoma | | |
| CAL-27 | Tongue | Tongue squamous | 3.17 | 3419.19 |
| | | cell carcinoma | | |
| SCC-9 | Tongue | Tongue squamous | 3.31 | 26125.3 |
| | | cell carcinoma | | |
| OSC-19 | Tongue | Tongue squamous | 3.85 | 34452.9 |
| | | cell carcinoma | | |
| SCC-4 | Tongue | Tongue squamous | 3.69 | 832827.35 |
| | | cell carcinoma | | |
| Ishikawa | Uterus | Endometrial | 2.44 | 297.46 |
| | | adenocarcinoma | | |
| MES-SA/DX5 | Uterus | Uterine corpus | 2.17 | 342.05 |
| | | sarcoma | | |
| RL95-2 | Uterus | Endometrial | 2.63 | 415.52 |
| | | adenosquamous | | |
| | | carcinoma | | |
| HEC-1-B | Uterus | Endometrial | 2.85 | 438.31 |
| | | adenocarcinoma | | |
| MES-SA | Uterus | Uterine corpus | 2.67 | 480.38 |
| | | sarcoma | | |
| ME-180 | Uterus | Human | 2.65 | 562.17 |
| | | papillomavirus- | | |
| | | related cervical | | |
| | | squamous cell | | |
| | | carcinoma | | |
| HCC 94 | Uterus | Cervical squamous | 2.92 | 806.31 |
| | | cell carcinoma | | |
| Ca Ski | Uterus | Human | 4.35 | 41303.43 |
| | | papillomavirus- | | |
| | | related cervical | | |
| | | squamous cell | | |
| | | carcinoma | | |
| AN3 CA | Uterus | Endometrial | 3.88 | 45573.49 |
| | | adenocarcinoma | | |
| HT-3 | Uterus/Cervix | Cervical carcinoma | 3.91 | 30080.19 |
| L-82 | Blood | | 2.94 | NA |
| CoC1 | Ovary | | 2.95 | NA |
| GTL-16 | Stomach/Gastric | | 3.18 | NA |
| ARH-77 | Blood | | 3.22 | NA |
| SF268 | Brain&Nerves | | 3.24 | NA |
| OVCAR-3 | Ovary | | 3.29 | NA |
| SNU-638 | Stomach/Gastric | | 3.32 | NA |
| CoC1/DDP | Ovary | | 3.33 | NA |
| YCC-11 | Stomach/Gastric | | 3.34 | NA |
| KHOS/NP | Bone | | 3.37 | NA |
| HeLa 229 | Cervix | | 3.37 | NA |
| SNU-354 | Liver | | 3.39 | NA |
| ASH-3 | H&N/Thyroid | | 3.41 | NA |
| SCH | Stomach/Gastric | | 3.48 | NA |
| MS751 | Uterus/Cervix | | 3.52 | NA |
| YCC-1 | Stomach/Gastric | | 3.54 | NA |
| NCI-H1688 | Lung | | 3.57 | NA |
| UO.31 | Kidney | | 3.58 | NA |
| A-427 | Lung | | 3.59 | NA |
| C4-2 | Prostate | | 3.61 | NA |
| HCCC-9810 | Liver | | 3.64 | NA |
| YCC-2 | Stomach/Gastric | | 3.66 | NA |
| TJ905 | Brain&Nerves | | 3.68 | NA |
| C-33 A | Uterus | | 3.7 | NA |
| Y-79 | Eye | | 3.72 | NA |
| SW684 | Soft tissue | | 3.77 | NA |
| A875 | Skin | | 3.84 | NA |
| HCCLM3 | Liver | | 3.87 | NA |
| DoTc2 4510 | Uterus/Cervix | | 3.9 | NA |
| MKN74 | Stomach/Gastric | | 2.89 | NA |
| SK-HEP-1 | Liver | | 3.05 | NA |
| NCI-N87 | Stomach/Gastric | | 3.09 | NA |
| DV-90 | Lung | | 3.1 | NA |
| NCI-H1581 | Lung | | 3.1 | NA |
| SW1990 | Pancreas | | 3.12 | NA |
| NCI-H522 | Lung | | 3.12 | NA |
| NCI-H460 | Lung | | 3.13 | NA |
| Daoy | Brain&Nerves | | 3.14 | NA |
| NCI-H292 | Lung | | 3.17 | NA |
| NCI-H747 | Large | | 3.19 | NA |
| | intestine/Colorectum | | | |
| NCI-H2087 | Lung | | 3.19 | NA |
| BT-20 | Breast | | 3.2 | NA |
| SNU-423 | Liver | | 3.2 | NA |
| KATO III | Stomach/Gastric | | 3.2 | NA |
| Calu-6 | Lung | | 3.21 | NA |
| NCI-H2009 | Lung | | 3.22 | NA |
| TE-1 | Esophagus | | 3.23 | NA |
| HuCCT1 | Liver | | 3.24 | NA |
| MFE-296 | Uterus | | 3.24 | NA |
| TF-1 | Blood | | 3.24 | NA |
| MDA-MB-453 | Breast | | 3.26 | NA |
| HCC1500 | Breast | | 3.27 | NA |
| Capan-2 | Pancreas | | 3.28 | NA |
| D283 Med | Brain&Nerves | | 3.28 | NA |
| HEC-1-A | Uterus | | 3.28 | NA |
| LS1034 | Colorectum | | 3.29 | NA |
| A-172 | Brain&Nerves | | 3.29 | NA |
| SCC-25 | Tongue | | 3.29 | NA |
| HLE | Liver | | 3.3 | NA |
| NCI-H929 | Blood | | 3.32 | NA |
| A253 | Submaxillary gland | | 3.32 | NA |
| MDA-MB-436 | Breast | | 3.33 | NA |
| T47D | Breast | | 3.33 | NA |
| NCI-H1915 | Lung | | 3.33 | NA |
| NCI-H1781 | Lung | | 3.34 | NA |
| NCI-H1048 | Lung | | 3.35 | NA |
| HCC1937 | Breast | | 3.36 | NA |
| SW948 | Colorectum | | 3.37 | NA |
| SF-126 | Brain&Nerves | | 3.39 | NA |
| HCC1806 | Breast | | 3.39 | NA |
| U266B1 | Blood | | 3.41 | NA |
| NCI-H661 | Lung | | 3.43 | NA |
| BT-483 | Breast | | 3.43 | NA |
| HLF | Liver | | 3.43 | NA |
| HCC1954 | Breast | | 3.44 | NA |
| U-2 OS | Bone | | 3.44 | NA |
| ZR-75-30 | Breast | | 3.44 | NA |
| OE19 | Esophagus/gastric | | 3.44 | NA |
| | cardia | | | |
| MM.1S | Blood | | 3.46 | NA |
| EBC-1 | Lung | | 3.47 | NA |
| NCI-H209 | Lung | | 3.47 | NA |
| HCC2218 | Breast | | 3.48 | NA |
| SNU-668 | Stomach/Gastric | | 3.48 | NA |
| HCC2935 | Lung | | 3.49 | NA |
| MDA-MB-415 | Breast | | 3.49 | NA |
| SNU-C5 | Large | | 3.5 | NA |
| | intestine/Colorectum | | | |
| Hep3B | Liver | | 3.5 | NA |
| NCI-H1666 | Lung | | 3.51 | NA |
| KYSE-410 | Esophagus | | 3.51 | NA |
| UACC-812 | Breast | | 3.52 | NA |
| NCI-H727 | Lung | | 3.53 | NA |
| Caki-2 | Kidney | | 3.53 | NA |
| MKN45 | Stomach/Gastric | | 3.54 | NA |
| SNU-81 | Colorectum | | 3.54 | NA |
| Detroit 562 | Pharynx | | 3.54 | NA |
| SCC-15 | Tongue | | 3.56 | NA |
| CFPAC-1 | Pancreas | | 3.56 | NA |
| JHH-5 | Liver | | 3.56 | NA |
| DMS 114 | Lung | | 3.56 | NA |
| T84 | Colorectum | | 3.57 | NA |
| LN-229 | Brain&Nerves | | 3.59 | NA |
| AsPC-1 | Pancreas | | 3.59 | NA |
| NCI-H1975 | Lung | | 3.6 | NA |
| OCI-AML3 | Blood/Leukemia | | 3.62 | NA |
| NCI-H1838 | Lung | | 3.62 | NA |
| SW480 | Colorectum | | 3.62 | NA |
| JIMT-1 | Breast | | 3.62 | NA |
| HCC1187 | Breast | | 3.63 | NA |
| A2058 | Skin | | 3.64 | NA |
| Caov-3 | Ovary | | 3.65 | NA |
| SK-UT-1 | Uterus | | 3.67 | NA |
| COV644 | Ovary | | 3.69 | NA |
| NCI-H1563 | Lung | | 3.71 | NA |
| SW48 | Colorectum | | 3.74 | NA |
| SNU-620 | Stomach/Gastric | | 3.75 | NA |
| NCI-H69 | Lung | | 3.75 | NA |
| MCF7 | Breast | | 3.76 | NA |
| 8505C | H&N/Thyroid | | 3.78 | NA |
| NCI-H596 | Lung | | 3.81 | NA |
| HUH-1 | Liver | | 3.81 | NA |
| PC-3 | Prostate | | 3.82 | NA |
| SW1116 | Colorectum | | 3.82 | NA |
| Capan-1 | Pancreas | | 3.82 | NA |
| NCI-H441 | Lung | | 3.85 | NA |
| KU812 | Blood | | 3.91 | NA |
| NCI-H2052 | Lung | | 3.92 | NA |
| LS123 | Colorectum | | 4 | NA |
| HT-55 | Large | | 4 | NA |
| | intestine/Colorectum | | | |
| HT-1376 | Bladder | | 4.11 | NA |
| NCI-H1650 | Lung | | 4.13 | NA |

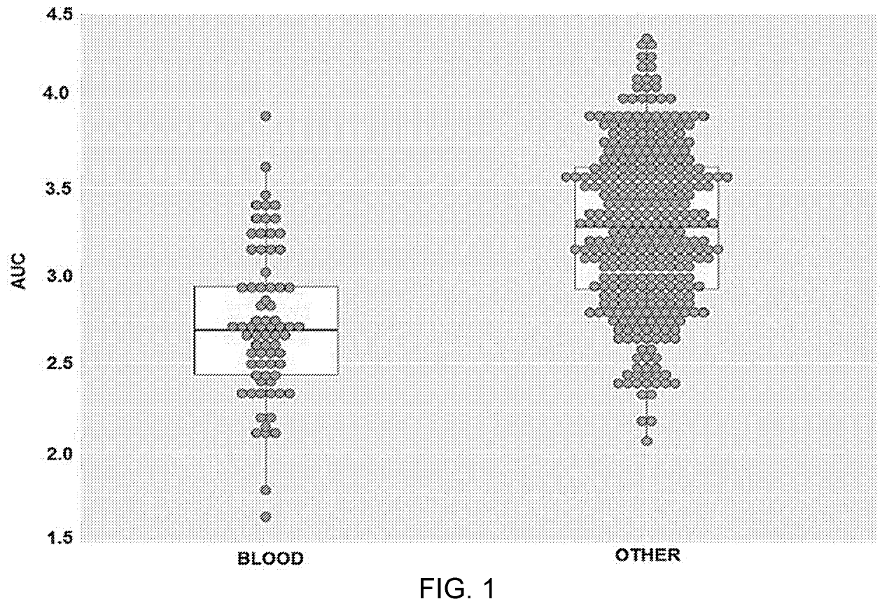

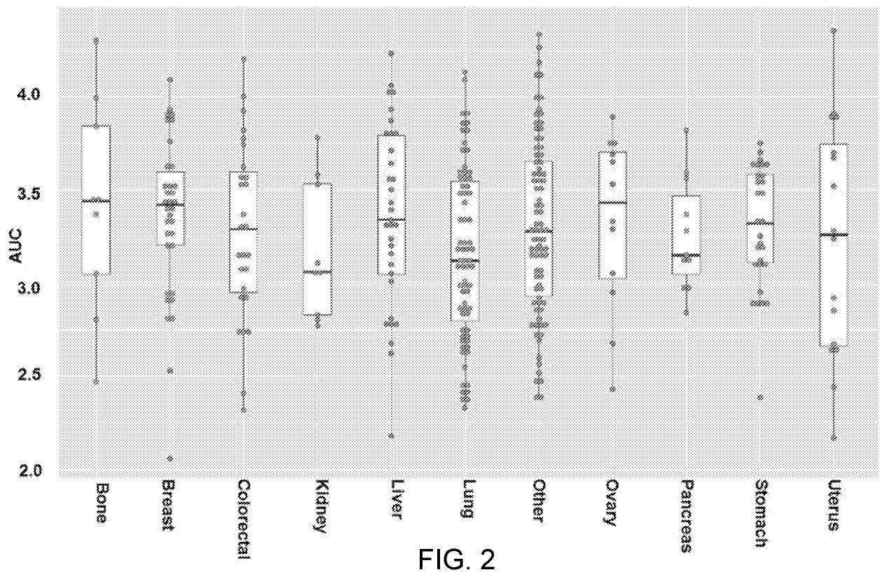

The 406 cancer cell lines of the study include 73 blood tumors and 333 solid tumors. The responses of blood and solid tumor cell lines are significantly different (Welch's t-test P-value=2.3e−16, FIG. 1). The analysis on blood tumors and on solid tumors was performed separately. In solid tumor cell lines, the average AUCs among different tumor types were not significantly different (one-way ANOVA P-value=0.54, FIG. 2). All solid cell lines were considered together.

Among the 406 cell lines, 311 have gene expression data, 308 have gene copy number data, and 286 have their mutation status detected in 1561 genes. For blood tumor cell lines, 57, 56 and 54 have expression, copy number, and mutation data. For solid tumor cell lines, 254, 252 and 232 have expression, copy number, and mutation data.

### Biomarker Discovery in Hematopoietic Cancer

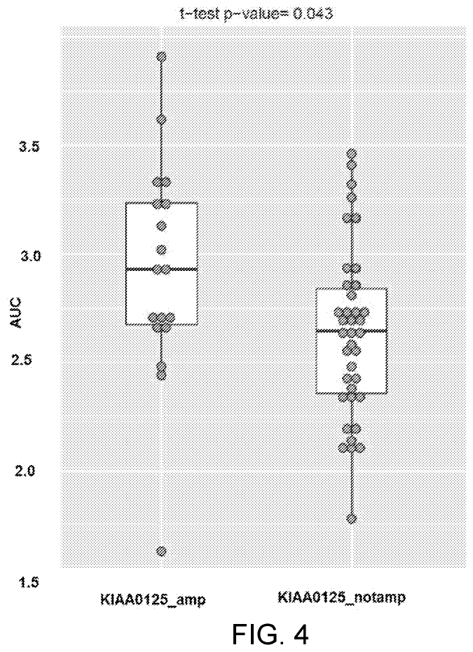

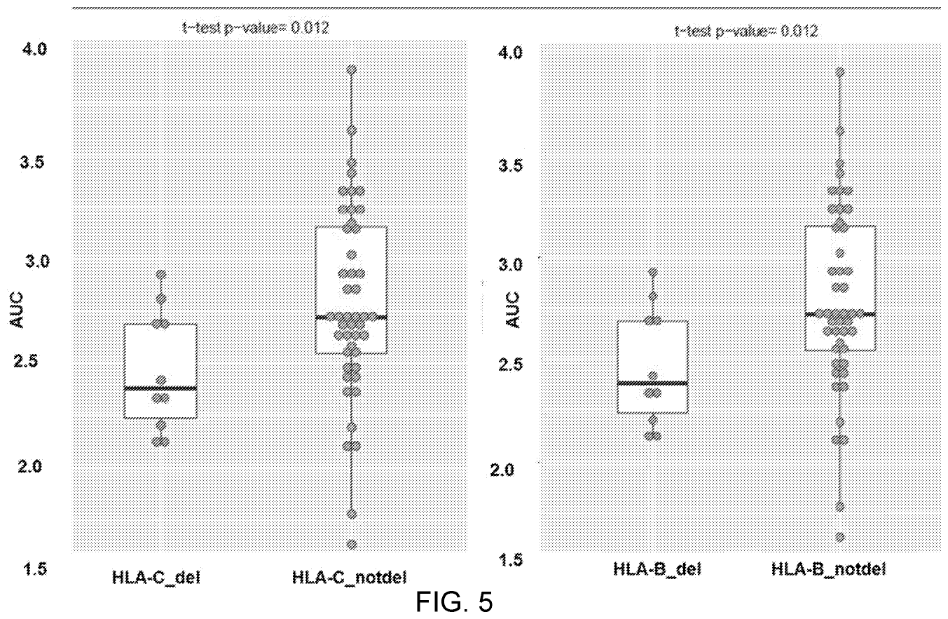

After removing genes with high ratio of low expressed cell lines (>90% cell lines with expression level <5) and low expression variation, 12,822 genes were kept for correlation analysis. Gene copy number was converted into integer (CN<0.5 to 0; CN<1.5 to 1; CN<2.5 to 2; CN<3.5 to 3; CN<4.5 to 4; CN≥4.5 to 5). Genes with CN≥4 were defined as amplified, and genes with CN=0 were defined as deleted. KIAA0125 was amplified in 18 cell lines and the amplified and unamplified cell lines had significantly different average AUCs (Welch's t-test P-value=0.043, FIG. 4). The cell lines with deleted and undeleted genes of HLA-B and HLA-C had significantly different average AUCs as well (Welch's t-test P-value<0.05, FIG. 5).

The cell lines were clustered into 2 groups according to each gene's mutation status, 12 genes were mutated in at least 4 cell lines. No genes had significantly different AUCs between mutated and wild type cell lines.

A Gene Set Enrichment Analysis (GSEA) using all genes was performed. The correlated genes were enriched in 5 pathways (nominal P-value<0.01).

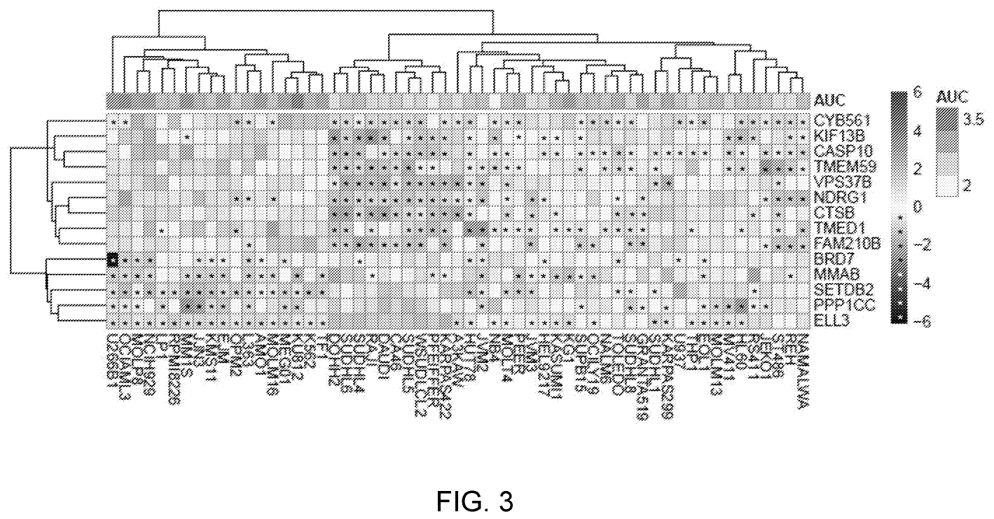

Fourteen genes had a Spearman rank correlation P-value less than 1e-4 (or R>0.493) between their mRNA expression level and AUCs. The 14 biomarker signature genes for hematopoietic cancer are shown in the table below. The clustering of 57 cell lines using these 14 genes shows that they are clustered into two groups, with the average AUCs of 3.11 and 2.56, separately (FIG. 3).

Biomarkers for hematopoietic cancer

| | Spearman | | Accession |
|---|---|---|---|
| Gene | Pvalue | Spearman R | Number |
| CASP10 | 6.36E−06 | 0.56 | NM_032977 |
| TMED1 | 1.06E−05 | 0.55 | NM_006858 |
| PPP1CC | 1.28E−05 | −0.54 | NM_002710 |
| TMEM59 | 1.37E−05 | 0.54 | NM_001305043 |
| BRD7 | 2.36E−05 | −0.53 | NM_001173984 |
| CYB561 | 3.25E−05 | 0.52 | NM_001915 |
| FAM210B | 3.88E−05 | 0.52 | NM_080821 |
| NDRG1 | 4.89E−05 | 0.51 | NM_001135242 |
| CTSB | 5.25E−05 | 0.51 | NM_001908 |
| MMAB | 6.92E−05 | −0.50 | NM_052845 |
| SETDB2 | 7.65E−05 | −0.50 | NM_031915 |
| VPS37B | 7.97E−05 | 0.50 | NM_024667 |
| ELL3 | 9.43E−05 | −0.49 | NM_025165 |
| KIF13B | 9.69E−05 | 0.49 | NM_015254 |

### Biomarker Discovery in Solid Tumor Cell Lines

Z-score normalization of AUCs on 254 solid tumor cell lines was performed. The cell lines with normalized value greater than 0.5 (corresponding to AUC>3.49) were defined as insensitive, and the ones with normalized value less than −0.5 (corresponding to AUC<3.05) were defined as sensitive. We obtained 77 sensitive and 89 insensitive solid cell lines. These 166 cell lines were randomly divided into 2 data sets: a training set with 52 sensitive and 60 insensitive cell lines, and a test set with 25 sensitive and 29 insensitive cell lines.

In the training group, after removing genes with high ratio of lowly expressed cell lines (>90% cell lines with expression level <5) 13,032 genes were kept. The expression of 258 genes having significant correlation with AUCs (Spearman correlation P-value<0.001), among which 9 (see table below) were selected as signature genes.

Biomarkers for Solid Tumor Cancer
Solid Tumor Cancer Biomarkers

| | | Spearman | Spearman |
|---|---|---|---|
| Gene | Accession Number | correlation P value | R value |
| LAMC3 | NM_006059 | 1.38E−04 | −0.29 |
| FAM210B | NM_080821 | 6.40E−10 | 0.45 |
| SENP8 | NM_001166340 | 7.20E−04 | −0.26 |
| ITGB3BP | NM_001206739 | 5.97E−05 | −0.30 |
| NUDT2 | NM_001161 | 6.98E−05 | −0.30 |
| HNRNPCL1 | NM_001013631 | 5.62E−07 | −0.37 |
| C20orf43 | NM_001283035 | 3.08E−06 | 0.35 |
| FRMD8 | NM_031904 | 7.37E−05 | 0.30 |
| STX16 | NM_001001433 | 3.80E−06 | 0.35 |

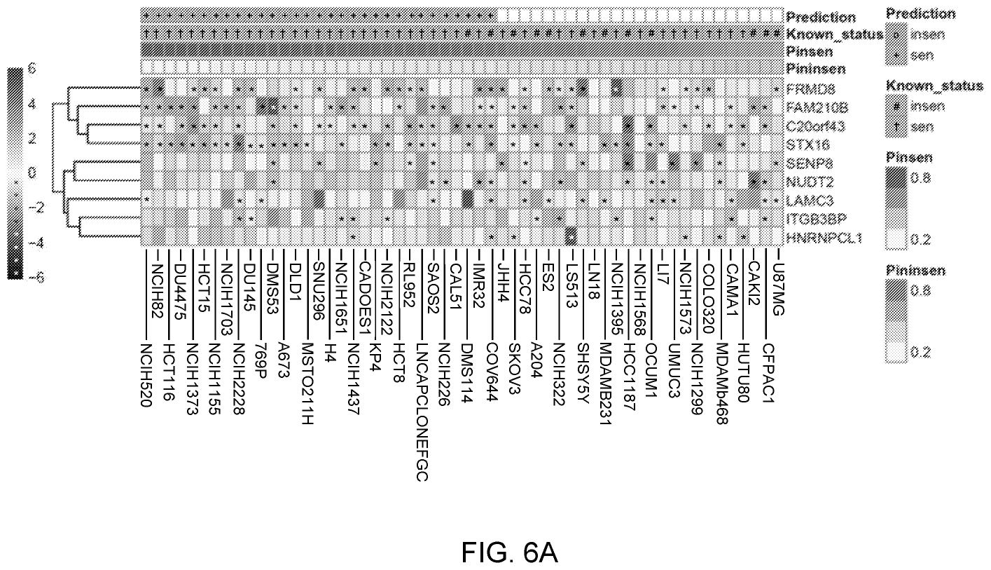

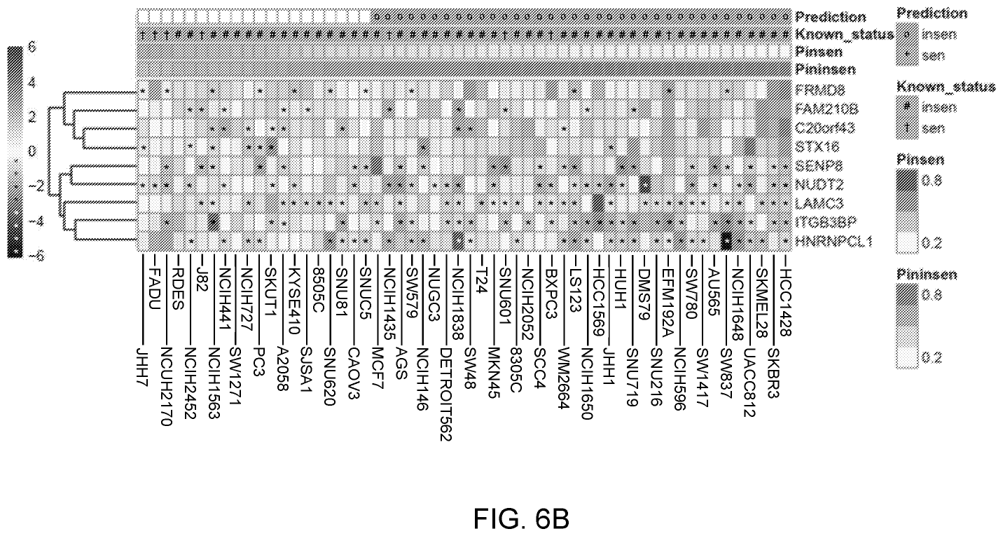

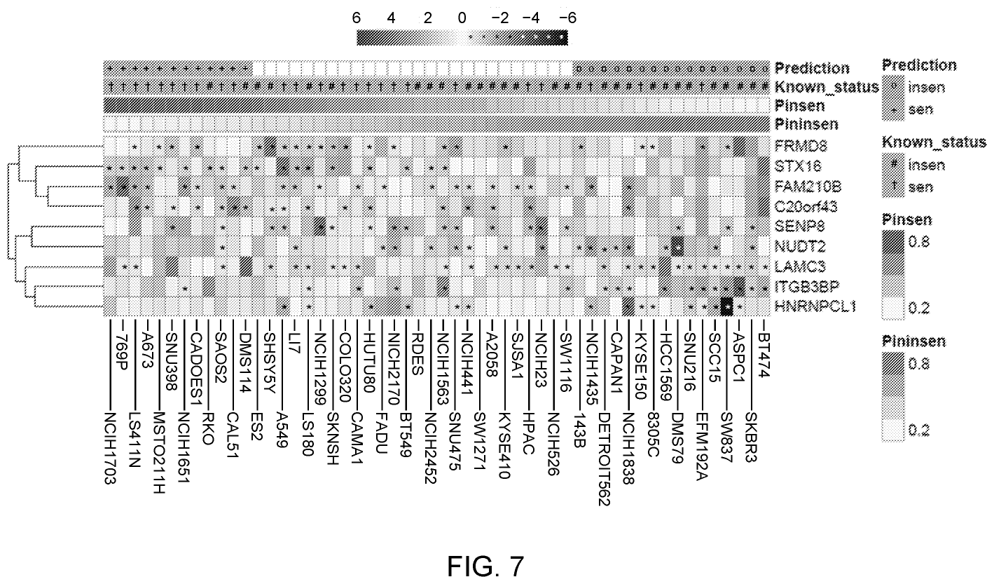

The prediction result in the training set using these 9 genes shows that 61 of 67 cell lines were correctly predicted (Probability in a subgroup >0.8), 5 cell lines were falsely predicted, and 45 cell lines failed to get their prediction result (Probability in either group <0.8, indicating no confidence to make a call). In the test group, 23 in 28 cell lines were correctly predicted (Probability in a subgroup >0.8), 5 cell lines were falsely predicted, and 26 failed to get their prediction result. The accuracy for drug response prediction is 91% in the training set (FIG. 6) and 82.1% in the test set (FIG. 7). If the training and test sets were combined, the accuracy is 88.4%.

Gene copy numbers were converted into integers (CN<0.5 to 0; CN<1.5 to 1; CN<2.5 to 2; CN<3.5 to 3, CN<4.5 to 4; CN>=4.5 to 5). Genes with CN=0 were defined as deleted, and genes with CN>=4 were defined as amplified. 14 genes (Welch's t-test P-value<0.01) have significantly different average AUC between deleted and undeleted cell lines. The average AUC is significantly different between the amplified and unamplified cell lines of 84 genes (Welch's t-test P-value<1e-5). These genes are clustered in the cytobands of 20p12 and 20p13. It is likely that the amplification of regions in these cytobands is related to drug response.

The cell lines were clustered into 2 groups according to each gene's mutation status, 193 genes are mutated in at least 4 cell lines. For 15 genes, the average AUC was significantly different between mutated and wild type cell lines (Welch's t-test P-value<0.05). A Gene Set Enrichment Analysis (GSEA) was performed in 254 solid cell lines. The correlated genes are enriched in 37 pathways (nominal P-value<0.01).

One of skill in the art will recognize that multiple accession numbers exist for variants of a gene and can be found in publicly available databases. Thus, not wishing to bound by only the accession number and associated sequence, gene variants of the genes of the hematopoietic cancers and solid tumor cancers tables above with 75% or more coverage are considered synonymous sequences.

### Example 3: ATF4 Pathway

Activating transcription factor 4 (ATF4) is a master regulator of genes essential for adaptation and regulation of gene expression in multiple cellular processes.

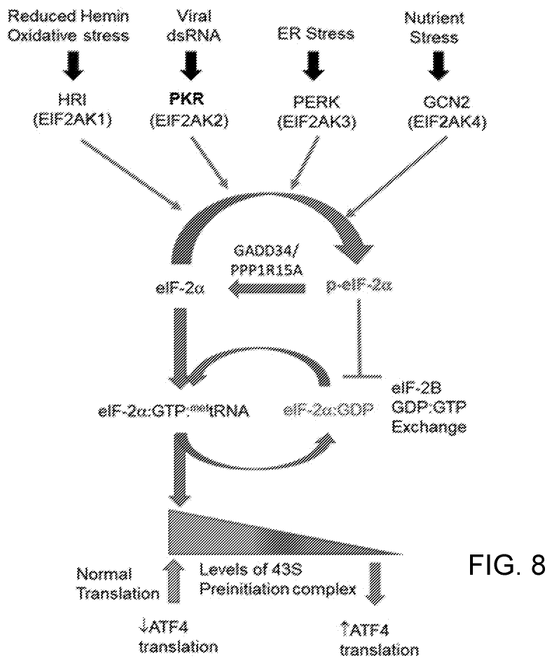

ATF4 encodes the transcription factor cAMP-response element binding protein 2 (CREB-2). Induction of ATF4 is governed by phosphorylation of the translation initiation factor eIF2α at the Ser51 residue, by one of four kinases. Phosphorylation of eIF2α reduces eIF2α:GTP:tRNAmet ternary complex formation. A reduction in ternary complex leads to reduced 43S preinitiation complex formation and cap-dependent mRNA translation with a concomitant increase in translation of mRNAs including ATF4 (FIG. 8).

ATF4 is induced in response to a wide range of cellular stresses including oxidative, nutrient, and endoplasmic reticulum (ER) stress. Importantly, cellular stress is a hallmark of multiple diseases, including cancers of the breast, lung, colorectal, and prostate. Induction of ATF4 via phosphorylation of translation factor eIF2α at residue Ser⁵¹ induces changes that result in tumor survival. ATF4 orchestrates a transcriptional program that results in improved nutrient utilization and transport, as well as increased expression of GADD34 resulting in a reduction of eIF2α phosphorylation and restoration of normal protein synthesis. Thus, transient ATF4 activation may aid in tumor survival. ATF4 is a novel and attractive therapeutic target in cancer treatment.

However, the adaptive nature of ATF4 activation is tempered by observations that persistent, elevated eIF2α phosphorylation and ATF4 induction will activate growth arrest and proapoptotic pathways. A key target of ATF4 is the transcription factor C/EBP homologous protein (CHOP/DDIT3). CHOP regulates apoptosis by increasing the expression of proapoptotic genes such as TRB3 and BIM while reducing the expression of anti-apoptotic genes such as Bcl-2, XIAP and Mcl1. Genes in the ATF4 pathway are shown in the table below. Pharmacological activation of ATF4 affords an approach to targeting a common oncogenic pathway that could provide improved anti-cancer therapeutics.

ATF4 Pathway Genes and Accession Numbers

| Gene | Accession Number |
|---|---|
| eIF2α | NM_004836.6 |
| ATF4 | NM_001675.4 |
| CHOP | NM_001195053.1 |
| GADD34 | NM_014330 |
| PPP1R15A | NM_014330.3 |
| PPP1R15B | NM_032833 |
| GADD153 | NM_001195053.1 |
| HRI (EIF2AK1) | NM_014413.3 |
| PRK (EIF2AK2) | NM_002759.3 |
| PERK (EIF2AK3) | NM_004836.6 |
| GCN2 (EIF2AK4) | NM_001013703.3 |
| TRB3 | NM_021158.4 |
| BIM | NM_138621.4 |
| Bcl-2 | NM_000633.2 |
| XIAP | NM_001167.3 |
| Mcl1 | NM_021960.4 |

Anti-proliferative activity of BTM compounds. The anti-proliferative activities of Compound A197 and Compound B19 were compared to 4EGI-1 (a known cancer cell growth inhibitor) in a panel of 99 tumor lines. Methods: A panel of 96 tumor and 3 normal cell lines were tested for sensitivity to the test compound. The cell lines were cultured in standard media and pipetted into 96-well plates at the required plating densities. The cells were acclimated for 24 hours prior to compound testing. Compound was prepared as a stock of 20 mM in DMSO. To prepare dose response curves compound was serially diluted in DMSO and dispensed into the plate wells using a Tecan D300e digital dispenser. The final DMSO concentration was 0.15%. After 72 hours of incubation cell number was determined using the CellTiter-Glo® protocol according to the manufacturers instructions (Promega). In this assay, ATP is measured as a surrogate of cell number. The activity of the compound is determined by comparing untreated cells with treated cells and calculating the % of signal retained. Compound activity is measured as an EC₅₀ of maximum level of efficacy and the two are used to compute an activity area. The table below provides a comparison of activity in a data sample.

Anti-proliferative activity of 4EGI-1 and Example compounds

| | | 4EGI-1, | A197, | B19, |
|---|---|---|---|---|
| | | IC₅₀ | IC₅₀ | IC₅₀ |
| Cell Line | Tumor Type | (μM) | (μM) | (μM) |
| A549 | non-small cell | 21.82 | 0.312 | 0.189 |
| | lung cancer | | | |
| HCT-116 | colorectal cancer | 10.53 | 0.295 | 0.343 |
| HT-1080 | fibrosarcoma | 4.661 | 0.725 | 0.528 |
| Jurkat | T cell leukemia | 13.21 | 0.179 | 0.166 |
| LoVo | colorectal cancer | 14.07 | 0.123 | 0.116 |
| MDA-MB-157 | breast cancer | 11.81 | (—) | (—) |
| MDA-MB-231 | breast cancer | 5.772 | (—) | (—) |
| MIA PaCa-2 | pancreactic cancer | 14.26 | 0.623 | 0.447 |
| | (carcinoma) | | | |
| NCI-H2122 | non-small cell | 13.76 | 0.2 | 0.184 |
| | lung cancer | | | |
| NCI-H460 | non-small cell | 9.459 | 0.156 | 0.167 |
| | lung cancer | | | |
| SK-MEL-28 | melanoma | 33.4 | (—) | (—) |
| SU-DHL-10 | B-cell lymphoma | 19.47 | 0.136 | 0.119 |
| SU-DHL-6 | B-cell lymphoma | 7.262 | 0.521 | 0.538 |
| SU-DHL-4 | B-cell lymphoma | 8.418 | 0.288 | 0.36 |
| HUVEC | primary endolithial | 7.993 | (—) | (—) |
| | cells | | | |
| HK-2 | kidney epilithial | 45.97 | (—) | (—) |
| NHLF | human lung fibroblast | | (—) | (—) |

Results of dose-response curves for evaluation of compounds in 99 tumor cell lines. Cells were cultured in 96-well plates and treated with compound for 72 hours. Cell number was determined using Cell-Titre-Glo. All data reported as mean of 3 biological replicates.
(—) = IC₅₀ > 30 μM.

Overall, the potencies of Compound A197 and Compound B19 were 50-100×greater than 4EGI-1. Compound A197 and Compound B19 were active in 40% of the tested cell lines (IC₅₀<2 μM) with 90% of hematopoietic tumor lines, and 28% of solid tumor lines being responsive. Among solid tumor lines, 80% of NSCLC, 37% of colorectal and 40% of sarcoma tumor lines were responsive. Breast cancer and melanoma lines were largely unresponsive to the compounds (although there are examples of certain such lines being active). In responsive cell lines, the activity range of Compound A197 and Compound B19 was for many compounds 0.1-2 μM. A distinction exists between tumor types: all responsive tumor lines undergo G1 growth arrest, but apoptosis is observed only in hematopoietic tumor lines, specifically B-cell lymphoma (data not shown). Importantly, primary diploid cell lines (e.g. human umbilical vein epithelial cells and normal human lung fibroblasts (NHLF)) tested negative.

Pharmacokinetic Properties of Compound A197 Compound A197 levels in blood plasma of CD-1 mice given a single, oral dose of the agent and compared to a single intravenous dose, were measured to assess basic pharmacokinetic parameters. A197 was dissolved in 1% NMP, 0.3% Tween-80 in 0.5% methylcellulose at a dose volume of 10 ml/kg. Following compound dosing, blood was collected by tail vein bleed at 30 minutes, 1, 2, 4, 8, 12, 24 and 48 hrs following dosing. Data are provided in the table below. Plasma drug tₘₐₓ was observed at 6 hrs. Bioavailability and half-life were estimated at 59% and 5.6 hours respectively.

Pharmacokinetic properties of A197

| | Oral, 2.5 mpk | Intravenous, 0.5 mpk |
|---|---|---|
| Cₘₐₓ (ng/mL) | 2607.3 | 2877 |
| tₘₐₓ (hr) | 6 | |
| AUC_{0-inf} (ng/mL/hr) | 42885 | 14503 |
| % F | 59.1 | |
| t_{1/2} | | 5.6 |
| CL (mL-min/kg) | | 0.58 |
| Vₛₛ (mL/kg) | | 284.7 |

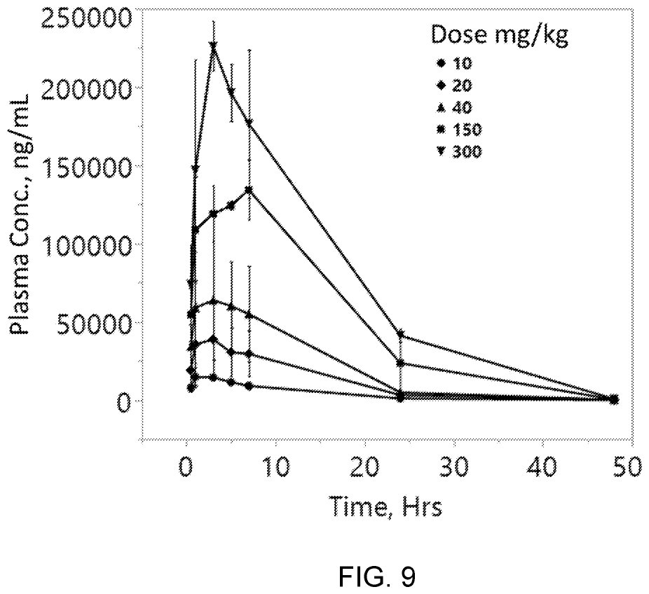

A single rising-dose mouse pharmacokinetics (PK) experiment was performed at PGP-52 TI 10, 20, 40, 150 and 300 mg/kg Compound A197. Female CD-1 mice (20-30 grams in weight) were dosed by oral gavage or by intravenous injection with A197 dissolved in 1% NMP, 0.3% Tween-80 in 0.5% methylcellulose at a dose volume of 10 ml/kg. Following compound dosing, blood was collected by tail vein bleed into K2EDTA tubes at 30 minutes, 1, 3, 5, 7, 24 an 48 hrs following dosing. Plasma (5 μL) from A197 dosed animals was acidified with 5 μL of 0.1% formic acid and 1% ammonium formate in methanol to precipitate protein and delipidate plasma. The material was centrifuged at 1000× and a 120 μL sample of the supernatant was then dried under vacuum. The deproteinated and delipidated residue was resuspended in 200 μL of 1% ammonium formate in methanol and the centrifugation and drying process repeated. The dried material is then resuspended in 100 μL of a mixture of 2 parts methanol: 1 part acetonitrile: 1 part water. A 10 μL sample of this material is then injected onto a Xbridge C18 2.5 μM, 3×30 mm, XP column attached to a TSQ Vantage LC/MS system for quantitation. The results of this experiment indicate that dose proportionality was observed up to 300 mg/kg (FIG. 9; Similar results were observed with Compound B19). The PK data demonstrate that Compound A197 and Compound B19 have drug-like PK properties suitable for once daily dosing.

Efficacy Demonstrated in Human Xenograft Models

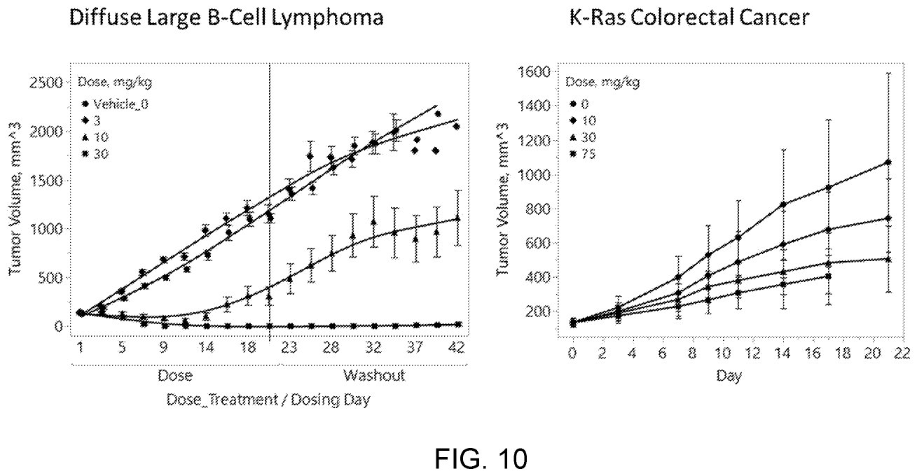

The positive results of the PK studies led to an evaluation of Compound A197 and Compound B19 in murine xenograft models of human tumors. Compound A197 was tested in a human tumor xenograft model using KRAS mutated colorectal cancer cell line HCT-116. Athymic nude mice (HSD:Athymic Nude-Foxn1 nu, Envigo) were inoculated subcutaneously with 5×10⁶ HCT-116 cells in the right rear flank. The animals wethen staged and randomized by tumor size to achieve dose groups of 10 animals each with an average tumor volume of 150 mm³. Tumor bearing mice were dosed once daily by oral gavage with a vehicle (5% NMP, 15% PEG400, 10% Solutol, and 70% D5W) or with A197 dissolved in vehicle to provide a dose concentration of 10 ml/kg. Compound B19 was tested in a human xenograft model using the human diffuse large B-cell lymphoma line SU-DHL-10 (ATCC). Female SCID beige mice (C.B-17/lcrHsd-PrkdcscidLystbg-J, Envigo) were inoculated subcutaneously with 5×10⁶ cells in Matrigel in the right rear flank. The animals were then staged and randomized by tumor size to achieve dose groups of 10 animals each with an average tumor volume of 150 mm³. Tumor bearing mice were dosed once daily by oral gavage with a vehicle (5% NMP, 15% PEG400, 10% Solutol, and 70% D5W) or with B19 dissolved in vehicle to provide a dose concentration of 10 mL/kg. The data clearly demonstrate the anti-tumor activity of both compounds in hematopoietic and solid tumors (FIG. 10). Compound B19 treatment resulted in a dose-dependent and durable regression in DLBCL tumors by day 21 (p<0.01 for 10 and 30 mpk doses). Compound A197 induced a similar level of regression in this model (data not shown). Compound A197 treatment resulted in significantly slower growth in the solid tumor model (p<01 for 75 and 30 mpk doses). In addition, Compound A197 treatment was well-tolerated with no overt signs of toxicity as determined using markers of liver function, metabolism, and myelopoiesis (data not shown).

Mechanism of Action Screens A series of broad high content imaging, transcriptomic, metabolomic, and CRISPR KO screens were performed to link Compound A197 activity to a probable method of action. The data from all studies is summarized in the table below. The outcome of these screens included two notable observations. The outcome of the transcriptomic analysis revealed that Compound A197 induced the ATF4 pathway. Second, Compound A197 induced specific alterations in redox (decreased GSH), energy (decrease in ATP/AMP ratio), and TCA cycle intermediates (decreased isocitrate/aconitate) in tumor cells but not NHLF (normal human lung fibroblast) cells.

Compound profiling screens and results.

| Mode | Target | Outcome |
|---|---|---|
| High Content | Cell cycle | G1 arrest associated with TP53 independent CDKN1A |
| | | induction |
| | DNA | No evidence of DNA damage using Histtone H2A.x |
| | Damage | phosphorylation as marker |
| | Autophagy | No detectable LC3 cleavage; no formation of |
| | | autophagic granules |
| | Cytoskeletal | No obvious changes in actin or microtubule localization |
| | | or structure |
| | Oxidative | No production of ROS |
| | Stress | |
| In vitro assays | GPCR | No activity on 69 family members |
| | Kinase | No activity on 63 kinase family members including Raf, |
| | | MEK, ERK, AKT, CDK1-4 |
| | HDAC | No activity on any HDAC |
| Transcriptomic | Gene | Induction of ATF pathway; no effect on cell cycle gene |
| | Expression | expression |
| Metabolomic | Metabolites | Decrease in total GSH levels; decrease in ATP/AMP |
| | | ratio; decrease in isocitrate and aconitate |
| CRISPR KO | 20,000 | Increased resistance to compound was associated with |
| | individual | inhibition of MAPK signaling as well as mitochondrial |
| | gene KO | biogenesis; Increased sensitivity was associated with |
| | | entry of pyruvatre into the TCA cycle as mitochondrial |
| | | OXPHOS |

ATF4-mediated gene expression profile is induced by compounds. ATF4 activation is closely aligned with activation of ER stress and the related TF's ATF6, ERN1 and XBP1. To determine the role of each transcription factor, a set of fifteen genes were selected to evaluate the specificity of the response as related ATF4 and to other transcription factors. The genes chosen have demonstrated the requirement for a specific transcription factor based on the use of gene deletion studies in the absence of specific genes (eg gene deletion of ATF6 largely eliminates the induction of HSPA6 by an ER stress inducer such as tunicamycin whereas ATF4 deletion has no such prominent effect). Cell cycle targets were also included in the panel as a discrete effect on progression through G1 was noted in the data. These genes can be regulated in a variety of ways and so reflect an outcome of compound action: cell cycle arrest.

Gene ID for mRNA Expression Profiling

| | | RefSeq |
|---|---|---|
| Target Name | TF Pathway[Target Name] | Gene ID |
| ASNS | ATF4 | NM_133436.3 |
| CDK1 | Cell Cycle | NM_001786 |
| CDKN3 | Cell Cycle | NM_005192 |
| CDKN1A | Cell Cycle | NM_000389 |
| DDIT3 | ATF4 | NM_000389 |
| DDIT4 | ATF4 | NM_000389 |
| DNAJC3 | ATF6 | NM_000389 |
| DNAJC7 | ERN1/XBP1 | NM_000389 |
| HSP90B1 | ATF6 | NM_003299 |
| HSPA5 | ATF6 | NM_003299 |
| HSPA6 | ATF6 | NM_002155 |
| PPP1R15A | ATF4 | NM_014330 |
| SARS | ATF4 | NM_006513.4 |
| SLC7A11 | ATF4 | NM_014331 |
| XBP1 | ERN1/XBP1 | NM_005080 |

Four cell lines were chosen for gene expression profiling. Normal Human Lung Fibroblasts were chosen as an example of a non-responsive cell line. The colorectal cancer cell line HCT-116 and the chronic myelogenous leukemia line HAP1 were chosen as examples of cell lines responsive to compound undergoing growth arrest. The diffuse large B-Cell lymphoma line SU-DHL-2 was chosen as a cell line undergoing growth arrest and apoptosis. To measure the levels of expressed genes, cells (3×10⁵) were plated into a 6-well tissue culture plates coated with and then cultured for 24 hours. After 24 hours, the media was exchanged and replaced with media containing Compound 197 at a final concentration of 5 μM. The cells were then allowed to incubate 8 hrs with compound following which the media was removed and cells were processed for RNA isolation using the Qiagen RNAEasy Mini Kit according to the manufacturer's instructions. Briefly lysis buffer was added to each well followed by homogenization using a QiaShredder column. An equal volume of 70% ethanol is added to the column eluate and then applied to a RNAEasy solid phase separation column. The column is washed twice to remove fragmented DNA and then the RNA is eluted using sterile RNase free water. RNA recovery is determined using a NanoDrop nucleic acid quantification device. RNA (400 μg) is used to create cDNA by standard methods using reagents and protocols from ThermoFisher. QPCR analysis of each gene was performed using standard methods. Probes and primer sequences for the genes are listed in the table below; ThermoFisher Scientific was the vendor for all assays. Three standard reference genes are used for normalizing data. The change in gene expression relative to vehicle is calculated using Expression Analysis software (ThermoFisher).

| | RefSeq | | Catalog |
|---|---|---|---|
| Target Name | Gene ID | Assay ID | number |
| ASNS | NM_133436.3 | Hs04186194_m1 | 4331182 |
| CDK1 | NM_001786 | Hs00938777_m1 | 4331182 |
| CDKN3 | NM_005192 | Hs00193192_m1 | 4331182 |
| CDKN1A | NM_000389 | Hs00355782_m1 | 4331182 |
| DDIT3 | NM_000389 | Hs01090850_m1 | 4331182 |
| DDIT4 | NM_000389 | Hs01111686_g1 | 4331182 |
| DNAJC3 | NM_000389 | Hs00939346_m1 | 4331182 |
| DNAJC7 | NM_000389 | Hs00268602_m1 | 4331182 |
| HSP90B1 | NM_003299 | Hs00427665_g1 | 4331182 |
| HSPA5 | NM_003299 | Hs00607129_gH | 4331182 |
| HSPA6 | NM_002155 | Hs00275682_s1 | 4331182 |
| PPP1R15A | NM_014330 | Hs00169585_m1 | 4331182 |
| SARS | NM_006513.4 | Hs00197856_m1 | 4331182 |
| SLC7A11 | NM_014331 | Hs00921938_m1 | 4331182 |
| XBP1 | NM_005080 | Hs00231936_m1 | 4331182 |

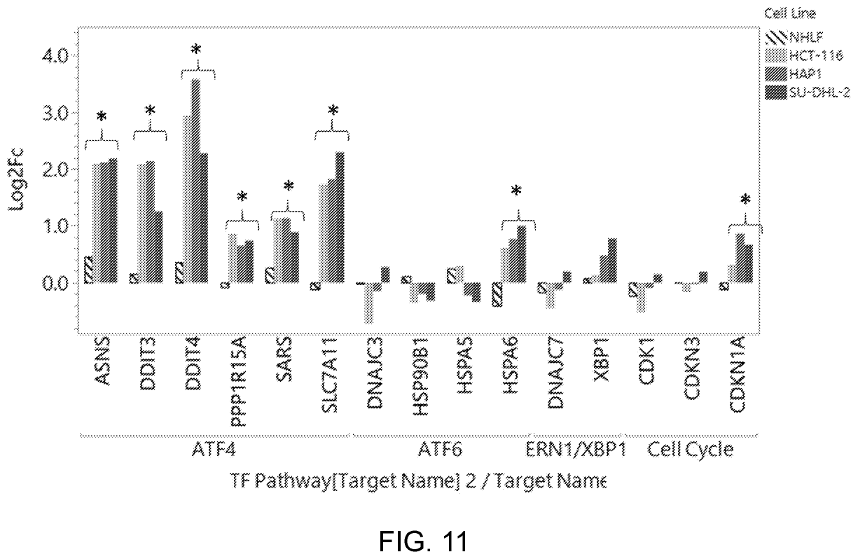

Compound 197 preferentially induces ATF4, but not ATF6 or IRE1/Xbp1 regulated genes in three responsive tumor cell lines (HCT-116, HAP-1 and SU-DHL-2) but not in primary NHLF (FIG. 11). Note that CDKN1A (p21/Waf1) mRNA expression is increased with compound treatment. Recent evidence suggests that CDKN1A expression may be regulated by eIF2α phosphorylation.

Treatment induces eIF2α phosphorylation in HCT-116 cells

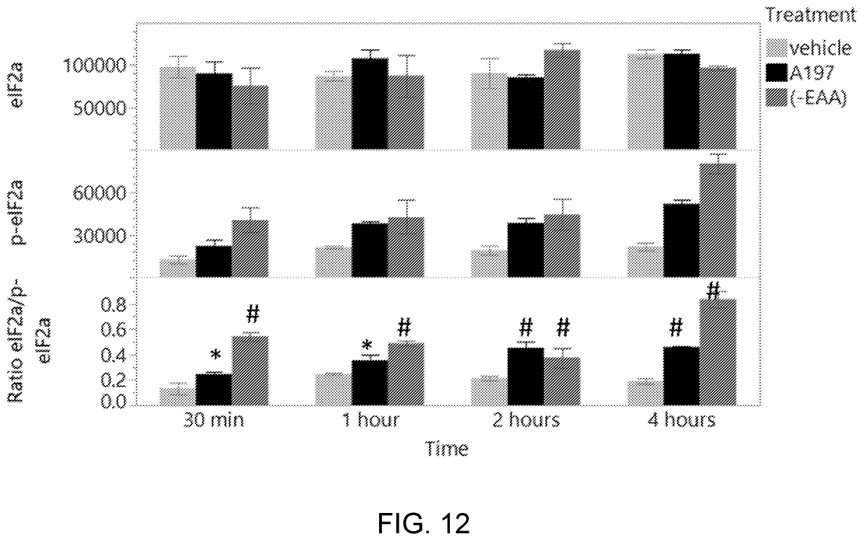

The canonical pathway of ATF4 induction involves eIF2α phosphorylation. To determine the levels of levels of eIF2α and p-eIF2α were determined using Western blotting. HCT-116 cells (1.5×10⁵ per well) were cultured in 12 well tissue culture plates and treated with 5 μM Compound A197 dissolved in McCoys complete medium with 10% FBS for 30 minutes, 1,2 or 4 hrs. Following treatment, media was removed, the cells were washed with PBS and then lysed following addition of RIPA lysis buffer. The cell lysate is then clarified by centrifugation at 14,000×g for 10 minutes. The clarified lysate protein levels are determined using BCA methodology. All lysates are diluted with RIPA buffer to give a final concentration of 200 μg/ml. As a control, HCT-116 cells were starved for essential amino acids (EAA), which increases eIF2α phosphorylation via EIF2AAK4 (GCN2). The HCT-116 cells were plated as above but the media was removed and replaced with Earls Balanced salt solution. All subsequent steps for lysate preparation were identical to those described above. Increased eIF2α phosphorylation within 30 minutes of compound treatment indicated that an eIF2 kinase has been activated (FIG. 12)

Total cellular GSH levels are reduced in tumor lines treated with Compound A197 The therapeutic compounds will cause a dose dependent reduction in total cellular glutathione in HCT-116, BJAB, SU-DHL-2, but not in NHLF cells. The maximum reduction of cellular GSH is in the range of 43-69%. The reduction In GSH levels appears independent of apoptosis. The IC₅₀ is consistent with the activity of the compound in cellular proliferation, suggesting a correlation between cell growth and redox status.

Effect of Compound A197 on Total Cellular GSH Levels

NHLF, BJAB, SU-DHL-2, and HCT-116 cells were treated with Compound A197 for 4 hours prior to lysate harvest. Total cellular glutathione (GSH) levels were determined using a luminescent endpoint (GSH-Glo, Promega). Cell proliferation was determined using Cell-Titre Glo following 72 hours of compound treatment. All data shown is mean±SD of three biological replicates.

Effect of A197 on total cellular GSH levels

| | GSH | | Proliferation | |
|---|---|---|---|---|
| | % Max | | | Apoptosis, |
| | reduction | IC₅₀ (Abs), μM | IC₅₀, μM | 72 hr |
| NHLF | 0 | NA | NA | No |
| BJAB | 69 ± 11 | 0.208 ± 0.08 | 0.295 ± 0.11 | Yes |
| SU-DHL-2 | 52 ± 4 | 0.12 ± 0.05 | 0.18 ± 0.06 | Yes |
| HCT-116 | 43 ± 9 | 0.36 ± 0.72 | 0.53 ± 0.21 | No |

All cells were treated with A197 for 4 hrs prior to lysate harvest. Total celluar GSH levels were determined using luminescent endpoint (GSH-Glo, Promega). Cell proliferation was determined using Cell-Titre Glo following 72 hours of compound treatment. All data are reported as mean ± std. dev. of three biological replicates.

### Example 4: ISR and Metabolite Biomarker Prediction of Anti-Proliferative Response of Tumor Cells to Therapeutic Compounds of the Disclosure

BCL tumor cell lines, six CRC lines with varying degrees of sensitivity to therapeutic compounds, and three normal primary cell lines with no anti-proliferative response are tested. The compounds A197 and B19 are tested along with compound A201a, a relatively inactive regioisomer of Compound A197 that serves as a control. Cells are tested over a range of concentrations from 0.01 to 10 μM. Cell proliferation and degree of apoptosis is determined using high-content cell imaging at 24 and 72 hours. RNA profiling on genes is performed using QPCR on RNA samples harvested at 8, 24, and 72 hours. Sampling for metabolite profiling occurs at 1 and 6 hours consistent with previous work demonstrating effects on ATP/AMP ratio and GSH levels.

ATP and AMP are extracted using hot ethanol and quantified by LC-MS. GSH is measured using GSH-Glo (Promega). Mitochondrial morphology and mitochondrial membrane potential are determined using confocal microscopy of cells stained with JC1 or TMRE. Mitochondrial staining studies are performed using the adherent CML line HAP1, CRC tumor lines, and NHLF. Time of exposure ranges from 30 minutes to 72 hours. All experimental data is collected as three biological replicates and technical duplicates for each data point.

Compound-mediated changes in biomarkers in tumor, but not normal, cell lines may be used as pharmacodynamics markers of compound activity but not as surrogates of compound efficacy. A correlation between gene response or metabolite profile to anti-proliferative activity (sensitivity and specificity of >70%) will provide sufficient preliminary evidence of prognostic value to expand screening to a greater number of cell lines. Additional transcript or metabolite markers can be evaluated to improve the result as can an increase in the number of cells screened.

### Example 5: Transcriptomic and Metabolomics Functional Pathways and Biomarkers Associated with the Anti-Proliferative Response to the Therapeutic Compounds

Biomarkers associated with functional response to a single active therapeutic compound are identified by an analysis of global gene expression and metabolite profiles in responsive KRAS mutant (HCT-116, LoVo) and non-responsive KRAS mutant (SW480) CRC cell lines. Comparison controls include a vehicle and a negative control compound. Samples are prepared using one concentration of compound after 8 and 24 hours of treatment. Global gene expression profiles are determined using RNASeq. Metabolite profiling samples are prepared using hot ethanol extraction followed by LC-MS detection of metabolites. All data points include three biological replicates. Data is analyzed using standard statistical approaches by comparing responses at each time point to vehicle.

RNA or metabolite biomarkers that are found in responsive but not in non-responsive cell lines are identified. Identified markers are further evaluated in a broader panel of CRC cell lines and expanded to other solid tumor types.

### Example 6: Cell Viability of Cancer Cell Lines (IC50)

The viability of 407 cancer cell lines was determined after treatment with Compound A197, a standard chemotherapy drug as a reference control (Cisplatin), or a culture medium vehicle control culture medium containing 0.25% (v/v) DMSO. Viability was determined by using the 50% inhibitory concentration (IC50) as determined with the CellTiter-Glo® Viability Assay (Promega). All cells were cultured under standard conditions in media supplemented with 10-15% Fetal Bovine Serum, at a temperature of 37° C., 5% CO₂ and 95% humidity.

Experiments were initiated by first thawing and equilibrating the CellTiter-Glo® Buffer to room temperature. The lyophilized CellTiter-Glo® Substrate was also equilibrated to room temperature. The lyophilized substrate was reconstituted with the CellTiter-Glo® buffer to form the CellTiter-Glo® Reagent.

Cells were harvested during the logarithmic growth period, counted, and the cell concentration adjusted to 4.44×10⁴ cells/mL using culture medium. A final cell density of 4×10³ cells/well was added in a 90 μL cell suspension to plates A and B as shown.

Plate Map for T0 plate reading (Plate A)

| Row | 1 | 2 | 3 | 4 | 5 | 6 | 7 | 8 | 9 | 10 | 11 | 12 |
|---|---|---|---|---|---|---|---|---|---|---|---|---|
| A | E | Cell | Cell | Cell | Cell | Cell | Cell | Cell | Cell | Cell | Cell | E |
| | | line1 | line2 | line3 | line4 | line5 | line6 | line7 | line8 | line9 | line10 | |
| B | M | N | N | N | N | N | N | N | N | N | N | E |
| C | M | N | N | N | N | N | N | N | N | N | N | E |
| D | M | N | N | N | N | N | N | N | N | N | N | E |
| E | M | N | N | N | N | N | N | N | N | N | N | E |
| F | M | N | N | N | N | N | N | N | N | N | N | E |
| G | M | N | N | N | N | N | N | N | N | N | N | E |
| H | E | E | E | E | E | E | E | E | E | E | E | E |

Plate Map for Test Compounds and Reference Control (Plate B)

| Row | 1 | 2 | 3 | 4 | 5 | 6 | 7 | 8 | 9 | 10 | 11 | 12 | |
|---|---|---|---|---|---|---|---|---|---|---|---|---|---|
| A | E | E | E | E | E | E | E | E | E | E | E | E | |
| B | M | C1 | C2 | C3 | C4 | C5 | C6 | C7 | C8 | C9 | N | E | Cpd |
| C | M | C1 | C2 | C3 | C4 | C5 | C6 | C7 | C8 | C9 | N | E | A197 |
| D | M | C1 | C2 | C3 | C4 | C5 | C6 | C7 | C8 | C9 | N | E | |
| E | M | C1 | C2 | C3 | C4 | C5 | C6 | C7 | C8 | C9 | N | E | Cisplat- |
| F | M | C1 | C2 | C3 | C4 | C5 | C6 | C7 | C8 | C9 | N | E | in |
| G | M | C1 | C2 | C3 | C4 | C5 | C6 | C7 | C8 | C9 | N | E | |
| H | E | E | E | E | E | E | E | E | E | E | E | E | |

E: Empty well containing complete culture medium or PBS
M: Medium control (blank control)
N: Vehicle control with culture medium containing 0.25% (v/v) DMSO
C1-C9: Nine concentration levels of test articles

Cells were allowed to grow overnight and the following day, the TO reading was obtained by adding 10 μL culture medium to each well of Plate A. The plate was allowed to equilibrate at room temperature for thirty minutes, after which 50 μL CellTiter-Glo® reagent was added to each well. The contents were mixed for 5 min on an orbital shaker to induce cell lysis. Plates were incubated for 20 minutes to stabilize the luminescent signal and the TO luminescence recorded.

The IC50 of test compounds and reference controls, Plate B, was determined by first preparing a 10× solution of Compound A197 to achieve nine dosage levels. A 10× reference control solution of Cisplatin was also prepared. Compound A197 and Cisplatin were dispensed in the appropriate wells of Plate B with the drug concentration dispensed in triplicate. Test plate B was incubated for 96 h in the humidified incubator at 37° C. with 5% CO₂.

Following the incubation, the plate was equilibrated at room temperature for thirty minutes. CellTiter-Glo® (50 μL) was added to each well of the plate and contents mixed for five minutes on an orbital shaker to induce cell lysis. The luminescent signal was allowed to stabilize at room temperature for 20 minutes and the luminescence recorded.

IC₅₀ (EC₅₀) was calculated using a dose-response curve, fitted using a nonlinear regression model with a sigmoidal dose response. Survival rate was calculated using the formula:

The Surviving rate (%)=(Lum_{Test article}−Lum_{Medium control})/(Lum_{Non-treated}−Lum_{Medium control})×100%.

Absolute IC50 (EC50) was calculated according to the dose-response curve generated by the statistical software (GraphPad Prism 5.0). IC50 and maximal inhibition for the tested cell lines is provided in Tables 6-1 through 6-16.

Summary of Absolute IC50s & Maximal inhibition
in 15 cell lines for Batch1

| | | | | % inhibition at top | | |
|---|---|---|---|---|---|---|
| | | Absolute IC50(μM) | | conc. | | |
| Cell | | Cpd | | Cpd | | Double |
| No. | Cell lines | A197 | Cisplatin | A197 | Cisplatin | time |
| 1 | OE19 | >30 | 19.38 | 53.21% | 81.07% | 51.49 |
| 2 | COLO | 0.49 | 3.18 | 72.92% | 99.94% | 22.39 |
| | 320DM | | | | | |
| 3 | YCC-10 | >30 | 0.69 | 38.86% | 93.96% | 33.39 |
| 4 | NCI-N87 | 1.47 | 0.95 | 54.19% | 97.87% | 50.10 |
| 5 | HCC4006 | 0.46 | 5.76 | 60.70% | 99.90% | 36.63 |
| 6 | Hep G2/C3A | >30 | 1.84 | 23.83% | 97.82% | 31.58 |
| 7 | COLO 205 | 1.14 | 1.97 | 60.57% | 99.26% | 31.01 |
| 8 | MKN45 | >30 | 5.47 | 14.51% | 96.51% | 32.71 |
| 9 | SNU-5 | 1.83 | 2.10 | 54.08% | 99.66% | 34.64 |
| 10 | MDA-MB- | >30 | 0.50 | 54.72% | 95.59% | 44.37 |
| | 436 | | | | | |
| 11 | ZR-75-30 | >30 | 2.99 | 10.01% | 99.13% | 82.34 |
| 12 | EBC-1 | >30 | 4.61 | 42.21% | 95.80% | 30.71 |
| 13 | HCC2218 | >30 | 10.84 | 33.43% | 99.70% | 66.76 |
| 14 | HM-7 | >30 | 6.26 | 43.55% | 94.91% | 53.18 |
| 15 | KATO III | >30 | 10.51 | 45.89% | 67.05% | 38.48 |

Summary of Absolute IC50s & Maximal inhibition
in 18 cell lines for Batch2

| | | | | % inhibition at top | | |
|---|---|---|---|---|---|---|
| | | Absolute IC50(μM) | | conc. | | |
| Cell | | Cpd | | Cpd | | Double |
| No. | Cell lines | A197 | Cisplatin | A197 | Cisplatin | time |
| 16 | HuCCT1 | >30 | 10.00 | 51.19% | 93.21% | 27.45 |
| 17 | FaDu | 0.38 | 1.49 | 62.82% | 99.73% | 21.06 |
| 18 | HT-1080 | 1.04 | 3.53 | 68.42% | 99.69% | 19.35 |
| 19 | SK-MEL-28 | >30 | 5.62 | 13.20% | 99.79% | 30.84 |
| 20 | Ishikawa | 0.29 | 2.75 | 82.51% | 99.95% | 21.26 |
| 21 | Calu-3 | 1.75 | 0.35 | 65.15% | 77.35% | 96.50 |
| 22 | A-375 | >30 | 1.04 | 52.11% | 99.96% | 17.54 |
| 23 | CAMA-1 | 0.69 | 9.44 | 54.72% | 98.71% | 53.72 |
| 24 | AN3 CA | >30 | 3.00 | 35.67% | 99.91% | 26.42 |
| 25 | NCI-H82 | 0.76 | 1.83 | 92.52% | 99.93% | 23.76 |
| 26 | A-431 | 1.15 | 2.07 | 66.83% | 99.94% | 37.77 |
| 27 | A2058 | >30 | 0.77 | 29.09% | 99.95% | 21.22 |
| 28 | DU 145 | 0.85 | 1.15 | 66.37% | 95.60% | 25.53 |
| 29 | SCC-4 | >30 | 2.96 | 26.22% | 99.06% | 33.41 |
| 30 | SK-MEL-5 | >30 | 3.20 | 25.67% | 99.94% | 28.03 |
| 31 | C4-2 | >30 | 14.86 | 45.07% | 99.39% | 36.83 |
| 32 | LNCaP | 0.18 | 3.43 | 76.07% | 99.62% | 30.74 |
| | clone FGC | | | | | |
| 33 | EFM-192A | 0.52 | 11.30 | 49.39% | 99.01% | 35.04 |

Summary of Absolute IC50s & Maximal inhibition
in 23 cell lines for Batch3

| | | | | % inhibition at top | | |
|---|---|---|---|---|---|---|
| | | Absolute IC50(μM) | | conc. | | |
| Cell | | Cpd | | Cpd | | Double |
| No. | Cell lines | A197 | Cisplatin | A197 | Cisplatin | time |
| 34 | DMS 53 | 1.07 | 2.85 | 85.18% | 99.56% | 57.49 |
| 35 | NCI-H69 | >30 | 3.28 | 21.37% | 95.76% | 59.38 |
| 36 | NCI-H526 | >30 | 0.33 | 44.57% | 99.95% | 22.35 |
| 37 | NCI-H209 | >30 | 0.44 | 45.11% | 99.74% | 58.40 |
| 38 | NCI-H146 | >30 | 1.81 | 37.67% | 97.87% | 30.83 |
| 39 | NCI-H460 | >30 | 2.77 | 49.03% | 99.81% | 27.58 |
| 40 | SW948 | >30 | 17.75 | 46.17% | 92.70% | 36.80 |
| 41 | RT4 | 0.83 | 4.20 | 49.68% | 99.97% | 29.34 |
| 42 | NCI-H820 | 0.35 | 0.79 | 78.69% | 99.97% | 27.02 |
| 43 | NCI-H1993 | 1.20 | 10.42 | 78.76% | 99.81% | 32.82 |
| 44 | A2780 | 0.40 | 1.38 | 69.48% | 99.96% | 24.86 |
| 45 | NCI-H446 | >30 | 1.93 | 51.21% | 99.94% | 30.27 |
| 46 | NCI-H520 | 0.30 | 10.71 | 76.45% | 99.34% | 28.40 |
| 47 | SNU-2535 | 0.75 | 16.28 | 46.87% | 94.30% | 47.45 |
| 48 | PC-9 | 1.11 | 1.97 | 52.79% | 99.68% | 28.35 |
| 49 | UACC-812 | >30 | 4.61 | 20.05% | 96.71% | 151.53 |
| 50 | NCI-H292 | >30 | 2.25 | 54.61% | 99.95% | 20.66 |
| 51 | 22Rv1 | 0.43 | 3.05 | 68.15% | 99.00% | 26.56 |
| 52 | NCI-H1048 | >30 | 1.37 | 66.49% | 99.76% | 45.17 |
| 53 | 5637 | 0.49 | 1.55 | 68.06% | 99.60% | 32.96 |
| 54 | NCI-H1975 | >30 | 3.24 | 25.02% | 99.05% | 27.18 |
| 55 | NCI-H358 | 0.38 | 6.07 | 68.44% | 97.31% | 27.42 |
| 56 | NCI-H2228 | 0.61 | 4.60 | 54.12% | 87.94% | 54.97 |

Summary of Absolute IC50s & Maximal inhibition
in 17 cell lines for Batch4

| | | | | % inhibition at top | | |
|---|---|---|---|---|---|---|
| | | Absolute IC50(μM) | | conc. | | |
| Cell | | Cpd | | Cpd | | Double |
| No. | Cell lines | A197 | Cisplatin | A197 | Cisplatin | time |
| 57 | SK-OV-3 | >30 | 2.88 | 34.62% | 96.61% | 26.82 |
| 58 | COV644 | >30 | 14.23 | 31.57% | 99.82% | 51.12 |
| 59 | NCI-H1299 | 0.78 | 2.67 | 83.01% | 99.47% | 19.01 |
| 60 | A875 | >30 | 1.86 | 10.68% | 99.91% | 18.23 |
| 61 | OVCAR-3 | >30 | 5.11 | 46.65% | 96.56% | 40.80 |
| 62 | RPMI-2650 | 0.81 | 1.89 | 52.41% | 99.85% | 23.59 |
| 63 | C666-1 | >30 | 18.08 | 36.20% | 73.03% | 33.34 |
| 64 | NCI-H1155 | 0.46 | 2.15 | 79.14% | 93.99% | 23.75 |
| 65 | JEG-3 | 1.08 | 0.52 | 52.54% | 99.97% | 19.75 |
| 66 | OVCAR-8 | 1.02 | 3.13 | 75.04% | 99.65% | 19.71 |
| 67 | NCI-H727 | >30 | 13.20 | 35.60% | 90.87% | 38.62 |
| 68 | NCI-H1703 | 0.36 | 6.11 | 73.10% | 99.93% | 24.61 |
| 69 | AsPC-1 | >30 | 4.94 | 34.71% | 78.18% | 36.73 |
| 70 | A-204 | >30 | 1.05 | 37.77% | 99.84% | 31.15 |
| 71 | SNU-761 | 1.11 | 1.39 | 59.03% | 99.97% | 35.06 |
| 72 | SW684 | >30 | 33.43 | 4.22% | 88.14% | 52.82 |
| 73 | HCCLM3 | >30 | 10.76 | 29.79% | 60.88% | 52.45 |

Summary of Absolute IC50s & Maximal inhibition
in 20 cell lines for Batch5

| | | | | % inhibition at top | | |
|---|---|---|---|---|---|---|
| | | Absolute IC50(μM) | | conc. | | |
| Cell | | Cpd | | Cpd | | Double |
| No. | Cell lines | A197 | Cisplatin | A197 | Cisplatin | time |
| 74 | NCI-H2087 | >30 | 1.01 | 50.14% | 96.83% | 35.66 |
| 75 | SNU-719 | >30 | 13.26 | 32.64% | 96.91% | 33.06 |
| 76 | OCUM-2D | 1.15 | 2.59 | 65.33% | 97.08% | 23.05 |
| 77 | SNU-484 | >30 | 4.96 | 45.94% | 99.93% | 32.56 |
| 78 | ES-2 | >30 | 3.51 | 44.22% | 99.90% | 33.54 |
| 79 | YCC-6 | 0.54 | 2.62 | 56.86% | 97.20% | 22.21 |
| 80 | OCUM-2M | 0.83 | 3.62 | 66.13% | 99.75% | 22.23 |
| 81 | AGS | >30 | 4.99 | 28.18% | 99.48% | 20.46 |
| 82 | NCI-H1648 | >30 | 8.25 | 57.33% | 98.53% | 59.01 |
| 83 | SNU-638 | >30 | 4.48 | 56.77% | 93.66% | 20.18 |
| 84 | SNU-601 | 0.49 | 0.60 | 67.28% | 91.98% | 26.94 |
| 85 | YCC-1 | >30 | 6.62 | 29.02% | 93.71% | 36.65 |
| 86 | NUGC-4 | 1.53 | 3.65 | 62.77% | 91.15% | 41.66 |
| 87 | NCI-H2170 | 0.50 | 1.02 | 75.97% | 98.78% | 30.68 |
| 88 | OCUM-1 | >30 | 9.94 | 19.71% | 95.06% | 42.78 |
| 89 | GTL-16 | >30 | 1.09 | 54.64% | 99.49% | 20.62 |
| 90 | NCI-H1792 | 1.27 | 1.48 | 68.85% | 99.91% | 26.06 |
| 91 | HGC-27 | 0.51 | 0.77 | 90.57% | 99.98% | 15.71 |
| 92 | MKN74 | >30 | 11.23 | 52.90% | 84.20% | 31.08 |
| 93 | HCC827 | >30 | 8.58 | 63.74% | 99.07% | 26.83 |

TABLE 6-6
Summary of Absolute IC50s & Maximal inhibition
in 33 cell lines for Batch6

| | | | | % inhibition at top | | |
|---|---|---|---|---|---|---|
| | | Absolute IC50(μM) | | conc. | | |
| Cell | | Cpd | | Cpd | | Double |
| No. | Cell lines | A197 | Cisplatin | A197 | Cisplatin | time |
| 94 | SNU-668 | >30 | 4.96 | 46.62% | 99.78% | 27.40 |
| 95 | NUGC-3 | 22.87 | 0.25 | 63.39% | 99.07% | 22.33 |
| 96 | MKN1 | 2.93 | 1.46 | 53.97% | 98.16% | 30.68 |
| 97 | SNU-1 | 10.70 | 2.15 | 74.67% | 99.84% | 20.92 |
| 98 | NCI-H1651 | 0.86 | 0.62 | 67.37% | 99.81% | 38.10 |
| 99 | YCC-11 | >30 | 0.62 | 48.65% | 99.91% | 39.72 |
| 100 | SCH | >30 | 6.82 | 53.08% | 99.80% | 64.12 |
| 101 | DOHH-2 | 0.31 | 0.19 | 99.99% | 100.00% | 24.73 |
| 102 | NCI-H322 | 0.29 | 10.83 | 85.38% | 98.25% | 35.16 |
| 103 | CoC1/DDP | >30 | 5.78 | 54.22% | 99.96% | 30.30 |
| 104 | Ca Ski | >30 | 6.44 | 32.50% | 99.34% | 35.53 |
| 105 | A498 | 0.86 | 4.28 | 81.18% | 96.18% | 24.91 |
| 106 | HLE | >30 | 2.48 | 46.48% | 98.43% | 25.28 |
| 107 | SW982 | >30 | 1.59 | −4.12% | 99.65% | 38.59 |
| 108 | NCI-H2122 | 0.27 | 8.93 | 72.62% | 98.52% | 27.10 |
| 109 | NCI-H1915 | >30 | 5.23 | 42.21% | 87.49% | 43.71 |
| 110 | C-33 A | >30 | 0.92 | 34.93% | 99.90% | 20.74 |
| 111 | SCC-9 | >30 | 8.37 | 34.73% | 90.93% | 47.02 |
| 112 | CoC1 | 1.32 | 3.24 | 60.92% | 99.96% | 27.39 |
| 113 | UO.31 | >30 | 4.40 | 43.36% | 98.52% | 35.51 |
| 114 | ACHN | 0.79 | 1.74 | 76.05% | 96.71% | 31.81 |
| 115 | NCI-H1568 | 0.27 | 0.95 | 71.17% | 99.94% | 43.36 |
| 116 | NCI-H1781 | >30 | 0.64 | 39.06% | 94.73% | 91.70 |
| 117 | HCC-78 | 0.41 | 2.25 | 61.86% | 99.56% | 32.20 |
| 118 | SNU-620 | >30 | 7.03 | 31.98% | 98.88% | 46.64 |
| 119 | NCI-H1666 | >30 | 6.16 | 45.30% | 98.84% | 27.41 |
| 120 | HCC 94 | 0.82 | 1.98 | 67.14% | 99.43% | 24.97 |
| 121 | 769-P | 1.04 | 0.97 | 80.28% | 98.91% | 22.11 |
| 122 | YCC-2 | >30 | 0.72 | 31.78% | 95.24% | 23.90 |
| 123 | HLF | >30 | 1.71 | 33.90% | 97.83% | 21.77 |
| 124 | ASH-3 | >30 | 2.72 | 47.68% | 99.92% | 29.90 |
| 125 | IHH-4 | 0.90 | 2.65 | 77.52% | 99.93% | 19.21 |
| 126 | NCI-H3122 | 0.39 | 5.73 | 57.62% | 99.31% | 28.51 |

Summary of Absolute IC50s & Maximal inhibition
in 16 cell lines for Batch7

| | | | | % inhibition at top | | |
|---|---|---|---|---|---|---|
| | | Absolute IC50(μM) | | conc. | | |
| Cell | | Cpd | | Cpd | | Double |
| No. | Cell lines | A197 | Cisplatin | A197 | Cisplatin | time |
| 127 | MES-SA | 0.49 | 0.66 | 69.64% | 99.98% | 18.69 |
| 128 | NCI-H1838 | >30 | 6.41 | 35.77% | 92.89% | 44.56 |
| 129 | NCI-H23 | 0.76 | 0.75 | 75.98% | 99.81% | 30.69 |
| 130 | SW780 | 22.58 | 1.98 | 63.71% | 99.87% | 22.39 |
| 131 | HEC-1-B | 0.55 | 10.59 | 52.92% | 92.96% | 32.58 |
| 132 | SK-UT-1 | >30 | 6.94 | 26.45% | 98.85% | 35.55 |
| 133 | MFE-296 | >30 | 3.31 | 53.41% | 99.64% | 22.58 |
| 134 | NCI-H441 | >30 | 6.18 | 28.35% | 74.99% | 57.00 |
| 135 | RBE | 0.84 | 4.14 | 79.87% | 92.87% | 32.82 |
| 136 | CA46 | 0.60 | 1.61 | 91.88% | 97.32% | 17.96 |
| 137 | WSU-NHL | 0.48 | 0.23 | 99.01% | 99.99% | 28.72 |
| 138 | JJN-3 | 1.34 | 4.08 | 90.80% | 99.35% | 30.52 |
| 139 | NB4 | 0.11 | 0.84 | 99.94% | 99.99% | 23.03 |
| 140 | Pfeiffer | 0.18 | 1.99 | 99.07% | 99.95% | 28.96 |
| 141 | ST486 | 0.22 | 0.26 | 96.04% | 99.98% | 18.66 |
| 142 | U-937 | 0.41 | 1.51 | 79.83% | 99.98% | 23.67 |

Summary of Absolute IC50s & Maximal inhibition
in 15 cell lines for Batch8

| | | | | % inhibition at top | | |
|---|---|---|---|---|---|---|
| | | Absolute IC50(μM) | | conc. | | |
| Cell | | Cpd | | Cpd | | Double |
| No. | Cell lines | A197 | Cisplatin | A197 | Cisplatin | time |
| 143 | MC/CAR | 3.28 | 3.15 | 54.69% | 99.97% | 35.00 |
| 144 | OCI-LY7 | 0.68 | 1.81 | 99.82% | 99.98% | 15.19 |
| 145 | NCI-H522 | >30 | 4.49 | 44.66% | 99.69% | 54.00 |
| 146 | SU-DHL-5 | 0.42 | 0.50 | 99.99% | 99.99% | 15.06 |
| 147 | BT474 | >30 | 59.04 | 16.28% | 87.37% | 61.66 |
| 148 | RPMI 8226 | 0.75 | 4.04 | 82.63% | 99.88% | 42.04 |
| 149 | MOLP8 | 0.99 | 1.75 | 49.02% | 99.97% | 34.03 |
| 150 | NAMALWA | 0.51 | 0.63 | 71.01% | 99.96% | 34.67 |
| 151 | AMO-1 | 1.48 | 1.92 | 50.29% | 99.94% | 39.55 |
| 152 | BT-549 | 0.45 | 4.70 | 56.25% | 99.92% | 32.59 |
| 153 | BT-20 | >30 | 2.15 | 46.55% | 99.41% | 44.24 |
| 154 | HEC-1-A | >30 | 13.34 | 43.57% | 83.97% | 30.93 |
| 155 | THP-1 | 0.21 | 1.66 | 75.93% | 99.97% | 28.79 |
| 156 | SU-DHL-6 | 1.05 | 1.85 | 99.91% | 99.98% | 21.21 |
| 157 | Ramos | 0.76 | 0.64 | 99.62% | 99.99% | 33.01 |

Summary of Absolute IC50s & Maximal inhibition
in 30 cell lines for Batch9

| | | | | % inhibition at top | | |
|---|---|---|---|---|---|---|
| | | Absolute IC50(μM) | | conc. | | |
| Cell | | Cpd | | Cpd | | Double |
| No. | Cell lines | A197 | Cisplatin | A197 | Cisplatin | time |
| 158 | NCI-H661 | >30 | 6.42 | 39.71% | 99.77% | 40.18 |
| 159 | DV-90 | >30 | 3.25 | 48.09% | 93.78% | 148.16 |
| 160 | RL95-2 | 0.42 | 1.50 | 78.60% | 99.96% | 25.39 |
| 161 | MS751 | >30 | 4.61 | 45.85% | 99.87% | 31.10 |
| 162 | JHH-5 | >30 | 0.85 | 56.52% | 95.33% | 20.44 |
| 163 | CAL-27 | 3.95 | 0.89 | 62.32% | 99.97% | 21.99 |
| 164 | SW1116 | >30 | 7.94 | 17.66% | 94.57% | 43.65 |
| 165 | A253 | >30 | 1.24 | 57.27% | 98.77% | 21.88 |
| 166 | JIMT-1 | >30 | 6.96 | 37.70% | 90.65% | 33.37 |
| 167 | A2780cis | 0.54 | 5.55 | 64.04% | 99.13% | 22.14 |
| 168 | HT-3 | >30 | 4.85 | 47.29% | 99.73% | 34.43 |
| 169 | SW1417 | >30 | 6.91 | 30.43% | 91.66% | 35.21 |
| 170 | SNU-216 | >30 | 6.07 | 33.93% | 98.24% | 25.28 |
| 171 | SCC-15 | >30 | 3.30 | 43.03% | 98.26% | 53.78 |
| 172 | ME-180 | 0.55 | 0.84 | 77.52% | 99.93% | 21.74 |
| 173 | WM-266-4 | >30 | 3.12 | 47.47% | 99.92% | 27.81 |
| 174 | DMS 114 | >30 | 2.02 | 48.48% | 99.92% | 29.19 |
| 175 | OSC-19 | >30 | 8.56 | 51.04% | 99.25% | 38.16 |
| 176 | HUH-7 | 1.05 | 1.63 | 61.34% | 98.01% | 25.76 |
| 177 | KYSE-410 | >301 | 13.45 | 43.89% | 90.48% | 22.86 |
| 178 | SW756 | 28.43 | 1.54 | 54.98% | 84.79% | 29.16 |
| 179 | Detroit 562 | >30 | 1.35 | 50.88% | 99.83% | 24.71 |
| 180 | SW626 | >30 | 3.97 | 25.09% | 88.38% | 46.41 |
| 181 | SW579 | >30 | 1.41 | 40.57% | 99.93% | 18.86 |
| 182 | TT | 2.07 | 28.06 | 53.44% | 94.17% | 54.25 |
| 183 | Raji | 0.45 | 2.07 | 92.32% | 93.71% | 44.99 |
| 184 | Daudi | 0.81 | 0.21 | 99.37% | 99.39% | 37.71 |
| 185 | EJM | 3.00 | 6.74 | 48.12% | 99.92% | 40.98 |
| 186 | A3/KAW | 0.29 | 0.93 | 99.34% | 99.99% | 24.05 |
| 187 | MOLM-16 | 0.35 | 0.87 | 52.41% | 99.32% | 36.65 |

Summary of Absolute IC50s & Maximal inhibition
in 39 cell lines for Batch10

| | | | | % inhibition at top | | |
|---|---|---|---|---|---|---|
| | | Absolute IC50(μM) | | conc. | | |
| Cell | | Cpd | | Cpd | | Double |
| No. | Cell lines | A197 | Cisplatin | A197 | Cisplatin | time |
| 188 | LS1034 | >30 | 6.52 | 45.27% | 92.75% | 25.82 |
| 189 | HOS | >30 | 1.70 | 36.46% | 99.27% | 17.75 |
| 190 | Caco-2 | >30 | 1.60 | 25.45% | 92.68% | 23.01 |
| 191 | A-172 | >30 | 8.58 | 41.29% | 97.59% | 19.49 |
| 192 | SiHa | >30 | 10.47 | 20.18% | 99.93% | 26.82 |
| 193 | HeLa | 1.74 | 0.28 | 55.51% | 99.49% | 18.91 |
| 194 | U-2 OS | 16.76 | 1.65 | 50.83% | 99.66% | 18.26 |
| 195 | LS123 | >30 | 12.02 | 12.00% | 94.40% | 59.32 |
| 196 | SK-HEP-1 | >30 | 13.15 | 61.02% | 99.90% | 27.03 |
| 197 | MDA-MB- | 0.52 | 0.27 | 84.39% | 99.91% | 28.15 |
| | 468 | | | | | |
| 198 | MES- | 0.34 | 2.77 | 94.79% | 99.95% | 16.50 |
| | SA/DX5 | | | | | |
| 199 | NCI-H747 | >30 | 7.30 | 55.44% | 94.19% | 27.96 |
| 200 | SF-126 | >30 | 1.95 | 52.94% | 98.90% | 23.87 |
| 201 | H4 | 0.73 | 2.26 | 70.53% | 99.95% | 17.54 |
| 202 | TJ905 | >30 | 9.46 | 13.96% | 92.20% | 24.17 |
| 203 | JHH-4 | 1.50 | 1.07 | 70.79% | 93.49% | 22.05 |
| 204 | Y-79 | >30 | 4.56 | 24.07% | 99.61% | 33.67 |
| 205 | JHH-7 | 0.66 | 1.83 | 79.46% | 98.65% | 18.12 |
| 206 | KHOS/NP | >30 | 1.33 | 44.49% | 99.39% | 19.16 |
| 207 | HCC1806 | >30 | 2.23 | 60.01% | 99.77% | 24.47 |
| 208 | KMH-2 | 1.44 | 2.65 | 45.62% | 99.95% | 26.84 |
| 209 | NCI-H508 | 1.33 | 8.02 | 62.91% | 98.99% | 25.72 |
| 210 | 786-O | 1.15 | 1.43 | 71.09% | 99.38% | 20.66 |
| 211 | T.Tn | 0.84 | 1.57 | 72.55% | 99.98% | 22.29 |
| 212 | U251 | 0.28 | 3.52 | 83.01% | 94.40% | 19.87 |
| 213 | J82 | 1.11 | 0.31 | 66.24% | 99.19% | 20.48 |
| 214 | T24 | >30 | 1.04 | 54.57% | 99.60% | 15.84 |
| 215 | M059K | 1.66 | 2.66 | 73.67% | 99.84% | 30.13 |
| 216 | A-673 | 0.68 | 0.26 | 74.34% | 99.93% | 20.38 |
| 217 | SNU-475 | 2.51 | 0.52 | 91.99% | 99.98% | 18.33 |
| 218 | UM-UC-3 | 0.93 | 1.62 | 85.71% | 99.82% | 17.30 |
| 219 | SW 156 | 2.39 | 1.03 | 65.19% | 99.31% | 24.60 |
| 220 | NCI-H716 | 1.57 | 8.99 | 70.97% | 98.19% | 35.94 |
| 221 | G-292 clone | 1.86 | 1.11 | 76.98% | 99.25% | 24.51 |
| | A141B1 | | | | | |
| 222 | HeLa 229 | >30 | 7.25 | 49.43% | 99.20% | 24.87 |
| 223 | SW837 | >30 | 4.21 | 30.80% | 95.14% | 30.25 |
| 224 | SNU-423 | >30 | 6.06 | 47.43% | 99.91% | 32.57 |
| 225 | SK-CO-1 | 1.12 | 0.21 | 59.76% | 99.26% | 30.58 |
| 226 | NCI-H1437 | 0.31 | 5.67 | 72.19% | 99.48% | 25.96 |

Summary of Absolute IC50s & Maximal inhibition
in 31 cell lines for Batch11

| | | | | % inhibition at top | | |
|---|---|---|---|---|---|---|
| | | Absolute IC50(μM) | | conc. | | |
| Cell | | Cpd | | Cpd | | Double |
| No. | Cell lines | A197 | Cisplatin | A197 | Cisplatin | time |
| 227 | LN-18 | 0.52 | 0.70 | 84.85% | 99.90% | 13.64 |
| 228 | SNU-C5 | >30 | 2.36 | 38.30% | 92.76% | 20.03 |
| 229 | DoTc2 4510 | >30 | 2.84 | 0 | 99.74% | 23.68 |
| 230 | CCK-81 | 0.32 | 4.51 | 69.60% | 99.96% | 22.42 |
| 231 | SK-LU-1 | 1.05 | 1.38 | 56.90% | 92.06% | 23.12 |
| 232 | NCI-H1573 | 0.46 | 10.59 | 62.17% | 99.72% | 23.37 |
| 233 | LS513 | 0.70 | 2.81 | 58.29% | 99.95% | 18.87 |
| 234 | A-427 | >30 | 5.78 | 35.55% | 99.57% | 21.69 |
| 235 | Caov-3 | >30 | 3.61 | 27.09% | 97.53% | 30.79 |
| 236 | KP4 | 0.53 | 6.25 | 61.69% | 98.97% | 19.96 |
| 237 | SK-NEP-1 | 1.06 | 0.60 | 65.19% | 99.98% | 22.04 |
| 238 | SK-N-FI | 1.19 | 1.34 | 45.05% | 99.95% | 29.53 |
| 239 | SCC-25 | >30 | 1.64 | 50.51% | 98.82% | 46.55 |
| 240 | WSU- | 0.68 | 1.37 | 99.98% | 99.99% | 22.75 |
| | DLCL2 | | | | | |
| 241 | NCI- | >30 | 2.98 | 54.04% | 99.94% | 25.57 |
| | H1581 | | | | | |
| 242 | IMR-32 | 0.80 | 0.06 | 84.11% | 99.98% | 35.66 |
| 243 | IM95m | 0.78 | 8.43 | 46.66% | 99.87% | 28.48 |
| 244 | HCC1187 | >30 | 2.01 | 30.08% | 99.94% | 107.85 |
| 245 | Daoy | >30 | 0.39 | 59.05% | 99.96% | 44.24 |
| 246 | SW48 | >30 | 1.27 | 32.72% | 96.23% | 28.44 |
| 247 | LN-229 | >30 | 7.68 | 35.32% | 94.79% | 25.77 |
| 248 | Toledo | 1.45 | 0.49 | 66.43% | 99.98% | 20.46 |
| 249 | JHH-1 | >30 | 6.04 | 29.40% | 99.80% | 38.71 |
| 250 | U-2932 | 0.46 | 0.79 | 85.31% | 98.99% | 24.57 |
| 251 | SF268 | >30 | 1.43 | 62.94% | 99.20% | 24.23 |
| 252 | U-87 MG | 24.90 | 4.05 | 56.64% | 91.64% | 25.92 |
| 253 | A549 | 1.04 | 4.35 | 76.14% | 97.65% | 18.18 |
| 254 | JeKo-1 | 0.79 | 1.36 | 99.78% | 99.99% | 19.47 |
| 255 | MOLM-13 | 0.87 | 0.78 | 70.99% | 99.99% | 16.42 |
| 256 | MV-4-11 | 0.32 | 0.41 | 93.93% | 99.99% | 21.46 |
| 257 | KARPAS- | >30 | 0.89 | 69.69% | 99.98% | 19.49 |
| | 299 | | | | | |

Summary of Absolute IC50s & Maximal inhibition
in 34 cell lines for Batch12

| | | | | % inhibition at top | | |
|---|---|---|---|---|---|---|
| | | Absolute IC50(μM) | | conc. | | |
| Cell | | Cpd | | Cpd | | Double |
| No. | Cell lines | A197 | Cisplatin | A197 | Cisplatin | time |
| 258 | OCI-LY-19 | 0.95 | 0.16 | 92.77% | 99.98% | 21.13 |
| 259 | JVM-2 | 0.73 | 1.46 | 82.46% | 99.86% | 45.33 |
| 260 | Kasumi-1 | 1.09 | 2.92 | 43.21% | 99.97% | 41.73 |
| 261 | BxPC-3 | 0.54 | 1.27 | 68.19% | 99.97% | 21.53 |
| 262 | SNU-354 | >30 | 13.80 | 51.25% | 99.84% | 31.07 |
| 263 | JVM-13 | 0.39 | 0.54 | 78.25% | 99.16% | 53.88 |
| 264 | PC-3 | >30 | 4.66 | 8.80% | 91.26% | 29.53 |
| 265 | MIA PaCa-2 | 2.89 | 2.74 | 65.08% | 99.87% | 16.73 |
| 266 | BT-483 | >30 | 48.25 | 20.40% | 75.40% | 54.08 |
| 267 | Caki-2 | >30 | 3.35 | 45.41% | 96.39% | 23.03 |
| 268 | Jurkat clone | 0.64 | 0.47 | 86.77% | 99.98% | 22.23 |
| | E6-1 | | | | | |
| 269 | SU-DHL-8 | 0.75 | 0.70 | 99.99% | 99.99% | 20.34 |
| 270 | SU-DHL-1 | 0.33 | 0.28 | 77.89% | 99.98% | 19.14 |
| 271 | HBL-1 | 0.71 | 1.21 | 91.83% | 99.95% | 22.57 |
| 272 | JVM-3 | 0.27 | 1.12 | 90.18% | 99.94% | 39.25 |
| 273 | KARPAS- | 0.78 | 1.11 | 99.97% | 99.99% | 23.05 |
| | 422 | | | | | |
| 274 | EOL-1 | 0.74 | 0.46 | 94.43% | 99.99% | 24.26 |
| 275 | SNU-398 | 0.22 | 4.90 | 82.17% | 99.89% | 23.75 |
| 276 | NCI-H226 | 2.08 | 3.56 | 71.67% | 98.81% | 29.35 |
| 277 | PANC-1 | 2.70 | 4.61 | 61.48% | 96.95% | 28.03 |
| 278 | KG-1 | 1.05 | 2.02 | 52.09% | 99.96% | 39.85 |
| 279 | DU4475 | 0.32 | 1.35 | 92.20% | 99.91% | 28.79 |
| 280 | AU565 | 10.06 | 1.48 | 84.39% | 99.76% | 34.59 |
| 281 | ZR-75-1 | 7.81 | 5.73 | 64.79% | 99.35% | 28.36 |
| 282 | HCC1954 | >30 | 7.18 | 50.77% | 99.66% | 24.91 |
| 283 | MX-1 | >30 | 2.39 | 53.03% | 99.09% | 40.54 |
| 284 | HCT-116 | 0.92 | 4.03 | 80.82% | 96.35% | 14.18 |
| 285 | Capan-1 | >30 | 0.68 | 25.58% | 86.88% | 41.68 |
| 286 | LS411N | 0.70 | 15.09 | 58.44% | 93.32% | 23.43 |
| 287 | ARH-77 | >30 | 1.54 | 50.36% | 99.88% | 42.64 |
| 288 | MCF7 | >30 | 11.82 | 44.04% | 93.33% | 38.17 |
| 289 | SNU-368 | >30 | 20.16 | 27.89% | 81.47% | 65.63 |
| 290 | NCI-H2052 | >30 | 5.77 | 27.53% | 98.81% | 37.65 |
| 291 | MDA-MB- | >30 | 22.98 | 28.22% | 87.60% | 32.37 |
| | 231 | | | | | |

Summary of Absolute IC50s & Maximal inhibition
in 29 cell lines for Batch13

| | | | | % inhibition at top | | |
|---|---|---|---|---|---|---|
| | | Absolute IC50(μM) | | conc. | | |
| Cell | | Cpd | | Cpd | | Double |
| No. | Cell lines | A197 | Cisplatin | A197 | Cisplatin | time |
| 292 | NCI-H596 | >30 | 2.06 | 16.24% | 99.54% | 65.40 |
| 293 | SK-MES-1 | 1.21 | 2.63 | 51.86% | 93.78% | 25.62 |
| 294 | Hutu 80 | 0.86 | 1.70 | 84.92% | 99.98% | 16.32 |
| 295 | JAR | 0.67 | 0.61 | 84.32% | 99.89% | 18.29 |
| 296 | HPAC | 0.46 | 14.34 | 64.63% | 89.59% | 24.77 |
| 297 | Hep G2 | >30 | 4.51 | 17.00% | 96.93% | 36.37 |
| 298 | SW480 | >30 | 1.82 | 36.27% | 97.10% | 31.91 |
| 299 | MsTo 211H | 0.55 | 2.50 | 71.87% | 99.74% | 24.80 |
| 300 | Calu-1 | 1.04 | 4.77 | 65.93% | 99.01% | 36.29 |
| 301 | Capan-2 | >30 | 21.91 | 38.18% | 91.05% | 38.27 |
| 302 | SNU-387 | >30 | 43.38 | 23.54% | 97.32% | 43.12 |
| 303 | CFPAC-1 | >30 | 4.13 | 23.41% | 94.78% | 26.56 |
| 304 | SW1990 | 18.07 | 0.85 | 59.20% | 99.59% | 30.79 |
| 305 | Calu-6 | >30 | 0.71 | 53.82% | 94.87% | 36.43 |
| 306 | NCI-H1688 | >30 | 13.84 | 29.72% | 99.33% | 70.22 |
| 307 | NCI-H1650 | >30 | 5.12 | 33.26% | 99.46% | 40.70 |
| 308 | NCI-H1563 | >30 | 16.95 | 34.40% | 99.48% | 42.17 |
| 309 | NCI-H2452 | >30 | 35.50 | 39.71% | 80.91% | 52.96 |
| 310 | Hep3B | >30 | 3.08 | 51.64% | 99.75% | 31.48 |
| 311 | PL45 | 1.19 | 1.76 | 37.82% | 92.67% | 28.60 |
| 312 | SW1271 | >30 | 17.14 | 29.18% | 85.04% | 36.09 |
| 313 | K-562 | 0.57 | 5.73 | 56.13% | 97.43% | 24.54 |
| 314 | Molt-4 | 0.62 | 0.70 | 80.73% | 99.97% | 28.76 |
| 315 | NAMALWA | 0.49 | 0.28 | 93.22% | 99.98% | 26.13 |
| | CSN/70 | | | | | |
| 316 | ML-2 | 0.46 | 1.21 | 92.80% | 99.99% | 29.50 |
| 317 | GRANTA- | 1.00 | 1.71 | 72.42% | 86.21% | 35.46 |
| | 519 | | | | | |
| 318 | DMS 79 | >30 | 0.81 | 7.16% | 89.21% | 72.40 |
| 319 | NALM-6 | 0.70 | 1.56 | 78.84% | 98.78% | 26.08 |
| 320 | NCI-H929 | 18.96 | 3.07 | 59.89% | 99.97% | 43.18 |

Summary of Absolute IC50s & Maximal inhibition
in 34 cell lines for Batch14

| | | | | % inhibition at top | | |
|---|---|---|---|---|---|---|
| | | Absolute IC50(μM) | | conc. | | |
| Cell | | Cpd | | Cpd | | Double |
| No. | Cell lines | A197 | Cisplatin | A197 | Cisplatin | time |
| 321 | LP-1 | 0.49 | 7.62 | 77.36% | 99.83% | 24.13 |
| 322 | RS4;11 | 0.69 | 0.33 | 97.56% | 99.98% | 27.58 |
| 323 | T47D | >30 | 5.25 | 47.67% | 97.51% | 31.26 |
| 324 | HEL 92.1.7 | 0.38 | 0.48 | 94.58% | 99.98% | 20.75 |
| 325 | SK-BR-3 | 29.59 | 0.60 | 51.69% | 98.66% | 33.54 |
| 326 | SK-N-SH | >30 | 0.36 | 38.87% | 99.95% | 28.81 |
| 327 | Z-138 | 1.50 | 1.02 | 99.77% | 99.98% | 23.76 |
| 328 | MM.1S | >30 | 1.02 | 47.83% | 99.97% | 30.81 |
| 329 | U266B1 | >30 | 4.53 | 44.58% | 99.79% | 27.98 |
| 330 | KMS-11 | 0.63 | 2.16 | 76.01% | 99.98% | 25.76 |
| 331 | HCC1569 | >30 | 5.78 | 27.44% | 88.36% | 51.02 |
| 332 | L-363 | 0.88 | 1.83 | 70.57% | 99.97% | 22.07 |
| 333 | SUP-B15 | 1.22 | 1.39 | 99.14% | 99.99% | 23.67 |
| 334 | HL-60 | 0.99 | 1.15 | 90.26% | 99.99% | 23.52 |
| 335 | NK-92MI | 6.58 | 1.20 | 69.67% | 99.93% | 41.72 |
| 336 | HT-1376 | >30 | 3.28 | 23.07% | 97.55% | 25.64 |
| 337 | HCC1428 | >30 | 34.86 | 47.55% | 93.38% | 53.10 |
| 338 | CADO-ES1 | 0.21 | 1.03 | 83.69% | 99.68% | 18.13 |
| 339 | MDA-MB- | >30 | 37.89 | 26.78% | 91.17% | 60.79 |
| | 415 | | | | | |
| 340 | NCI-H187 | 0.33 | 0.68 | 33.73% | 99.92% | 39.23 |
| 341 | OPM2 | 0.37 | 0.79 | 91.89% | 99.99% | 24.85 |
| 342 | SH-SY5Y | >30 | 0.27 | 19.45% | 99.93% | 37.04 |
| 343 | Reh | 0.31 | 0.43 | 99.48% | 99.99% | 18.21 |
| 344 | SW-13 | 0.35 | 1.38 | 76.12% | 99.97% | 25.94 |
| 345 | CAL-120 | >30 | 7.91 | 47.51% | 90.81% | 30.08 |
| 346 | HCC1937 | >30 | 5.07 | 50.29% | 95.00% | 42.35 |
| 347 | HuT 78 | 0.63 | 0.50 | 78.95% | 99.77% | 24.80 |
| 348 | TF-1 | >30 | 5.11 | 58.31% | 99.66% | 26.56 |
| 349 | MEG-01 | 1.56 | 1.90 | 58.47% | 97.96% | 30.77 |
| 350 | Saos-2 | 0.82 | 1.50 | 72.40% | 99.93% | 28.65 |
| 351 | MM.1R | 1.19 | 0.81 | 60.01% | 99.98% | 31.78 |
| 352 | CAL-51 | 1.68 | 1.64 | 73.20% | 98.25% | 16.42 |
| 353 | CCRF-CEM | 0.58 | 0.55 | 84.32% | 99.98% | 21.10 |
| 354 | L-82 | 3.78 | 1.16 | 65.45% | 99.97% | 19.80 |

Summary of Absolute IC50s & Maximal inhibition
in 45 cell lines for Batch15

| | | | | % inhibition at top | | |
|---|---|---|---|---|---|---|
| | | Absolute IC50(μM) | | conc. | | |
| Cell | | Cpd | | Cpd | | Double |
| No. | Cell lines | A197 | Cisplatin | A197 | Cisplatin | time |
| 355 | KYSE-270 | 4.78 | 2.15 | 99.93% | 99.97% | 22.37 |
| 356 | NCI-H1373 | 0.80 | 1.45 | 85.74% | 98.87% | 34.80 |
| 357 | RT112/84 | 12.53 | 0.93 | 79.64% | 99.97% | 16.81 |
| 358 | PEER | 1.87 | 2.04 | 83.38% | 99.97% | 33.49 |
| 359 | HUH-1 | >30 | 1.59 | 35.12% | 93.48% | 29.33 |
| 360 | HT-29 | 1.27 | 2.89 | 71.37% | 92.78% | 19.61 |
| 361 | TE-1 | 9.77 | 1.49 | 65.45% | 99.80% | 26.39 |
| 362 | OCI-AML3 | >30 | 2.34 | 13.18% | 99.95% | 26.31 |
| 363 | SJSA-1 | >30 | 3.29 | 34.92% | 95.54% | 27.45 |
| 364 | NCI-H2009 | 4.84 | 16.68 | 52.84% | 92.25% | 32.09 |
| 365 | NCI-H1435 | 0.39 | 14.78 | 61.83% | 90.42% | 43.09 |
| 366 | MDA-MB- | >30 | 4.86 | 60.43% | 99.13% | 27.38 |
| | 453 | | | | | |
| 367 | NCI-H1395 | 0.37 | 2.91 | 70.60% | 99.74% | 40.24 |
| 368 | KYSE-70 | 0.46 | 6.11 | 70.08% | 99.76% | 25.50 |
| 369 | LoVo | >30 | 8.27 | 34.23% | 90.04% | 33.49 |
| 370 | FTC-133 | 0.84 | 0.98 | 63.41% | 99.81% | 21.84 |
| 371 | Li-7 | 0.85 | 0.63 | 88.04% | 94.13% | 26.80 |
| 372 | NOZ | 0.71 | 1.19 | 70.47% | 96.48% | 16.47 |
| 373 | DLD-1 | 0.42 | 2.43 | 81.93% | 99.08% | 15.13 |
| 374 | SNU-81 | >30 | 3.70 | 34.93% | 98.15% | 24.89 |
| 375 | PLC/PRF/5 | 1.24 | 8.47 | 47.93% | 99.41% | 28.49 |
| 376 | SNU-449 | 24.26 | 3.52 | 57.68% | 97.66% | 21.65 |
| 377 | SNU-739 | 20.94 | 0.81 | 65.98% | 99.86% | 22.76 |
| 378 | HCC38 | >30 | 2.86 | 42.65% | 99.21% | 49.73 |
| 379 | SNU-C2A | 0.58 | 1.77 | 65.64% | 98.49% | 33.88 |
| 380 | HT-55 | >30 | 17.69 | 0 | 93.87% | 47.80 |
| 381 | T84 | >30 | 3.17 | 35.26% | 96.62% | 28.48 |
| 382 | LS 180 | >30 | 6.27 | 16.21% | 98.02% | 29.92 |
| 383 | RD-ES | >30 | 0.80 | 18.53% | 99.62% | 37.16 |
| 384 | HCT-8 | 0.77 | 9.04 | 71.47% | 97.31% | 17.32 |
| 385 | RKO | 23.59 | 3.08 | 58.89% | 99.92% | 18.28 |
| 386 | HCC1500 | >30 | 7.64 | 45.32% | 99.65% | 102.31 |
| 387 | HCCC-9810 | >30 | 2.96 | 26.03% | 99.26% | 28.30 |
| 388 | SF-763 | 0.76 | 7.56 | 72.50% | 98.11% | 18.03 |
| 389 | HCC2935 | >30 | 51.72 | 20.15% | 87.55% | 53.88 |
| 390 | 8505C | >30 | 2.01 | 22.16% | 98.36% | 40.26 |
| 391 | KPL-4 | 0.25 | 1.37 | 90.74% | 99.88% | 26.47 |
| 392 | OS-RC-2 | >30 | 3.50 | 27.86% | 97.02% | 27.54 |
| 393 | SU-DHL-4 | 1.07 | 0.34 | 99.99% | 99.99% | 20.04 |
| 394 | MHCC97-H | 3.25 | 7.89 | 60.59% | 83.30% | 32.54 |
| 395 | OCUG-1 | 1.25 | 16.30 | 82.41% | 91.85% | 23.08 |
| 396 | KYSE-150 | 0.75 | 1.79 | 99.94% | 97.06% | 20.17 |
| 397 | SUM159PT | 17.84 | 3.15 | 77.63% | 98.87% | 21.45 |
| 398 | HCT-15 | 0.30 | 3.26 | 87.71% | 99.33% | 17.84 |
| 399 | OZ | >30 | 14.99 | 16.27% | 90.35% | 37.51 |

Summary of Absolute IC50s & Maximal inhibition
in 7 cell lines for Batch16

| | | | | % inhibition at top | | |
|---|---|---|---|---|---|---|
| | | Absolute IC50(μM) | | conc. | | |
| Cell | | Cpd | | Cpd | | Double |
| No. | Cell lines | A197 | Cisplatin | A197 | Cisplatin | time |
| 400 | JHH-6 | >30 | 3.12 | 45.19% | 95.32% | 22.01 |
| 401 | HUH-6 | >30 | 6.33 | 11.18% | 99.43% | 35.05 |
| | CLONE5 | | | | | |
| 402 | D283 Med | >30 | 0.70 | 66.67% | 99.39% | 35.12 |
| 403 | KU812 | >30 | 1.95 | 27.53% | 99.20% | 38.13 |
| 404 | 8305C | >30 | 6.14 | 33.79% | 99.17% | 40.48 |
| 405 | 143B | >30 | 3.20 | 27.55% | 99.94% | 36.68 |
| 406 | LS 174T | >30 | 5.89 | 15.79% | 96.60% | 28.47 |
| 407 | LS 174T | >30 | 8.54 | 27.00% | 96.74% | 33.97 |

### Example 7: FAM210B Expression Regulates Induction of ATF4 by Compounds of the Disclosure

FAM210B, identified in Example 2 as a biomarker for both hematopoietic cancers and solid tumor cancers, was further investigated.

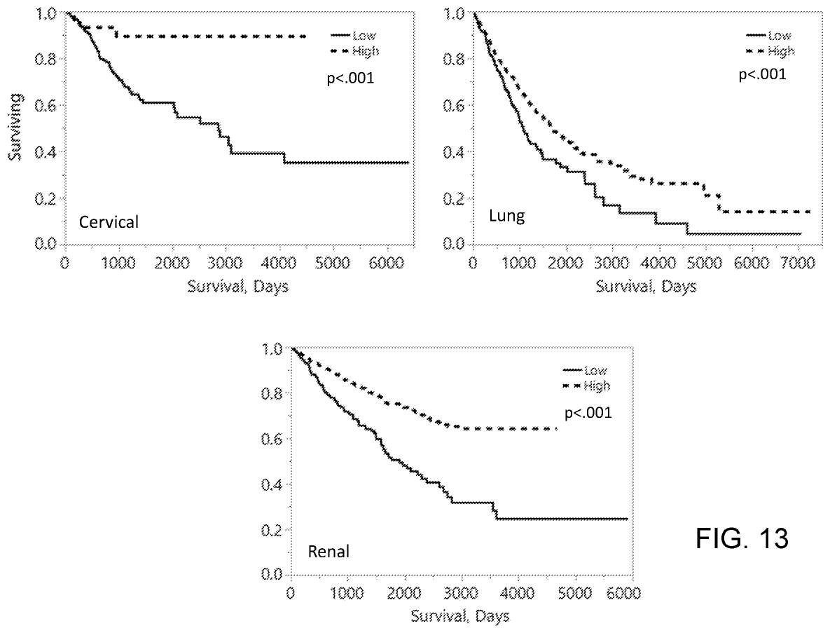

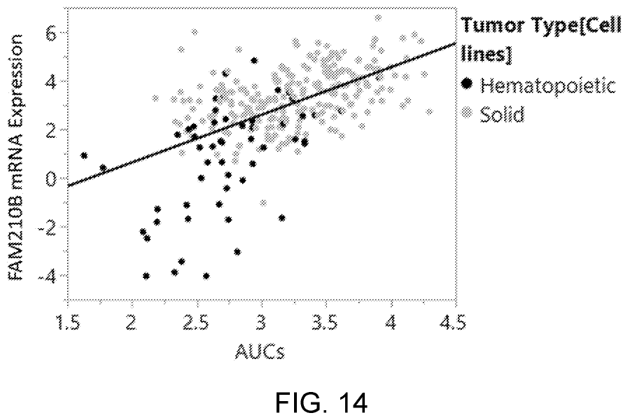

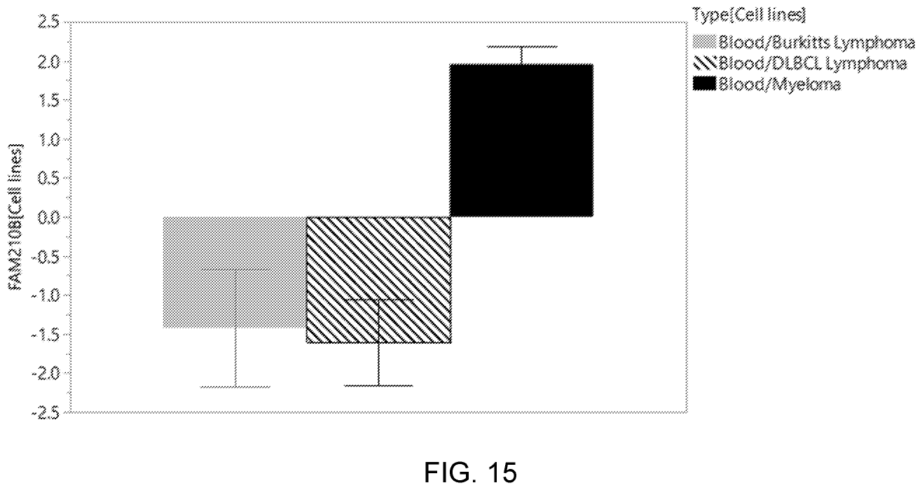

Analysis of data from Protein Atlas demonstrated that low levels of FAM210B protein are correlated with unfavorable therapeutic outcomes using conventional therapies in solid tumors (FIG. 13). And as shown in FIG. 14, FAM210B CCLE expression data for solid tumors correlates well with AUC (see, e.g., Example 2) (coefficient of regression /Spearman correlation R value=0.43 for solid tumors); FAM210B expression for hematopoietic tumors, however, does not correlate with AUC. Furthermore, cell line profiling demonstrated that expression of FAM210B is significantly lower in hematopoietic tumors relative to solid tumors (FIG. 15), with diffuse large B-cell lymphoma (DLBCL) and Burkitts lymphoma exhibiting the lowest levels of FAM210B expression.

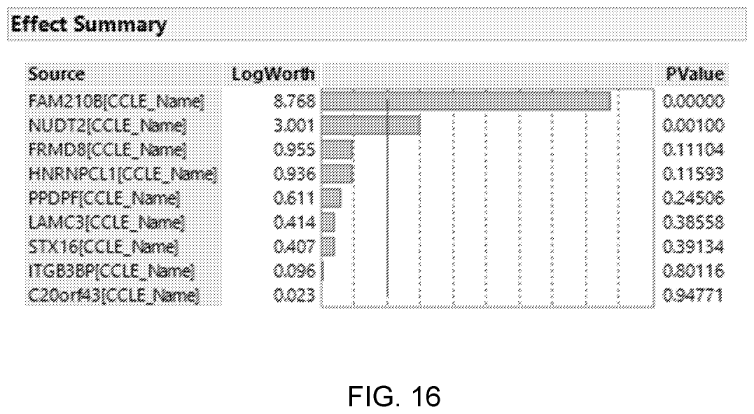

The present inventors have determined that FAM210B is a dominant marker for predicting response to compounds of the disclosure, such as compounds A197 and B19, in solid tumors. As shown in FIG. 16, two genes—FAM210B and NUDT2—account for the bulk of the partitioning that predicts response to compounds of the disclosure.

FAM210B Transfected Cells have Reduced Compound B5-Mediated ATF4 Induction

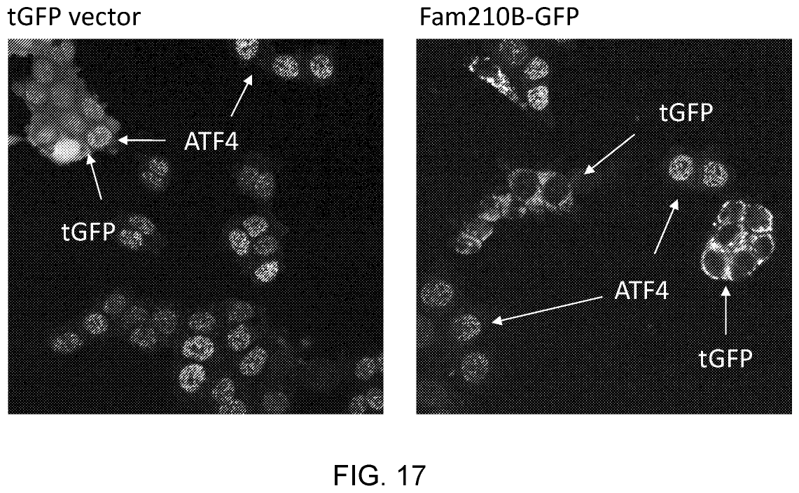

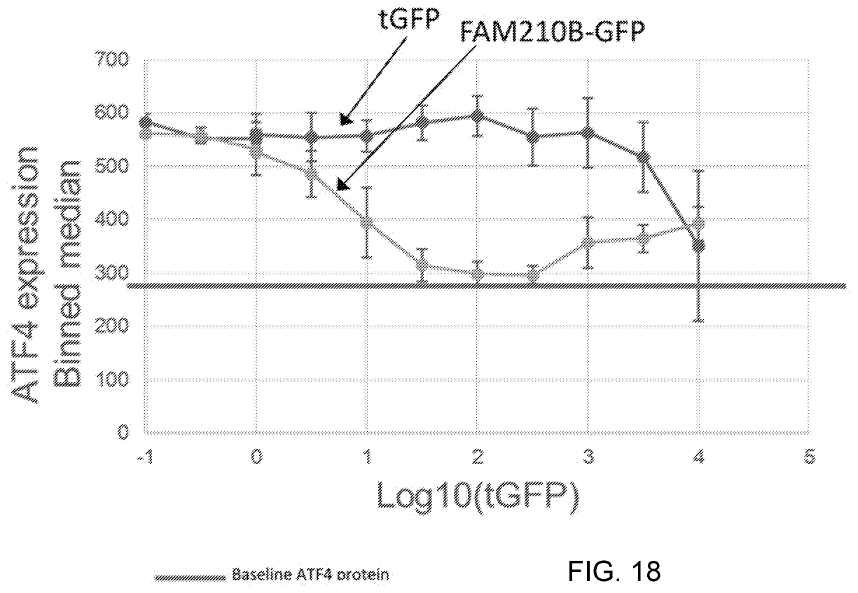

HCT-116 cells were transfected with a vector expressing tGFP or FAM210B-GFP. After 48 hours, the cells were treated with 3 mM Compound B5. Following 4 hrs of drug treatment the cells were fixed, permeabilized, and ATF4 levels determined by immunofluorescence (IF), and FAM210B levels were determined by detection of GFP. Cells were binned based on median ATF4 protein expression and ranked relative to level of FAM210B-tGFP. As shown in FIG. 17 and FIG. 18, ATF4 expression decreases in a dose-dependent manner with rising FAM210B expression. These results demonstrate that Compound B5 induction of ATF4 is inhibited by FAM210B expression in HCT-116 cells, and demonstrate a dose-dependent effect of FAM210B expression on Compound B5 mediated ATF4 gene induction.

Selectivity of effect of FAM210B

FAM210B expression has no effect on induction of ATF4 by tunicamycin, arsenite, or nutrient withdrawal (see FIG. 8). The lack of effect on alternate pathways of ATF4 induction suggests a lack of interactions with at least two pathways leading to eIF2α phosphorylation: PERK and GCN2. The lack of interactions with arsenite-mediated ATF4 expression suggests that Compound B5 does not function via activation of HRI.

Follow-up studies indicated that PERK inhibitors have no effect on Compound B5 mediated induction of ATF4, suggesting that ER stress plays no role in the activities of compounds of the disclosure.

The roles of the HRI and PRK pathways in induction of ATF4 can be confirmed using knockdown studies.

Overall, the data suggests that FAM210B expression uniquely regulates induction of ATF4 by the therapeutic compounds of the disclosure.

The disclosure further provides the following enumerated embodiments, which can be combined in any number and in any fashion not technically or logically inconsistent to form other embodiments of the disclosure,

Embodiment 1. A method for treating a cancer in a human individual, comprising:
determining the level of expression of a plurality of genes of the cancer;
determining a gene expression fold change as compared to the level of expression of the plurality of genes in a reference cell; and
if the gene expression fold change is significant with respect to a first number of the plurality of genes, administering an effective amount of a therapeutic compound of the disclosure to the human individual, the first number being five or more.
   Embodiment 2. The method of embodiment 1, wherein the cancer is a hematopoietic cancer.
   Embodiment 3. The method of embodiment 2, wherein the hematopoietic cancer is a chronic myeloproliferative neoplasm.
   Embodiment 4. The method of embodiment 2, wherein the hematopoietic cancer is a lymphoma.
   Embodiment 5. The method of embodiment 4, wherein the lymphoma is Burkitt's lymphoma, Hodgkin's lymphoma, non-Hodgkin's lymphoma, hairy cell lymphoma, mantle cell lymphoma, T-cell lymphoma, cutaneous T-cell lymphoma, B-cell lymphoma, diffuse large B-cell lymphoma, double-hit lymphoma, Waldenstrom macroglobulinemia, primary central nervous System (CNS) lymphoma, or intravascular large B-cell lymphoma (ILBCL) Embodiment 6. The method of embodiment 2, wherein the hematopoietic cancer is a leukemia.
   Embodiment 7. The method of embodiment 6, wherein the leukemia is acute lymphoblastic leukemia (ALL), acute myeloid leukemia (AML), acute myeloblastic leukemia, chronic lymphocytic leukemia (CLL), chronic myelogenous leukemia (CML), chronic neutrophilic leukemia (CNL), chronic myelomonocytic leukaemia (CMML), aggressive NK-cell leukemia (acute biphenotypic leukaemia, and polycythemia vera), or acute and chronic T-cell and B-cell leukemia Embodiment 8. The method of embodiment 2, wherein the hematopoietic cancer is a plasma cell neoplasm.
   Embodiment 9. The method of embodiment 8, wherein the plasma cell neoplasm is a multiple myeloma, a chronic myeloproliferative neoplasm, a myelodysplastic syndrome, a myelodysplastic/myeloproliferative neoplasms, or chronic myeloproliferative neoplasms Embodiment 10. The method of any of embodiments 2-9, wherein the reference cell is a non-cancerous cell of the human individual (e.g., of the same type as the hematopoietic cancer).
   Embodiment 11. The method of any of embodiments 2-9, wherein the reference cell is a non-cancerous cell from a different human (e.g., of the same type as the hematopoietic cancer).
   Embodiment 12. The method of any of embodiments 2-9, wherein the reference cell is a non-cancerous cell from a cell line (e.g., of the same type as the hematopoietic cancer).
   Embodiment 13. The method of any of embodiments 2-9, wherein the reference cell is a cell from a cell line having an IC₅₀ of at least 30 μM for the therapeutic compound (e.g., of the same type as the hematopoietic cancer).
   Embodiment 14. The method of any of embodiments 2-13, wherein a gene expression fold change of at least 1.5 is a significant change in gene expression.
   Embodiment 15. The method of any of embodiments 2-13, wherein a gene expression fold change of at least 2 is a significant change in gene expression.
   Embodiment 16. The method of any of embodiments 2-13, wherein a gene expression fold change of at least 3 is a significant change in gene expression.
   Embodiment 17. The method of any of embodiments 2-16, wherein the plurality of genes are selected from CASP10, TMED1, PPP1CC, TMEM59, BRD7, CYB561, FAM210B, NDRG1, CTSB, MMAB, SETDB2, VPS37B, ELL3, and KIF13B.
   Embodiment 18. The method of embodiment 17, wherein the first number is seven or more, e.g., eight or more, nine or more, or ten or more.
   Embodiment 19. The method of embodiment 17, wherein the first number is eleven or more, twelve or more, or thirteen or more.
   Embodiment 20. The method of any of embodiments 17-19, wherein at least one of the plurality of genes is CASP10 (e.g., wherein CASP10 is one of the first number of genes).
   Embodiment 21. The method of any of embodiments 17-20, wherein at least one of the plurality of genes is TMED1 (e.g., wherein TMED1 is one of the first number of genes).
   Embodiment 22. The method of any of embodiments 17-21, wherein at least one of the plurality of genes is PPP1CC (e.g., wherein PPP1CC is one of the first number of genes).
   Embodiment 23. The method of any of embodiments 17-22, wherein at least one of the plurality of genes is TMEM59 (e.g., wherein TMEM59 is one of the first number of genes).
   Embodiment 24. The method of any of embodiments 17-23, wherein at least one of the plurality of genes is BRD7 (e.g., wherein BRD7 is one of the first number of genes).
   Embodiment 25. The method of any of embodiments 17-24, wherein at least one of the plurality of genes is CYB561 (e.g., wherein CYB561 is one of the first number of genes).
   Embodiment 26. The method of any of embodiments 17-25, wherein at least one of the plurality of genes is FAM210B (e.g., wherein FAM210B is one of the first number of genes).
   Embodiment 27. The method of any of embodiments 17-26, wherein at least one of the plurality of genes is NDRG1 (e.g., wherein NDRG1 is one of the first number of genes).
   Embodiment 28. The method of any of embodiments 17-27, wherein at least one of the plurality of genes is CTSB (e.g., wherein CTSB is one of the first number of genes).
   Embodiment 29. The method of any of embodiments 17-28, wherein at least one of the plurality of genes is MMAB (e.g., wherein MMAB is one of the first number of genes).
   Embodiment 30. The method of any of embodiments 17-29, wherein at least one of the plurality of genes is SETDB2 (e.g., wherein SETDB2 is one of the first number of genes).
   Embodiment 31. The method of any of embodiments 17-30, wherein at least one of the plurality of genes is VPS37B (e.g., wherein VPS37B is one of the first number of genes).
   Embodiment 32. The method of any of embodiments 17-31, wherein at least one of the plurality of genes is ELL3 (e.g., wherein ELL3 is one of the first number of genes).
   Embodiment 33. The method of any of embodiments 17-32, wherein at least one of the plurality of genes is KIF13B (e.g., wherein KIF13B is one of the first number of genes).
   Embodiment 34. The method of embodiment 17, wherein the first number is fourteen.
   Embodiment 35. The method of embodiment 1, wherein the cancer is a solid tumor cancer.
   Embodiment 36. The method of embodiment 35, wherein the solid tumor cancer is adrenal gland(s) cancer, bile duct cancer, a bone or muscle cancer, cervical cancer, colorectal cancer, esophageal cancer, eye cancer, a head or neck cancer (e.g. a cancer of the nose, of the tongue, of the thyroid, or of a submaxillary gland), a kidney cancer, liver cancer, large intestine cancer, small cell lung cancer or non-small cell lung cancer, nervous system cancer, ovarian cancer, pancreatic cancer, placental cancer, prostate cancer, skin cancer, small intestine cancer, stomach/gastric cancer, or uterine cancer.
   Embodiment 37. The method of embodiment 35, wherein the solid tumor cancer is a soft tissue cancer.
   Embodiment 38. The method of any of embodiments 35-37, wherein the reference cell is a non-cancerous cell of the human individual (e.g., of the same type as the solid tumor cancer).
   Embodiment 39. The method of any of embodiments 35-38, wherein the reference cell is a non-cancerous cell of a different human (e.g., of the same type as the solid tumor cancer).
   Embodiment 40. The method of any of embodiments 35-38, wherein the reference cell is a non-cancerous cell from a cell line (e.g., of the same type as the solid tumor cancer).
   Embodiment 41. The method of any of embodiments 35-38, wherein the reference cell is a cell from a cancer cell line having an IC₅₀ of at least 30 μM for the therapeutic compound (e.g., of the same type as the solid tumor cancer).
   Embodiment 42. The method of any of embodiments 35-41, wherein a gene expression fold change of at least 1.5 is a significant change in gene expression.
   Embodiment 43. The method of any of embodiments 35-41, wherein a gene expression fold change of at least 2 is a significant change in gene expression.
   Embodiment 44. The method of any of embodiments 35-41, wherein a gene expression fold change of at least 3 is a significant change in gene expression.
   Embodiment 45. The method of any of embodiments 35-44, wherein the plurality of genes is selected from the group consisting of LAMC3, FAM210B, SENP8, ITGB3BP, NUDT2, HNRNPCL1, C20orf43, FRMD8, and STX16.
   Embodiment 46. The method of embodiment 45, wherein the first number is five or more, e.g., six or more.
   Embodiment 47. The method of embodiment 45, wherein the first number is seven or more, e.g., eight or more.
   Embodiment 48. The method of any of embodiments 45-47, wherein at least one of the plurality of genes is LAMC3 (e.g., wherein LAMC3 is one of the first number of genes).
   Embodiment 49. The method of any of embodiments 45-48, wherein at least one of the plurality of genes is FAM210B (e.g., wherein FAM210B is one of the first number of genes).
   Embodiment 50. The method of any of embodiments 45-49, wherein at least one of the plurality of genes is SENP8 (e.g., wherein SENP8 is one of the first number of genes).
   Embodiment 51. The method of any of embodiments 45-50, wherein at least one of the plurality of genes is ITGB3BP (e.g., wherein ITGB3BP is one of the first number of genes).
   Embodiment 52. The method of any of embodiments 45-51, wherein at least one of the plurality of genes is NUDT2 (e.g., wherein NUDT2 is one of the first number of genes).
   Embodiment 53. The method of any of embodiments 45-52, wherein at least one of the plurality of genes is HNRNPCL1 (e.g., wherein HNRNPCL1 is one of the first number of genes).
   Embodiment 54. The method of any of embodiments 45-53, wherein at least one of the plurality of genes is C20orf43 (e.g., wherein C20orf43 is one of the first number of genes).
   Embodiment 55. The method of any of embodiments 45-54, wherein at least one of the plurality of genes is FRMD8 (e.g., wherein FRMD8 is one of the first number of genes).
   Embodiment 56. The method of any of embodiments 45-55, wherein at least one of the plurality of genes is STX16 (e.g., wherein STX16 is one of the first number of genes).
   Embodiment 57. The method of embodiment 45, wherein first number is nine.
   Embodiment 58. A method for treating a hematopoietic cancer in a human individual, comprising
determining a gene copy number for KIAA0125 of the hematopoietic cancer; and
if the gene copy number is at least a second number, administering an effective amount of a therapeutic compound of the disclosure to the human individual, wherein the second number is at least 2.
   Embodiment 59. The method of embodiment 58, wherein the second number is at least 4.
   Embodiment 60. The method of embodiment 58 or embodiment 59, wherein the hematopoietic cancer is as described in any of embodiments 3-9.
   Embodiment 61. A method for treating a hematopoietic cancer in a human individual, comprising
determining a gene copy number for HLA-B and/or HLA-C of the hematopoietic cancer; and
if the gene copy number is no more than a third number, administering an effective amount of a therapeutic compound of the disclosure to the human individual, wherein the third number is no more than 0.40.
   Embodiment 62. The method of embodiment 61, wherein the third number is no more than 0.1, or no more than 0.07.
   Embodiment 63. The method of embodiment 61 or embodiment 62, wherein the hematopoietic cancer is as described in any of embodiments 3-9.
   Embodiment 64. A method for determining whether a cancer is responsive to a therapeutic compound of the disclosure, the method comprising:
determining the level of expression of a plurality of genes of the cancer;
determining a gene expression fold change as compared to the level of expression of the one or more genes in a reference cell; and
if the gene expression fold change is significant with respect to a first number of the plurality of genes, identifying the cancer as likely to be responsive to the therapeutic compound, wherein the first number is five or more.
   Embodiment 65. The method of embodiment 64, wherein the cancer is a hematopoietic cancer (e.g., as described with respect to any of embodiments 3-9), and wherein the method is performed as described in any of embodiments 10-34.
   Embodiment 66. The method of embodiment 64, wherein the cancer is a solid tumor cancer (e.g., as described with respect to any of embodiments 36 and 37) and wherein the method is performed as described in any of embodiments 38-57.
   Embodiment 67. A method for determining whether a hematopoietic cancer (e.g., as described in any of embodiments 3-9) is responsive to a therapeutic compound of the disclosure, the method comprising
determining a gene copy number for KIAA0125 of the hematopoietic cancer; and
if the gene copy number is at least a second number, identifying the cancer as likely to be responsive to the therapeutic compound, wherein the second number is at least 2.
   Embodiment 68. The method of embodiment 67, wherein the second number is at least 4.
   Embodiment 69. A method for determining whether a hematopoietic cancer (e.g., as described in any of embodiments 3-9) is responsive to a therapeutic compound of the disclosure, the method comprising
determining a gene copy number for HLA-B and/or HLA-C of the hematopoietic cancer; and
if the gene copy number is no more than a third number, identifying the cancer as likely to be responsive to the therapeutic compound, wherein the third number is no more than 0.10.
   Embodiment 70. The method of embodiment 69, wherein the third number is no more than 0.07.
   Embodiment 71. A method for treating a cancer in a human individual, the method comprising administering to the human individual an effective amount of a therapeutic compound of the disclosure.
   Embodiment 72. The method of embodiment 71, wherein the cancer is a hematopoietic cancer (e.g., as described with respect to any of embodiments 3-9) that exhibits a significant gene expression fold change as compared to a reference cell with respect to a first number of a plurality of genes selected from CASP10, TMED1, PPP1CC, TMEM59, BRD7, CYB561, FAM210B, NDRG1, CTSB, MMAB, SETDB2, VPS37B, ELL3, and KIF13B, wherein the first number is at least five.
   Embodiment 73. The method of embodiment 72, wherein the details of the determination of the gene expression fold changes are as described in any of embodiments 10-34.
   Embodiment 74. The method of embodiment 71, wherein the cancer is a solid tumor cancer (e.g., as described with respect to any of embodiments 36 and 37) that exhibits a significant gene expression fold change as compared to a reference cell with respect to a first number of a plurality of genes selected from LAMC3, FAM210B, SENP8, ITGB3BP, NUDT2, HNRNPCL1, C20orf43, FRMD8, and STX16, wherein the first number is at least five.
   Embodiment 75. The method of embodiment 74, wherein the details of the determination of the gene expression fold changes are as described in any of embodiments 38-57.
   Embodiment 76. The method of embodiment 71, wherein the cancer is a hematopoietic cancer than exhibits a gene copy number for HLA-B and/or HLA-C that is no more than 0.10 (e.g., no more than 0.07), e.g., wherein the hematopoietic cancer is as described in any of embodiments 3-9.
   Embodiment 77. The method of embodiment 71, wherein the cancer is a hematopoietic cancer that exhibits a gene copy number for KIAA0125 that is at least 2 (e.g., at least 4), e.g., wherein the hematopoietic cancer is as described in any of embodiments 3-9.
   Embodiment 78. A method for treating a solid tumor cancer in a human individual using a therapeutic compound of the disclosure, the method comprising:
determining the level of expression of FAM210B of the cancer;
determining a FAM210B expression fold change as compared to the level of expression of FAM210B in a reference cell; and
if the FAM210B expression fold change is significant, and if FAM210B expression in the cancer is lower than FAM210B expression in the reference cell, administering an effective amount of the therapeutic compound to the human individual.
   Embodiment 79. A method for treating a solid tumor cancer in a human individual, the method comprising administering to the human individual an effective amount of a therapeutic compound of the disclosure, the solid tumor cancer exhibiting a significant FAM210B expression fold change as compared to the level of expression of FAM210B in a reference cell, FAM210B expression in the cancer being lower than FAM210B expression in the reference cell.
   Embodiment 80. A method for determining whether a solid tumor cancer is responsive to a therapeutic compound of the disclosure, the method comprising:
determining the level of expression of FAM210B of the cancer;
determining a FAM210B expression fold change as compared to the level of expression of FAM210B in a reference cell; and
if the FAM210B expression fold change is significant, and if FAM210B expression in the cancer is lower than FAM210B expression in the reference cell, identifying the cancer as likely to be responsive to the therapeutic compound.
   Embodiment 80. The method of any of embodiments 78-80, wherein the solid tumor cancer is adrenal gland(s) cancer, bile duct cancer, a bone or muscle cancer, cervical cancer, colorectal cancer, esophageal cancer, eye cancer, a head or neck cancer (e.g. a cancer of the nose, of the tongue, of the thyroid, or of a submaxillary gland), a kidney cancer, liver cancer, large intestine cancer, small cell lung cancer or non-small cell lung cancer, nervous system cancer, ovarian cancer, pancreatic cancer, placental cancer, prostate cancer, skin cancer, small intestine cancer, stomach/gastric cancer, or uterine cancer.
   Embodiment 81. The method of any of embodiments 78-80, wherein the solid tumor cancer is a soft tissue cancer.
   Embodiment 82. The method of any of embodiments 78-81, wherein the reference cell is a non-cancerous cell of the human individual (e.g., of the same type as the solid tumor cancer).
   Embodiment 83. The method of any of embodiments 78-81, wherein the reference cell is a non-cancerous cell of a different human (e.g., of the same type as the solid tumor cancer).
   Embodiment 84. The method of any of embodiments 78-81, wherein the reference cell is a non-cancerous cell from a cell line (e.g., of the same type as the solid tumor cancer).
   Embodiment 85. The method of any of embodiments 78-81, wherein the reference cell is a cell from a cancer cell line having an IC₅₀ of at least 30 μM for the therapeutic compound (e.g., of the same type as the solid tumor cancer).
   Embodiment 86. The method of any of embodiments 78-85, wherein a gene expression fold change of at least 1.5 is a significant change in gene expression.
   Embodiment 87. The method of any of embodiments 78-85, wherein a gene expression fold change of at least 2 is a significant change in gene expression.
   Embodiment 88. The method of any of embodiments 78-85, wherein a gene expression fold change of at least 3 is a significant change in gene expression.
   Embodiment 89. The method of any of embodiments 1-88, wherein the therapeutic compound is a compound having the formula

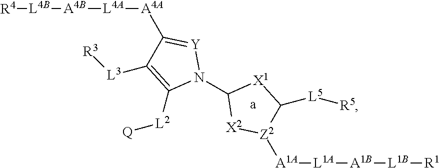

in which formula (I) the ring system denoted by “a” is defined as being heteroaromatic, optionally in the form of a pharmaceutically acceptable salt or N-oxide, or a solvate or hydrate, wherein
A^{1A} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
L^{1A} is selected from the group consisting of a bond, unsubstituted C₁-C₄ alkylene, unsubstituted C₁-C₄ alkenylene and unsubstituted C₁-C₄ alkynylene;
A^{1B} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
L^{1B} is selected from the group consisting of a bond, unsubstituted C₁-C₄ alkylene, unsubstituted C₁-C₄ alkenylene and unsubstituted C₁-C₄ alkynylene;
R¹ is selected from the group consisting of
   hydrogen,
   optionally substituted C₁-C₈ alkyl, optionally-substituted C₁-C₈ alkenyl and optionally substituted C₁-C₈ alkynyl,
   cycloalkyl and heterocycloalkyl, each optionally substituted with 1-5 R^{1E}, and aryl and heteroaryl, each optionally substituted with 1-5 R^{1E},
   in which
      each R^{1E} is independently selected from oxo, optionally-substituted C₁-C₄ alkyl, C₁-C₄ fluoroalkyl, halogen, —CN, SF₅, —N₃, —C(O)R^{1F}, —SR^{1F}, —S(O)₁₋₂R^{1F}, —OR^{1F}, —NR^{1G}R^{1F}, —C(O)R^{1F}, —C(O)NR^{1G}R^{1F}, —NR^{1G}C(O)R^{1F}, —C(S)NR^{1G}R^{1F}, —NR^{1G}C(S)R^{1F}, —C(O)OR^{1F}, —OC(O)R^{1F}, —C(O)SR^{1F}, —SC(O)R^{1F}, —C(S)OR^{1F}, —OC(S)R^{1F}, —C(S)SR^{1F}, —SC(S)R^{1F}, —S(O)₁₋₂OR^{1F}, —OS(O)₁₋₂R^{1F}, —S(O)₁₋₂NR^{1G}R^{1F}, —NR^{1G}S(O)₁₋₂R^{1F};
      each R^{1F} is independently selected from H, C₁-C₃ alkyl, C₁-C₃ fluoroalkyl, C₁-C₃ hydroxyalkyl and (C₁-C₃ alkoxy)C₁-C₃ alkyl, (C₁-C₃ alkoxy(C₁-C₃ alkoxy))C₁-C₃ alkyl, (C₁-C₃ alkoxy(C₁-C₃ alkoxy(C₁-C₃ alkoxy)))C₁-C₃ alkyl, and
      each R^{1G} is independently selected from H, C₁-C₃ alkyl, C₁-C₃ fluoroalkyl, C₁-C₃ hydroxyalkyl, (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl);
L² is selected from the group consisting of a bond, —CH₂—, —CH(CH₃)— or —CH₂CH₂—;
Q is selected from the group consisting of H, —CH₂OH, —C(O)OH, —C(O)OR^{2A}, —C(O)NR^{2B}R^{2A}, —C(O)NR^{2B}S(O)₂R^{2A}, —C(O)NR^{2B}S(O)₂NR^{2B}R^{2A}, —C(O)R^{2A}, —S(O)₂OH, —P(O)(OH)₂, —C(OH)(CF₃)₂, S(O)₂R^{2A}, —N(R^{2B})S(O)₂R^{2A}, —S(O)₂NR^{2B}R^{2A}, —C(O)NHOH, —C(O)NH—O(C₁-C₃ alkyl), —CO(NH)CN,

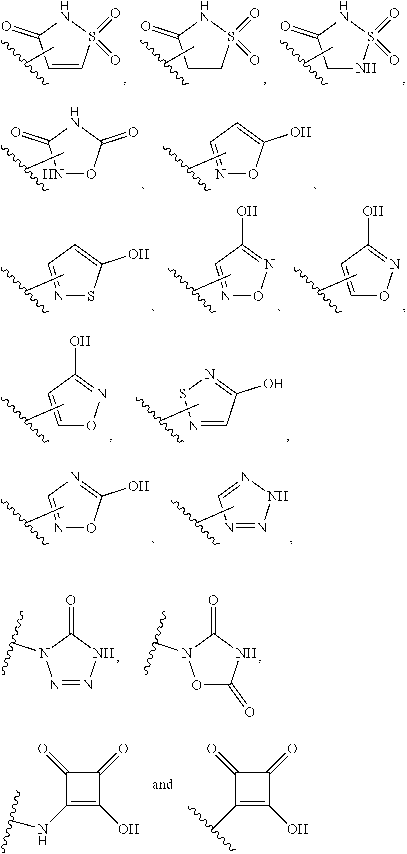

in which
   each R^{2A} is independently selected from H, C₁-C₃ alkyl, C₁-C₃ fluoroalkyl, C₁-C₃ hydroxyalkyl C₁-C₃ aminoalkyl, C₁-C₃ thioalkyl, (C₁-C₃ alkoxy)C₁-C₃ alkyl, —(CH₂CH₂O)₂₋₅-(optionally substituted C₁-C₃ alkyl)- and heteroaryl optionally substituted with 1-2 groups selected from substituents selected from C₁-C₃ alkyl, C₁-C₃ fluoroalkyl, hydroxyl, amino, thio, C₁-C₃ hydroxyalkyl, C₁-C₃ aminoalkyl, C₁-C₃ thioalkyl and —C(O)C₁-C₃ alkyl, and
   each R^{2B} is independently selected from H, C₁-C₃ alkyl, C₁-C₃ fluoroalkyl, C₁-C₃ hydroxyalkyl, C₁-C₃ aminoalkyl, C₁-C₃ thioalkyl, (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl),
   or R^{2A} and R^{2B} come together with a nitrogen to which they are both directly bound to form a heterocycloalkyl optionally substituted with 1-3 substituents selected from C₁-C₃ alkyl, C₁-C₃ fluoroalkyl, hydroxyl, amino, thio, C₁-C₃ hydroxyalkyl, C₁-C₃ aminoalkyl, C₁-C₃ thioalkyl and —C(O)C₁-C₃ alkyl;
L³ is a bond, —C(O)—, —S—, —S(O)₁₋₂—, —O—, —NR⁶—, —CH₂—, —CH(CH₃)(OH)—, —CH(OH)—, —CH₂CH₂—, —C(O)NR⁶—, —NR⁶C(O)—, —C(S)NR⁶—, —NR⁶C(S)—, —C(O)O—, —OC(O)—, —S(O)₁₋₂O—, —OS(O)₁₋₂—, —S(O)₁₋₂NR⁶— or —NR⁶S(O)₁₋₂—;
R³ is selected from the group consisting of
   cycloalkyl and heterocycloalkyl, each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E}, and
   aryl and heteroaryl each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E},
   in which
      each L^{3C} is a bond, methylene,
         ethylene, —C(O)—, —S—, —S(O)₁₋₂—, —O— or —NR^{3G}—;
      each R^{3D} is independently selected from optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F}, —OC(O)OR^{3F}, —OC(O)NR^{3G}R^{3F}, —NR^{3G}C(O)OR^{3F}, —NR^{3G}C(O)NR^{3G}R^{3F}, —SC(O)OR^{3F}, —OC(O)SR^{3F}, —SC(O)SR^{3F}, —SC(O)NR^{3G}R^{3F}, —NR^{3G}C(O)SR^{3F}, —OC(S)OR^{3F}, —OC(S)NR^{3G}R^{3F}, —NR^{3G}C(S)OR^{3F}, —NR^{3G}C(S)NR^{3G}R^{3F}, —SC(S)OR^{3F}, —OC(S)SR^{3F}, —SC(S)SR^{3F}, —SC(S)NR^{3G}R^{3F}, —NR^{3G}C(S)SR^{3F}, —NR^{3G}C(NR^{3G})NR^{3G}R^{3F} and —NR^{3G}S(O)₁₋₂NR^{3G}R^{3F};
      each R^{3E} is independently selected from oxo, optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F}, —OC(O)OR^{3F}, —OC(O)NR^{3G}R^{3F}, —NR^{3G}C(O)OR^{3F}, —NR^{3G}C(O)NR^{3G}R^{3F}, —SC(O)OR^{3F}, —OC(O)SR^{3F}, —SC(O)SR^{3F}, —SC(O)NR^{3G}R^{3F}, —NR^{3G}C(O)SR^{3F}, —OC(S)OR^{3F}, —OC(S)NR^{3G}R^{3F}, —NR^{3G}C(S)OR^{3F}, —NR^{3G}C(S)NR^{3G}R^{3F}, —SC(S)OR^{3F}, —OC(S)SR^{3F}, —SC(S)SR^{3F}, —SC(S)NR^{3G}R^{3F}, —NR^{3G}C(S)SR^{3F}, —NR^{3G}C(NR^{3G})NR^{3G}R^{3F} and —NR^{3G}S(O)₁₋₂NR^{3G}R^{3F};
      each R^{3F} is independently selected from H, C₁-C₃ alkyl, C₁-C₃ fluoroalkyl and C₁-C₃ hydroxyalkyl and
         each R^{3G} is independently selected from H, C₁-C₃ alkyl, C₁-C₃ fluoroalkyl, C₁-C₃ hydroxyalkyl, —S(O)₁₋₂(C₁-C₃ alkyl), —C(O)(C₁-C₃ alkyl) and —C(O)O(C₁-C₃ alkyl);
A^{4A} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
L^{4A} is selected from the group consisting of a bond, unsubstituted C₁-C₄ alkylene, unsubstituted C₁-C₄ alkenylene and unsubstituted C₁-C₄ alkynylene;
A^{4B} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
L^{4B} is selected from the group consisting of a bond, unsubstituted C₁-C₄ alkylene, unsubstituted C₁-C₄ alkenylene and unsubstituted C₁-C₄ alkynylene;
R⁴ is selected from the group consisting of hydrogen,
   optionally substituted C₁-C₈ alkyl, optionally-substituted C₁-C₈ alkenyl and
   optionally substituted C₁-C₈ alkynyl,
   cycloalkyl and heterocycloalkyl, each optionally substituted with 1-5 R^{4E}, and in which
      each R^{4E} is independently selected from oxo, optionally-substituted C₁-C₄ alkyl, C₁-C₄ fluoroalkyl, halogen, —CN, —SF₅, —N₃, —C(O)R^{4F}, —SR^{4F}, —S(O)₁₋₂R^{4F}, —OR^{4F}, —NR^{4G}R^{4F}, —C(O)R^{4F}, —C(O)NR^{4G}R^{4F}, —NR^{4G}C(O)R^{4F}, —C(S)NR^{4G}R^{4F}, —NR^{1G}C(S)R^{4F}, —C(O)OR^{4F}, —OC(O)R^{4F}, B—C(O)SR^{4F}, —SC(O)R^{4F}, —C(S)OR^{4F}, —OC(S)R^{4F}, —C(S)SR^{4F}, —SC(S)R^{4F}, —S(O)₁₋₂OR^{4F}, —OS(O)₁₋₂R^{4F}, —S(O)₁₋₂NR^{4G}R^{4F}, —NR^{4G}S(O)₁₋₂R^{4F}, —OC(O)OR^{4F}, —OC(O)NR^{4G}R^{4F}, —NR^{4G}C(O)OR^{4F}, —NR^{4G}C(O)NR^{4G}R^{4F}, —SC(O)OR^{4F}, —OC(O)SR^{4F}, —SC(O)SR^{4F}, —SC(O)NR^{4G}R^{4F}, —NR^{4G}C(O)SR^{4F}, —OC(S)OR^{4F}, —OC(S)NR^{4G}R^{4F}, —NR^{4G} C(S)OR^{4F}, —NR^{4G}C(S)NR^{4G}R^{4F}, —SC(S)OR^{4F}, —OC(S)SR^{4F}, —SC(S)SR^{4F}, —SC(S)NR^{4G}R^{4F}, —NR^{4G}C(S)SR^{4F}, —NR^{4G}C(NR^{4G})NR^{4G}R^{4F} and —NR^{4G}S(O)₁₋₂NR^{4G}R^{4F};
      each R^{4F} is independently selected from H, C₁-C₃ alkyl, C₁-C₃ fluoroalkyl, C₁-C₃ hydroxyalkyl and (C₁-C₃ alkoxy)C₁-C₃ alkyl and
      each R^{4G} is independently selected from H, C₁-C₃ alkyl, C₁-C₃ fluoroalkyl, C₁-C₃ hydroxyalkyl, and (C₁-C₃ alkoxy)C₁-C₃ alkyl, —S(O)₁₋₂(C₁-C₂ alkyl), —C(O)(C₁-C₃ alkyl) and —C(O)O(C₁-C₃ alkyl);
L⁵ is —C(O)—, —S—, —S(O)₁₋₂—, —O—, —NR⁶—, —CH₂—, —CH(CH₃)(OH)—, —CH(OH)—, —CH₂CH₂—, —C(O)NR⁶—, —NR⁶C(O)—, —C(S)NR⁶—, —NR⁶C(S)—, —C(O)O—, —OC(O)—, —S(O)₁₋₂O—, —OS(O)₁₋₂—, —S(O)₁₋₂NR⁶— or —NR⁶S(O)₁₋₂—;
R⁵ is selected from the group consisting of
   cycloalkyl and heterocycloalkyl, each optionally substituted with 1-5 R^{5E}, and aryl and heteroaryl each optionally substituted with 1-5 R^{5E}, in which
      each R^{5E} is independently selected from oxo, optionally-substituted C₁-C₄ alkyl, C₁-C₄ fluoroalkyl, halogen, —CN, —SF₅, —N₃, —C(O)R^{5F}, —SR^{5F}, —S(O)₁₋₂R^{5F}, —OR^{5F}, —NR^{5G}R^{5F}, —C(O)R^{5F}, —C(O)NR^{5G}R^{5F}, —NR^{5G}C(O)R^{5F}, —C(S)NR^{5G}R^{5F}, —NR^{1G}C(S)R^{5F}, —C(O)OR^{5F}, —OC(O)R^{5F}, —C(O)SR^{5F}, —SC(O)R^{5F}, —C(S)OR^{5F}, —OC(S)R^{5F}, —C(S)SR^{5F}, —SC(S)R^{5F}, —S(O)₁₋₂OR^{5F}, —OS(O)₁₋₂R^{5F}, —S(O)₁₋₂NR^{5G}R^{5F}, —NR^{5G}S(O)₁₋₂R^{5F}, —OC(O)OR^{5F}, —OC(O)NR^{5G}R^{5F}, —NR^{5G}C(O)OR^{5F}, —NR^{5G}C(O)NR^{5G}R^{5F}, —SC(O)OR^{5F}, —OC(O)SR^{5F}, —SC(O)SR^{5F}, —SC(O)NR^{5G}R^{5F}, —NR^{5G}C(O)SR^{5F}, —OC(S)OR^{5F}, —OC(S)NR^{5G}R^{5F}, —NR^{5G} C(S)OR^{5F}, —NR^{5G}C(S)NR^{5G}R^{5F}, —SC(S)OR^{5F}, —OC(S)SR^{5F}, —SC(S)SR^{5F}, —SC(S)NR^{5G}R^{5F}, —NR^{5G}C(S)SR^{5F}, —NR^{5G}C(NR^{5G})NR^{5G}R^{5F} and —NR^{5G}S(O)₁₋₂NR^{5G}R^{5F};
      each R^{5F} is independently selected from H, C₁-C₃ alkyl, C₁-C₃ fluoroalkyl, C₁-C₃ hydroxyalkyl and (C₁-C₃ alkoxy)C₁-C₃ alkyl and
      each R^{5G} is independently selected from H, C₁-C₃ alkyl, C₁-C₃ fluoroalkyl, C₁-C₃ hydroxyalkyl, and (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl);
X¹ is selected from the group consisting of CR^{XA}, S, O, NRX^{B} and N and
X² is selected from the group consisting of CR^{XA}, S, O, NRX^{B} and N in which
   each R^{XA} is independently selected from the group consisting of hydrogen, C₁-C₄ alkyl, C₁-C₄ fluoroalkyl, C₁-C₃ hydroxyalkyl, (C₁-C₃ alkoxy)C₁-C₃ alkyl, halo, —CN, oxo, —SF₅, —N₃, —C(O)R^{XC}, —SR^{XC}, —S(O)₁₋₂R^{XC}, —OR^{XC}, —NR^{XD}R^{XC}, in which each R^{XC} is independently selected from H, C₁-C₃ alkyl, C₁-C₃ fluoroalkyl, C₁-C₃ hydroxyalkyl and (C₁-C₃ alkoxy)C₁-C₃ alkyl and each R^{XD} is independently selected from H, C₁-C₃ alkyl, C₁-C₃ fluoroalkyl, C₁-C₃ hydroxyalkyl, and (C₁-C₃ alkoxy)C₁-C₃ alkyl, —C(O)(C₁-C₃ alkyl) and —C(O)O(C₁-C₃ alkyl);
   each R^{XB} is independently selected from the group consisting of H, C₁-C₃ alkyl, C₁-C₃ fluoroalkyl, C₁-C₃ hydroxyalkyl, (C₁-C₃ alkoxy)C₁-C₃ alkyl, —C(O)(C₁-C₃ alkyl) and —C(O)O(C₁-C₃ alkyl and C₁-C₄ alkyl-S(O)₁₋₂—;
Z¹ and Z² are independently selected from C and N;
provided that at least one of X¹, X², Z¹ and Z² is not C or CR^{XA}; and
Y is CR^{Y} or N, in which R^{Y} is selected from the group consisting of hydrogen, C₁-C₃ alkyl, C₁-C₃ fluoroalkyl, C₁-C₃ hydroxyalkyl, C₁-C₃ aminoalkyl, C₁-C₃ thioalkyl, (C₁-C₃ alkoxy)C₁-C₃ alkyl, —S(O)₁₋₂(C₁-C₃ alkyl), halogen, —CN, —SF₅, —N₃, —C(O)R^{YC}, —SR^{YC}, —S(O)₁₋₂R^{YC}, —OR^{YC} and —NR^{YD}R^{YC}, in which each R^{YC} is independently selected from H, C₁-C₃ alkyl and C₁-C₃ fluoroalkyl, and each R^{YD} is independently selected from H, C₁-C₃ alkyl and C₁-C₃ fluoroalkyl;
   wherein
each R⁶ is selected from the group consisting of hydrogen, C₁-C₃ alkyl, C₁-C₃ fluoroalkyl, C₁-C₃ hydroxyalkyl, C₁-C₃ aminoalkyl, C₁-C₃ thioalkyl, (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl);
each alkyl, alkylene, alkenyl, alkenylene, alkynyl and alkynylene is straight-chain or branched;
each optionally substituted alkyl, alkenyl, alkynyl, alkylene, alkenylene and alkynylene is unsubstituted or substituted with 1-5 substituents independently selected from oxo, halogen, —CN, —SF₅, —N₃, —C(O)R⁸, —SR⁸, —S(O)₁₋₂R⁸, —OR⁸, —NR⁹R⁸, —C(O)NR⁹R⁸, —NR⁹C(O)R⁸, —C(S)NR⁹R⁸, —NR⁹C(S)R⁸, —C(O)OR⁸, —OC(O)R⁸, —C(O)SR⁸, —SC(O)R⁸, —C(S)OR⁸, —OC(S)R⁸, —C(S)SR⁸, —SC(S)R⁸, —S(O)₁₋₂OR⁸, —OS(O)₁₋₂R⁸, —S(O)₁₋₂NR⁹R⁸, —NR⁹S(O)₁₋₂R⁸, —OC(O)OR⁸, —OC(O)NR⁹R⁸, —NR⁹C(O)OR⁸, —NR⁹C(O)NR⁹R⁸, —SC(O)OR⁸, —OC(O)SR⁸, SC(O)SR⁸, —SC(O)NR⁹R⁸, —NR⁹C(O)SR⁸, —OC(S)OR⁸, —OC(S)NR⁹R⁸, —NR⁹C(S)OR⁸, —NR⁹C(S)NR⁹R⁸, —SC(S)OR⁸, —OC(S)SR⁸, —SC(S)SR⁸, —SC(S)NR⁹R⁸, —NR⁹C(S)SR⁸, —NR⁹C(NR⁹)NR⁹R⁸ and —NR⁹S(O)₁₋₂NR⁹R⁸, in which
   each R⁸ is independently selected from H, C₁-C₃ alkyl, C₁-C₃ fluoroalkyl, C₁-C₃ hydroxyalkyl and (C₁-C₃ alkoxy)C₁-C₃ alkyl and
   each R⁹ is independently selected from H, C₁-C₃ alkyl, C₁-C₃ fluoroalkyl, C₁-C₃ hydroxyalkyl, and (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl);
each cycloalkyl has 3-10 ring carbons and is unsaturated or partially unsaturated, and optionally includes one or two fused cycloalkyl rings, each fused ring having 3-8 ring members;
each heterocylcloalkyl has 3-10 ring members and 1-3 heteroatoms independently selected from nitrogen, oxygen and sulfur and is unsaturated or partially unsaturated, and optionally includes one or two fused cycloalkyl rings, each having 3-8 ring members;
each aryl is a phenyl or a naphthyl, and optionally includes one or two fused cycloalkyl or heterocycloalkyl rings, each fused cycloalkyl or heterocycloalkyl ring having 4-8 ring members;
each heteroaryl is a 5-6 membered monocyclic heteroaryl ring having 1-4 heteroatoms independently selected from nitrogen, oxygen and sulfur or a 8-10 membered bicyclic heteroaryl having 1-5 heteroatoms independently selected from nitrogen, oxygen or sulfur, and optionally includes one or two fused cycloalkyl or heterocycloalkyl rings, each fused cycloalkyl or heterocycloalkyl ring having 4-8 ring members.
Embodiment 90. The method according to embodiment 88, wherein Y is N.
   Embodiment 91. The method according to embodiment 88, wherein the compound has the structural formula

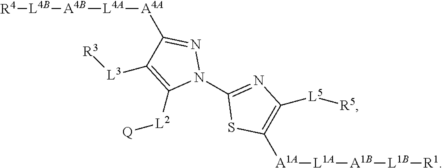

Embodiment 92. The method according to any of embodiments 88-91, wherein L is a bond.
Embodiment 93. The method according to any of embodiments 88-91, wherein L² is a bond, —CH₂—, —CH(CH₃)— or —CH₂CH₂—.
Embodiment 94. The method according to any of embodiments 89-93, wherein Q is —C(O)OH.
Embodiment 95. The method according to any of embodiments 89-93, wherein Q is selected from the group consisting of —C(O)OH, —CH₂OH, —C(O)OR^{2A}, —C(O)NR^{2B}R^{2A}, —C(O)NR^{2B}S(O)₂R^{2A}, —C(O)NR^{2B}S(O)₂NR^{2B}R^{2A}, —C(O)R^{2A}, —S(O)₂OH, —P(O)(OH)₂, —C(OH)(CF₃)₂, —S(O)₂R^{2A}, —N(R^{2B})S(O)₂R^{2A}, —S(O)₂NR^{2B}R^{2A}, —C(O)NH—O(C₁-C₃ alkyl), —CO(NH)CN,

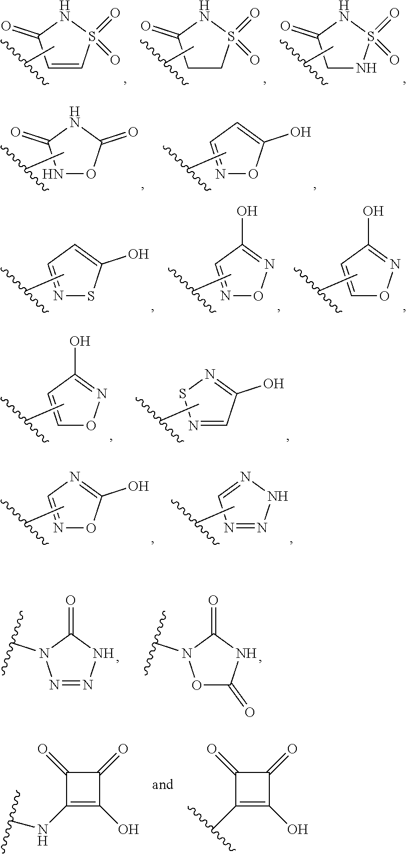

which
   each R^{2A} is independently selected from hydrogen, C₁-C₃ alkyl, C₁-C₃ fluoroalkyl, C₁-C₃ hydroxyalkyl C₁-C₃ aminoalkyl, C₁-C₃ thioalkyl, (C₁-C₃ alkoxy)C₁-C₃ alkyl and heteroaryl optionally substituted with 1-2 groups selected from substituents selected from C₁-C₃ alkyl, C₁-C₃ fluoroalkyl, hydroxyl, amino, thio, C₁-C₃ hydroxyalkyl, C₁-C₃ aminoalkyl, C₁-C₃ thioalkyl and —C(O)C₁-C₃ alkyl, and
      each R^{2B} is independently selected from hydrogen, C₁-C₃ alkyl, C₁-C₃ fluoroalkyl, C₁-C₃ hydroxyalkyl, C₁-C₃ aminoalkyl, C₁-C₃ thioalkyl, (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl),
      or R^{2A} and R^{2B} come together with a nitrogen to which they are both directly bound to form a heterocycloalkyl optionally substituted with 1-3 substituents selected from C₁-C₃ alkyl, C₁-C₃ fluoroalkyl, hydroxyl, amino, thio, C₁-C₃ hydroxyalkyl, C₁-C₃ aminoalkyl, C₁-C₃ thioalkyl and —C(O)C₁-C₃ alkyl.
         Embodiment 96. The method according to any of embodiments 89-93, wherein Q is —C(O)O(C₁-C₃ alkyl);
—C(O)NR^{2B}R^{2A}, in which R^{2A} is C₁-C₃ alkyl, C₁-C₃ hydroxyalkyl, C₁-C₃ aminoalkyl or C₁-C₃ thioalkyl and R^{2B} is H or C₁-C₃ alkyl;
—C(O)NR^{2B}R^{2A}, in which R^{2A} and R^{2B} come together with a nitrogen to which they are both directly bound to form a heterocycloalkyl optionally substituted with 1-3 substituents selected from C₁-C₃ alkyl, C₁-C₃ fluoroalkyl, hydroxyl, amino, thio, C₁-C₃ hydroxyalkyl, C₁-C₃ aminoalkyl, C₁-C₃ thioalkyl and —C(O)C₁-C₃ alkyl; or
—C(O)NR^{2B}R^{2A}, in which R^{2A} is —S(O)₁₋₂(C₁-C₃ alkyl), —S(O)₁₋₂(C₁-C₃ fluoroalkyl), or heteroaryl optionally substituted with 1-2 groups selected from substituents selected from C₁-C₃ alkyl, C₁-C₃ fluoroalkyl, hydroxyl, amino, thio, C₁-C₃ hydroxyalkyl, C₁-C₃ aminoalkyl, C₁-C₃ thioalkyl and —C(O)C₁-C₃ alkyl and R^{2B} is H or C₁-C₃ alkyl.
   Embodiment 97. The method according to any of embodiments 89-96, wherein R¹ is selected from the group consisting of hydrogen, optionally substituted C₁-C₈ alkyl, optionally-substituted C₁-C₈ alkenyl and optionally substituted C₁-C₈ alkynyl.
   Embodiment 98. The method according to any of embodiments 89-96, wherein R¹ is selected from the group consisting of unsubstituted C₁-C₈ alkyl, unsubstituted C₁-C₈ alkenyl and unsubstituted C₁-C₈ alkynyl, for example, methyl, ethyl, propyl, butenyl or butyl.
   Embodiment 99. The method according to embodiment 97 or embodiment 98, wherein A^{1A} and L^{1B} are each a bond.
   Embodiment 100. The method according to embodiment 99, wherein A^{1A}-L^{1A}-A^{1B}-L^{1b}- is —S—, —S(O)— or —S(O)₂—.
   Embodiment 101. The method according to any of embodiments 89-100, wherein L³ is a bond.
   Embodiment 102. The method according to any of embodiments 89-100, wherein L³ is optionally substituted C₁-C₄ alkylene, optionally substituted C₁-C₄ alkenylene or optionally substituted C₁-C₄ alkynylene.
   Embodiment 103. The method according to embodiment 102, wherein L³ is C₁-C₃ alkylene, optionally substituted with a hydroxyl.
   Embodiment 104. The method according to any of embodiments 89-100, wherein L³ is —C(O)—, —S—, —S(O)₁₋₂—, —O—, —NR⁶—, —CH₂—, —CH(CH₃)(OH)—, —CH(OH)— or —CH₂CH₂—.
   Embodiment 105. The method according to embodiment any of embodiments 89-104, wherein R³ is phenyl optionally substituted with 1-5 R^{3E}.
   Embodiment 106. The method according to any of embodiments 89-104, wherein R³ is phenyl, (i) substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E}.
   Embodiment 107. The method according to any of embodiments 89-104, wherein R³ is aryl or heteroaryl each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E}.
   Embodiment 108. The method according to embodiment 106 or embodiment 107, wherein the aryl is not substituted with any R^{3E}.
   Embodiment 109. The method according to any of embodiments 89-104, wherein R³ is heteroaryl (e.g., an isothiazole, a pyridone, a thiadiazole, a pyrazine, a pyrazolopyrimidine, a pyrazolopyridine, an imidazole, a benzofuran, an indole, an imidazopyridine, a pyridine, a pyrazole, an isoxazole, a triazolopyridine, a benzimidazole, a thiophene, a benzothiophene, a furan or a pyrimidine) optionally substituted with 1-5 R^{3E}.
   Embodiment 110. The method according to any of embodiments 89-104, wherein R³ is heteroaryl (e.g., an isothiazole, a pyridone, a thiadiazole, a pyrazine, a pyrazolopyrimidine, a pyrazolopyridine, an imidazole, a benzofuran, an indole, an imidazopyridine, a pyridine, a pyrazole, an isoxazole, a triazolopyridine, a benzimidazole, a thiophene, a benzothiophene, a furan or a pyrimidine) (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E}.
   Embodiment 111. The method according to any of embodiments 89-110, wherein R⁴ is selected from the group consisting of unsubstituted C₁-C₈ alkyl, unsubstituted C₁-C₈ alkenyl and unsubstituted C₁-C₈ alkynyl.
   Embodiment 112. The method according to any of embodiments 89-110, wherein R⁴ is selected from the group consisting of hydrogen, optionally substituted C₁-C₈ alkyl, optionally-substituted C₁-C₈ alkenyl and optionally substituted C₁-C₈ alkynyl.
   Embodiment 113. The method according to embodiment 111 or embodiment 112, wherein A^{4A}, L^{4B} and L^{4A} are each a bond.
   Embodiment 114. The method according to embodiment 113, wherein A^{4B} is —C(O)—, —S—, —S(O)₁₋₂—, —O—, —NR⁶—, —C(O)NR⁶—, —NR⁶C(O)—, —C(O)O— or —OC(O)—.
   Embodiment 115. The method according to embodiment 114, wherein A^{4B} is a bond.
   Embodiment 116. The method according to any of embodiments 89-115, wherein L⁵ is a bond.
   Embodiment 117. The method according to any of embodiments 89-115, wherein L⁵ is —C(O)—, —S—, —S(O)₁₋₂—, —O—, —NR⁶—, —CH═CH—, —C═C—, —CH₂— or —CH₂CH₂—.
   Embodiment 118. The method according to any of embodiments 89-117, wherein R⁵ is aryl (e.g., phenyl) (i) optionally substituted with a single substituent selected from -L^{5C}-(aryl optionally substituted with 1-5 R^{5D}), -L^{5C}-(heteroaryl optionally substituted with 1-5 R^{5D}), -L^{5C}-(cycloalkyl optionally substituted with 1-5 R^{5E}), -L^{5C}-(heterocycloalkyl optionally substituted with 1-5 R^{5E}) and (ii) optionally substituted with 1-5 R^{5E}.
   Embodiment 119. The method according to any of embodiments 89-117, wherein R⁵ is aryl (e.g., phenyl) optionally substituted with 1-5 R^{5E}.
   Embodiment 120. The method according to any of embodiments 89-117, wherein R⁵ is heteroaryl (e.g., an isoxazolyl, a pyridyl, an imidazopyridyl, a pyrazolyl, a benzoxazole, an indolyl, a pyrimidinyl) (i) optionally substituted with a single substituent selected from -L^{5C}-(aryl optionally substituted with 1-5 R^{5D}), -L^{5C}-(heteroaryl optionally substituted with 1-5 R^{5D}), -L^{5C}-(cycloalkyl optionally substituted with 1-5 R^{5E}), -L^{5C}-(heterocycloalkyl optionally substituted with 1-5 R^{5E}) and (ii) optionally substituted with 1-5 R^{5E}.
   Embodiment 121. The method according to embodiment 89, wherein the compound has the structural formula

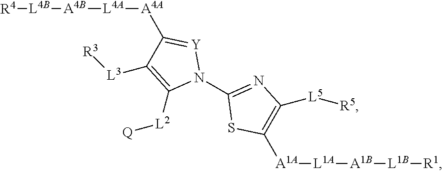

wherein
L² is selected from the group consisting of a bond, —CH₂—, —CH(CH₃)— or —CH₂CH₂—;
Q is selected from the group consisting of —C(O)OH, H, —CH₂OH, —C(O)OR^{2A}, —C(O)NR^{2B}R^{2A}, —C(O)NR^{2B}S(O)₂R^{2A}, —C(O)NR^{2B}S(O)₂NR^{2B}R^{2A}, —C(O)R^{2A}, —S(O)₂OH, —P(O)(OH)₂, —C(OH)(CF₃)₂, S(O)₂R^{2A}, —S(Q)₂NR^{2B}R^{2A}, —C(O)NHOH, —CO(NH)CN,

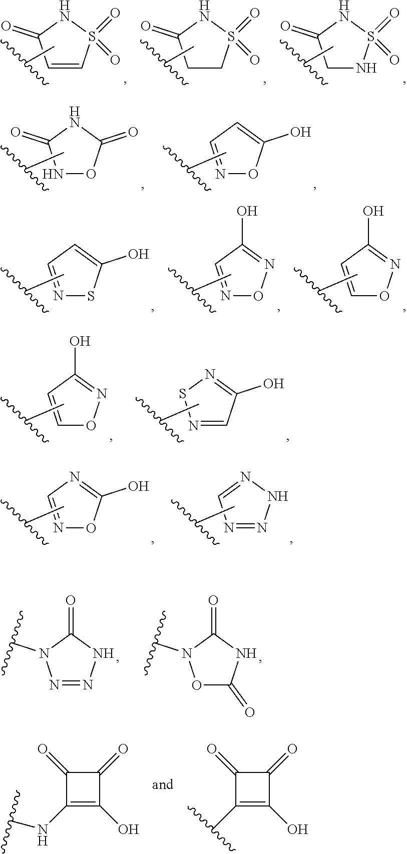

in which
   each R^{2A} is independently selected from H, C₁-C₃ alkyl, C₁-C₃ fluoroalkyl, C₁-C₃ hydroxyalkyl C₁-C₃ aminoalkyl, C₁-C₃ thioalkyl, (C₁-C₃ alkoxy)C₁-C₃ alkyl and heteroaryl optionally substituted with 1-2 groups selected from substituents selected from C₁-C₃ alkyl, C₁-C₃ fluoroalkyl, hydroxyl, amino, thio, C₁-C₃ hydroxyalkyl, C₁-C₃ aminoalkyl, C₁-C₃ thioalkyl and —C(O)C₁-C₃ alkyl, and
   each R^{2B} is independently selected from H, C₁-C₃ alkyl, C₁-C₃ fluoroalkyl, C₁-C₃ hydroxyalkyl, C₁-C₃ aminoalkyl, C₁-C₃ thioalkyl, (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl),
   or R^{2A} and R^{2B} come together with a nitrogen to which they are both directly bound to form a heterocycloalkyl optionally substituted with 1-3 substituents selected from C₁-C₃ alkyl, C₁-C₃ fluoroalkyl, hydroxyl, amino, thio, C₁-C₃ hydroxyalkyl, C₁-C₃ aminoalkyl, C₁-C₃ thioalkyl and —C(O)C₁-C₃ alkyl;
A^{1A} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
L^{1A} is selected from the group consisting of a bond, unsubstituted C₁-C₄ alkylene, unsubstituted C₁-C₄ alkenylene and unsubstituted C₁-C₄ alkynylene;
A^{1B} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
L^{1B} is selected from the group consisting of a bond, unsubstituted C₁-C₄ alkylene, unsubstituted C₁-C₄ alkenylene and unsubstituted C₁-C₄ alkynylene;
R¹ is selected from the group consisting of
   optionally substituted C₁-C₈ alkyl, optionally-substituted C₁-C₈ alkenyl and optionally substituted C₁-C₈ alkynyl,
   hydrogen, and
   cycloalkyl and heterocycloalkyl, each optionally substituted with 1-5 R^{1E}
   in which
      each R^{1E} is independently selected from oxo, optionally-substituted C₁-C₄ alkyl, C₁-C₄ fluoroalkyl, halogen, —CN, SF₅, —N₃, —C(O)R^{1F}, —SR^{1F}, —S(O)₁₋₂R^{1F}, —OR^{1F}, —NR^{1G}R^{1F}, —C(O)R^{1F}, —C(O)NR^{1G}R^{1F}, —NR^{1G}C(O)R^{1F}, —C(S)NR^{1G}R^{1F}, —NR^{1G}C(S)R^{1F}, —C(O)OR^{1F}, —OC(O)R^{1F}, —C(O)SR^{1F}, —SC(O)R^{1F}, —C(S)OR^{1F}, —OC(S)R^{1F}, —C(S)SR^{1F}, —SC(S)R^{1F}, —S(O)₁₋₂OR^{1F}, —OS(O)₁₋₂R^{1F}, —S(O)₁₋₂NR^{1G}R^{1F}, —NR^{1G}S(O)₁₋₂R^{1F};
      each R^{1F} is independently selected from H, C₁-C₃ alkyl, C₁-C₃ fluoroalkyl, C₁-C₃ hydroxyalkyl and (C₁-C₃ alkoxy)C₁-C₃ alkyl and
      each R^{1G} is independently selected from H, C₁-C₃ alkyl, C₁-C₃ fluoroalkyl, C₁-C₃ hydroxyalkyl, (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl);
L³ is a bond, —C(O)—, —S—, —S(O)₁₋₂—, —O—, —NR⁶—, —CH₂—, —CH(CH₃)(OH)—, —CH(OH)—, —CH₂CH₂—, —C(O)NR⁶—, —NR⁶C(O)—, —C(S)NR⁶—, —NR⁶C(S)—, —C(O)O—, —OC(O)—, —S(O)₁₋₂O—, —OS(O)₁₋₂—, —S(O)₁₋₂NR⁶— or —NR⁶S(O)₁₋₂—;
R³ is selected from the group consisting of
   aryl and heteroaryl each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E}, and
   cycloalkyl and heterocycloalkyl, each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E},
   in which
      each L^{3C} is a bond, methylene,
         ethylene, —C(O)—, —S—, —S(O)₁₋₂—, —O— or —NR^{3G}—;
      each R^{3D} is independently selected from optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F}, —OC(O)OR^{3F}, —OC(O)NR^{3G}R^{3F}, —NR^{3G}C(O)OR^{3F}, —NR^{3G}C(O)NR^{3G}R^{3F}, —SC(O)OR^{3F}, —OC(O)SR^{3F}, —SC(O)SR^{3F}, —SC(O)NR^{3G}R^{3F}, —NR^{3G}C(O)SR^{3F}, —OC(S)OR^{3F}, —OC(S)NR^{3G}R^{3F}, —NR^{3G}C(S)OR^{3F}, —NR^{3G}C(S)NR^{3G}R^{3F}, —SC(S)OR^{3F}, —OC(S)SR^{3F}, —SC(S)SR^{3F}, —SC(S)NR^{3G}R^{3F}, —NR^{3G}C(S)SR^{3F}, —NR^{3G}C(NR^{3G})NR^{3G}R^{3F} and —NR^{3G}S(O)₁₋₂NR^{3G}R^{3F};
      each R^{3E} is independently selected from oxo, optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F}, —OC(O)OR^{3F}, —OC(O)NR^{3G}R^{3F}, —NR^{3G}C(O)OR^{3F}, —NR^{3G}C(O)NR^{3G}R^{3F}, —SC(O)OR^{3F}, —OC(O)SR^{3F}, —SC(O)SR^{3F}, —SC(O)NR^{3G}R^{3F}, —NR^{3G}C(O)SR^{3F}, —OC(S)OR^{3F}, —OC(S)NR^{3G}R^{3F}, —NR^{3G}C(S)OR^{3F}, —NR^{3G}C(S)NR^{3G}R^{3F}, —SC(S)OR^{3F}, —OC(S)SR^{3F}, —SC(S)SR^{3F}, —SC(S)NR^{3G}R^{3F}, —NR^{3G}C(S)SR^{3F}, —NR^{3G}C(NR^{3G})NR^{3G}R^{3F} and —NR^{3G}S(O)₁₋₂NR^{3G}R^{3F};
      each R^{3F} is independently selected from H, C₁-C₃ alkyl, C₁-C₃ fluoroalkyl and C₁-C₃ hydroxyalkyl and
      each R^{3G} is independently selected from H, C₁-C₃ alkyl, C₁-C₃ fluoroalkyl, C₁-C₃ hydroxyalkyl, —S(O)₁₋₂(C₁-C₃ alkyl), —C(O)(C₁-C₃ alkyl) and —C(O)O(C₁-C₃ alkyl);
A^{4A} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
L^{4A} is selected from the group consisting of a bond, unsubstituted C₁-C₄ alkylene, unsubstituted C₁-C₄ alkenylene and unsubstituted C₁-C₄ alkynylene;
A^{4B} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
L^{4B} is selected from the group consisting of a bond, unsubstituted C₁-C₄ alkylene, unsubstituted C₁-C₄ alkenylene and unsubstituted C₁-C₄ alkynylene;
R⁴ is selected from the group consisting of optionally substituted C₁-C₈ alkyl, optionally-substituted C₁-C₈ alkenyl and optionally substituted C₁-C₈ alkynyl,
   hydrogen, and
   cycloalkyl and heterocycloalkyl, each optionally substituted with 1-5 R^{4E},
   in which
      each R^{4E} is independently selected from oxo, optionally-substituted C₁-C₄ alkyl, C₁-C₄ fluoroalkyl, halogen, —CN, —SF₅, —N₃, —C(O)R^{4F}, —SR^{4F}, —S(O)₁₋₂R^{4F}, —OR^{4F}, —NR^{4G}R^{4F}, —C(O)R^{4F}, —C(O)NR^{4G}R^{4F}, —NR^{4G}C(O)R^{4F}, —C(S)NR^{4G}R^{4F}, —NR^{1G}C(S)R^{4F}, —C(O)OR^{4F}, —OC(O)R^{4F}, —C(O)SR^{4F}, —SC(O)R^{4F}, —C(S)OR^{4F}, —OC(S)R^{4F}, —C(S)SR^{4F}, —SC(S)R^{4F}, —S(O)₁₋₂OR^{4F}, —OS(O)₁₋₂R^{4F}, —S(O)₁₋₂NR^{4G}R^{4F}, —NR^{4G}S(O)₁₋₂R^{4F}, —OC(O)OR^{4F}, —OC(O)NR^{4G}R^{4F}, —NR^{4G}C(O)OR^{4F}, —NR^{4G}C(O)NR^{4G}R^{4F}, —SC(O)OR^{4F}, —OC(O)SR^{4F}, —SC(O)SR^{4F}, —SC(O)NR^{4G}R^{4F}, —NR^{4G}C(O)SR^{4F}, —OC(S)OR^{4F}, —OC(S)NR^{4G}R^{4F}, —NR^{4G} C(S)OR^{4F}, —NR^{4G}C(S)NR^{4G}R^{4F}, —SC(S)OR^{4F}, —OC(S)SR^{4F}, —SC(S)SR^{4F}, —SC(S)NR^{4G}R^{4F}, —NR^{4G}C(S)SR^{4F}, —NR^{4G}C(NR^{4G})NR^{4G}R^{4F} and —NR^{4G}S(O)₁₋₂NR^{4G}R^{4F};
      each R^{4F} is independently selected from H, C₁-C₃ alkyl, C₁-C₃ fluoroalkyl, C₁-C₃ hydroxyalkyl and (C₁-C₃ alkoxy)C₁-C₃ alkyl and
      each R^{4G} is independently selected from H, C₁-C₃ alkyl, C₁-C₃ fluoroalkyl, C₁-C₃ hydroxyalkyl, and (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl);
L⁵ is a bond, —C(O)—, —S—, —S(O)₁₋₂—, —O—, —NR⁶—, —CH₂—, —CH(CH₃)(OH)—, —CH(OH)—, —CH₂CH₂—, —CH═CH—, —C═C—, —C(O)NR⁶—, —NR⁶C(O)—, —C(S)NR⁶—, —NR⁶C(S)—, —C(O)O—, —OC(O)—, —S(O)₁₋₂O—, —OS(O)₁₋₂—, —S(O)₁₋₂NR⁶— or —NR⁶S(O)₁₋₂—;
R⁵ is selected from the group consisting of
   aryl and heteroaryl each optionally substituted with 1-5 R^{5E}, and cycloalkyl and heterocycloalkyl, each optionally substituted with 1-5 R^{5E},
   in which
      each R^{5E} is independently selected from oxo, optionally-substituted C₁-C₄ alkyl, C₁-C₄ fluoroalkyl, halogen, —CN, —SF₅, —N₃, —C(O)R^{5F}, —SR^{5F}, —S(O)₁₋₂R^{5F}, —OR^{5F}, —NR^{5G}R^{5F}, —C(O)R^{5F}, —C(O)NR^{5G}R^{5F}, —NR^{5G}C(O)R^{5F}, —C(S)NR^{5G}R^{5F}, —NR^{1G}C(S)R^{5F}, —C(O)OR^{5F}, —OC(O)R^{5F}, —C(O)SR^{5F}, —SC(O)R^{5F}, —C(S)OR^{5F}, —OC(S)R^{5F}, —C(S)SR^{5F}, —SC(S)R^{5F}, —S(O)₁₋₂OR^{5F}, —OS(O)₁₋₂R^{5F}, —S(O)₁₋₂NR^{5G}R^{5F}, —NR^{5G}S(O)₁₋₂R^{5F}, —OC(O)OR^{5F}, —OC(O)NR^{5G}R^{5F}, —NR^{5G}C(O)OR^{5F}, —NR^{5G}C(O)NR^{5G}R^{5F}, —SC(O)OR^{5F}, —OC(O)SR^{5F}, —SC(O)SR^{5F}, —SC(O)NR^{5G}R^{5F}, —NR^{5G}C(O)SR^{5F}, —OC(S)OR^{5F}, —OC(S)NR^{5G}R^{5F}, —NR^{5G} C(S)OR^{5F}, —NR^{5G}C(S)NR^{5G}R^{5F}, —SC(S)OR^{5F}, —OC(S)SR^{5F}, —SC(S)SR^{5F}, —SC(S)NR^{5G}R^{5F}, —NR^{5G}C(S)SR^{5F}, —NR^{5G}C(NR^{5G})NR^{5G}R^{5F} and —NR^{5G}S(O)₁₋₂NR^{5G}R^{5F};
      each R^{5F} is independently selected from H, C₁-C₃ alkyl, C₁-C₃ fluoroalkyl, C₁-C₃ hydroxyalkyl and (C₁-C₃ alkoxy)C₁-C₃ alkyl and
      each R^{5G} is independently selected from H, C₁-C₃ alkyl, C₁-C₃ fluoroalkyl, C₁-C₃ hydroxyalkyl, and (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl);
Y is N or CR^{Y}, in which R^{Y} is selected from the group consisting of hydrogen, C₁-C₃ alkyl, C₁-C₃ fluoroalkyl, C₁-C₃ hydroxyalkyl, C₁-C₃ aminoalkyl, C₁-C₃ thioalkyl, (C₁-C₃ alkoxy)C₁-C₃ alkyl, —S(O)₁₋₂(C₁-C₃ alkyl), halogen, —CN, —SF₅, —N₃, —C(O)R^{YC}, —SR^{YC}, —S(O)₁₋₂R^{YC}, —OR^{YC} and —NR^{YD}R^{YC}, in which each R^{YC} is independently selected from H, C₁-C₃ alkyl and C₁-C₃ fluoroalkyl, and each R^{YD} is independently selected from H, C₁-C₃ alkyl and C₁-C₃ fluoroalkyl;
in which
   R⁶ is selected from the group consisting of hydrogen, C₁-C₃ alkyl, C₁-C₃ fluoroalkyl, C₁-C₃ hydroxyalkyl, C₁-C₃ aminoalkyl, C₁-C₃ thioalkyl, (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl);
   each alkyl, alkylene, alkenyl, alkenylene, alkynyl and alkynylene is straight-chain or branched;
   each optionally substituted alkyl, alkene, alkyne, alkylene, alkenylene and alkynylene is unsubstituted or substituted with 1-5 substituents independently selected from oxo, halogen, —CN, —SF₅, —N₃, —C(O)R⁸, —SR⁸, —S(O)₁₋₂R⁸, —OR⁸, —NR⁹R⁸, —C(O)NR⁹R⁸, —NR⁹C(O)R⁸, —C(S)NR⁹R⁸, —NR⁹C(S)R⁸, —C(O)OR⁸, —OC(O)R⁸, —C(O)SR⁸, —SC(O)R⁸, —C(S)OR⁸, —OC(S)R⁸, —C(S)SR⁸, —SC(S)R⁸, —S(O)₁₋₂OR⁸, —OS(O)₁₋₂R⁸, —S(O)₁₋₂NR⁹R⁸, —NR⁹S(O)₁₋₂R⁸, —OC(O)OR⁸, —OC(O)NR⁹R⁸, —NR⁹C(O)OR⁸, —NR⁹C(O)NR⁹R⁸, —SC(O)OR⁸, —OC(O)SR⁸, SC(O)SR⁸, —SC(O)NR⁹R⁸, —NR⁹C(O)SR⁸, —OC(S)OR⁸, —OC(S)NR⁹R⁸, —NR⁹C(S)OR⁸, —NR⁹C(S)NR⁹R⁸, —SC(S)OR⁸, —OC(S)SR⁸, —SC(S)SR⁸, —SC(S)NR⁹R⁸, —NR⁹C(S)SR⁸, —NR⁹C(NR⁹)NR⁹R⁸ and —NR⁹S(O)₁₋₂NR⁹R⁸, in which
      each R⁸ is independently selected from H, C₁-C₃ alkyl, C₁-C₃ fluoroalkyl, C₁-C₃ hydroxyalkyl and (C₁-C₃ alkoxy)C₁-C₃ alkyl and
      each R⁹ is independently selected from H, C₁-C₃ alkyl, C₁-C₃ fluoroalkyl, C₁-C₃ hydroxyalkyl, and (C₁-C₃ alkoxy)C₁-C₃ alkyl, —S(O)₁₋₂(C₁-C₃ alkyl), —C(O)(C₁-C₃ alkyl) and —C(O)O(C₁-C₃ alkyl);
   each cycloalkyl has 3-10 ring carbons and is unsaturated or partially unsaturated, and optionally includes one or two fused aryl or heteroaryl rings, each fused ring having 3-8 ring members;
   each heterocylcloalkyl has 3-10 ring members and 1-3 heteroatoms independently selected from nitrogen, oxygen and sulfur and is unsaturated or partially unsaturated, and optionally includes one or two fused aryl or heteroaryl rings, each fused aryl or heteroaryl ring having 3-8 ring members;
   each optionally substituted aryl is a phenyl or a naphthyl, and optionally includes one or two fused cycloalkyl or heterocycloalkyl rings, each fused cycloalkyl or heterocycloalkyl ring having 4-8 ring members;
   each optionally substituted heteroaryl is a 5-6 membered monocyclic heteroaryl ring having 1-4 heteroatoms independently selected from nitrogen, oxygen and sulfur or a 8-10 membered bicyclic heteroaryl having 1-5 heteroatoms independently selected from nitrogen, oxygen or sulfur, optionally includes one or two fused cycloalkyl or heterocycloalkyl rings, each fused cycloalkyl or heterocycloalkyl ring having 4-8 ring members.
      Embodiment 122. The method according to embodiment 121, wherein Y is N.
      Embodiment 123. The method according to embodiment 121 or embodiment 122, wherein
A^{1A} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
L^{1A} is selected from the group consisting of a bond, unsubstituted C₁-C₄ alkylene, unsubstituted C₁-C₄ alkenylene and unsubstituted C₁-C₄ alkynylene;
A^{1B} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂— and
L^{1B} is a bond.
   Embodiment 124. The method according to embodiment 123, wherein A^{1A}, L^{1A} and L^{1B} are a bond.
   Embodiment 125. The method according to embodiment 124, wherein A^{1B} is —S—, —S(O)— or —S(O)₂—.
   Embodiment 126. The method according to any of embodiments 121-125, wherein R¹ is optionally substituted C₁-C₈ alkyl, optionally-substituted C₁-C₈ alkenyl or optionally substituted C₁-C₈ alkynyl.
   Embodiment 127. The method according to any of embodiments 121-125, wherein R¹ is unsubstituted C₁-C₈ alkyl, unsubstituted C₁-C₈ alkenyl or unsubstituted C₁-C₈ alkynyl, for example, methyl, ethyl, propyl, butenyl or butyl.
   Embodiment 128. The method according to any of embodiments 121-127, wherein L² is a bond.
   Embodiment 129. The method according to any of embodiments 121-128, wherein Q is —C(O)OH.
   Embodiment 130. The method according to any of embodiments 121-128, wherein Q is —C(O)O(C₁-C₃ alkyl);
—C(O)NR^{2B}R^{2A}, in which R^{2A} is C₁-C₃ alkyl, C₁-C₃ hydroxyalkyl, C₁-C₃ aminoalkyl or C₁-C₃ thioalkyl and R^{2B} is H or C₁-C₃ alkyl;
—C(O)NR^{2B}R^{2A}, in which R^{2A} and R^{2B} come together with a nitrogen to which they are both directly bound to form a heterocycloalkyl optionally substituted with 1-3 substituents selected from C₁-C₃ alkyl, C₁-C₃ fluoroalkyl, hydroxyl, amino, thio, C₁-C₃ hydroxyalkyl, C₁-C₃ aminoalkyl, C₁-C₃ thioalkyl and —C(O)C₁-C₃ alkyl; or
—C(O)NR^{2B}R^{2A}, in which R^{2A} is —S(O)₁₋₂(C₁-C₃ alkyl), —S(O)₁₋₂(C₁-C₃ fluoroalkyl), or heteroaryl optionally substituted with 1-2 groups selected from substituents selected from C₁-C₃ alkyl, C₁-C₃ fluoroalkyl, hydroxyl, amino, thio, C₁-C₃ hydroxyalkyl, C₁-C₃ aminoalkyl, C₁-C₃ thioalkyl and —C(O)C₁-C₃ alkyl and R^{2B} is H or C₁-C₃ alkyl.
   Embodiment 131. The method according to any of embodiments 121-130, wherein L³ is a bond.
   Embodiment 132. The method according to any of embodiments 121-130, wherein L³ is —C(O)—, —S—, —S(O)₁₋₂—, —O—, —NR⁶—, —CH₂—, —CH(CH₃)(OH)— or —CH(OH)—.
   Embodiment 133. The method according to any of embodiments 121-132, wherein R³ is aryl (e.g., a phenyl) (i) substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E}.
   Embodiment 134. The method according to any of embodiments 121-132, wherein R³ is aryl (e.g., a phenyl) optionally substituted with 1-5 R^{3E}.
   Embodiment 135. The method according to any of embodiments 121-132, wherein R³ is heteroaryl (e.g., an isothiazole, a pyridone, a thiadiazole, a pyrazine, a pyrazolopyrimidine, a pyrazolopyridine, an imidazole, a benzofuran, an indole, an imidazopyridine, a pyridine, a pyrazole, an isoxazole, a triazolopyridine, a benzimidazole, a thiophene, a benzothiophene, a furan or a pyrimidine) (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E}.
   Embodiment 136. The method according to any of embodiments 121-135, wherein
A^{4A} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
L^{4A} is selected from the group consisting of a bond, unsubstituted C₁-C₄ alkylene, unsubstituted C₁-C₄ alkenylene and unsubstituted C₁-C₄ alkynylene;
A^{4B} is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂— and
L^{4B} is a bond.
   Embodiment 137. The method according to embodiment any of embodiments 121-135, wherein A^{4A}, L^{4A} and L^{4B} are a bond.
   Embodiment 138. The method according to embodiment 137, wherein A^{4B} is a bond.
   Embodiment 139. The method according to embodiment 138, wherein A^{4B} is —C(O)—, —S—, —S(O)₁₋₂—, —O—, —NR⁶—, —C(O)NR⁶—, —NR⁶C(O)—, —C(O)O—, —OC(O)—, —S(O)₁₋₂O—, —OS(O)₁₋₂—, —S(O)₁₋₂NR⁶— or —NR⁶S(O)₁₋₂—.
   Embodiment 140. The method according to any of embodiments 121-139, wherein R⁴ is optionally substituted C₁-C₈ alkyl, optionally-substituted C₁-C₈ alkenyl or optionally substituted C₁-C₈ alkynyl, for example, methyl, ethyl, propyl, butyl or pentyl.
   Embodiment 141. The method according to any of embodiments 121-140, wherein L⁵ is a bond.
   Embodiment 142. The method according to any of embodiments 121-141, wherein R⁵ is aryl (e.g., phenyl) optionally substituted with 1-5 R^{5E}.
   Embodiment 143. The method according to any of embodiments 121-141, wherein R⁵ is heteroaryl (e.g., an isoxazolyl, a pyridyl, an imidazopyridyl, a pyrazolyl) optionally substituted with 1-5 R^{5E}.
   Embodiment 144. The method according to embodiment 89, wherein the compound has the structural formula

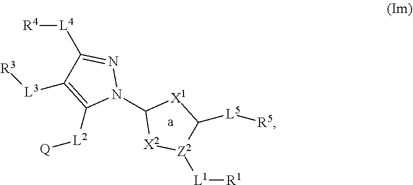

in which formula (Im) the ring system denoted by “a” is heteroaromatic,

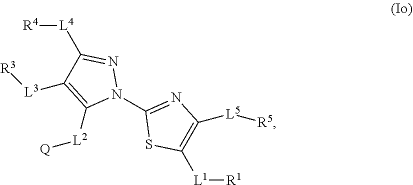

optionally in the form of a pharmaceutically acceptable salt or N-oxide, or a solvate or hydrate, wherein
L¹ is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
R¹ is selected from the group consisting of
   hydrogen,
   C₁-C₈ alkyl, C₁-C₈ alkenyl and C₁-C₈ alkynyl, each unsubstituted orfluorinated, cycloalkyl and heterocycloalkyl, each optionally substituted with 1-2 R^{1E}
   in which
      each R^{1E} is independently selected from oxo, optionally-substituted C₁-C₄ alkyl, C₁-C₄ fluoroalkyl, halogen, —CN, SF₅, —N₃, —C(O)R^{1F}, —SR^{1F}, —S(O)₁₋₂R^{1F}, —OR^{1F}, —NR^{1G}R^{1F} and —C(O)R^{1F};
      each R^{1F} is independently selected from H, C₁-C₃ alkyl and C₁-C₃ fluoroalkyl and
      each R^{1G} is independently selected from H and C₁-C₃ alkyl;
L² is selected from the group consisting of a bond, —CH₂—, —CH(CH₃)— or —CH₂CH₂—;
Q is selected from the group consisting of H, —CH₂OH, —C(O)OH, —C(O)OR^{2A}, —C(O)NR^{2B}R^{2A}, —C(O)NR^{2B}S(O)₂R^{2A}, —C(O)NR^{2B}S(O)₂NR^{2B}R^{2A}, —C(O)R^{2A}, —S(O)₂OH, —P(O)(OH)₂, —C(OH)(CF₃)₂, S(O)₂R^{2A}, —S(O)₂NR^{2B}R^{2A}, —C(O)NHOH and —CO(NH)CN,
   in which
      each R^{2A} is independently selected from H and C₁-C₃ alkyl, and
      each R^{2B} is independently selected from H and C₁-C₃ alkyl;
L³ is a bond, —C(O)—, —S—, —S(O)₁₋₂—, —O—, —NR⁶—, —CH₂—, —CH(CH₃)(OH)— or —CH(OH)—;
R³ is aryl or heteroaryl each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E},
   in which
      each L^{3C} is a bond, methylene,
         ethylene, —C(O)—, —S—, —S(O)₁₋₂—, —O— or —NR^{3G}—;
         each R^{3D} is independently selected from optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F} and —NR^{3G}S(O)₁₋₂R^{3F};
         each R^{3E} is independently selected from oxo, optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F};
         each R^{3F} is independently selected from H, C₁-C₃ alkyl and C₁-C₃ fluoroalkyl and
         each R^{3G} is independently selected from H and C₁-C₃ alkyl, C₁-C₃ fluoroalkyl;
L⁴ is is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
R⁴ is selected from the group consisting of hydrogen, optionally substituted C₁-C₈ alkyl, optionally-substituted C₁-C₈ alkenyl and optionally substituted C₁-C₈ alkynyl, L⁵ is a bond, —C(O)—, —S—, —S(O)₁₋₂—, —O—, —NR⁶—, —CH₂CH₂—, —CH═CH—, —C═C—, —CH₂—, —CH(CH₃)(OH)— or —CH(OH)—;
R⁵ is aryl or heteroaryl each optionally substituted with 1-5 R^{5E},
   in which
      each R^{5E} is independently selected from oxo, optionally-substituted C₁-C₄ alkyl, C₁-C₄ fluoroalkyl, halogen, —CN, —SF₅, —N₃, —C(O)R^{5F}, —SR^{5F}, —S(O)₁₋₂R^{5F}, —OR^{5F}, —NR^{5G}R^{5F}, —C(O)R^{5F}, —C(O)NR^{5G}R^{5F}, —NR^{5G}C(O)R^{5F}, —C(S)NR^{5G}R^{5F}, —NR^{1G}C(S)R^{5F}, —C(O)OR^{5F}, —OC(O)R^{5F}, —C(O)SR^{5F}, —SC(O)R^{5F}, —C(S)OR^{5F}, —OC(S)R^{5F}, —C(S)SR^{5F}, —SC(S)R^{5F}, —S(O)₁₋₂OR^{5F}, —OS(O)₁₋₂R^{5F}, —S(O)₁₋₂NR^{5G}R^{5F} and —NR^{5G}S(O)₁₋₂R^{5F};
      each R^{5F} is independently selected from H, C₁-C₃ alkyl and C₁-C₃ fluoroalkyl and
      each R^{5G} is independently selected from H and C₁-C₃ alkyl;
Y is CR^{Y} or N, in which R^{Y} is selected from the group consisting of hydrogen, C₁-C₃ alkyl and C₁-C₃ fluoroalkyl;
X¹ is selected from the group consisting of CR^{XA}, S, O, NRX^{B} and N and
X² is selected from the group consisting of CR_{XA}, S, O, NRX^{B} and N in which
   each R^{XA} is independently selected from the group consisting of hydrogen, C₁-C₄ alkyl and C₁-C₄ fluoroalkyl; and
   each R^{XB} is independently selected from the group consisting of hydrogen, C₁-C₄ alkyl and C₁-C₄ fluoroalkyl, C₁-C₄ alkyl-C(O)—, C₁-C₄ alkyl-S(O)₁₋₂—;
Z¹ and Z² are independently selected from C and N;
   wherein
each R⁶ is selected from the group consisting of hydrogen, C₁-C₃ alkyl and —C(O)(C₁-C₃ alkyl);
each optionally substituted alkyl, alkenyl and alkynyl is unsubstituted, fluorinated or substituted with one or two hydroxyl groups;
each cycloalkyl has 3-10 ring carbons and is unsaturated or partially unsaturated, and optionally includes one or two fused cycloalkyl rings, each fused ring having 3-8 ring members;
each heterocylcloalkyl has 3-10 ring members and 1-3 heteroatoms independently selected from nitrogen, oxygen and sulfur and is unsaturated or partially unsaturated, and optionally includes one or two fused cycloalkyl rings, each having 3-8 ring members;
each aryl is a phenyl or a naphthyl, and optionally includes one or two fused cycloalkyl or heterocycloalkyl rings, each fused cycloalkyl or heterocycloalkyl ring having 4-8 ring members;
each heteroaryl is a 5-6 membered monocyclic heteroaryl ring having 1-4 heteroatoms independently selected from nitrogen, oxygen and sulfur or a 8-10 membered bicyclic heteroaryl having 1-5 heteroatoms independently selected from nitrogen, oxygen or sulfur, and optionally includes one or two fused cycloalkyl or heterocycloalkyl rings, each fused cycloalkyl or heterocycloalkyl ring having 4-8 ring members.
Embodiment 145. The method according to embodiment 144, having the structural formula (Io).
   Embodiment 146. The method according to embodiment 144 or embodiment 145, wherein R¹ is selected from the group consisting of hydrogen, optionally substituted C₁-C₈ alkyl and cycloalkyl optionally substituted with 1-5 R^{1E}.
   Embodiment 147. The method according to any of embodiments 144-146, wherein R¹ is unsubstituted C₁-C₈ alkyl or fluorinated C₁-C₈ alkyl.
   Embodiment 148. The method according to any of embodiments 144-147, wherein L¹ is a bond, —O—, —S—, —S(O)— or —S(O)₂—.
   Embodiment 149. The method according to any of embodiments 144-147, wherein L¹ is or —S—.
   Embodiment 150. The method according to any of embodiments 144-149, wherein L² is a bond.
   Embodiment 151. The method according to any of embodiments 144-150, wherein Q is —C(O)OH.
   Embodiment 152. The method according to any of embodiments 144-151, wherein L³ is a bond.
   Embodiment 153. The method according to any of embodiments 144-151, wherein L³ is —C(O)—, —S—, —S(O)₁₋₂—, —O—, —NR⁶—, —CH₂—, —CH(CH₃)(OH)— or —CH(OH)—.
   Embodiment 154. The method according to any of embodiments 144-153, wherein R³ is aryl or heteroaryl each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3E}), -L^{3C}-(heterocycloalkyl optionally substituted with 1-5 R^{3E}) and (ii) optionally substituted with 1-5 R^{3E}.
   Embodiment 155. The method according to any of embodiments 144-153, wherein R³ is aryl (e.g., a phenyl) optionally substituted with 1-5 R^{3E}.
   Embodiment 156. The method according to any of embodiments 144-153, wherein R³ is heteroaryl (e.g., an isothiazole, a pyridone, a thiadiazole, a pyrazine, a pyrazolopyrimidine, a pyrazolopyridine, an imidazole, a benzofuran, an indole, an imidazopyridine, a pyridine, a pyrazole, an isoxazole, a triazolopyridine, a benzimidazole, a thiophene, a benzothiophene, a furan or a pyrimidine) optionally substituted with 1-5 R^{3E}.
   Embodiment 157. The method according to any of embodiments 144-156, wherein R⁴ is optionally substituted C₁-C₈ alkyl, optionally-substituted C₁-C₈ alkenyl or optionally substituted C₁-C₈ alkynyl.
   Embodiment 158. The method according to any of embodiments 144-157, wherein L⁴ is a bond.
   Embodiment 159. The method according to any of embodiments 144-158 wherein L⁵ is a bond.
   Embodiment 160. The method according to any of embodiments 144-159, wherein R⁵ is phenyl optionally substituted with 1-5 R^{5E}.
   Embodiment 161. The method according to any of embodiments 144-160, wherein R⁵ is heteroaryl (e.g., an isoxazolyl, a pyridyl, an imidazopyridyl, a pyrazolyl), each optionally substituted with 1-5 R^{5E}.
   Embodiment 162. The method according to embodiment 89, wherein the compound has the structural formula

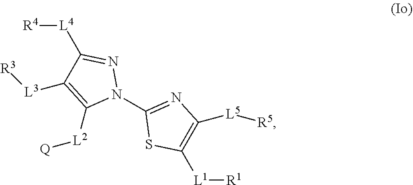

optionally in the form of a pharmaceutically acceptable salt or N-oxide, or a solvate or hydrate, wherein
L¹ is a —S—, —O—, —S(O)—, —S(O)₂— or a bond;
R¹ is unsubstituted or fluorinated C₁-C₈ alkyl, unsubstituted or fluorinated C₁-C₈ alkenyl and unsubstituted or fluorinated C₁-C₈ alkynyl
L² is a bond or —CH₂—;
Q is —COOH;
L³ is a bond, —C(O)—, —S—, —S(O)₁₋₂—, —O—, —NR⁶—, —CH₂—, —CH(CH₃)(OH)— or —CH(OH)—;
R³ is phenyl or monocyclic heteroaryl each (i) optionally substituted with a single substituent selected from -L^{3C}-(phenyl optionally substituted with 1-5 R^{3D}), -L^{3C}-(monocyclic heteroaryl optionally substituted with 1-5 R^{3D}), -L^{3C}-(monocyclic C3-C6 cycloalkyl optionally substituted with 1-5 R^{3E}), -L^{3C}-(monocyclic C4-C6 heterocycloalkyl optionally substituted with 1-5 R^{3E}) and (ii) optionally substituted with 1-5 R^{3E},
   in which
      each L^{3C} is a bond, methylene, ethylene, —C(O)—, —S—, —S(O)₁₋₂—, —O— or —NR^{3G}—;
      each R^{3D} is independently selected from optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F} and —NR^{3G}S(O)₁₋₂R^{3F};
      each R^{3E} is independently selected from oxo, optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F};
      each R^{3F} is independently selected from H, C₁-C₃ alkyl and C₁-C₃ fluoroalkyl and
      each R^{3G} is independently selected from H and C₁-C₃ alkyl, C₁-C₃ fluoroalkyl;
L⁴ is is selected from the group consisting of a bond, —C(O)—, —S—, —S(O)₁₋₂—, —O— and —NR⁶—;
R⁴ is selected from the group consisting of unsubstituted or fluorinated C₁-C₈ alkyl, unsubstituted or fluorinated C₁-C₈ alkenyl and unsubstituted or fluorinated C₁-C₈ alkynyl,
L⁵ is a bond, —C(O)—, —S—, —S(O)₁₋₂—, —O— or —NR⁶—;
R⁵ is phenyl or monocyclic heteroaryl each optionally substituted with 1-5 R^{5E},
   in which
      each R^{5E} is independently selected from oxo, optionally-substituted C₁-C₄ alkyl, C₁-C₄ fluoroalkyl, halogen, —CN, —SF₅, —N₃, —C(O)R^{5F}, —SR^{5F}, —S(O)₁₋₂R^{5F}, —OR^{5F}, —NR^{5G}R^{5F}, —C(O)R^{5F}, —C(O)NR^{5G}R^{5F}, —NR^{5G}C(O)R^{5F}, —C(S)NR^{5G}R^{5F}, —NR^{1G}C(S)R^{5F}, —C(O)OR^{5F}, —OC(O)R^{5F}, —C(O)SR^{5F}, —SC(O)R^{5F}, —C(S)OR^{5F}, —OC(S)R^{5F}, —C(S)SR^{5F}, —SC(S)R^{5F}, —S(O)₁₋₂OR^{5F}, —OS(O)₁₋₂R^{5F}, —S(O)₁₋₂NR^{5G}R^{5F} and —NR^{5G}S(O)₁₋₂R^{5F};
      each R^{5F} is independently selected from H, C₁-C₃ alkyl and C₁-C₃ fluoroalkyl and
      each R^{5G} is independently selected from H and C₁-C₃ alkyl;
         wherein
each R⁶ is selected from the group consisting of hydrogen, C₁-C₃ alkyl and —C(O)(C₁-C₃ alkyl);
each optionally substituted alkyl, alkenyl and alkynyl is unsubstituted, fluorinated or substituted with one or two hydroxyl groups;
each cycloalkyl has 3-10 ring carbons and is unsaturated or partially unsaturated, and optionally includes one or two fused cycloalkyl rings, each fused ring having 3-8 ring members;
each heterocylcloalkyl has 3-10 ring members and 1-3 heteroatoms independently selected from nitrogen, oxygen and sulfur and is unsaturated or partially unsaturated, and optionally includes one or two fused cycloalkyl rings, each having 3-8 ring members;
each aryl is a phenyl or a naphthyl, and optionally includes one or two fused cycloalkyl or heterocycloalkyl rings, each fused cycloalkyl or heterocycloalkyl ring having 4-8 ring members;
each heteroaryl is a 5-6 membered monocyclic heteroaryl ring having 1-4 heteroatoms independently selected from nitrogen, oxygen and sulfur or a 8-10 membered bicyclic heteroaryl having 1-5 heteroatoms independently selected from nitrogen, oxygen or sulfur, and optionally includes one or two fused cycloalkyl or heterocycloalkyl rings, each fused cycloalkyl or heterocycloalkyl ring having 4-8 ring members.
   Embodiment 163. The method according to embodiment 162, wherein
R³ is phenyl optionally substituted with 1-5 R^{3E}, in which
   each L^{3C} is a bond, methylene, ethylene, —C(O)—, —S—, —S(O)₁₋₂—, —O— or —NR^{3G}—;
   each R^{3D} is independently selected from optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F} and —NR^{3G}S(O)₁₋₂R^{3F};
   each R^{3E} is independently selected from oxo, optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F};
   each R^{3F} is independently selected from H, C₁-C₃ alkyl and C₁-C₃ fluoroalkyl and
   each R^{3G} is independently selected from H and C₁-C₃ alkyl, C₁-C₃ fluoroalkyl;
R⁵ is phenyl optionally substituted with 1-5 R^{5E}, in which
   each R^{5E} is independently selected from oxo, optionally-substituted C₁-C₄ alkyl, C₁-C₄ fluoroalkyl, halogen, —CN, —SF₅, —N₃, —C(O)R^{5F}, —SR^{5F}, —S(O)₁₋₂R^{5F}, —OR^{5F}, —NR^{5G}R^{5F}, —C(O)R^{5F}, —C(O)NR^{5G}R^{5F}, —NR^{5G}C(O)R^{5F}, —C(S)NR^{5G}R^{5F}, —NR^{1G}C(S)R^{5F}, —C(O)OR^{5F}, —OC(O)R^{5F}, —C(O)SR^{5F}, —SC(O)R^{5F}, —C(S)OR^{5F}, —OC(S)R^{5F}, —C(S)SR^{5F}, —SC(S)R^{5F}, —S(O)₁₋₂OR^{5F}, —OS(O)₁₋₂R^{5F}, —S(O)₁₋₂NR^{5G}R^{5F} and —NR^{5G}S(O)₁₋₂R^{5F};
   each R^{5F} is independently selected from H, C₁-C₃ alkyl and C₁-C₃ fluoroalkyl and
   each R^{5G} is independently selected from H and C₁-C₃ alkyl.
      Embodiment 164. The method according to embodiment 162 or embodiment 163, wherein
L¹ is —S—;
L² is a bond; and
L³ is a bond.
   Embodiment 165. The method according to any of embodiments 161-163, wherein
L⁴ is a bond; and
L⁵ is a bond.
   Embodiment 166. The method according to any applicable embodiment above, wherein R⁵ is trifluoromethylphenyl, halophenyl or dihalophenyl.
   Embodiment 167. The method according to any applicable embodiment above, wherein R⁵ is phenyl substituted (e.g., 3-substituted, 4-substituted, 3,4-disubstituted, 2,4-disubstituted, or 2,5-disubstituted) with one or two substituents selected from trifluoromethyl, fluorine and chlorine.
   Embodiment 168. The method according to any of embodiments 89-167, wherein each optionally substituted alkylene, alkenylene and alkynylene is unsubstituted.
   Embodiment 169. The method according to any of embodiments 89-168, wherein each optionally substituted alkyl, alkenyl and alkynyl is unsubstituted.
   Embodiment 170. The method according to any of embodiments 89-169, wherein each cycloalkyl is a 3-7 membered monocyclic cycloalkyl.
   Embodiment 171. The method according to any of embodiments 89-170, wherein each heterocycloalkyl is a 4-7 membered monocyclic heterocycloalkyl having 1-2 heteroatoms selected from O, S and N.
   Embodiment 172. The method according to any of embodiments 89-171, wherein each heteroaryl is a 5-6 membered monocyclic heteroaryl having 1-3 heteroatoms selected from O, S and N.
   Embodiment 173. The method according to any of embodiments 89-172, wherein each aryl is phenyl.
   Embodiment 174. The method according to any of embodiments 89-173, wherein each R^{XA} is hydrogen or C₁-C₄ alkyl.
   Embodiment 175. The method according to any of embodiments 89-173, wherein each R^{XA} is hydrogen.
   Embodiment 176. The method according to any of embodiments 89-175, wherein each R^{XB} is hydrogen or C₁-C₄ alkyl.
   Embodiment 177. The method according to any of embodiments 89-175, wherein each R^{XB} is hydrogen.
   Embodiment 178. The method according to any of embodiments 1-88, wherein the compound has any of structural formulae (IIa)-(IIe):

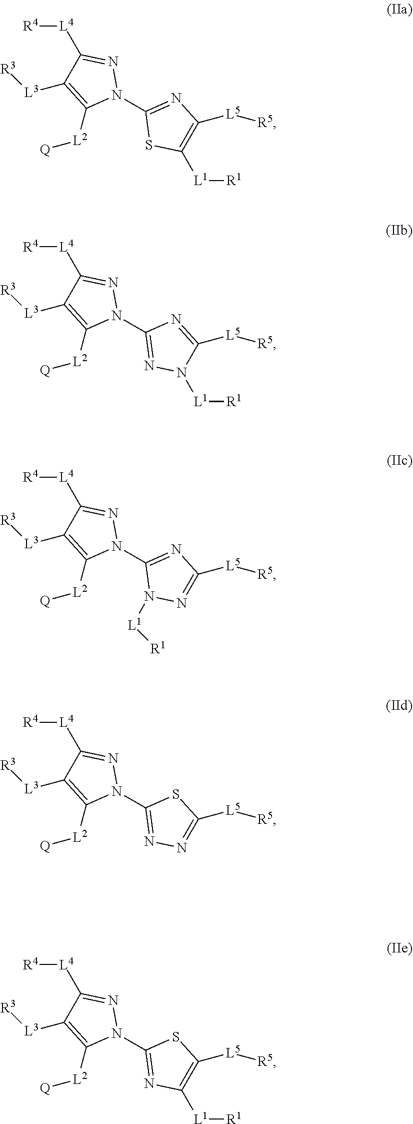

optionally in the form of a pharmaceutically acceptable salt or N-oxide, and/or a solvate or hydrate, wherein
L¹ is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
R¹ is selected from the group consisting of
   hydrogen,
   C₁-C₈ alkyl, C₁-C₈ alkenyl and C₁-C₈ alkynyl, each unsubstituted orfluorinated, cycloalkyl and heterocycloalkyl, each optionally substituted with 1-2 R^{1E}, and phenyl and monocyclic heteroaryl, each optionally substituted with 1-5 R^{1E},
   in which
      each R^{1E} is independently selected from oxo, optionally-substituted C₁-C₄ alkyl, C₁-C₄ fluoroalkyl, halogen, —CN, SF₅, —N₃, —C(O)R^{1F}, —SR^{1F}, —S(O)₁₋₂R^{1F}, —OR^{1F}, —(OCH₂CH₂O)ₙ—R^{1G} in which n is 1-4, —N(R^{1G})C(O)CH₂—O—(CH₂CH₂O)ₙR^{1G} in which n is 0-3, —C(O)NR^{1G}(CH₂CH₂O)ₙR^{1G}, —NR^{1G}R^{1F} and —C(O)R^{1F};
      each R^{1F} is independently selected from H, C₁-C₃ alkyl and C₁-C₃ fluoroalkyl and
      each R^{1G} is independently selected from H and C₁-C₃ alkyl;
L² is selected from the group consisting of a bond, —CH₂—, —CH(CH₃)— or —CH₂CH₂—;
Q is selected from the group consisting of H, —CH₂OH, —C(O)OH, —C(O)OR^{2A}, —C(O)NR^{2B}R^{2A}, —C(O)NR^{2B}S(O)₂R^{2A}, —C(O)NR^{2B}S(O)₂NR^{2B}R^{2A}, —C(O)R^{2A}, —S(O)₂OH, —P(O)(OH)₂, —C(OH)(CF₃)₂, S(O)₂R^{2A}, —N(R^{2B})S(O)₂R^{2A}, —S(O)₂NR^{2B}R^{2A}, —C(O)NHOH, —C(O)NH—O(C₁-C₃ alkyl), and —CO(NH)CN, in which
   each R^{2A} is independently selected from H and C₁-C₃ alkyl, and
   each R^{2B} is independently selected from H and C₁-C₃ alkyl;
L³ is a bond, —C(O)—, —S—, —S(O)₁₋₂—, —O—, —NR⁶—, —CH₂—, —CH(CH₃)(OH)— or —CH(OH)—;
R³ is aryl or heteroaryl each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3D}), -L^{3C}-(heterocycloalkyl optionally substituted with 1-5 R^{3D}) and (ii) optionally substituted with 1-5 R^{3E},
   in which
      each L^{3C} is a bond, methylene, ethylene, —C(O)—, —S—, —S(O)₁₋₂—, —O— or —NR^{3G}—;
      each R^{3D} is independently selected from oxo optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F} and —NR^{3G}S(O)₁₋₂R^{3F};
      each R^{3E} is independently selected from oxo, optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F};
      each R^{3F} is independently selected from H, C₁-C₃ alkyl and C₁-C₃ fluoroalkyl and
      each R^{3G} is independently selected from H and C₁-C₃ alkyl, C₁-C₃ fluoroalkyl;
L⁴ is is selected from the group consisting of a bond, —C(O)—, —S—, —S(O)₁₋₂—, —O—, —NR⁶—, —C(O)NR⁶—, —NR⁶C(O)—, —C(S)NR⁶—, —NR⁶C(S)—, —C(O)O—, —OC(O)—, —C(O)S—, —SC(O)—, —C(S)O—, —OC(S)—, —C(S)S—, —SC(S)—, —S(O)₁₋₂O—, —OS(O)₁₋₂—, —S(O)₁₋₂NR⁶— and —NR⁶S(O)₁₋₂—;
R⁴ is selected from the group consisting of hydrogen, optionally substituted C₁-C₈ alkyl, optionally-substituted C₁-C₈ alkenyl and optionally substituted C₁-C₈ alkynyl;
L⁵ is a bond, —C(O)—, —S—, —S(O)₁₋₂—, —O—, —NR⁶—, —CH₂CH₂—, —CH═CH—, —C═C—, —CH₂—, —CH(CH₃)(OH)— or —CH(OH)—; and
R⁵ is aryl, heteroaryl, cycloalkyl or heterocycloalkyl, each (i) optionally substituted with a single substituent selected from -L^{5C}-(phenyl optionally substituted with 1-5 R^{5D}), -L^{5C}-(monocyclic heteroaryl optionally substituted with 1-5 R^{5D}), and -L^{5C}-(monocyclic cycloalkyl optionally substituted with 1-5 R^{5D}), -L^{5C}-(monocyclic heterocycloalkyl optionally substituted with 1-5 R^{5D}) and (ii) optionally substituted with 1-5 R^{5E},
   in which
      each L^{5C} is a bond, methylene, ethylene, —C(O)—, —S—, —S(O)₁₋₂—, —O— or —NR^{3G}—;
      each R^{5D} is independently selected from oxo, optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F} and —NR^{3G}S(O)₁₋₂R^{3F};
      each R^{5E} is independently selected from oxo, optionally-substituted C₁-C₄ alkyl, C₁-C₄ fluoroalkyl, halogen, —CN, —SF₅, —N₃, —C(O)R^{5F}, —SR^{5F}, —S(O)₁₋₂R^{5F}, —OR^{5F}, —NR^{5G}R^{5F}, —C(O)R^{5F}, —C(O)NR^{5G}R^{5F}, —NR^{5G}C(O)R^{5F}, —C(S)NR^{5G}R^{5F}, —NR^{1G}C(S)R^{5F}, —C(O)OR^{5F}, —OC(O)R^{5F}, —C(O)SR^{5F}, —SC(O)R^{5F}, —C(S)OR^{5F}, —OC(S)R^{5F}, —C(S)SR^{5F}, —SC(S)R^{5F}, —S(O)₁₋₂OR^{5F}, —OS(O)₁₋₂R^{5F}, —S(O)₁₋₂NR^{5G}R^{5F} and —NR^{5G}S(O)₁₋₂R^{5F};
      each R^{5F} is independently selected from H, C₁-C₃ alkyl and C₁-C₃ fluoroalkyl and
      each R^{5G} is independently selected from H and C₁-C₃ alkyl;
         wherein
each R⁶ is selected from the group consisting of hydrogen, C₁-C₃ alkyl and —C(O)(C₁-C₃ alkyl);
each optionally substituted alkyl, alkenyl and alkynyl is unsubstituted, fluorinated or substituted with one or two hydroxyl groups;
each cycloalkyl has 3-10 ring carbons and is unsaturated or partially unsaturated, and optionally includes one or two fused cycloalkyl rings, each fused ring having 3-8 ring members;
each heterocylcloalkyl has 3-10 ring members and 1-3 heteroatoms independently selected from nitrogen, oxygen and sulfur and is unsaturated or partially unsaturated, and optionally includes one or two fused cycloalkyl rings, each having 3-8 ring members;
each aryl is a phenyl or a naphthyl, and optionally includes one or two fused cycloalkyl or heterocycloalkyl rings, each fused cycloalkyl or heterocycloalkyl ring having 4-8 ring members;
each heteroaryl is a 5-6 membered monocyclic heteroaryl ring having 1-4 heteroatoms independently selected from nitrogen, oxygen and sulfur or a 8-10 membered bicyclic heteroaryl having 1-5 heteroatoms independently selected from nitrogen, oxygen or sulfur, and optionally includes one or two fused cycloalkyl or heterocycloalkyl rings, each fused cycloalkyl or heterocycloalkyl ring having 4-8 ring members.
   Embodiment 179. The method according to embodiment 178, wherein the compound has the structural formula (IIa).
   Embodiment 180. The method according to embodiment 178, wherein the compound has the structural formula (IIb).
   Embodiment 181. The method according to embodiment 178, wherein the compound has the structural formula (IIe).
   Embodiment 182. The method according to embodiment 178, wherein the compound has the structural formula (IId).
   Embodiment 183. The method according to embodiment 178, wherein the compound has the structural formula (IIe).
   Embodiment 184. The method according to any of embodiments 178-183, wherein R¹ is selected from the group consisting of hydrogen, optionally substituted C₁-C₈ alkyl and cycloalkyl optionally substituted with 1-5 R^{1E}.
   Embodiment 185. The method according to any of embodiments 178-183, wherein R¹ is hydrogen.
   Embodiment 186. The method according to any of embodiments 178-183, wherein R¹ is optionally substituted C₁-C₈ alkyl.
   Embodiment 187. The method according to any of embodiments 178-183, wherein R¹ is unsubstituted C₁-C₈ alkyl or fluorinated C₁-C₈ alkyl.
   Embodiment 188. The method according to any of embodiments 178-183, wherein R¹ is unsubstituted cycloalkyl.
   Embodiment 189. The method according to any of embodiments 178-183, wherein R¹ is optionally substituted C₁-C₈ alkenyl, e.g. butenyl.
   Embodiment 190. The method according to any of embodiments 178-183, wherein R¹ is phenyl optionally substituted with 1-5 R^{E}.
   Embodiment 191. The method according to any of embodiments 178-183, wherein R¹ is trifluoromethyl-substituted phenyl, methoxy-substituted phenyl or fluoro-substituted phenyl.
   Embodiment 192. The method according to any of embodiments 178-183, wherein R¹ is phenyl substituted with —(OCH₂CH₂O)ₙ—R^{1G} in which n is 1-4, —N(R^{1G})C(O)CH₂—O—(CH₂CH₂O)ₙR^{1G} in which n is 0-3, or —C(O)NR^{1G}(CH₂CH₂O)ₙR^{1G} Embodiment 193. The method according to any of embodiments 178-183, wherein R¹ is hydroxymethyl, methoxymethyl, hydroxyethyl or methoxyethyl.
   Embodiment 194. The method according to any of embodiments 178-193, wherein L¹ is a bond, —O—, —S—, —S(O)— or —S(O)₂—.
   Embodiment 195. The method according to any of embodiments 178-193, wherein L¹ is —S—.
   Embodiment 196. The method according to any of embodiments 178-193, wherein L¹ is a bond.
   Embodiment 197. The method according to any of embodiments 178-193, wherein L¹ is is —NR⁶—.
   Embodiment 198. The method according to any of embodiments 178-197, wherein L² is a bond.
   Embodiment 199. The method according to any of embodiments 178-197, wherein L² is —CH₂—, —CH(CH₃)— or —CH₂CH₂—.
   Embodiment 200. The method according to any of embodiments 178-197, wherein L² is a bond or —CH₂—.
   Embodiment 201. The method according to any of embodiments 178-197, wherein Q is —C(O)OH.
   Embodiment 202. The method according to any of embodiments 178-197, wherein Q is selected from the group consisting of —CH₂OH, —C(O)OH, —C(O)OR^{2A}, —C(O)NR^{2B}R^{2A}, —C(O)NR^{2B}S(O)₂R^{2A}, —C(O)NR^{2B}S(O)₂NR^{2B}R^{2A}, —C(O)R^{2A}, —S(O)₂OH, —P(O)(OH)₂.
   Embodiment 203. The method according to any of embodiments 178-197, wherein Q is —CH₂OH, —C(O)OH or —C(O)OR^{2A}.
   Embodiment 204. The method according to any of embodiments 178-203, wherein L³ is a bond.
   Embodiment 205. The method according to any of embodiments 178-203, wherein L³ is —C(O)—, —S—, —S(O)₁₋₂—, —O—, —NR⁶—, —CH₂—, —CH(CH₃)(OH)— or —CH(OH)—.
   Embodiment 206. The method according to any of embodiments 178-203, wherein L³ is a bond, —CH₂—, —CH(CH₃)(OH)— or —CH(OH)—.
   Embodiment 207. The method according to any of embodiments 178-206, wherein R³ is aryl (e.g., phenyl) or heteroaryl (e.g., monocyclic heteroaryl) each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3D}), -L^{3C}-(heterocycloalkyl optionally substituted with 1-5 R^{3D}) and (ii) optionally substituted with 1-5 R^{3E}.
   Embodiment 208. The method according to any of embodiments 178-206, wherein R³ is aryl (e.g., a phenyl, a benzodioxole, or a dihydro-1H-isoquinoline) (i) substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3D}), -L^{3C}-(heterocycloalkyl optionally substituted with 1-5 R^{3D}) and (ii) optionally substituted with 1-5 R^{3E}.
   Embodiment 209. The method according to any of embodiments 178-206, wherein R³ is aryl (e.g., a phenyl, a benzodioxole, or a dihydro-1H-isoquinoline) (i) substituted with a single substituent selected from -L^{3C}-(phenyl optionally substituted with 1-5 R^{3D}), -L^{3C}-(monocyclic heteroaryl optionally substituted with 1-5 R^{3D}), -L^{3C}-(monocyclic cycloalkyl optionally substituted with 1-5 R^{3D}), -L^{3C}-(monocyclic heterocycloalkyl optionally substituted with 1-5 R^{3D}) and (ii) optionally substituted with 1-5 R^{3E}.
   Embodiment 210. The method according to any of embodiments 178-206, wherein R³ is heteroaryl (e.g., an isothiazole, a pyridone, a thiadiazole, a pyrazine, a pyrazolopyrimidine, a pyrazolopyridine, an imidazole, a benzofuran, an indole, an imidazopyridine, a pyridine, a pyrazole, an isoxazole, a triazolopyridine, a benzimidazole, a thiophene, a benzothiophene, a furan or a pyrimidine) (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3D}), -L^{3C}-(heterocycloalkyl optionally substituted with 1-5 R^{3D}) and (ii) optionally substituted with 1-5 R^{3E};
   Embodiment 211. The method according to any of embodiments 178-206, wherein R³ is heteroaryl (e.g., an isothiazole, a pyridone, a thiadiazole, a pyrazine, a pyrazolopyrimidine, a pyrazolopyridine, an imidazole, a benzofuran, an indole, an imidazopyridine, a pyridine, a pyrazole, an isoxazole, a triazolopyridine, a benzimidazole, a thiophene, a benzothiophene, a furan or a pyrimidine) (i) optionally substituted with a single substituent selected from -L^{3C}-(phenyl optionally substituted with 1-5 R^{3D}), -L^{3C}-(monocyclic heteroaryl optionally substituted with 1-5 R^{3D}), -L^{3C}-(monocyclic cycloalkyl optionally substituted with 1-5 R^{3D}), -L^{3C}-(monocyclic heterocycloalkyl optionally substituted with 1-5 R^{3D}) and (ii) optionally substituted with 1-5 R^{3E}.
   Embodiment 212. The method according to any of embodiments 178-206, wherein R³ is selected from the group consisting of: phenyl, benzodioxolyl, dihydro-1H-isoquinolinyl, imidazolyl, oxazolyl, isoxazolyl, isothiazolyl, thiazolyl, pyridinyl, and pyrazinyl, pyridonyl, thiadiazolyl, pyrazolopyrimidinyl, pyrazolopyridinyl, benzofuranyl, indolyl, imidazopyridinyl, pyrazolyl, triazolopyridinyl, benzimidazolyl, a benzimidazolyl, a thienyl, a benzothienyl, a furanyl and pyrimidinyl, each (i) optionally substituted with a single substituent selected from -L^{3C}-(aryl optionally substituted with 1-5 R^{3D}), -L^{3C}-(heteroaryl optionally substituted with 1-5 R^{3D}), -L^{3C}-(cycloalkyl optionally substituted with 1-5 R^{3D}), -L^{3C}-(heterocycloalkyl optionally substituted with 1-5 R^{3D}) and (ii) optionally substituted with 1-5 R^{3E}.
   Embodiment 213. The method according to any of embodiments 207-212, wherein the R³ substituent is not substituted with any R^{3E}.
   Embodiment 214. The method according to any of embodiments 207-212, wherein L^{3C} is methylene or —O—.
   Embodiment 215. The method according to any of embodiments 178-206, wherein R³ is aryl (e.g., a phenyl) optionally substituted with 1-5 R^{3E}.
   Embodiment 216. The method according to any of embodiments 178-206, wherein R³ is heteroaryl (e.g., an isothiazole, a pyridone, a thiadiazole, a pyrazine, a pyrazolopyrimidine, a pyrazolopyridine, an imidazole, a benzofuran, an indole, an imidazopyridine, a pyridine, a pyrazole, an isoxazole, a triazolopyridine, a benzimidazole, a thiophene, a benzothiophene, a furan or a pyrimidine) optionally substituted with 1-5 R^{3E}.
   Embodiment 217. The method according to any of embodiments 178-206, wherein R³ is selected from the group consisting of phenyl and monocyclic heteroaryl (e.g., pyridyl, pyrazolyl), optionally substituted with 1-5 R^{3E}.
   Embodiment 218. The method according to any of embodiments 178-217, wherein R⁴ is optionally substituted C₁-C₈ alkyl, optionally-substituted C₁-C₈ alkenyl or optionally substituted C₁-C₈ alkynyl.
   Embodiment 219. The method according to any of embodiments 178-217, wherein R⁴ is optionally substituted C₁-C₈ alkyl.
   Embodiment 220. The method according to any of embodiments 178-217, wherein R⁴ is hydrogen or unsubstituted C₁-C₆ alkyl.
   Embodiment 221. The method according to any of embodiments 178-217, wherein R⁴ is unsubstituted C₁-C₃ alkyl.
   Embodiment 222. The method according to any of embodiments 178-221, wherein L⁴ is a bond.
   Embodiment 223. The method according to any of embodiments 178-221, wherein L⁴ is selected from a bond, —C(O)—, —S—, —S(O)₁₋₂—, —O—, and —NR⁶—.
   Embodiment 224. The method according to any of embodiments 178-221, wherein L⁴ is —O—.
   Embodiment 225. The method according to any of embodiments 178-224, wherein L⁵ is a bond.
   Embodiment 226. The method according to any of embodiments 178-224, wherein L⁵ is a bond, —O—, —S—, —C(O)— or —S(O)₁₋₂—.
   Embodiment 227. The method of any of embodiments 178-226, wherein R⁵ is aryl (e.g., phenyl) or heteroaryl (e.g., an isoxazolyl, a pyridyl, an imidazopyridyl, a pyrazolyl), each optionally substituted with 1-5 R^{5E}.
   Embodiment 228. The method of any of embodiments 178-226, wherein R⁵ is phenyl optionally substituted with 1-5 R^{5E}.
   Embodiment 229. The method of any of embodiments 178-226, wherein R⁵ is selected from the group consisting of phenyl, isoxazolyl, pyridyl, imidazopyridyl, and pyrazolyl, each optionally substituted with 1-5 R^{5E}.
   Embodiment 230. The method of any of embodiments 178-226, wherein R⁵ is phenyl substituted with a single substituent selected from -L^{5C}-(phenyl optionally substituted with 1-5 R^{5D}), -L^{5C}-(monocyclic heteroaryl optionally substituted with 1-5 R^{5D}), and -L^{5C}-(monocyclic cycloalkyl optionally substituted with 1-5 R^{5D}) -L^{5C}-(monocyclic heterocycloalkyl optionally substituted with 1-5 R^{5D}) and (ii) optionally substituted with 1-5 R^{5E}.
   Embodiment 231. The method of any of embodiments 178-226, wherein R⁵ is phenyl substituted with a single -L^{5C}-(monocyclic heteroaryl optionally substituted with 1-5 R^{5D}) substituent and (ii) optionally substituted with 1-5 R^{5E}.
   Embodiment 232. The method of any of embodiments 178-226, wherein R⁵ is phenyl substituted with a single -L^{5C}-(monocyclic heterocycloalkyl optionally substituted with 1-5 R^{5D}) substituent and (ii) optionally substituted with 1-5 R^{5E}.
   Embodiment 233. The method of any of embodiments 230-232, wherein L^{5C} is a bond;
   Embodiment 234. The method of any of embodiments 230-232, wherein L^{5C} is —O— or —C(O)—.
   Embodiment 235. The method of any of embodiments 178-226, wherein R⁵ is heterocycloalkyl optionally substituted with 1-5 R^{5E}.
   Embodiment 236. The method of any of embodiments 178-226, wherein R⁵ is heterocycloalkyl substituted with a single -L^{5C}-(monocyclic cycloalkyl optionally substituted with 1-5 R^{5D}) substituent and (ii) optionally substituted with 1-5 R^{5E}.
   Embodiment 237. The method of any of embodiments 235-236, wherein the heterocycloalkyl is a nitrogen-containing heterocycloalkyl, attached to the -L⁵- through a nitrogen atom.
   Embodiment 238. The method of any of embodiments 235-236, wherein the heterocycloalkyl is monocyclic.
   Embodiment 239. The method of any of embodiments 235-236, wherein the heterocycloalkyl is bicyclic.
   Embodiment 240. The method of any of embodiments 235-239, wherein the heterocycloalkyl is saturated.
   Embodiment 241. The method of any of embodiments 178-226, wherein R⁵ is cycloalkyl optionally substituted with 1-5 R^{5E}.
   Embodiment 242. The method of embodiment 241, wherein the cycloalkyl is substituted with 1-5 R^{5E}.
   Embodiment 243. The method of embodiment 241 or embodiment 242, wherein the cycloalkyl is monocyclic.
   Embodiment 244. The method of any of embodiments 241-243, wherein the cycloalkyl is saturated.
   Embodiment 245. The method of any of embodiments 241-243, wherein the cycloalkyl is unsaturated.
   Embodiment 246. The method of any of embodiments 241-242, wherein the cycloalkyl is cyclohexen-1-yl.
   Embodiment 247. The method of embodiment 179, wherein
L¹ is a —S—, —O—, —S(O)—, —S(O)₂— or a bond;
R¹ is unsubstituted or fluorinated C₁-C₈ alkyl, unsubstituted or fluorinated C₁-C₈ alkenyl, unsubstituted or fluorinated C₁-C₈ alkynyl, or phenyl substituted with 1-5 R^{1E},
   in which
      each R^{1E} is independently selected from oxo, optionally-substituted C₁-C₄ alkyl, C₁-C₄ fluoroalkyl, halogen, —CN, SF₅, —N₃, —C(O)R^{1F}, —SR^{1F}, —S(O)₁₋₂R^{1F}, —OR^{1F}, —(OCH₂CH₂O)ₙ—R^{1G} in which n is 1-4, —N(R^{1G})C(O)CH₂—O—(CH₂CH₂O)ₙR^{1G} in which n is 0-3, —C(O)NR^{1G}(CH₂CH₂O)ₙR^{1G}, —NR^{1G}R^{1F} and —C(O)R^{1F};
      each R^{1F} is independently selected from H, C₁-C₃ alkyl and C₁-C₃ fluoroalkyl and
      each R^{1G} is independently selected from H and C₁-C₃ alkyl;
L² is a bond or —CH₂—;
Q is —COOH;
L³ is a bond, —C(O)—, —S—, —S(O)₁₋₂—, —O—, —NR⁶—, —CH₂—, —CH(CH₃)(OH)— or —CH(OH)—;
R³ is phenyl or monocyclic heteroaryl each (i) optionally substituted with a single substituent selected from -L^{3C}-(phenyl optionally substituted with 1-5 R^{3D}), -L^{3C}-(monocyclic heteroaryl optionally substituted with 1-5 R^{3D}), -L^{3C}-(monocyclic C3-C6 cycloalkyl optionally substituted with 1-5 R^{3E}), -L^{3C}-(monocyclic C4-C6 heterocycloalkyl optionally substituted with 1-5 R^{3E}) and (ii) optionally substituted with 1-5 R^{3E},
   in which
      each L^{3C} is a bond, methylene, ethylene, —C(O)—, —S—, —S(O)₁₋₂—, —O— or —NR^{3G}—;
      each R^{3D} is independently selected from optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F} and —NR^{3G}S(O)₁₋₂R^{3F};
      each R^{3E} is independently selected from oxo, optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F};
      each R^{3F} is independently selected from H, C₁-C₃ alkyl and C₁-C₃ fluoroalkyl and
      each R^{3G} is independently selected from H and C₁-C₃ alkyl, C₁-C₃ fluoroalkyl;
L⁴ is is selected from the group consisting of a bond, —C(O)—, —S—, —S(O)₁₋₂—, —O— and —NR⁶—;
R⁴ is selected from the group consisting of unsubstituted, hydroxylated, C₁-C₄ alkoxylated or fluorinated C₁-C₈ alkyl, unsubstituted or fluorinated C₁-C₈ alkenyl and unsubstituted or fluorinated C₁-C₈ alkynyl;
L⁵ is a bond, —C(O)—, —S—, —S(O)₁₋₂—, —O— or —NR⁶—;
R⁵ is phenyl, monocyclic heteroaryl, monocyclic heterocycloalkyl or monocyclic cycloalkyl each optionally substituted with 1-5 R^{5E},
   in which
      each R^{5E} is independently selected from oxo, optionally-substituted C₁-C₄ alkyl, C₁-C₄ fluoroalkyl, halogen, —CN, —SF₅, —N₃, —C(O)R^{5F}, —SR^{5F}, —S(O)₁₋₂R^{5F}, —OR^{5F}, —NR^{5G}R^{5F}, —C(O)R^{5F}, —C(O)NR^{5G}R^{5F}, —NR^{5G}C(O)R^{5F}, —C(S)NR^{5G}R^{5F}, —NR^{1G}C(S)R^{5F}, —C(O)OR^{5F}, —OC(O)R^{5F}, —C(O)SR^{5F}, —SC(O)R^{5F}, —C(S)OR^{5F}, —OC(S)R^{5F}, —C(S)SR^{5F}, —SC(S)R^{5F}, —S(O)₁₋₂OR^{5F}, —OS(O)₁₋₂R^{5F}, —S(O)₁₋₂NR^{5G}R^{5F} and —NR^{5G}S(O)₁₋₂R^{5F};
      each R^{5F} is independently selected from H, C₁-C₃ alkyl and C₁-C₃ fluoroalkyl and
      each R^{5G} is independently selected from H and C₁-C₃ alkyl;
wherein
each R⁶ is selected from the group consisting of hydrogen, C₁-C₃ alkyl and —C(O)(C₁-C₃ alkyl);
each optionally substituted alkyl, alkenyl and alkynyl is unsubstituted, fluorinated or substituted with one or two hydroxyl groups;
each cycloalkyl has 3-7 ring carbons and is unsaturated or partially unsaturated;
each heterocylcloalkyl has 3-7 ring members and 1-3 heteroatoms independently selected from nitrogen, oxygen and sulfur and is unsaturated or partially unsaturated;
each heteroaryl is a 5-6 membered monocyclic heteroaryl ring having 1-4 heteroatoms independently selected from nitrogen, oxygen and sulfur or a 8-10 membered bicyclic heteroaryl having 1-5 heteroatoms independently selected from nitrogen, oxygen or sulfur.
   Embodiment 248. The method of embodiment 247, wherein
R³ is phenyl optionally substituted with 1-5 R^{3E}, in which
   each L^{3C} is a bond, methylene, ethylene, —C(O)—, —S—, —S(O)₁₋₂—, —O— or —NR^{3G}—;
   each R^{3D} is independently selected from optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F} and —NR^{3G}S(O)₁₋₂R^{3F};
   each R^{3E} is independently selected from oxo, optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F};
   each R^{3F} is independently selected from H, C₁-C₃ alkyl and C₁-C₃ fluoroalkyl and
   each R^{3G} is independently selected from H and C₁-C₃ alkyl, C₁-C₃ fluoroalkyl;
R⁵ is phenyl, morpholinyl, cyclohexyl, cyclohexenyl, piperidinyl, piperazinyl or pyrrolidinyl optionally substituted with 1-5 R^{5E}, in which
   each R^{5E} is independently selected from oxo, optionally-substituted C₁-C₄ alkyl, C₁-C₄ fluoroalkyl, halogen, —CN, —SF₅, —N₃, —C(O)R^{5F}, —SR^{5F}, —S(O)₁₋₂R^{5F}, —OR^{5F}, —NR^{5G}R^{5F}, —C(O)R^{5F}, —C(O)NR^{5G}R^{5F}, —NR^{5G}C(O)R^{5F}, —C(S)NR^{5G}R^{5F}, —NR^{1G}C(S)R^{5F}, —C(O)OR^{5F}, —OC(O)R^{5F}, —C(O)SR^{5F}, —SC(O)R^{5F}, —C(S)OR^{5F}, —OC(S)R^{5F}, —C(S)SR^{5F}, —SC(S)R^{5F}, —S(O)₁₋₂OR^{5F}, —OS(O)₁₋₂R^{5F}, —S(O)₁₋₂NR^{5G}R^{5F} and —NR^{5G}S(O)₁₋₂R^{5F};
   each R^{5F} is independently selected from H, C₁-C₃ alkyl and C₁-C₃ fluoroalkyl and
   each R^{5G} is independently selected from H and C₁-C₃ alkyl.
      Embodiment 249. The method according to embodiment 247 or embodiment 248, wherein
L¹ is —S—;
L² is a bond; and
L³ is a bond.
   Embodiment 250. The method of any of embodiments 247-249, wherein
L⁴ is a bond; and
L⁵ is a bond.
   Embodiment 251. The method of any of embodiments 178 and 180-183, wherein
L¹ is a —S—, —O—, —S(O)—, —S(O)₂— or a bond;
R¹ is unsubstituted or fluorinated C₁-C₈ alkyl, unsubstituted or fluorinated C₁-C₈ alkenyl, unsubstituted or fluorinated C₁-C₈ alkynyl, or phenyl substituted with 1-5 R^{1E},
   in which
      each R^{1E} is independently selected from oxo, optionally-substituted C₁-C₄ alkyl, C₁-C₄ fluoroalkyl, halogen, —CN, SF₅, —N₃, —C(O)R^{1F}, —SR^{1F}, —S(O)₁₋₂R^{1F}, —OR^{1F}, —(OCH₂CH₂O)ₙ—R^{1G} in which n is 1-4, —N(R^{1G})C(O)CH₂—O—(CH₂CH₂O)ₙR^{1G} in which n is 0-3, —C(O)NR^{1G}(CH₂CH₂O)ₙR^{1G}, —NR^{1G}R^{1F} and —C(O)R^{1F};
      each R^{1F} is independently selected from H, C₁-C₃ alkyl and C₁-C₃ fluoroalkyl and
      each R^{1G} is independently selected from H and C₁-C₃ alkyl;
L² is a bond or —CH₂—;
Q is —COOH;
L³ is a bond, —C(O)—, —S—, —S(O)₁₋₂—, —O—, —NR⁶—, —CH₂—, —CH(CH₃)(OH)— or —CH(OH)—;
R³ is phenyl or monocyclic heteroaryl each (i) optionally substituted with a single substituent selected from -L^{3C}-(phenyl optionally substituted with 1-5 R^{3D}), -L^{3C}-(monocyclic heteroaryl optionally substituted with 1-5 R^{3D}), -L^{3C}-(monocyclic C3-C6 cycloalkyl optionally substituted with 1-5 R^{3E}), -L^{3C}-(monocyclic C4-C6 heterocycloalkyl optionally substituted with 1-5 R^{3E}) and (ii) optionally substituted with 1-5 R^{3E},
   in which
      each L^{3C} is a bond, methylene,
         ethylene, —C(O)—, —S—, —S(O)₁₋₂—, —O— or —NR^{3G}—;
      each R^{3D} is independently selected from optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F} and —NR^{3G}S(O)₁₋₂R^{3F};
      each R^{3E} is independently selected from oxo, optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F};
      each R^{3F} is independently selected from H, C₁-C₃ alkyl and C₁-C₃ fluoroalkyl and
      each R^{3G} is independently selected from H and C₁-C₃ alkyl, C₁-C₃ fluoroalkyl;
L⁴ is is selected from the group consisting of a bond, —C(O)—, —S—, —S(O)₁₋₂—, —O— and —NR⁶—;
R⁴ is selected from the group consisting of unsubstituted, hydroxylated, C₁-C₄ alkoxylated or fluorinated C₁-C₈ alkyl, unsubstituted or fluorinated C₁-C₈ alkenyl and unsubstituted or fluorinated C₁-C₈ alkynyl;
L⁵ is a bond, —C(O)—, —S—, —S(O)₁₋₂—, —O— or —NR⁶—;
R⁵ is phenyl, monocyclic heteroaryl, monocyclic heterocycloalkyl or monocyclic cycloalkyl each optionally substituted with 1-5 R^{5E},
   in which
      each R^{5E} is independently selected from oxo, optionally-substituted C₁-C₄ alkyl, C₁-C₄ fluoroalkyl, halogen, —CN, —SF₅, —N₃, —C(O)R^{5F}, —SR^{5F}, —S(O)₁₋₂R^{5F}, —OR^{5F}, —NR^{5G}R^{5F}, —C(O)R^{5F}, —C(O)NR^{5G}R^{5F}, —NR^{5G}C(O)R^{5F}, —C(S)NR^{5G}R^{5F}, —NR^{1G}C(S)R^{5F}, —C(O)OR^{5F}, —OC(O)R^{5F}, —C(O)SR^{5F}, —SC(O)R^{5F}, —C(S)OR^{5F}, —OC(S)R^{5F}, —C(S)SR^{5F}, —SC(S)R^{5F}, —S(O)₁₋₂OR^{5F}, —OS(O)₁₋₂R^{5F}, —S(O)₁₋₂NR^{5G}R^{5F} and —NR^{5G}S(O)₁₋₂R^{5F};
      each R^{5F} is independently selected from H, C₁-C₃ alkyl and C₁-C₃ fluoroalkyl and
      each R^{5G} is independently selected from H and C₁-C₃ alkyl;
         wherein
each R⁶ is selected from the group consisting of hydrogen, C₁-C₃ alkyl and —C(O)(C₁-C₃ alkyl);
each optionally substituted alkyl, alkenyl and alkynyl is unsubstituted, fluorinated or substituted with one or two hydroxyl groups;
each cycloalkyl has 3-7 ring carbons and is unsaturated or partially unsaturated;
each heterocylcloalkyl has 3-7 ring members and 1-3 heteroatoms independently selected from nitrogen, oxygen and sulfur and is unsaturated or partially unsaturated;
each heteroaryl is a 5-6 membered monocyclic heteroaryl ring having 1-4 heteroatoms independently selected from nitrogen, oxygen and sulfur or a 8-10 membered bicyclic heteroaryl having 1-5 heteroatoms independently selected from nitrogen, oxygen or sulfur.
   Embodiment 252. The method of embodiment 251, wherein
R³ is phenyl optionally substituted with 1-5 R^{3E}, in which
   each L^{3C} is a bond, methylene, ethylene, —C(O)—, —S—, —S(O)₁₋₂—, —O— or —NR^{3G}—;
   each R^{3D} is independently selected from optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F} and —NR^{3G}S(O)₁₋₂R^{3F};
   each R^{3E} is independently selected from oxo, optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F};
   each R^{3F} is independently selected from H, C₁-C₃ alkyl and C₁-C₃ fluoroalkyl and
   each R^{3G} is independently selected from H and C₁-C₃ alkyl, C₁-C₃ fluoroalkyl;
R⁵ is phenyl, morpholinyl, cyclohexyl, cyclohexenyl, piperidinyl, piperazinyl or pyrrolidinyl optionally substituted with 1-5 R^{5E}, in which
   each R^{5E} is independently selected from oxo, optionally-substituted C₁-C₄ alkyl, C₁-C₄ fluoroalkyl, halogen, —CN, —SF₅, —N₃, —C(O)R^{5F}, —SR^{5F}, —S(O)₁₋₂R^{5F}, —OR^{5F}, —NR^{5G}R^{5F}, —C(O)R^{5F}, —C(O)NR^{5G}R^{5F}, —NR^{5G}C(O)R^{5F}, —C(S)NR^{5G}R^{5F}, —NR^{1G}C(S)R^{5F}, —C(O)OR^{5F}, —OC(O)R^{5F}, —C(O)SR^{5F}, —SC(O)R^{5F}, —C(S)OR^{5F}, —OC(S)R^{5F}, —C(S)SR^{5F}, —SC(S)R^{5F}, —S(O)₁₋₂OR^{5F}, —OS(O)₁₋₂R^{5F}, —S(O)₁₋₂NR^{5G}R^{5F} and —NR^{5G}S(O)₁₋₂R^{5F};
   each R^{5F} is independently selected from H, C₁-C₃ alkyl and C₁-C₃ fluoroalkyl and
   each R^{5G} is independently selected from H and C₁-C₃ alkyl.
      Embodiment 253. The method according to embodiment 251 or embodiment 252, wherein
L¹ is —S—;
L² is a bond; and
L³ is a bond.
   Embodiment 254. The method of any of embodiments 251-253, wherein
L⁴ is a bond; and
L⁵ is a bond.
   Embodiment 255. The method of any of embodiments 178-226 and 247-254, wherein R⁵ is trifluoromethylphenyl, halophenyl or dihalophenyl.
   Embodiment 256. The method of any of embodiments 178-226 and 247-254, wherein R⁵ is phenyl substituted (e.g., 3-substituted, 4-substituted, 3,4-disubstituted, 2,4-disubstituted, or 2,5-disubstituted) with one or two substituents selected from trifluoromethyl, fluorine and chlorine.
   Embodiment 257. The method of any of embodiments 178-226 and 247-254, wherein R⁵ is cyclohexen-1-yl, optionally substituted with 1-3 R^{5E}.
   Embodiment 258. The method of any of embodiments 178-226 and 247-254, wherein R⁵ is 4-(C₁-C₅ alkyl)cyclohexen-1-yl, e.g., 4-methylcyclohexen-1-yl
   Embodiment 259. The method of any of embodiments 178-258, wherein each optionally substituted alkylene, alkenylene and alkynylene is unsubstituted.
   Embodiment 260. The method of any of embodiments 178-258, wherein each optionally substituted alkyl, alkenyl and alkynyl is unsubstituted.
   Embodiment 261. The method of any of embodiments 178-260, wherein each cycloalkyl is a 3-7 membered monocyclic cycloalkyl.
   Embodiment 262. The method of any of embodiments 178-261, wherein each heterocycloalkyl is a 4-7 membered monocyclic heterocycloalkyl having 1-2 heteroatoms selected from O, S and N.
   Embodiment 263. The method of any of embodiments 178-261, wherein each heteroaryl is a 5-6 membered monocyclic heteroaryl having 1-3 heteroatoms selected from O, S and N.
   Embodiment 264. The method of any of embodiments 178-261, wherein each aryl is phenyl.
   Embodiment 265. The method of any of embodiments 1-88, wherein the therapeutic compound is selected from
1-(4-(4-chloro-2-(oxetan-3-yloxy)phenyl)-5-(isopropylthio)thiazol-2-yl)-3-methyl-1H-pyrazole-5-carboxylic acid;
1-(4-(4-chloro-3-(oxetan-3-yloxy)phenyl)-5-(isopropylthio)thiazol-2-yl)-3-methyl-1H-pyrazole-5-carboxylic acid;
4-(3-fluorophenyl)-1-(5-(isopropylthio)-4-(4-(5-methyl-1,3,4-oxadiazol-2-yl)phenyl)thiazol-2-yl)-3-methyl-1H-pyrazole-5-carboxylic acid;
4-(3-fluorophenyl)-1-(5-(isopropylthio)-4-(4-methylcyclohex-1-en-1-yl)thiazol-2-yl)-3-methyl-1H-pyrazole-5-carboxylic acid;
4-(3-fluorophenyl)-1-(5-(isopropylthio)-4-(4-(trifluoromethyl)cyclohex-1-en-1-yl)thiazol-2-yl)-3-methyl-1H-pyrazole-5-carboxylic acid;
1-(4-(4,4-dimethylcyclohex-1-en-1-yl)-5-(isopropylthio)thiazol-2-yl)-4-(3-fluorophenyl)-3-methyl-1H-pyrazole-5-carboxylic acid;
4-(3-fluorophenyl)-1-(5-(isopropylthio)-4-(1,4-dioxaspiro[4.5]dec-7-en-8-yl)thiazol-2-yl)-3-methyl-1H-pyrazole-5-carboxylic acid;
1-(4-(4-chloro-3-(morpholine-4-carbonyl)phenyl)-5-(isopropylthio)thiazol-2-yl)-4-(3-fluorophenyl)-3-methyl-1H-pyrazole-5-carboxylic acid;
4-(3,4-dichlorophenyl)-2-(4-(2,6-dimethylpyridin-4-yl)-3-methyl-1H-pyrazol-1-yl)-5-(isopropylthio)thiazole;
2-(4-(3-fluorophenyl)-3,5-dimethyl-1H-pyrazol-1-yl)-5-(isopropylthio)-4-(4-(trifluoromethyl)phenyl)thiazole;
4-(3-fluorophenyl)-1-(5-(isopropylthio)-4-(piperidin-1-yl)thiazol-2-yl)-3-methyl-1H-pyrazole-5-carboxylic acid;
4-(3-fluorophenyl)-1-(5-(isopropylthio)-4-(4-(trifluoromethyl)piperidin-1-yl)thiazol-2-yl)-3-methyl-1H-pyrazole-5-carboxylic acid;
4-(3-fluorophenyl)-1-(5-(isopropylthio)-4-(4-(trifluoromethyl)phenyl)thiazol-2-yl)-3-methoxy-1H-pyrazole-5-carboxylic acid;
4-(3-fluorophenyl)-1-(5-(isopropylthio)-4-morpholinothiazol-2-yl)-3-methyl-1H-pyrazole-5-carboxylic acid;
4-(3-fluorophenyl)-3-hydroxy-1-(5-(isopropylthio)-4-(4-(trifluoromethyl)phenyl)thiazol-2-yl)-1H-pyrazole-5-carboxylic acid;
1-(5-(3,4-dichlorophenyl)-1-isobutyl-1H-1,2,4-triazol-3-yl)-4-(3-fluorophenyl)-3-methyl-1H-pyrazole-5-carboxylic acid;
1-(3-(3,4-dichlorophenyl)-1-isobutyl-1H-1,2,4-triazol-5-yl)-4-(3-fluorophenyl)-3-methyl-1H-pyrazole-5-carboxylic acid;
1-(4-(4,4-difluoropiperidin-1-yl)-5-(isopropylthio)thiazol-2-yl)-4-(3-fluorophenyl)-3-methyl-1H-pyrazole-5-carboxylic acid;
methyl 4-(3-fluorophenyl)-1-(5-(isopropylthio)-4-(4-(trifluoromethyl)cyclohex-1-en-1-yl)thiazol-2-yl)-3-methyl-1H-pyrazole-5-carboxylate;
methyl 4-(3-fluorophenyl)-1-(5-(isopropylthio)-4-(4-(trifluoromethyl)cyclohex-1-en-1-yl)thiazol-2-yl)-3-methyl-1H-pyrazole-5-carboxylate;
4-(3-fluorophenyl)-3-methyl-1-(4-(4-(trifluoromethyl)cyclohexyl)thiazol-2-yl)-1H-pyrazole-5-carboxylic acid
4-(3-fluorophenyl)-1-(5-isobutyl-4-(4-(trifluoromethyl)phenyl)thiazol-2-yl)-3-methyl-1H-pyrazole-5-carboxylic acid;
4-(3-fluorophenyl)-1-(5-(isopropylthio)-4-(4-(2,2,2-trifluoroethyl)piperazin-1-yl)thiazol-2-yl)-3-methyl-1H-pyrazole-5-carboxylic acid;
1-(4-(4-cyanopiperidin-1-yl)-5-(isopropylthio)thiazol-2-yl)-4-(3-fluorophenyl)-3-methyl-1H-pyrazole-5-carboxylic acid;
1-(4-(4-cyclopropylpiperazin-1-yl)-5-(isopropylthio)thiazol-2-yl)-4-(3-fluorophenyl)-3-methyl-1H-pyrazole-5-carboxylic acid;
1-(4-(4-ethylpiperazin-1-yl)-5-(isopropylthio)thiazol-2-yl)-4-(3-fluorophenyl)-3-methyl-1H-pyrazole-5-carboxylic acid;
1-(4-(4-acetylpiperazin-1-yl)-5-(isopropylthio)thiazol-2-yl)-4-(3-fluorophenyl)-3-methyl-1H-pyrazole-5-carboxylic acid;
4-(3-fluorophenyl)-1-(5-(isopropylthio)-4-(4-methylpiperidin-1-yl)thiazol-2-yl)-3-methyl-1H-pyrazole-5-carboxylic acid;
4-(3-fluorophenyl)-1-(5-(isopropylthio)-4-(4-methylpiperazin-1-yl)thiazol-2-yl)-3-methyl-1H-pyrazole-5-carboxylic acid;
4-(3-fluorophenyl)-3-methyl-1-(5-(4-(trifluoromethyl)phenyl)-1,3,4-thiadiazol-2-yl)-1H-pyrazole-5-carboxylic acid;
4-(3-fluorophenyl)-1-(5-((2-methoxyethyl)(methyl)amino)-4-(4-(trifluoromethyl)phenyl)thiazol-2-yl)-3-methyl-1H-pyrazole-5-carboxylic acid
1-(4-(4,4-dimethylpiperidin-1-yl)-5-(isopropylthio)thiazol-2-yl)-4-(3-fluorophenyl)-3-methyl-1H-pyrazole-5-carboxylic acid;
1-(4-(4-(tert-butoxycarbonyl)piperazin-1-yl)-5-(isopropylthio)thiazol-2-yl)-4-(3-fluorophenyl)-3-methyl-1H-pyrazole-5-carboxylic acid;
4-(3-fluorophenyl)-1-(5-(isopropylthio)-4-(3-(trifluoromethyl)pyrrolidin-1-yl)thiazol-2-yl)-3-methyl-1H-pyrazole-5-carboxylic acid;
4-(3-fluorophenyl)-1-(5-(isopropylthio)-4-(piperazin-1-yl)thiazol-2-yl)-3-methyl-1H-pyrazole-5-carboxylic acid;
4-(3-fluorophenyl)-3-methyl-1-(5-(2-methylprop-1-en-1-yl)-4-(4-(trifluoromethyl)phenyl)thiazol-2-yl)-1H-pyrazole-5-carboxylic acid;
4-(3-fluorophenyl)-3-methyl-1-(4-(2-methylprop-1-en-1-yl)-5-(4-(trifluoromethyl)phenyl)thiazol-2-yl)-1H-pyrazole-5-carboxylic acid;
1-(4,5-bis(4-(trifluoromethyl)phenyl)thiazol-2-yl)-4-(3-fluorophenyl)-3-methyl-1H-pyrazole-5-carboxylic acid;
2-(4-(3-fluorophenyl)-3-methyl-1H-pyrazol-1-yl)-4,5-bis(4-(trifluoromethyl)phenyl)thiazole
1-(4-(4-(tert-butyl)piperidin-1-yl)-5-(isopropylthio)thiazol-2-yl)-4-(3-fluorophenyl)-3-methyl-1H-pyrazole-5-carboxylic acid;
4-(3-fluorophenyl)-1-(5-(isopropylthio)-4-(6-azaspiro[2.5]octan-6-yl)thiazol-2-yl)-3-methyl-1H-pyrazole-5-carboxylic acid;
4-(3-fluorophenyl)-1-(5-(isopropylthio)-4-(4-methoxy-4-(trifluoromethyl)piperidin-1-yl)thiazol-2-yl)-3-methyl-1H-pyrazole-5-carboxylic acid;
4-(3-fluorophenyl)-1-(4-(4-methoxyphenyl)-5-(4-(trifluoromethyl)phenyl)thiazol-2-yl)-3-methyl-1H-pyrazole-5-carboxylic acid;
1-(4,5-bis(4-methoxyphenyl)thiazol-2-yl)-4-(3-fluorophenyl)-3-methyl-1H-pyrazole-5-carboxylic acid;
4-(3-fluorophenyl)-1-(5-(4-methoxyphenyl)-4-(4-(trifluoromethyl)phenyl)thiazol-2-yl)-3-methyl-1H-pyrazole-5-carboxylic acid;
1-(4-(4-(tert-butyl)-3-oxopiperazin-1-yl)-5-(isopropylthio)thiazol-2-yl)-4-(3-fluorophenyl)-3-methyl-1H-pyrazole-5-carboxylic acid;
4-(3-fluorophenyl)-3-methyl-1-(5-(3-(methylamino)-3-oxopropyl)-4-(4-(trifluoromethyl)phenyl)thiazol-2-yl)-1H-pyrazole-5-carboxylic acid;
4-(3-fluorophenyl)-1-(5-(isopropylthio)-4-(4-(2-methoxyethoxy)-4-(trifluoromethyl)piperidin-1-yl)thiazol-2-yl)-3-methyl-1H-pyrazole-5-carboxylic acid;
4-(3-fluorophenyl)-1-(5-(4-((2-methoxyethyl)carbamoyl)phenyl)-4-(4-(trifluoromethyl)phenyl)thiazol-2-yl)-3-methyl-1H-pyrazole-5-carboxylic acid;
4-(3-fluorophenyl)-1-(5-(4-((2-methoxyethyl)(methyl)carbamoyl)phenyl)-4-(4-(trifluoromethyl)phenyl)thiazol-2-yl)-3-methyl-1H-pyrazole-5-carboxylic acid;
4-(3-fluorophenyl)-1-(5-(4-(2-methoxyacetamido)phenyl)-4-(4-(trifluoromethyl)phenyl)thiazol-2-yl)-3-methyl-1H-pyrazole-5-carboxylic acid;
4-(3-fluorophenyl)-1-(5-(4-(2-(2-methoxyethoxy)acetamido)phenyl)-4-(4-(trifluoromethyl)phenyl)thiazol-2-yl)-3-methyl-1H-pyrazole-5-carboxylic acid;
4-(3-fluorophenyl)-1-(5-(3-((2-methoxyethyl)amino)-3-oxopropyl)-4-(4-(trifluoromethyl)phenyl)thiazol-2-yl)-3-methyl-1H-pyrazole-5-carboxylic acid;
4-(3-fluorophenyl)-1-(5-(3-((2-methoxyethyl)(methyl)amino)-3-oxopropyl)-4-(4-(trifluoromethyl)phenyl)thiazol-2-yl)-3-methyl-1H-pyrazole-5-carboxylic acid;
4-(3-fluorophenyl)-1-(5-(4-(2-methoxy-N-methylacetamido)phenyl)-4-(4-(trifluoromethyl)phenyl)thiazol-2-yl)-3-methyl-1H-pyrazole-5-carboxylic acid;
4-(3-fluorophenyl)-1-(5-(4-(2-(2-methoxyethoxy)-N-methylacetamido)phenyl)-4-(4-(trifluoromethyl)phenyl)thiazol-2-yl)-3-methyl-1H-pyrazole-5-carboxylic acid;
4-(3-fluorophenyl)-1-(5-(methoxymethyl)-4-(4-(trifluoromethyl)phenyl)thiazol-2-yl)-3-methyl-1H-pyrazole-5-carboxylic acid;
1-(5-(4-(2-(2-ethoxyethoxy)ethoxy)phenyl)-4-(4-(trifluoromethyl)phenyl)thiazol-2-yl)-4-(3-fluorophenyl)-3-methyl-1H-pyrazole-5-carboxylic acid;
4-(3-fluorophenyl)-1-(5-(3-fluorophenyl)-4-(4-(trifluoromethyl)phenyl)thiazol-2-yl)-3-methyl-1H-pyrazole-5-carboxylic acid;
4-(3-fluorophenyl)-1-(5-(hydroxymethyl)-4-(4-(trifluoromethyl)phenyl)thiazol-2-yl)-3-methyl-1H-pyrazole-5-carboxylic acid;
4-(3-fluorophenyl)-3-methyl-1-(5-(4-(trifluoromethyl)phenyl)-4-(4-(trifluoromethyl)piperidin-1-yl)thiazol-2-yl)-1H-pyrazole-5-carboxylic acid;
4-(3-fluorophenyl)-1-(4-(3-fluorophenyl)-5-(4-(trifluoromethyl)phenyl)thiazol-2-yl)-3-methyl-1H-pyrazole-5-carboxylic acid;
4-(3-fluorophenyl)-1-(5-(1-hydroxyethyl)-4-(4-(trifluoromethyl)phenyl)thiazol-2-yl)-3-methyl-1H-pyrazole-5-carboxylic acid;
4-(3-fluorophenyl)-1-(5-(2-hydroxyethyl)-4-(4-(trifluoromethyl)phenyl)thiazol-2-yl)-3-methyl-1H-pyrazole-5-carboxylic acid;
4-(3-fluorophenyl)-1-(5-(4-(2-methoxyethoxy)phenyl)-4-(4-(trifluoromethyl)phenyl)thiazol-2-yl)-3-methyl-1H-pyrazole-5-carboxylic acid;
4-(3-fluorophenyl)-1-(5-(1-methoxyethyl)-4-(4-(trifluoromethyl)phenyl)thiazol-2-yl)-3-methyl-1H-pyrazole-5-carboxylic acid;
4-(3-fluorophenyl)-1-(4-(4-isopropylpiperidin-1-yl)-5-(isopropylthio)thiazol-2-yl)-3-methyl-1H-pyrazole-5-carboxylic acid; and
4-(3-fluorophenyl)-1-(5-(isopropylthio)-4-(3-methoxy-3-(trifluoromethyl)-8-azabicyclo[3.2.1]octan-8-yl)thiazol-2-yl)-3-methyl-1H-pyrazole-5-carboxylic acid, optionally in the form of a pharmaceutically acceptable salt or N-oxide, or a solvate or hydrate.
   Embodiment 266. The method of any of embodiments 1-88, wherein the therapeutic compound is selected from compounds identified in the specification as having activity “A”, “B” or “C,” optionally in the form of a pharmaceutically acceptable salt or N-oxide, or a solvate or hydrate
   Embodiment 267. The method of any of embodiments 1-88, wherein the therapeutic compound is selected from compounds identified in the specification as having activity “A”, or “B” optionally in the form of a pharmaceutically acceptable salt or N-oxide, or a solvate or hydrate
   Embodiment 268. The method of any of embodiments 1-88, wherein the therapeutic compound is selected from compounds identified in the specification as having activity “A”, optionally in the form of a pharmaceutically acceptable salt or N-oxide, or a solvate or hydrate
   Embodiment 269. The method of any of embodiments 1-88, wherein the therapeutic compound is a compound as described in any embodiment or genus of International Patent Application Publication No. 2015/196644, or of International Patent Application Publication No. 2018/102453.
   Embodiment 270. The method of any applicable embodiment above, wherein the cancer is acute lymphoblastic leukemia, acute promyelocytic leukemia, adrenal cortex carcinoma, acute monocytic leukemia, acute myeloid leukemia, B acute lymphoblastic leukemia, amelanotic melanoma, anaplastic large cell lymphoma, astrocytoma, B-cell prolymphocytic leukemia, biphasic synovial sarcoma, bladder carcinoma, chronic myeloid leukemia, breast adenocarcinoma, breast carcinoma, Burkitt's lymphoma, cecum adenocarcinoma, cervical carcinoma, cervical squamous cell carcinoma, T acute lymphoblastic leukemia, chronic eosinophilic leukemia, chronic myelogenous leukemia, colon adenocarcinoma, colon carcinoma, cutaneous melanoma, diffuse gastric adenocarcinoma, diffuse large B-cell lymphoma, diffuse large B-cell lymphoma activated B-cell type, diffuse large B-cell lymphoma germinal center B-Cell type, ductal breast carcinoma, duodenal adenocarcinoma, embryonal rhabdomyosarcoma, endometrial adenocarcinoma, endometrial adenosquamous carcinoma, Epstein-Barr virus-related Burkitt lymphoma, erythroleukemia, esophageal squamous cell carcinoma, Ewing sarcoma, fibrosarcoma, follicular lymphoma, gallbladder carcinoma, gastric adenocarcinoma, gastric adenosquamous carcinoma, gastric carcinoma, gastric tubular adenocarcinoma, gestational choriocarcinoma, glioblastoma, head and neck squamous cell carcinoma, hepatoblastoma, hepatocellular carcinoma, thyroid gland medullary carcinoma, ovarian serous adenocarcinoma, human papillomavirus-related cervical squamous cell carcinoma, human papillomavirus-related endocervical adenocarcinoma, hypopharyngeal squamous cell carcinoma, thyroid gland undifferentiated (anaplastic) carcinoma, inflammatory breast carcinoma, intrahepatic cholangiocarcinoma, invasive ductal carcinoma, large B-cell lymphoma, large cell lung carcinoma, lung adenocarcinoma, mantle cell lymphoma, melanoma, minimally invasive lung adenocarcinoma, nasopharyngeal carcinoma, natural killer cell lymphoblastic leukemia/lymphoma, neuroblastoma, non-small cell lung carcinoma, osteosarcoma, ovarian clear cell adenocarcinoma, ovarian endometrioid adenocarcinoma, ovarian serous cystadenocarcinoma, pancreatic adenocarcinoma, pancreatic carcinoma, pancreatic ductal adenocarcinoma, papillary lung adenocarcinoma, papillary renal cell carcinoma, plasma cell myeloma, plasmacytoma, pleomorphic breast carcinoma, pleural biphasic mesothelioma, pleural epithelioid mesothelioma, prostate carcinoma, rectal adenocarcinoma, rectosigmoid adenocarcinoma, renal cell carcinoma, Sezary Syndrome, signet ring cell gastric adenocarcinoma, small cell lung carcinoma, squamous cell lung carcinoma, thyroid gland follicular carcinoma, thyroid gland papillary carcinoma, thyroid gland squamous cell carcinoma, thyroid gland undifferentiated (anaplastic) carcinoma, tongue squamous cell carcinoma, uterine corpus sarcoma, or vulvar squamous cell carcinoma.
   Embodiment 271. The method of any of any applicable embodiment above, wherein the cancer is acute promyelocytic leukemia, acute monocytic leukemia, acute myeloid leukemia, B acute lymphoblastic leukemia, Anaplastic large cell lymphoma, B-cell prolymphocytic leukemia, chronic myeloid leukemia, Burkitt lymphoma, chronic eosinophilic leukemia, chronic myelogenous leukemia, diffuse large B-cell lymphoma, diffuse large B-cell lymphoma activated B-cell type, diffuse large B-cell lymphoma germinal center B-Cell type, Epstein-Barr virus-related Burkitt lymphoma, erythroleukemia, follicular lymphoma, large B-cell lymphoma acute lymphoblastic leukemia, mantle cell lymphoma, natural killer cell lymphoblastic leukemia/lymphoma plasma cell myeloma, plasmacytoma, or Sezary syndrome.

It is understood that the examples and embodiments described herein are for illustrative purposes only and that various modifications or changes in light thereof will be suggested to persons skilled in the art and are to be incorporated within the spirit and purview of this application and scope of the appended claims.

What is claimed is:

1. A method for treating a solid tumor cancer in a human individual, the method comprising administering to the human individual an effective amount of a therapeutic compound of any of formule (I)-(Io) or (IIa)-(IIe), the solid tumor cancer exhibiting a significant FAM210B expression fold change as compared to the level of expression of FAM210B in a reference cell, FAM210B expression in the cancer being lower than FAM210B expression in the reference cell.

2. A method for treating a solid tumor cancer in a human individual using a therapeutic compound of any of formule (I)-(Io) or (IIa)-(IIe), the method comprising:
determining the level of expression of FAM210B of the cancer;
determining a FAM210B expression fold change as compared to the level of expression of FAM210B in a reference cell; and
if the FAM210B expression fold change is significant, and if FAM210B expression in the cancer is lower than FAM210B expression in the reference cell, administering an effective amount of the therapeutic compound to the human individual.

3. A method for determining whether a solid tumor cancer is responsive to a therapeutic compound of any of formule (I)-(Io) or (IIa)-(IIe), the method comprising:
determining the level of expression of FAM210B of the cancer;
determining a FAM210B expression fold change as compared to the level of expression of FAM210B in a reference cell; and
if the FAM210B expression fold change is significant, and if FAM210B expression in the cancer is lower than FAM210B expression in the reference cell, identifying the cancer as likely to be responsive to the therapeutic compound.

4. The method of any of claims 1-3, wherein the solid tumor cancer is adrenal gland(s) cancer, bile duct cancer, a bone or muscle cancer, cervical cancer, colorectal cancer, esophageal cancer, eye cancer, a head or neck cancer (e.g. a cancer of the nose, of the tongue, of the thyroid, or of a submaxillary gland), a kidney cancer, liver cancer, large intestine cancer, small cell lung cancer or non-small cell lung cancer, nervous system cancer, ovarian cancer, pancreatic cancer, placental cancer, prostate cancer, skin cancer, small intestine cancer, stomach/gastric cancer, or uterine cancer.

5. The method of any of claims 1-4, wherein a gene expression fold change of at least 1.5 is a significant change in gene expression.

6. The method of any of claims 1-5, wherein a gene expression fold change of at least 2 (e.g., at least 3) is a significant change in gene expression.

7. A method for treating a hematopoietic cancer in a human individual, the method comprising administering to the human individual an effective amount of a therapeutic compound of any of formule (I)-(Io) or (IIa)-(IIe), wherein the cancer is a hematopoietic cancer that exhibits a significant gene expression fold change as compared to a reference cell with respect to a first number of a plurality of genes selected from CASP10, TMED1, PPP1CC, TMEM59, BRD7, CYB561, FAM210B, NDRG1, CTSB, MMAB, SETDB2, VPS37B, ELL3, and KIF13B, wherein the first number is at least five.

8. A method for treating a hematopoietic cancer in a human individual, comprising:
determining the level of expression of a plurality of genes of the cancer;
determining a gene expression fold change as compared to the level of expression of the plurality of genes in a reference cell; and
if the gene expression fold change is significant with respect to a first number of the plurality of genes, administering an effective amount of a therapeutic compound of any of formule (I)-(Io) or (IIa)-(IIe) to the human individual, the first number being five or more,
wherein the cancer is a hematopoietic cancer that exhibits a significant gene expression fold change as compared to a reference cell with respect to a first number of a plurality of genes selected from CASP10, TMED1, PPP1CC, TMEM59, BRD7, CYB561, FAM210B, NDRG1, CTSB, MMAB, SETDB2, VPS37B, ELL3, and KIF13B, wherein the first number is at least five.

9. A method for determining whether a hematopoietic cancer is responsive to a therapeutic compound of any of formule (I)-(Io) or (IIa)-(IIe), the method comprising:
determining the level of expression of a plurality of genes of the cancer;
determining a gene expression fold change as compared to the level of expression of the one or more genes in a reference cell; and
if the gene expression fold change is significant with respect to a first number of the plurality of genes, identifying the cancer as likely to be responsive to the therapeutic compound, wherein the first number is five or more,
wherein the cancer is a hematopoietic cancer that exhibits a significant gene expression fold change as compared to a reference cell with respect to a first number of a plurality of genes selected from CASP10, TMED1, PPP1CC, TMEM59, BRD7, CYB561, FAM210B, NDRG1, CTSB, MMAB, SETDB2, VPS37B, ELL3, and KIF13B, wherein the first number is at least five.

10. The method of any of claims 7-9, wherein the first number is seven or more, e.g., eight or more, nine or more, or ten or more.

11. The method of any of claims 7-9, wherein the first number is eleven or more, twelve or more, or thirteen or more.

12. A method for treating a hematopoietic cancer in a human individual, comprising determining a gene copy number for KIAA0125 of the hematopoietic cancer; and
if the gene copy number is at least a second number, administering an effective amount of a therapeutic compound of any of formule (I)-(Io) or (IIa)-(IIe) to the human individual, wherein the second number is at least 2 (e.g., at least 4).

13. A method for treating a hematopoietic cancer in a human individual, comprising determining a gene copy number for HLA-B and/or HLA-C of the hematopoietic cancer; and
if the gene copy number is no more than a third number, administering an effective amount of a therapeutic compound of any of formule (I)-(Io) or (IIa)-(IIe) to the human individual, wherein the third number is no more than 0.40 (e.g., no more than 0.10 or even no more than 0.07).

14. A method for treating a cancer in a human individual, the method comprising administering to the human individual an effective amount of a therapeutic compound of any of formulae (I)-(1o) or (IIa)-(IIe), the cancer being a hematopoietic cancer than exhibits a gene copy number for HLA-B and/or HLA-C that is no more than 0.40 (e.g., no more than 0.10 or even no more than 0.07), or is a hematopoietic cancer that exhibits a gene copy number for KIAA0125 that is at least 2 (e.g., at least 4).

15. The method of any of claims 1-14, wherein the hematopoietic cancer is a chronic myeloproliferative neoplasm, a lymphoma, a leukemia, or a plasma cell neoplasm.

16. The method of any of claims 1-14, wherein the hematopoietic cancer is Burkitt's lymphoma, Hodgkin's lymphoma, non-Hodgkin's lymphoma, hairy cell lymphoma, mantle cell lymphoma, T-cell lymphoma, cutaneous T-cell lymphoma, B-cell lymphoma, diffuse large B-cell lymphoma, double-hit lymphoma, Waldenstrom macroglobulinemia, primary central nervous System (CNS) lymphoma, intravascular large B-cell lymphoma (ILBCL), acute lymphoblastic leukemia (ALL), acute myeloid leukemia (AML), acute myeloblastic leukemia, chronic lymphocytic leukemia (CLL), chronic myelogenous leukemia (CML), chronic neutrophilic leukemia (CNL), chronic myelomonocytic leukaemia (CMML), aggressive NK-cell leukemia (acute biphenotypic leukaemia, and polycythemia vera), or acute and chronic T-cell and B-cell leukemia, a multiple myeloma, a chronic myeloproliferative neoplasm, a myelodysplastic syndrome, a myelodysplastic/myeloproliferative neoplasms, or chronic myeloproliferative neoplasms.

17. A method for treating a solid tumor cancer in a human individual, the method comprising administering to the human individual an effective amount of a therapeutic compound of any of formule (I)-(Io) or (IIa)-(IIe), wherein a solid tumor cancer that exhibits a significant gene expression fold change as compared to a reference cell with respect to a first number of a plurality of genes selected from LAMC3, FAM210B, SENP8, ITGB3BP, NUDT2, HNRNPCL1, C20orf43, FRMD8, and STX16, wherein the first number is at least five.

18. A method for treating a solid tumor cancer in a human individual, comprising:
determining the level of expression of a plurality of genes of the cancer;
determining a gene expression fold change as compared to the level of expression of the plurality of genes in a reference cell; and
if the gene expression fold change is significant with respect to a first number of the plurality of genes, administering an effective amount of a therapeutic compound of any of formule (I)-(Io) or (IIa)-(IIe) to the human individual, the first number being five or more,
wherein the cancer is a solid tumor cancer that exhibits a significant gene expression fold change as compared to a reference cell with respect to a first number of a plurality of genes selected from LAMC3, FAM210B, SENP8, ITGB3BP, NUDT2, HNRNPCL1, C20orf43, FRMD8, and STX16, wherein the first number is at least five.

19. A method for determining whether a solid tumor cancer is responsive to a therapeutic compound of any of formule (I)-(Io) or (IIa)-(IIe), the method comprising:
determining the level of expression of a plurality of genes of the cancer;
determining a gene expression fold change as compared to the level of expression of the one or more genes in a reference cell; and
if the gene expression fold change is significant with respect to a first number of the plurality of genes, identifying the cancer as likely to be responsive to the therapeutic compound, wherein the first number is five or more,
wherein the cancer is a solid tumor cancer that exhibits a significant gene expression fold change as compared to a reference cell with respect to a first number of a plurality of genes selected from LAMC3, FAM210B, SENP8, ITGB3BP, NUDT2, HNRNPCL1, C20orf43, FRMD8, and STX16, wherein the first number is at least five.

20. The method of any of claims 17-19, wherein the first number is five or more, e.g., six or more.

21. The method of any of claims 17-19, wherein the first number is seven or more, e.g., eight or more.

22. The method of any of claims 17-21, wherein the solid tumor cancer is adrenal gland(s) cancer, bile duct cancer, a bone or muscle cancer, cervical cancer, colorectal cancer, esophageal cancer, eye cancer, a head or neck cancer (e.g. a cancer of the nose, of the tongue, of the thyroid, or of a submaxillary gland), a kidney cancer, liver cancer, large intestine cancer, small cell lung cancer or non-small cell lung cancer, nervous system cancer, ovarian cancer, pancreatic cancer, placental cancer, prostate cancer, skin cancer, small intestine cancer, stomach/gastric cancer, or uterine cancer.

23. The method of any of claims 1-11 and 15-22, wherein a gene expression fold change of at least 1.5 is a significant change in gene expression.

24. The method of any of claims 1-11 and 15-22, wherein a gene expression fold change of at least 2 is a significant change in gene expression.

25. The method of any of claims 1-11 and 15-22, wherein a gene expression fold change of at least 3 is a significant change in gene expression.

26. The method of any of claims 1-11 and 15-25, wherein the reference cell is a non-cancerous cell of the human individual (e.g., of the same type as the cancer), a non-cancerous cell of a different human (e.g., of the same type as the cancer), a non-cancerous cell from a cell line (e.g., of the same type as the cancer), or a cell from a cancer cell line having an IC₅₀ of at least 30 μM for the therapeutic compound (e.g., of the same type as the cancer).

27. The method of any of claims 1-26, wherein the therapeutic compound has the structural formula 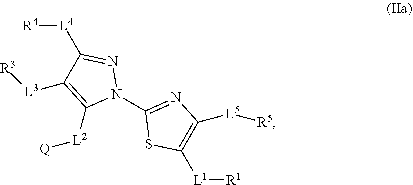
wherein
L¹ is a —S—, —O—, —S(O)—, —S(O)₂— or a bond;
R¹ is unsubstituted or fluorinated C₁-C₈ alkyl, unsubstituted or fluorinated C₁-C₈ alkenyl, unsubstituted or fluorinated C₁-C₈ alkynyl, or phenyl substituted with 1-5 R^{1E},
in which
each R^{1E} is independently selected from oxo, optionally-substituted C₁-C₄ alkyl, C₁-C₄ fluoroalkyl, halogen, —CN, SF₅, —N₃, —C(O)R^{1F}, —SR^{1F}, —S(O)₁₋₂R^{1F}, —OR^{1F}, —(OCH₂CH₂O)ₙ—R^{1G} in which n is 1-4, —N(R^{1G})C(O)CH₂—O—(CH₂CH₂O)ₙR^{1G} in which n is 0-3, —C(O)NR^{1G}(CH₂CH₂O)ₙR^{1G}, —NR^{1G}R^{1F} and —C(O)R^{1F};
each R^{1F} is independently selected from H, C₁-C₃ alkyl and C₁-C₃ fluoroalkyl and
each R^{1G} is independently selected from H and C₁-C₃ alkyl;
L² is a bond or —CH₂—;
Q is —COOH;
L³ is a bond, —C(O)—, —S—, —S(O)₁₋₂—, —O—, —NR⁶—, —CH₂—, —CH(CH₃)(OH)— or —CH(OH)—;
R³ is phenyl or monocyclic heteroaryl each (i) optionally substituted with a single substituent selected from -L^{3C}-(phenyl optionally substituted with 1-5 R^{3D}), -L^{3C}-(monocyclic heteroaryl optionally substituted with 1-5 R^{3D}), -L^{3C}-(monocyclic C3-C6 cycloalkyl optionally substituted with 1-5 R^{3E}), -L^{3C}-(monocyclic C4-C6 heterocycloalkyl optionally substituted with 1-5 R^{3E}) and (ii) optionally substituted with 1-5 R^{3E},
in which
each L^{3C} is a bond, methylene,
ethylene, —C(O)—, —S—, —S(O)₁₋₂—, —O— or —NR^{3G}—;
each R^{3D} is independently selected from optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F} and —NR^{3G}S(O)₁₋₂R^{3F};
each R^{3E} is independently selected from oxo, optionally-substituted C₁-C₄ alkyl, C₁-C₄ fluoroalkyl, halogen, —CN, SF₅, —N₃, —C(O)R^{3F}, —SR^{3F}, —S(O)₁₋₂R^{3F}, —OR^{3F}, —NR^{3G}R^{3F}, —C(O)R^{3F}, —C(O)NR^{3G}R^{3F}, —NR^{3G}C(O)R^{3F}, —C(S)NR^{3G}R^{3F}, —NR^{3G}C(S)R^{3F}, —C(O)OR^{3F}, —OC(O)R^{3F}, —C(O)SR^{3F}, —SC(O)R^{3F}, —C(S)OR^{3F}, —OC(S)R^{3F}, —C(S)SR^{3F}, —SC(S)R^{3F}, —S(O)₁₋₂OR^{3F}, —OS(O)₁₋₂R^{3F}, —S(O)₁₋₂NR^{3G}R^{3F}, —NR^{3G}S(O)₁₋₂R^{3F};
each R^{3F} is independently selected from H, C₁-C₃ alkyl and C₁-C₃ fluoroalkyl and
each R^{3G} is independently selected from H and C₁-C₃ alkyl, C₁-C₃ fluoroalkyl;
L⁴ is is selected from the group consisting of a bond, —C(O)—, —S—, —S(O)₁₋₂—, —O— and —NR⁶—;
R⁴ is selected from the group consisting of unsubstituted, hydroxylated, C₁-C₄ alkoxylated or fluorinated C₁-C₈ alkyl, unsubstituted or fluorinated C₁-C₈ alkenyl and unsubstituted or fluorinated C₁-C₈ alkynyl;
L⁵ is a bond, —C(O)—, —S—, —S(O)₁₋₂—, —O— or —NR⁶—;
R⁵ is phenyl, monocyclic heteroaryl, monocyclic heterocycloalkyl or monocyclic cycloalkyl each optionally substituted with 1-5 R^{5E},
in which
each R^{5E} is independently selected from oxo, optionally-substituted C₁-C₄ alkyl, C₁-C₄ fluoroalkyl, halogen, —CN, —SF₅, —N₃, —C(O)R^{5F}, —SR^{5F}, —S(O)₁₋₂R^{5F}, —OR^{5F}, —NR^{5G}R^{5F}, —C(O)R^{5F}, —C(O)NR^{5G}R^{5F}, —NR^{5G}C(O)R^{5F}, —C(S)NR^{5G}R^{5F}, —NR^{1G}C(S)R^{5F}, —C(O)OR^{5F}, —OC(O)R^{5F}, —C(O)SR^{5F}, —SC(O)R^{5F}, —C(S)OR^{5F}, —OC(S)R^{5F}, —C(S)SR^{5F}, —SC(S)R^{5F}, —S(O)₁₋₂OR^{5F}, —OS(O)₁₋₂R^{5F}, —S(O)₁₋₂NR^{5G}R^{5F} and —NR^{5G}S(O)₁₋₂R^{5F};
each R^{5F} is independently selected from H, C₁-C₃ alkyl and C₁-C₃ fluoroalkyl and
each R^{5G} is independently selected from H and C₁-C₃ alkyl;
wherein
each R⁶ is selected from the group consisting of hydrogen, C₁-C₃ alkyl and —C(O)(C₁-C₃ alkyl);
each optionally substituted alkyl, alkenyl and alkynyl is unsubstituted, fluorinated or substituted with one or two hydroxyl groups;
each cycloalkyl has 3-7 ring carbons and is unsaturated or partially unsaturated;
each heterocylcloalkyl has 3-7 ring members and 1-3 heteroatoms independently selected from nitrogen, oxygen and sulfur and is unsaturated or partially unsaturated;
each heteroaryl is a 5-6 membered monocyclic heteroaryl ring having 1-4 heteroatoms independently selected from nitrogen, oxygen and sulfur or a 8-10 membered bicyclic heteroaryl having 1-5 heteroatoms independently selected from nitrogen, oxygen or sulfur.

28. The method according to any of claims 1-26, wherein the therapeutic compound is Compound A197, Compound B5, Compound B19, or Compound B20.
